(19) United States
(12) Patent Application Publication
(10) Pub. No.: US 2021/0293693 A1
(43) Pub. Date: Sep. 23, 2021

(54) METHODS AND DEVICES FOR DETECTING AND SORTING DROPLETS OR PARTICLES

(71) Applicant: 10X Genomics, Inc., Pleasanton, CA (US)

(72) Inventors: Rajiv BHARADWAJ, Pleasanton, CA (US); Jianying CAO, San Jose, CA (US); Mohammad Rahimi LENJI, Livermore, CA (US); Anthony MAKAREWICZ, Livermore, CA (US); Alireza SALMANZADEH, San Ramon, CA (US)

(21) Appl. No.: 17/343,281

(22) Filed: Jun. 9, 2021

Related U.S. Application Data
(63) Continuation of application No. PCT/US2019/065735, filed on Dec. 11, 2019.
(60) Provisional application No. 62/932,761, filed on Nov. 8, 2019.
(60) Provisional application No. 62/889,178, filed on Aug. 20, 2019.
(60) Provisional application No. 62/876,362, filed on Jul. 19, 2019.
(60) Provisional application No. 62/839,287, filed on Apr. 26, 2019.
(60) Provisional application No. 62/812,102, filed on Feb. 28, 2019.
(60) Provisional application No. 62/811,895, filed on Feb. 28, 2019.
(60) Provisional application No. 62/812,018, filed on Feb. 28, 2019.
(60) Provisional application No. 62/795,184, filed on Jan. 22, 2019.
(60) Provisional application No. 62/778,058, filed on Dec. 11, 2018.

(51) Int. Cl. G01N 15/14; B01L 3/00

(52) U.S. Cl. CPC G01N 15/1484; B01L 3/502715; B01L 3/502761; G01N 2015/1006; G01N 15/147; B01L 2200/0652; B01L 2300/0654; G01N 15/1434

(57) ABSTRACT

Devices, systems, and their methods of use, for detecting and/or sorting droplets or particles are provided. One or more sorters can be selected to sort droplets or particles of a desired and predictable property or droplet size. The invention involves detection of droplets or particles from a droplet or particle source through a detection region.

### BACKGROUND OF THE INVENTION

Many biomedical applications rely on high-throughput assays of samples combined with one or more reagents in droplets or particles. For example, in both research and clinical applications, high-throughput genetic tests using target-specific reagents are able to provide information about samples in drug discovery, biomarker discovery, and clinical diagnostics, among others. Many of these applications, following the formation of a droplet or particle, rely on the presence of a reagent or material within the droplet or particle.

Efficient development of new droplet or particle formation technologies and improvement of existing technologies relies on the ability to detect and characterize droplet or particle quality. In general, current devices, systems, and methods for droplet or particle formation are poorly equipped to detect droplets or particles, such as when as they are being formed.

Accordingly, devices and methods for detecting or sorting droplets or particles would be beneficial.

### SUMMARY OF THE INVENTION

The invention provides devices, systems, and methods for sorting droplets or particles. The invention also provides devices, systems, and methods for detecting droplets or particles. In some embodiments, the invention provides devices, systems, and methods for detecting and sorting droplets or particles.

In one aspect, the invention features a device for sorting droplets or particles including a droplet or particle source, configured to provide droplets or particles within a continuous phase; a sorting region, in fluid communication with the droplet or particle source, including a sorter configured to sort one or more of the droplets or particles; and a collection region, in fluid communication with the sorting region, including two or more partitions configured for collection of the droplets or particles after sorting.

In some embodiments, the droplet or particle source includes a droplet or particle formation region.

In some embodiments, the sorter employs mechanical or electromagnetic force to sort the one or more droplets or particles.

In some embodiments, the sorter includes a dielectrophoretic actuator, an acoustic actuator, a fluidic (e.g., pneumatic) actuator, a mechanical actuator, a bubble generator, an optical tweezer, a magnet, a thermal actuator, or an electrostatic charger.

In some embodiments, the sorter includes a divider.

In some embodiments, the dielectrophoretic actuator or the electrostatic charger includes an electrode. The electrode may include a fluid channel or a solid conductor.

In some embodiments, the sorting region includes a surface configured to laterally deflect one or more of the droplets or particles to one of the partitions.

In some embodiments, the device includes a detection region to allow detection of the droplets or particles as they pass therethrough. The detection region may be configured to provide feedback to the sorting region, e.g., to actuate the sorter.

In some embodiments, the collection region includes 3, 4, 5, 6, 7, 8 9, 10, or more partitions.

In some embodiments, each cross-sectional dimension of the sorting region has a length of at least 1 mm (e.g., 2 mm, 3 mm, 4 mm, 5 mm, 6 mm, 7 mm, 8 mm, 9 mm, 10 mm, or more). In some embodiments, the sorting region has a volume of at least 1 μL, 100 μL, 500 μL, or 1 mL.

In some embodiments, the droplet includes a particle. The particle may include a biological particle, a bead, or a combination thereof. The biological particle may include a cell or one or more constituents of a cell. The biological particle may include a matrix.

In another aspect, the invention features a method of sorting droplets or particles by providing a device as described herein including a continuous phase in the sorting region; allowing droplets or particles to enter the sorting region; and sorting droplets or particles into one of the partitions in the collection region (e.g., by actuating the sorter).

In some embodiments, the method further includes detecting the droplets or particles prior to sorting. The detection may provide feedback to the sorter, e.g., to actuate the sorter to sort the droplets or particles.

In one aspect, the invention features a device for sorting droplets or particles including a droplet or particle source configured to provide droplets or particles within a continuous phase; a first channel having a first depth, a first width, a first proximal end, and a first distal end, wherein the first channel is in fluid communication with the droplet or particle source; and a barrier configured to operatively connect one or more electrodes with the first channel.

In some embodiments, the device may further include a collection region. The collection region may include two or more (e.g., 3, 4, 5, 6, 7, 8, 9, 10, or more) partitions configured for collection of the droplets or particles, wherein the collection region is in fluid communication with the first channel.

In some embodiments, the droplet or particle source includes a droplet formation region configured to form droplets from a first liquid in a second liquid.

In some embodiments, the device may further include a second channel having a second depth, a second width, a second proximal end, and a second distal end, and a third channel having a third depth, a third width, a third proximal end, and a third distal end, wherein the third channel intersects the second channel between the second proximal and second distal ends, wherein the second distal end is in fluid communication with the first proximal end, and wherein the second channel and the droplet formation region are configured to produce droplets of the first liquid in the second liquid.

In some embodiments, the droplet formation region includes a shelf region and a step region. The shelf region may have a fourth depth and a fourth width, and the step region may have a fifth depth. The shelf region is configured to allow the first liquid to expand in at least one dimension and has at least one inlet and at least one outlet, and the shelf region is disposed between the second distal end and the step region.

In some embodiments, the barrier has a thickness of at least 1 μm (e.g., from about 1 μm to about 100 μm, e.g., from about 5 μm to about 50 μm).

In some embodiments, the droplet includes a particle. The particle may include a biological particle, a bead, or a combination thereof. The biological particle may include a cell or one or more constituents of a cell. The biological particle may include a matrix.

In some embodiments, the barrier is configured to operatively connect 2, 3, 4, 5, 6, 7, 8, 9, 10, or more electrodes with the first channel.

In some embodiments, the device includes one or more (e.g., 2, 3, 4, 5, 6, 7, 8, 9, 10, or more) cavities configured to receive the one or more electrodes and position the one or more electrodes adjacent to the barrier.

In another aspect, the invention features a system including the device as described herein, the one or more electrodes, and a housing configured to position the one or more electrodes in operative connection with the first channel.

In some embodiments, at least one of the one or more electrodes is a dielectrophoretic electrode.

In some embodiments, at least one of the one or more electrodes includes a fluid channel or a solid conductor.

In some embodiments, the system may further include a conductive fluid (e.g., a gel) disposed between at least one of the one or more electrodes and the barrier.

In another aspect, the invention features a method of sorting droplets or particles by providing a system as described herein including the continuous phase in the first channel and the barrier operatively connecting the one or more electrodes with the first channel; allowing the droplets or particles to enter the first channel; and sorting one or more of the droplets or particles into one or more of the two or more partitions in the collection region by applying an electromagnetic field to the droplets or particles via the one or more electrodes.

In one aspect, the invention features a device for detecting a droplet (e.g., by imaging). The device includes a droplet source, a deflecting surface, a detection region, and a collection region. The droplet source is configured to provide droplets within a continuous phase, e.g., after they are formed by a droplet forming device such as any of those described herein or known in the art. The deflecting surface is in fluid communication with the droplet source and configured to laterally deflect the droplet. The detection region is configured to allow detection of the droplet as it passes therethrough, and the collection region is configured for collection of the droplet after detection. In some embodiments, the device includes one or more openings (e.g., one, two, three, four, or more openings) configured to shunt the continuous phase to a continuous phase reservoir. For example, the one or more openings can be on the deflecting surface or elsewhere on the device (e.g., at a point configured to be positioned below the surface of the continuous phase to allow the continuous phase to pass therethrough, e.g., while restricting the droplets to the collection region).

In some embodiments, the deflecting surface is configured to deflect droplets having a density lower than the continuous phase. For example, the droplets can be deflected as they float to the top of the continuous phase. In other embodiments, the droplets have a density higher than the continuous phase and are deflected as they sink to the bottom of the continuous phase.

In some embodiments, the deflecting surface includes (e.g., is wholly or partially made from) a transparent material. In some embodiments, the deflecting surface can have a refractive index almost the same as the refractive index of the continuous phase. For example, the refractive index can be within 10%, within 9%, within 8%, within 7%, within 6%, within 5%, within 4%, within 3%, within 2%, within 1%, within 0.1%, or within 0.01% of the refractive index of the continuous phase. In some embodiments, the refractive index of the deflecting surface can be from 1.3 to 1.6 (e.g., from 1.4 to 1.55 or from 1.45 to 1.50, e.g., from 1.3 to 1.35, from 1.35 to 1.40, from 1.40 to 1.45, from 1.45 to 1.50, from 1.50 to 1.55, or from 1.55 to 1.60, e.g., about 1.30, about 1.31, about 1.32, about 1.33, about 1.333, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, or about 1.60). Alternatively, the deflecting surface can be black or otherwise opaque (e.g., for fluorescent droplet detection).

The deflecting surface can be at an angle, can have multiple angles (e.g., two, three, four, five, six, or more angles), or be curved. For example, in embodiments in which the droplets float in the continuous phase, the deflecting surface can have an angle (e.g., an angle β₀, or an angle of 90°-α) between 0° and 80° above a horizontal plane (e.g., from 10° to 70°, from 15° to 60°, from 20° to 50°, from 25° to 45°, or from 30° to 40° above a horizontal plane, e.g., from 10° to 15°, from 15° to 20°, from 20° to 25°, from 25° to 30°, from 30° to 35°, from 35° to 40°, from 40° to 45°, from 45° to 50°, from 50° to 55°, from 55° to 60°, from 60° to 65°, from 65° to 70°, from 70° to 75°, or from 75° to 80° above a horizontal plane, e.g., less than 1°, about 1°, about 2°, about 3°, about 4°, about 5°, about 6°, about 7°, about 8°, about 9°, about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 31°, about 32°, about 33°, about 34°, about 35°, about 36°, about 37°, about 38°, about 39°, about 40°, about 41°, about 42°, about 43°, about 44°, about 45°, about 46°, about 47°, about 48°, about 49°, about 50°, about 51°, about 52°, about 53°, about 54°, about 55°, about 56°, about 57°, about 58°, about 59°, about 60°, about 61°, about 62°, about 63°, about 64°, about 65°, about 66°, about 67°, about 68°, about 69°, about 70°, about 71°, about 72°, about 73°, about 74°, about 75°, about 76°, about 77°, about 78°, about 79°, or about 80° above a horizontal plane). In embodiments in which the droplets sink in the continuous phase, the deflecting surface can have an angle (e.g., an angle β₀, or an angle of 90°-α) from 0° to 80° below a horizontal plane (e.g., from 10° to 70°, from 15° to 60°, from 20° to 50°, from 25° to 45°, or from 30° to 40° below a horizontal plane, e.g., from 10° to 15°, from 15° to 20°, from 20° to 25°, from 25° to 30°, from 30° to 35°, from 35° to 40°, from 40° to 45°, from 45° to 50°, from 50° to 55°, from 55° to 60°, from 60° to 65°, from 65° to 70°, from 70° to 75°, or from 75° to 80° below a horizontal plane, e.g., less than 1°, about 1°, about 2°, about 3°, about 4°, about 5°, about 6°, about 7°, about 8°, about 9°, about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 31°, about 32°, about 33°, about 34°, about 35°, about 36°, about 37°, about 38°, about 39°, about 40°, about 41°, about 42°, about 43°, about 44°, about 45°, about 46°, about 47°, about 48°, about 49°, about 50°, about 51°, about 52°, about 53°, about 54°, about 55°, about 56°, about 57°, about 58°, about 59°, about 60°, about 61°, about 62°, about 63°, about 64°, about 65°, about 66°, about 67°, about 68°, about 69°, about 70°, about 71°, about 72°, about 73°, about 74°, about 75°, about 76°, about 77°, about 78°, about 79°, or about 80° below a horizontal plane). The one or more angles can be configured to control the speed and/or position of the droplet to be suitable for detection (e.g., frame-rate of image acquisition). In some embodiments, the deflecting surface is horizontal (e.g., at an angle of 0°).

In some embodiments, the detection region includes a reflector. Additionally, or alternatively, the deflecting surface can include the reflector. The reflector can be configured to reflect light toward a detector, such as in the case of bright-field microscopy, and can be made of any suitable material for reflection. For example, the reflector can be configured to reflect light toward a second reflector that reflects the light toward the detector. In some embodiments, the reflector is about horizontal, e.g., within an otherwise angled deflecting surface. In other embodiments, the reflector is angled as necessary according to known principles and designs.

In some embodiments, the device includes a reservoir (e.g., a well, e.g., a cylindrical well) and an insert. The insert defines one or more boundaries of the collection region. In some embodiments, the reflector is at or above the top of the reservoir. In some embodiments, the insert includes the deflecting surface and defines a boundary of the collection region.

In some embodiments, the insert is configured to occupy a low volume of the reservoir to provide a suitable collection region volume. In some embodiments, the collection region is from 10% to 99% of the lateral area of the reservoir (e.g., from 15% to 98%, from 20%, to 97%, from 25% to 96%, from 30% to 95%, from 35% to 90%, from 40% to 85% from 45% to 80%, or from 50% to 75% of the lateral area of the reservoir, e.g., from 10% to 15%, from 15% to 20%, from 20% to 25%, from 25% to 30%, from 30% to 35%, from 35% to 40%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 99% of the lateral area of the reservoir, e.g., about 40%, about 45%, about 50%, about 55%, about 60%, about 65%, about 70%, about 75%, about 80%, about 85%, about 90%, or about 95% of the lateral area of the reservoir).

In some embodiments, the detection region is optically detectable. For example, the device is configured for optical detection of a droplet as it passes through the detection region, e.g., by objective lens and/or camera, e.g., video camera. In some embodiments, the optical detection includes fluorescent detection. The device can be adapted for additional or alternative detection means, such as electrical and/or magnetic detection.

In another aspect, the invention features a system having a device as described herein. A system of the invention can include one or more devices and reagents (e.g., reagents necessary for droplet formation as described herein). In some embodiments, the reagents include the continuous phase, which, in some embodiments, is contained in the detection region and all or a portion of the collection region. The continuous phase may be in fluid communication with the droplet source and the deflecting surface. In some embodiments, the system includes a plurality of devices that are connected (e.g., as a chip or plate having an array of reservoirs).

In another aspect, the invention features methods of detecting one or more droplets. A method of detecting a droplet includes, for example, providing a device or system as described herein, allowing a droplet to enter the detection region, and detecting the droplet in the detection region. In some embodiments, the device includes a continuous phase in the detection region and all or a portion of the collection region, wherein the continuous phase is in fluid communication with the droplet source and the deflecting surface. In some embodiments, the detection is optical detection (e.g., by imaging or video recording, e.g., by detection of light scattering, fluorescence, etc.). In some embodiments, the droplet is aqueous or miscible with water and immiscible with the continuous phase. For example, the continuous phase can be an oil. Alternatively, the continuous phase can be aqueous or miscible with water and immiscible with the droplet, and the droplet can be an oil.

In one aspect, the invention features a device for detecting a droplet or particle including a droplet or particle source; and a detection region in fluid communication with the droplet or particle source. The detection region includes a wave plate (e.g., a quarter wave plate).

In some embodiments, the device further includes a polarizer configured to polarize light from a light source. The polarizer may be a polarization beam splitter.

In some embodiments, the detection region includes a reservoir.

In some embodiments, the droplet or particle source includes a droplet formation region configured to form droplets of a first liquid in a second liquid.

In some embodiments, the device further includes a reflector configured to reflect light passing through the wave plate.

In another aspect, the invention features a system for detecting a droplet or particle including a device as described herein, a light source, and a light detector.

In some embodiments, the system further includes a polarizer configured to polarize light from the light source in a first polarization and direct it to the detection region. The polarizer may be a polarization beam splitter. In some embodiments, the polarizer is configured to direct light polarized in a second polarization that is orthogonal to the first polarization to the detector.

In another aspect, the invention features a method of detecting a droplet or particle or a characteristic thereof by providing a device or system as described herein; allowing the droplet or particle to enter the detection region; illuminating the droplet or particle in the detection region with light polarized in a first polarization, wherein the light passes through the wave plate; and measuring light that passes through the wave plate, thereby detecting the droplet or particle or the characteristic thereof.

In some embodiments, the light passes through the wave plate and is reflected by a reflector back through the wave plate, thereby rotating the polarization to a second polarization orthogonal to the first polarization.

In some embodiments, light having the second polarization passes through the polarizer to the detector.

In some embodiments, the characteristic is the size of the droplet or particle, the velocity of the droplet or particle, the presence of one or more particles in the droplet, and/or the location of one or more particles in the droplet. In some embodiments, the droplet includes a particle. In some embodiments, the characteristic of the droplet is a characteristic of the particle in the droplet. In some embodiments, the particle includes a biological particle, a bead, or a combination thereof. In some embodiments, the biological particle includes a cell or one or more constituents of a cell. In some embodiments, the characteristic is a type of cell, a composition of the cell, and/or a size of the cell.

In one aspect, the invention features a device for detecting light from a droplet or particle including a droplet or particle source, a detection channel in fluid communication with the droplet or particle source, the detection channel having a proximal end and a distal end, a light source or first light guide, and one or more (e.g., 2, 3, 4, 5, 6, 7, 8, 9, 10, or more) detectors or second light guides. The detection channel, the light source or first light guide, and the one or more detectors or second light guides may be configured to detect light as a droplet or particle passes from the proximal end to the distal end of the detection channel.

In some embodiments, the device further includes a collection region including two or more partitions configured for collection of the droplets or particles. The collection region may be in fluid communication with the first channel.

In some embodiments, the collection region includes 3 or more (e.g., 4, 5, 6, 7, 8, 9, 10, or more) partitions.

In some embodiments, the droplet or particle source includes a droplet formation region.

In some embodiments, the one or more detectors or second light guides are placed on one side of the detection channel or multiple sides of the detection channel.

In some embodiments, the device further includes a controller operatively coupled to direct the transport of the droplet or particle through the detection channel.

In some embodiments, the device further includes a sorting region. The sorting region may be configured to sort the droplets or particles, e.g., after detection.

In another aspect, the invention features a system for detecting light from a droplet or particle. The system includes a device for detecting light and a fluidic device. The device for detecting light includes a light source or first light guide and/or one or more detectors or second light guides. The fluidic device may include a droplet or particle source and a detection channel having a proximal end and a distal end in fluid communication with the droplet or particle source. The fluidic device may mate with the device for detecting light, such that the light source or first light guide and the one or more detectors or second light guides are configured for the detection of light as a droplet or particle passes from the proximal end to the distal end of the detection channel. The system, as a whole, includes the light source and the one or more detectors.

In certain embodiments, the device for detecting light includes the light source and/or the one or more detectors. In these embodiments, the light source and/or one or more detectors may mate with the fluidic device in the absence of first or second light guides. In other embodiments, the fluidic device includes a first and/or one or more second light guides that mate with the light source and/or one or more detectors. Combinations of these embodiments are also included, e.g., where the device for detecting light includes the light source and the fluidic device includes the one or more detectors or vice versa.

In another aspect, the invention features a method of detecting a droplet or particle or a characteristic thereof, by providing a device or system of the invention, illuminating the droplet or particle as it passes along the detection channel from the proximal end to the distal end, and measuring light as the droplet or particle passes through the detection channel, thereby detecting the droplet or particle or the characteristic thereof.

In some embodiments, the characteristic is the size of the droplet or particle, the velocity of the droplet or particle, the presence of one or more particles in the droplet, and/or the location of one or more particles in the droplet.

In some embodiments, the droplet includes one or more (e.g., 4, 5, 6, 7, 8, 9, 10, or more) particles. The one or more particles may include a cell, a bead, or a combination thereof.

In some embodiments, the characteristic of the droplet is a characteristic of the particle (e.g., cell) in the droplet.

In some embodiments, the characteristic is the type of cell, the composition of the cell, and/or the size of the cell.

In one aspect, the invention provides a device including a droplet or particle source (e.g., a droplet formation region) and a channel. The channel has a slanted portion, and the droplet or particle source provides droplets or particles that flow along the slanted portion. The slanted portion is aligned with a detector or lens configured for detection of the droplets or particles as the droplets or particles pass through a focal plane in the slanted portion. The slanted portion may be at an angle relative to the focal plane (e.g., from 1° to 80° to the focal plane, from 5° to 90° to the focal plane, or from 20° to 40° to the focal plane, e.g., from 1° to 10°, 10° to 20°, 20° to 30°, 30° to 40°, 40° to 50°, 50° to 60°, 60° to 70°, 70° to 80°, or 80° to 90° to the focal plane). In some embodiments, the focal plane is horizontal. The device can include a channel having a first portion running parallel to the focal plane, wherein the first portion is disposed between the droplet or particle source and the slanted portion of the channel.

In another aspect, the invention provides a system having the device with a channel having a slanted portion and a detector or lens, wherein the detector or lens is disposed for detection of the slanted portion of the channel.

In another aspect, the invention provides methods for detecting a droplet or particle (or a plurality of droplets or particles). The method includes providing a device as described herein and a detector disposed to detect the slanted portion of the channel or a system as described herein. The method further includes passing a droplet or particle to the slanted portion of the channel and detecting multiple cross sections of the droplet or particle as the droplet or particle flows along the slanted portion of the channel.

In a further aspect, the invention provides a device having a droplet or particle source (e.g., a droplet or particle formation region), a channel, and a plurality of lenses (e.g., micro-lenses) or detectors. Each of the plurality of lenses or detectors has a different focal plane, and the channel is aligned with the plurality of lenses or detectors to position the different focal planes for detection of multiple planes of a droplet or particle as it flows along the channel. In some embodiments, the lenses or detectors have different focal distances (and may be positioned at the same or different distances relative to the channel).

In yet another aspect, the invention provides a system having a device as described herein and a plurality of lenses or detectors having different focal planes, wherein the plurality of lenses or detectors is disposed to detect droplets or particles at the different focal planes.

In another aspect, the invention provides a method of detecting a droplet or particle by providing a device of the invention (e.g., a device having a droplet or particle source (e.g., a droplet or particle formation region), a channel, and a plurality of lenses (e.g., micro-lenses)) and one or more detectors or the equivalent system. The method includes passing a droplet or particle from the droplet or particle source to the channel, and using the one or more detectors to detect the droplet or particle at the different focal planes as the droplet or particle flows along the channel.

In a further aspect, the invention provides a system having a device including a droplet or particle source (e.g., a droplet or particle formation region) and a channel, wherein the channel is aligned with a detector or lens configured for detection of droplets or particles at a plurality of focal planes as the droplets or particles flow along the channel. The detector or lens can be moved (e.g., oscillated) acoustically or piezoelectrically. In a related aspect, the invention features a method of detecting a droplet or particle using this system. A droplet or particle can be passed from the droplet or particle source (e.g., the droplet or particle formation region) to the channel, where it is detected at the plurality of focal planes using the detector.

In another aspect, the invention provides a method of detecting a droplet or particle by providing a device having a droplet or particle source (e.g., the droplet or particle formation region) and a channel aligned with one or more detectors. The method further includes passing a droplet or particle in the droplet or particle source to the channel and detecting the droplet or particle with the one or more detectors at a plurality of focal planes. Multiple planes of the droplet or particle are thereby detected.

In some embodiments, any method of detecting droplets or particles may further include determining a number of particles (e.g., a number of beads or biological particles) in a droplet. In some embodiments, the droplet includes one or more particles, one of which has a dimension that is less than 50% of the corresponding dimension of the droplet. For example, the droplet may include a bead selected from the group consisting of a solid bead, a semi-solid bead, a gel bead, a polymeric bead, or a magnetic bead. Additionally or alternatively, the droplet may include a biological particle, such as a cell (e.g., a prokaryotic cell, eukaryotic cell, a healthy cell, or a diseased or disease-associated cell (such as a cancer cell)), a constituent of a cell (such as an organelle (e.g., a nucleus)), or a virus. In some embodiments, the droplet includes a cell and a bead.

In another aspect, the invention provides a device having a droplet or particle source (e.g., a droplet or particle formation region) and a channel aligned with a focal plane of a lens or detector. A portion of the channel is constricted to position droplets or particles in the focal plane of the detector or lens for detection of the droplets or particles as the droplets or particles flow along the channel. In some embodiments, a depth of the channel is reduced in the constricted portion of the channel.

In a further aspect, the invention provides a system that includes a device having a droplet or particle source and a channel and a detector or lens, wherein a portion of the channel is constricted to position droplets or particles in the focal plane of the detector or lens for detection.

In yet another aspect, the invention features a method of detecting a droplet or particle by providing a device having a constricted portion of the channel and aligned with a focal plane of a detector or lens or the equivalent system; passing the droplet or particle to the constricted portion of the channel; and detecting the droplet or particle at the focal plane as it flows along the constricted portion of the channel. In some embodiments, the droplet or particle source includes a droplet or particle formation region. In some embodiments, the droplet includes one or more particles (e.g., one or more beads and/or biological particles). For example, the particles can be one or more beads, e.g., solid beads, semi-solid beads, gel beads, polymeric beads, and magnetic beads. In embodiments in which droplets contain one or more biological particles, such as a cell (e.g., a prokaryotic cell, eukaryotic cell, a healthy cell, or a diseased or disease-associated cell (such as a cancer cell)), a constituent of a cell (such as an organelle (e.g., a nucleus)), or a virus. In some embodiments, the droplet includes a cell and a bead.

In one aspect, the invention provides a system for detecting light simultaneously from a plurality of detection points in a microfluidic device, wherein the system includes: (a) a plurality of light guides with a receiving end and a transmitting end, wherein the receiving end of each of the plurality of light guides is configured to receive light from one of the plurality of detection points; and (b) a detector with an array sensor (e.g., a charge-coupled device (CCD) or a complementary metal oxide semiconductor (CMOS) sensor) having a plurality of regions, wherein each region is configured to receive light transmitted from one of the transmitting ends of the plurality of light guides. The regions may be spatially distinct to allow simultaneous detection of each region without overlapping signal.

In some embodiments, the system further includes one or more light sources (e.g., laser) to illuminate the plurality of detection points in the microfluidic device. In some embodiments, the plurality of light guides includes wave guides. In some embodiments, the plurality of light guides includes a plurality of lenses (e.g., micro-lenses). In some embodiments, the plurality of light guides includes multi-mode optical fibers. In some embodiments, the system is programmed to produce a single digital image or a video from light transmitted by the plurality of light guides to the array sensor. Additionally or alternatively, the system of the first aspect further includes an emission filter disposed between the array sensor and the plurality of detection points. In some embodiments of the first aspect, the detector records 2-2000 frames per second (e.g., 5-1500, 10-1000, 20-800, 40-500, or 60-200 frames per second, e.g., 2-5, 5-10, 10-15, 15-20, 20-25, 25-30, 30-35, 35-40, 40-45, 45-50, 50-60, 60-70, 70-80, 80-90, 90-100, 100-110, 110-120, 120-130, 130-140, 140-150, 150-160, 160-170, 170-180, 180-190, 200-250, 250-300, 300-350, 350-400, 400-450, 450-500, 500-600, 600-700, 700-800, 800-900, 900-1000, 1000-1500, or 1500-2000 frames per second, e.g., 2, 3, 4, 5, 6, 7, 8, 9, 10, 15, 20, 25, 30, 35, 40, 45, 50, 60, 70, 80, 90, 100, 200, 250, 300, 400, 500, 600, 700, 800, 900, 1000, 1100, 1200, 1300, 1400, 1500, 1600, 1700, 1800, 1900, or 2000 frames per second).

In some embodiments, the system further includes a holder for the microfluidic device. In some embodiments, the system further includes the microfluidic device.

In some embodiments, the microfluidic device includes a plurality of droplet or particle sources, and the plurality of detection points are disposed to detect droplets or particles provided, e.g., formed by the plurality of droplet or particle sources, e.g., with at least one point of detection positioned to detect droplets or particles from each of the droplet or particle sources.

In an aspect, the invention features a method for detecting light simultaneously at a plurality of detection points in a microfluidic device by: (a) providing a system of the invention; and (b) simultaneously detecting light from the plurality of detection points using the array sensor.

In an aspect, the invention features a method for simultaneously detecting a plurality of droplets or particles in a microfluidic device by: (a) providing a system of the invention, wherein the system includes the microfluidic device with a plurality of droplet or particle sources; (b) passing a liquid containing the plurality of droplets or particles through the plurality of detection points; and (c) simultaneously detecting light from the plurality of detection points using the array sensor, thereby simultaneously detecting the plurality of droplets or particles. In some embodiments, step (c) includes producing a digital image or a video of light detected from the plurality of detection points, e.g., a single digital image with each region spatially distinct to allow simultaneous detection of each region without overlapping signal.

In an aspect, the invention provides a system for detecting light simultaneously from a plurality of channels (e.g., at least 2, 4, or 8 channels) in a microfluidic device, wherein the system includes a linear detector (e.g., a line scan camera) aligned with the plurality of channels to detect light from the plurality of channels simultaneously. The channels are disposed to allow detection without overlapping signals in the linear array.

In some embodiments, the linear detector includes a CCD or a CMOS sensor. In some embodiments, each of the plurality of channels is positioned at a distance of 50 μm to 2.0 cm from another channel. In some embodiments, the linear detector includes at least 12,000 (e.g., at least 12,000 pixels, 13,000 pixels, 14,000 pixels, 15,000 pixels, 16,000 pixels, 17,000 pixels, 18,000 pixels, 19,000 pixels, 20,000 pixels, 21,000 pixels, 22,000 pixels, 23,000 pixels, 24,000 pixels, 25,000 pixels, 26,000 pixels, 27,000 pixels, 28,000 pixels, 29,000 pixels, or 30,000 pixels, or more) pixels.

In some embodiments, the system further includes one or more light sources (e.g., laser) disposed to illuminate the plurality of channels in the microfluidic device.

In some embodiments, the system further includes a holder for the microfluidic device. In some embodiments, the system further includes the microfluidic device, such as a microfluidic device with a plurality of droplet or particle sources. In some embodiments, the plurality of channels is disposed to allow detection of droplets or particles provided by the plurality of droplet sources.

In an aspect, the invention features a method for detecting light simultaneously from a plurality of channels in a microfluidic device by: (a) providing a system of the invention; and (b) simultaneously detecting light from the plurality of channels using the linear detector.

In an aspect, the invention features a method for simultaneously detecting a plurality of droplets or particles in a microfluidic device by: (a) providing a system of the invention that includes a microfluidic device with a plurality of droplet or particle sources; (b) passing a liquid containing the plurality of droplets or particles through the plurality of channels; and (c) simultaneously detecting light from the plurality of channels using the linear detector, thereby simultaneously detecting the plurality of droplets.

In some embodiments of any method of the invention, the light detected is indicative of presence or absence of droplets or particles (e.g., droplets with one or more particles). In some embodiments, particles include biological particles (e.g., a cell, such as a cell with a fluorescent label) and/or beads. In some embodiments, the light detected is indicative of a number of particles in a droplet. In some embodiments, the plurality of droplets is aqueous or miscible with water and immiscible with the continuous phase. Some embodiments of these methods of the invention further include sorting the plurality of droplets or particles.

In one aspect, the invention provides is a system for detecting light in a microfluidic device. The system includes a stage, a light source (e.g., a laser), an aspherical lens pair, and a detector. The stage includes a location for placement of the microfluidic device. The light source (e.g., laser) is disposed to transmit a beam of light (e.g., a laser beam) to a detection point within the location and on a plane parallel to the stage. The angle α of the beam to the plane is from 25° to 40° to the stage (e.g., angle α can be equal to 90° minus Brewster's angle). The aspherical lens pair has an input focal point and an output focal point, wherein the aspherical lens pair is disposed to co-localize the input focal point with the detection point, and the detector is configured to detect the light transmitted from the aspherical lens. In some embodiments, the beam intersects the surface of the microfluidic device at angle α. In some embodiments, the system further includes the microfluidic device.

In another aspect, the invention provides a system for detecting light in a microfluidic device, wherein the system includes a microfluidic device having a planar surface, a light source (e.g., a laser), an aspherical lens pair, and a detector configured to detect light from the aspherical lens. The light source (e.g., laser) is disposed to transmit a beam of light (e.g., a laser beam) to a detection point within the microfluidic device, wherein the angle α of the beam to the planar surface is from 25° to 40° (e.g., angle α can be equal to 90° minus Brewster's angle). The aspherical lens pair has an input focal point and an output focal point and is disposed to co-localize the input focal point with the detection point.

In either of the preceding aspects, the system may further include an optical fiber (e.g., a multi-mode optical fiber) having a receiving end and a transmitting end, wherein the receiving end is disposed to co-localize with the output focal point of the aspherical lens pair, and wherein the detector detects the light transmitted from the transmitting end of the optical fiber. In some embodiments, the system further includes a stage having a location for placement of the microfluidic device. In some embodiments, the stage is an X-Y-Z stage. In some embodiments, the light source is disposed above the detection point, and angle α is above the stage. In some embodiments, angle α is from 30° to 35° (e.g., about 30°, about 31°, about 32°, about 33°, about 34°, or about 35°).

In some embodiments, the system includes a dichroic mirror between the aspherical lens pair and the output focal point. In some embodiments, the system includes a reflected light detector configured to detect the reflected light from the dichroic mirror.

In some embodiments, the system further includes a polarizer (e.g., a polarizer disposed between the light source (e.g., laser) and the microfluidic device and/or stage). In some embodiments, the system includes an emission filter (e.g., disposed between the microfluidic device and the detector, or as part of the detector).

In some embodiments, the optical detector is a camera. In some embodiments, the camera records 2-2000 frames per second (e.g., 5-1500, 10-1000, 20-800, 40-500, or 60-200 frames per second, e.g., 2-5, 5-10, 10-15, 15-20, 20-25, 25-30, 30-35, 35-40, 40-45, 45-50, 50-60, 60-70, 70-80, 80-90, 90-100, 100-110, 110-120, 120-130, 130-140, 140-150, 150-160, 160-170, 170-180, 180-190, 200-250, 250-300, 300-350, 350-400, 400-450, 450-500, 500-600, 600-700, 700-800, 800-900, 900-1000, 1000-1500, or 1500-2000 frames per second, e.g., 2, 3, 4, 5, 6, 7, 8, 9, 10, 15, 20, 25, 30, 35, 40, 45, 50, 60, 70, 80, 90, 100, 200, 250, 300, 400, 500, 600, 700, 800, 900, 1000, 1100, 1200, 1300, 1400, 1500, 1600, 1700, 1800, 1900, or 2000 frames per second).

In another aspect, the invention provides a method of detecting light in a microfluidic device. The method includes providing a system as described herein; transmitting light from the light source to the detection point within the microfluidic device at angle α, wherein the light is redirected at the detection point, and wherein the redirected light is transmitted through the aspherical lens pair (and, in some embodiments, also the optical fiber); and detecting the light using the detector. In some embodiments, the microfluidic device is a droplet-forming device. In some embodiments, the light at the detection point is redirected by a droplet formed in the droplet forming device.

In another aspect, the invention provides a method of detecting a droplet in a microfluidic device, wherein the method includes providing any of the systems of the previous embodiments; transmitting light from the light source to the detection point within the microfluidic device at angle α, wherein the light is redirected by a droplet at the detection point, and wherein the redirected light is transmitted through the aspherical lens pair and the optical fiber; and detecting the light using the detector, thereby detecting the droplet.

In some embodiments of any of the methods of the previous aspects, the droplet includes one or more particles. In some embodiments, the one or more particles includes a biological particle (e.g., a cell) and/or a bead. In some embodiments, the biological particle is a cell including a fluorescent label. In some embodiments, the method further includes determining a number of particles in the droplet based on the detection. In some embodiments, the droplet is aqueous or miscible with water, and wherein the droplet is immiscible with the continuous phase. In some embodiments, any of the previous methods include sorting the droplet based on one or more characteristics of the droplet. In some embodiments, the detection is by image or video recording.

In some embodiments of any of the aforementioned systems or methods, the microfluidic device is multiplexed. In other embodiments of the systems or methods herein, the microfluidic device includes a droplet formation region, as described herein.

In one aspect, the invention features a device for detecting droplets or particles electrically (e.g., by impedance). The device includes a droplet or particle source, a detection channel having a proximal end and a distal end, and two or more electrodes. The droplet or particle source is configured to provide droplets or particles within a continuous phase. The detection channel is in fluid communication with the droplet or particle source, and the detection channel and the two or more electrodes are configured to allow detection of the droplet or particle as it passes therethrough. In some embodiments, the two or more electrodes are on the same side of the detection channel. In another embodiment, two of the electrodes are on opposing walls, e.g., in direct opposition. In certain embodiments, the device includes more than two electrodes. In some embodiments, the detection channel has a width and a depth, and the two or more electrodes have a width from 0.5% and 300% of the width or depth of the detection channel. In further embodiments, the width of each of the two or more electrodes is from 0.5 μm to 300 μm. In certain embodiments, the lateral distance (from the proximal end to the distal end of the detection channel) between the two or more electrodes is from 0.5 μm to 300 μm.

In some embodiments, the device includes a sorting region in fluid communication with the detection channel, having a sorter configured to sort the droplet or particle based on the detection. In certain embodiments, the sorting region includes 3 or more (e.g., 4, 5, 6, 7, 8, 9, 10, or more) partitions.

In further embodiments, the device has a droplet formation region. In some embodiments, the droplet formation region has a shelf region and a step region.

In another embodiment, the device includes one or more additional detection channels, each having two or more electrodes configured to detect a droplet or particle as it passes from the proximal end to the distal end in the channels.

In another aspect, the invention features methods of detecting a characteristic of one or more droplets or particles. A method of detecting a droplet or particle includes, for example, providing a device described herein, allowing the droplet or particle to pass through the detection channel from the proximal to the distal end, and measuring an electrical property (e.g., impedance) as it passes through the detection channel. In certain embodiments, the method further includes sorting the droplet or particle after it passes through the detection channel. In some embodiments, the method further includes forming a droplet or particle. In further embodiments, the droplet includes a particle (e.g., a bead or a biological particle). In another embodiment, the droplet includes a first and a second particle. In certain embodiments, the first particle is a bead, and the second particle is a biological particle, e.g., a cell.

In certain embodiments, the characteristic is the size or the velocity of a droplet or particle, the occupancy of a droplet (e.g., the presence or absence of a specified number of particles in a droplet), the presence or location of a particle in a droplet, or the liquid composition. In some embodiments when a cell may be present, the characteristic is the type of cell, the viability of the cell, the size of the cell, or the permeability of the cell.

In some embodiments, the impedance is measured at one or more frequencies. In further embodiments, the ratio of the difference in impedance of the droplet or particle at the one or more frequencies is indicative of the characteristics of the droplet or particle.

In another aspect, the invention features a device for sorting droplets or particles, including a droplet or particle source configured to provide droplets or particles to a region in which each cross-sectional dimension is greater than about 1 mm; a sorter configured to sort one or more of the droplets or particles in the region; and a collection region configured to receive the droplets or particles from the region, wherein each cross-sectional dimension of the collection region is less than about 1 mm (e.g., less than about 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 5 nm, 1 nm, 900 μm, 800 μm, 700 μm, 600 μm, 500 μm, 400 μm, 300 μm, 200 μm, 100 μm, 50 μm, 10 μm, 5 μm, 2 μm, 1 μm, or less).

In another aspect, the invention features a device including three regions in fluidic communication with each other. The first region has at least one cross-sectional dimension, e.g., two or all, less than about 1 mm, e.g., a channel; the second region has cross-sectional dimensions greater than about 1 mm, e.g., a reservoir; and the third region has at least one cross-sectional dimension, e.g., two or all, less than about 1 mm, e.g., a channel. The second region is disposed between the first and third regions. Liquids flowing from the first region may form droplets or particles in the second region, which are reentrained in the third region. Dimensions less that 1 mm are, e.g., less than about 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 5 nm, 1 nm, 900 μm, 800 μm, 700 μm, 600 μm, 500 μm, 400 μm, 300 μm, 200 μm, 100 μm, 50 μm, 10 μm, 5 μm, 2 μm, 1 μm, or less).

In another aspect, the invention features a method of sorting droplets or particles by providing a device as described herein; allowing the droplets or particles to enter the sorting region; and sorting one or more of the droplets or particles into one or more of the two or more partitions in the collection region.

In some embodiments, the method further includes detecting the droplets or particles prior to sorting.

The detection may provide feedback to the sorter, e.g., to actuate the sorter to sort the one or more droplets or particles.

It will be understood that the devices, systems, and methods described herein may, in addition to features specified, include any feature described herein that is not inconsistent with the structure of the underlying device, system, or method.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “about”, as used herein, refers to +/−10% of a recited value or +/−10° of a position.

The terms “adaptor(s)”, “adapter(s)” and “tag(s)” may be used synonymously. An adaptor or tag can be coupled to a polynucleotide sequence to be “tagged” by any approach including ligation, hybridization, or other approaches.

As used herein, the value of an “angle to” a planar object (e.g., 25° to 40° to a stage) refers to an angle taken along a plane perpendicular to the planar object. For example, the phrase “25° to 40° to the stage” refers to either 25° to 40° above the stage or 25° to 40° below the stage.

As used herein, a detector is said to be “aligned” with, or “in alignment” with, a channel (or vice-versa) when an interior portion of the channel is detectable by the detector, such that a droplet flowing through the channel occupies a portion of the focal plane of the detector.

The term “barcode,” as used herein, generally refers to a label, or identifier, that conveys or is capable of conveying information about an analyte. A barcode can be part of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads in real time.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “biological particle,” as used herein, generally refers to a discrete biological system derived from a biological sample. The biological particle may be a virus. The biological particle may be a cell or derivative of a cell. The biological particle may be an organelle from a cell. Examples of an organelle from a cell include, without limitation, a nucleus, endoplasmic reticulum, a ribosome, a Golgi apparatus, an endoplasmic reticulum, a chloroplast, an endocytic vesicle, an exocytic vesicle, a vacuole, and a lysosome. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or another organelle of a cell. A cell may be a live cell. The live cell may be capable of being cultured, for example, being cultured when enclosed in a gel or polymer matrix or cultured when comprising a gel or polymer matrix. Alternatively, the biological particle may be a virus.

As used herein, the term “depth” may refer to the dimension perpendicular to the focal plane (as in depth of field). Thus, the depth of a channel is vertical when the focal plane is horizontal, and the depth is horizontal if the focal plane is vertical.

The term “focal distance,” as used herein, refers to the axial distance between a detector or lens and a focal plane thereof.

The term “focal plane,” as used herein, refers to a plane having a depth of focus and being perpendicular to the axis of a detector or lens. For example, “focal plane of a detector”, as used herein, refers to a plane having a depth of focus and being perpendicular to the axis of that detector; a focused image of an object (e.g., a droplet) is obtained by the detector when the object (e.g., the droplet) is positioned in the focal plane of the detector.

The term “fluidically connected”, as used herein, refers to a direct connection between at least two device elements, e.g., a channel, reservoir, etc., that allows for fluid to move between such device elements without passing through an intervening element.

The term “genome,” as used herein, generally refers to genomic information from a subject, which may be, for example, at least a portion or an entirety of a subject's hereditary information. A genome can be encoded either in DNA or in RNA. A genome can comprise coding regions that code for proteins as well as non-coding regions. A genome can include the sequence of all chromosomes together in an organism. For example, the human genome has a total of 46 chromosomes. The sequence of all of these together may constitute a human genome.

The term “in fluid communication with”, as used herein, refers to a connection between at least two device elements, e.g., a channel, reservoir, etc., that allows for fluid to move between such device elements with or without passing through one or more intervening device elements.

The term “laterally deflect,” or any grammatical derivation thereof, refers to an induced change in direction of a moving object (e.g., a droplet or particle) from a first direction to a second direction, wherein the second direction includes a vector component orthogonal to the first direction. For example, a droplet or particle rising vertically through a continuous phase is laterally deflected when it contacts a downward-facing deflecting surface having an angle of 45° from the horizontal plane, because the direction of the droplet includes a horizontal component upon contacting the surface.

The term “linear array,” as used herein, refers to an array having at least one row or column of detecting elements. Linear arrays may be one dimensional or two dimensional.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA or a DNA molecule. The macromolecular constituent may comprise RNA or an RNA molecule. The RNA may be coding or non-coding. The RNA may be messenger RNA (mRNA), ribosomal RNA (rRNA) or transfer RNA (tRNA), for example. The RNA may be a transcript. The RNA molecule may be (i) a clustered regularly interspaced short palindromic (CRISPR) RNA molecule (crRNA) or (ii) a single guide RNA (sgRNA) molecule. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide or a protein. The polypeptide or protein may be an extracellular or an intracellular polypeptide or protein. The macromolecular constituent may also comprise a metabolite. These and other suitable macromolecular constituents (also referred to as analytes) will be appreciated by those skilled in the art (see U.S. Pat. Nos. 10,011,872 and 10,323,278, and WO/2019/157529 each of which is incorporated herein by reference in its entirety).

As used herein, a “micro-lens” refers to a lens having a diameter of less than 1.0 mm. In some embodiments, a micro-lens is part of a micro-lens array.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise an oligonucleotide or polypeptide sequence. The molecular tag may comprise a DNA aptamer. The molecular tag may be or comprise a primer. The molecular tag may be or comprise a protein. The molecular tag may comprise a polypeptide. The molecular tag may be a barcode.

The term “oil,” as used herein, generally refers to a liquid that is not miscible with water. An oil may have a density higher or lower than water and/or a viscosity higher or lower than water.

The terms “operative contact” and “operatively connected,” as used herein, generally refers to a functional relationship between components. In one example, an electrode and barrier are positioned in a manner to allow an electric field from the electrode to interact with substances, e.g., liquids or particles, on the other side of the barrier. The electrode may or may not be in conformal physical contact with the barrier.

The term “without overlapping signal,” as used herein, generally refers to a signal having less than 10% of the undesired signal.

The term “particulate component of a cell” refers to a discrete biological system derived from a cell or fragment thereof and having at least one dimension of 0.1 μm (e.g., at least 0.1 μm, at least 1 μm, at least 10 μm, or at least 100 μm). A particulate component of a cell may be, for example, an organelle, such as a nucleus, endoplasmic reticulum, a ribosome, a Golgi apparatus, an endoplasmic reticulum, a chloroplast, an endocytic vesicle, an exocytic vesicle, a vacuole, a lysosome or a mitochondrion.

The term “particulate component of a cell” refers to a discrete biological system derived from a cell or fragment thereof and having at least one dimension of 0.1 μm (e.g., at least 0.1 μm, at least 1 μm, at least 10 μm, or at least 100 μm). A particulate component of a cell may be, for example, an organelle, such as a nucleus, endoplasmic reticulum, a ribosome, a Golgi apparatus, an endoplasmic reticulum, a chloroplast, an endocytic vesicle, an exocytic vesicle, a vacuole, a lysosome or a mitochondrion.

The term “sample,” as used herein, generally refers to a biological sample of a subject. The biological sample may be a nucleic acid sample or protein sample. The biological sample may be derived from another sample. The sample may be a tissue sample, such as a biopsy, core biopsy, needle aspirate, or fine needle aspirate. The sample may be a liquid sample, such as a blood sample, urine sample, or saliva sample. The sample may be a skin sample. The sample may be a cheek swap. The sample may be a plasma or serum sample. The sample may include a biological particle, e.g., a cell or virus, or a population thereof, or it may alternatively be free of biological particles. A cell-free sample may include polynucleotides. Polynucleotides may be isolated from a bodily sample that may be selected from the group consisting of blood, plasma, serum, urine, saliva, mucosal excretions, sputum, stool and tears.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by ILLUMINA®, Pacific Biosciences (PACBIO®), Oxford NANOPORE®, or Life Technologies (ION TORRENT®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “sorter,” as used herein, generally refers to a mechanism that causes movement of one or more droplets or particles into one of two or more partitions (e.g., channels or regions), e.g., in a collection region. A sorter may be active or passive. In active sorting, actuation of the sorter moves a droplet to a partition. In passive sorting, droplets are moved to a partition based on an intrinsic property, e.g., mass, buoyancy, size, magnetic properties, or electrical properties.

As used herein, the term “slanted portion” or “slanted portion of the channel” refers to a portion of a channel having a wall (e.g., one or more walls, such as an upper wall and/or a lower wall) that is at a non-zero angle (e.g., at an angle from 1° to 90°) relative to a focal plane. Thus, a slanted portion has a slanted wall configured to allow droplets (e.g., droplets flowing along the channel) to pass through the focal plane, thereby enabling detection of multiple, distinct cross sections of the droplets at a single focal plane to be detected at different times.

The term “subject,” as used herein, generally refers to an animal, such as a mammal (e.g., human) or avian (e.g., bird), or other organism, such as a plant. The subject can be a vertebrate, a mammal, a mouse, a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, or an individual that is in need of therapy or suspected of needing therapy. A subject can be a patient.

The term “substantially stationary”, as used herein with respect to droplet or particle formation, generally refers to a state when motion of formed droplets or particles in the continuous phase is passive, e.g., resulting from the difference in density between the dispersed phase and the continuous phase.

As used herein, to “transmit light,” and grammatical variations thereof, means to direct or facilitate the passage of light in one or more directions. A light source transmits the light generated therein, a lens transmits input light as output light, and light reflected from a mirror is said to be transmitted. For example, light emitted by a sample can be transmitted through an aspherical lens pair and collected by an optical cable, which in turn can transmit the light to a detector. In this scenario, the detector has detected light transmitted from the optical cable, and the detector has also detected light transmitted from the aspherical lens pair. In other words, where light is transmitted from elements A to B to C, the light transmitted to C can be said to have been transmitted by A, B, or both.

The term “width”, as used herein with respect to an electrode, refers to the dimension along the proximal to distal axis of the channel. The width of an electrode may be a dimension orthogonal to the width of a channel.

As used herein, “X-Y-Z coordinates” refer to length-width-depth, respectively. An X-Y plane is a horizontal plane.

The term “X-Y-Z stage” refers to a stage designed to be moved in the X, Y, and Z planes (i.e., in three dimensions) relative to a light source or one or more components of a system in which it operates.

### BRIEF DESCRIPTION OF THE DRAWINGS

FIG. 1 is a schematic drawing showing an example of a microfluidic device for the introduction of particles, e.g., beads, into discrete droplets.

FIG. 2 is a schematic drawing showing an example of a microfluidic device for increased droplet formation throughput.

FIG. 3 is a schematic drawing showing another example of a microfluidic device for increased droplet formation throughput.

FIG. 4 is a schematic drawing showing another example of a microfluidic device for the introduction of particles, e.g., beads, into discrete droplets.

FIGS. 5A-5B are schematic drawings showing cross-section (FIG. 5A) and perspective (FIG. 5B) views an embodiment according to the invention of a microfluidic device with a geometric feature for droplet formation.

FIGS. 6A-6B are schematic drawings showing a cross-section view and a top view, respectively, of another example of a microfluidic device with a geometric feature for droplet formation.

FIGS. 7A-7B are schematic drawings showing a cross-section view and a top view, respectively, of another example of a microfluidic device with a geometric feature for droplet formation.

FIGS. 8A-8B are schematic drawings showing a cross-section view and a top view, respectively, of another example of a microfluidic device with a geometric feature for droplet formation.

FIGS. 9A-9B are schematic drawings showing views of another device of the invention. FIG. 9A is top view of a device of the invention with reservoirs. FIG. 9B is a micrograph of a first channel intersected by a second channel adjacent a droplet formation region.

FIGS. 10A-10E are schematic drawings showing views of droplet formation regions including shelf regions.

FIGS. 11A-11D are schematic drawings showing views of droplet formation regions including shelf regions including additional channels to deliver continuous phase.

FIG. 12 is a schematic drawing showing another device according to the invention having a pair of intersecting channels that lead to a droplet formation region and collection reservoir.

FIGS. 13A-13B are schematic drawings showing views of a device of the invention. FIG. 13A is an overview of a device with four droplet formation regions. FIG. 13B is a zoomed in view of an exemplary droplet formation region within the dotted line box in FIG. 13A.

FIGS. 14A-14B are schematic drawings showing views of devices according to the invention. FIG. 14A shows a device with three reservoirs employed in droplet formation. FIG. 14B is a device of the invention with four reservoirs employed in the droplet formation.

FIG. 15 is a schematic drawing showing a view of a device according to the invention with four reservoirs.

FIGS. 16A-16B are schematic drawings showing views of an embodiment according to the invention. FIG. 16A is a top view of a device having two liquid channels that meet adjacent to a droplet formation region. FIG. 16B is a zoomed in view of the droplet formation region showing the individual droplet formations regions.

FIGS. 17A-17B are schematic drawings showing schematic representations of a method according to the invention for applying a differential coating to a surface of a device of the invention. FIG. 17A is an overview of the method, and FIG. 17B is a micrograph showing the use of a blocking fluid to protect a channel from a coating agent.

FIGS. 18A-18B are schematic drawings showing cross-sectional views of a microfluidic device including a piezoelectric element for droplet formation. FIG. 18A shows the piezoelectric element in a first state. FIG. 18B shows the piezoelectric element in a second state.

FIG. 19 is a schematic drawing showing a microfluidic device including a piezoelectric element for droplet formation.

FIG. 20 is a schematic drawing showing a microfluidic device including a piezoelectric element for droplet formation. The droplets are collected in a circulating bath after formation.

FIG. 21 is a schematic drawing showing a microfluidic device including a piezoelectric element for droplet formation including a particle. The droplets contain a particle and are collected in a bath after formation.

FIG. 22 is a schematic drawing showing a microfluidic device including a piezoelectric element for droplet formation. The droplets contain a particle and are collected in a bath after formation.

FIG. 23 is a schematic drawing showing a sorter configured to sort two types of droplets. The droplets are formed at the generation point and move to the sorter, which sorts one type of droplet to one partition and another type of droplet to another partition. Inset on the right is an enlarged optical micrograph showing droplet generation.

FIG. 24 is a micrograph showing a sorter configured to sort droplets using dielectrophoresis. The droplet passes through a detection zone and is deflected to a partition by the dielectrophoretic force generated by the patterned electrodes. Both electrodes are patterned on the chip. Droplets that are not deflected are sorted to another partition.

FIG. 25 is a micrograph showing a sorter configured to sort droplets using dielectrophoresis. The electrodes are patterned with one on a sorter and one on a chip with a distance between them to allow passage of the sorted droplet while providing sufficient dielectrophoretic force. The droplet passes through a detection zone and is deflected to a partition by the patterned electrodes. Droplets that are not deflected are sorted to another partition.

FIG. 26 is a micrograph showing a sorter configured to sort droplets using an acoustic actuator. The droplet passes through a detection zone and is deflected to a partition by an acoustic force from the acoustic actuator. Droplets that are not deflected are sorted to another partition.

FIG. 27 is a micrograph showing a sorter configured to sort droplets using a mechanical arm. The droplet passes through a detection zone and is deflected to a partition by a mechanical arm that can physically push the droplet. Droplets that are not deflected are sorted to another partition.

FIG. 28 is a micrograph showing a sorter configured to sort droplets using a fluidic (e.g., pneumatic) actuator. The droplet passes through a detection zone and is deflected to a partition by a fluidic (e.g., pneumatic) force from the fluidic (e.g., pneumatic) actuator. Droplets that are not deflected are sorted to another partition.

FIG. 29 is a micrograph showing a sorter configured to sort droplets using a deflecting bubble generator. The droplet passes through a detection zone and is deflected to a partition by a bubble from the bubble generator. Droplets that are not deflected are sorted to another partition.

FIG. 30 is a micrograph showing a sorter configured to sort droplets using an optical tweezer. The droplet passes through a detection zone and is deflected to a partition by a force generated from the optical tweezer. Droplets that are not deflected are sorted to another partition.

FIG. 31 is a micrograph showing a moving sorter configured to sort droplets using a mechanical divider. The droplet passes through a detection zone and is placed in a partition by the divider that moves laterally. A rising droplet is sorted into one of two partitions depending on the location of the divider.

FIGS. 32A-32B are a schematic drawing (FIG. 32A) and micrograph (FIG. 32B) showing a sorter configured to sort droplets using electrostatic charge. FIG. 32A shows droplets with a negative charge sorted to a partition, while droplets without a charge (or opposite charge) are sorted to another partition. FIG. 32B shows the droplet passing through a channel where the nascent droplet is electrostatically charged right before it pinches off. Droplets with a negative charge are sorted into one partition. Droplets that are not negatively charged are sorted to another partition.

FIG. 33 is a schematic drawing showing droplets produced at a generation point and moving into a single channel. The droplets in the channel then reach a droplet sorter which deflects one type of droplet into one partition and another type of droplet into a different partition.

FIG. 34 is a micrograph showing a sorter configured to sort droplets using multi-way sorting. The droplet passes through a detection zone and is deflected to one of three different partitions by a deflecting force. Droplets that are not deflected are sorted to one partition while droplets that are deflected are sorted to one of two other partitions.

FIG. 35 is a micrograph showing a sorter configured to sort droplets using a thermal actuator. The droplet passes through a detection zone and is deflected to a partition by a force generated from the thermal actuator. Droplets that are not deflected are sorted to another partition.

FIGS. 36A-36B are schematic drawings showing a top view (FIG. 36A) and a cross-sectional side view (FIG. 36B) of a device with a sample channel and a barrier positioned to operatively connect two electrodes with the sample channel. In FIG. 36A, the sample flows from top to bottom.

FIG. 37A is a schematic drawing showing an electrode inserted from the side of a microfluidic device and a barrier positioned to operatively connect the electrode with the sample channel.

FIG. 37B is a schematic drawing showing an electrode with conductive gel inserted from the side of a microfluidic device and configured to operatively connect the electrode with the sample channel.

FIG. 38 is a schematic drawing showing an electrode and conductive gel inserted from the top of a microfluidic device and configured to operatively connect the electrode with the sample channel.

FIGS. 39A-39C are a set of schematic drawings showing an electrode with or without a conductive gel inserted from the bottom of a microfluidic device. The electrode is inserted into a cavity on the bottom of the device that is sized and shaped to receive the electrode. FIG. 39A shows a top view and FIGS. 39B-39C show cross-sectional side views. FIG. 39B shows an off-device electrode inserted into the cavity. FIG. 39C shows an electrode with conductive gel inserted into the cavity.

FIGS. 40A-40C are a set of schematic drawings showing an electrode with or without conductive gel in contact with the bottom of a microfluidic device. The electrode is in contact with a thin barrier on the bottom of the device. FIG. 40A shows a top view and FIGS. 40B-40C show cross-sectional side views. FIG. 40B shows an off-device electrode positioned against the barrier, which operatively connects the electrode and the sample channel. FIG. 40C shows a conductive gel positioned against the barrier, which operatively connects the conductive gel, in fluid connection with an electrode, with the sample channel.

FIG. 41 is a schematic drawing of a vertical cross-section of a device lacking a deflecting surface. Arrows indicate the optical/light pathway, which is obstructed by the droplets as they accumulate in the well.

FIGS. 42A and 42B are schematic drawings of vertical cross-sections of devices having an insert with a deflecting surface angled to laterally deflect droplets as they flow from a droplet source toward the surface of the continuous phase in a well. The deflecting surface can be made of a transparent material to render the optical pathway at the detection region unobstructed. The insert is positioned on the side of the well and deflects the droplets and continuous phase to a collection region between the outside of the insert and the inner walls of the well. The device of FIG. 42A has a deflecting surface characterized by its angle α from a vertical plane (or 90°-α from a horizontal plane). The device of FIG. 42B has a deflecting surface characterized by its angle β from a horizontal plane (or 90°-β from a horizontal plane).

FIG. 43 is a bright-field photomicrograph showing droplets being laterally deflected from a droplet source by a transparent deflecting surface.

FIG. 44 is a schematic drawing of a vertical cross-section of a device having a deflecting surface with an angle that laterally deflects droplets as they flow from a droplet source toward the surface of the continuous phase in a well. The deflecting surface is made of a transparent material to render the optical pathway at the detection region unobstructed. The insert is positioned at the periphery of the well to deflect the droplets and continuous phase to a collection region within the insert.

FIGS. 45A-45F show inserts having the general configuration shown in the schematic drawing of FIG. 44, in which the inserts are positioned to fit within the well and collect the droplets within the inner volume. FIG. 45A is a drawing of an oblique view of such an insert, in which the collection region is partially bounded by an outer cylindrical wall of the insert. FIGS. 45B and 45C are photographs showing inserts having a semi-ring-shaped top portion configured to secure the deflecting surface peripheral to an open collection region. The inserts of FIGS. 45B and 45C are configured to create a collection region bounded partially by the insert and partially by the wall of the well in which the insert resides. FIGS. 45D and 45E are drawings showing a further embodiment of an insert, in which the collection region is partially bounded by an outer cylindrical wall of the insert and the inner wall of the well in which the insert resides. The device shown in FIGS. 45D and 45E includes a crown (e.g., a reflector crown) configured to hold and/or stabilize a reflector. FIG. 45F is a drawing of a similar embodiment featuring a crown. The embodiment shown in FIG. 45F includes additional support members spanning the opening at the top of the device.

FIGS. 46A and 46B are drawings of another embodiment of an insert having a deflecting surface peripheral to an open collection region. The insert further includes openings configured to shunt the continuous phase to a continuous phase reservoir within the insert, separate from the collection region.

FIG. 47 is a schematic drawing of a vertical cross-section of a device similar to that shown in FIG. 44 but additionally includes a reflective surface where the droplets are laterally deflected, allowing for enhanced bright-field microscope detection.

FIG. 48 is a photograph of the deflecting surface of a device similar to that shown in the schematic drawing of FIG. 47. A flat surface, which can be coated with a reflective material, is configured to be positioned near the droplet source, above the droplet detection region.

FIG. 49 is a schematic drawing showing a system including a microfluidic device with an insert and a quarter wave plate. Collimated or slightly focused illumination light is directed by the optical polarizer at a first polarization through the detection region and a quarter wave plate. The light is then reflected back through the wave plate becoming polarized in a second polarization orthogonal to the first polarization. The light then passes through the detection region and the polarizer to the imaging optics.

FIG. 50 is a schematic drawing showing a system including a microfluidic device with an insert and a quarter wave plate. The optical setup includes an objective lens, a tube lens, and a detector. The quarter wave plate includes a high reflection coating on one end and an anti-reflection coating on the other end.

FIG. 51 is a schematic drawing showing a system including a microfluidic device with an insert, a reflective plate, and a quarter wave plate. The optical setup includes an objective lens, a tube lens, and a detector.

FIG. 52 is a schematic drawing showing a system including a microfluidic device with an insert and a quarter wave plate. The optical setup includes an objective lens, a tube lens, and a detector. In this configuration, the polarizer is positioned between the device and the objective lens.

FIG. 53 is a schematic drawing showing unpolarized light being polarized by a polarization beam splitter. Some of the light is reflected to a quarter wave plate and is s-polarized; some of the light passes through the polarizer and is p-polarized.

FIG. 54 is a schematic drawing showing light reflecting back from the quarter wave plate as p-polarized light and passing through the polarizer.

FIGS. 55A and 55B are schematic drawings showing p-polarized light (FIG. 55A) and unpolarized light (FIG. 55B). FIG. 55A shows an optical polarization beam splitter in which the p-polarized light passes through. FIG. 55B shows a 50% beam splitter in which 50% of the unpolarized light is reflected by the beam splitter and does not pass through.

FIGS. 56A and 56B are schematic drawings showing how optical noise is reduced by the quarter wave plate. FIG. 56A shows p-polarized useful image signal that passes through the polarization beam splitter and reaches the detector, while the s-polarized background noise does not. Background optical noise is mainly created from reflection of illumination light before reaching the quarter wave plate. FIG. 56B shows a similar schematic as FIG. 56A but with unpolarized light and a reflective plate. This arrangement does not reduce background noise.

FIGS. 57A-57B are schematic drawings of cross-sectional view of a device that includes a light source and a plurality of detectors. FIG. 57A shows a channel sandwiched between two layers of detectors and a light source illuminating a particle or droplet. The thin arrows from the droplet or particle indicate scattered light, which is detected by the detectors. FIG. 57B shows an alternative embodiment in which a light guide directs light from the light source to the channel.

FIGS. 58A-58B are schematic drawings showing a cross-sectional view of a device that includes a light source and a plurality of detectors. FIG. 58A shows the channel sandwiched between two layers of detectors and a light source illuminating a droplet in the channel. The droplet contains a cell and a gel bead (GB). The thin arrows from the droplet indicate scattered light from the droplet, the cell, or the gel bead. FIG. 58B shows an alternative embodiment in which a light guide directs the light source to the channel.

FIGS. 59A-59C are schematic drawings showing a top view of various geometric arrangements of a channel sandwiched between a plurality of detectors and a light source. FIG. 59A shows a plurality of detectors packed to minimize gaps between the detectors. FIG. 59B shows a similar arrangement as in FIG. 59A in which a light source is located within the detector array. FIG. 59C shows an alternative embodiment in which a light source is located (or illuminates) in a gap between detectors.

FIGS. 60A-60C are schematic drawings showing a top view of various geometric arrangements of a channel sandwiched between a plurality of detectors. FIG. 60A shows a diamond arrangement of detectors, FIG. 60B shows a hexagonal arrangement of detectors, and FIG. 60C shows a linear arrangement of detectors.

FIGS. 61A-61C are photographs showing optical setups of a device, a laser light source, and detectors. FIG. 61A shows a top right perspective view, FIG. 61B shows a top left perspective view, and FIG. 61C shows a bottom left perspective view.

FIGS. 62A-62C are micrographs showing a top view of various geometrical arrangements of a channel located between a light source and a plurality of detectors. FIG. 62A shows an embodiment including a linear arrangement of detectors on the bottom of the channel and a laser on the top. FIG. 62B shows an embodiment including a hexagonal arrangement of detectors and a light emitting diode (LED) light source on the bottom of the channel. FIG. 62C shows an embodiment including a plurality of detectors on the bottom of the channel and a laser light source on the top.

FIGS. 63A-63B are schematic drawings of cross-sectional views of a microfluidic device according to the invention that includes a channel with a slanted portion that is tilted in Z direction. The X-Y plane of the droplet formation region is displayed vertically for visual clarity. FIG. 63A depicts the cross-sectional view of a device, where the slanted portion of the channel has a constant depth. FIG. 63B depicts the cross-sectional view of a device, where the slanted portion of the channel has a variable depth. In the particular embodiment shown in FIG. 63B, the depth at the distal end of the slanted portion is greater than the depth at the proximal portion.

FIG. 64 is a schematic representation of cross-sectional view of a microfluidic device according to the invention that includes a channel which is aligned with two lenses having different focal planes.

FIG. 65 is a schematic representation of cross-sectional view of a microfluidic device according to the invention that includes a channel which is aligned with an oscillating detector.

FIG. 66 is a schematic drawing of a vertical cross-section of a device according to the invention that includes a channel which is constricted at least in some portions. FIG. 66 shows that a droplet is constricted, and cell and bead within the droplet is positioned in the focal plane of a detector while the droplet flows along the constricted portion of the channel.

FIG. 67 is a schematic illustration of a detection system in which multiple light guides are configured to transmit light from multiple detection points of a microfluidic device to spatially distinct regions of an array detector to combine signal from the multiple detection points into a single image or video.

FIG. 68 is a schematic illustration of a detection system in which a line scan camera is configured to simultaneously detect signals from multiple channels of a microfluidic device without overlapping.

FIG. 69 is a schematic illustration of a detection system in which a laser positioned above a microfluidics device transmits a beam at an angle to an interrogation volume having a detection point within a microfluidic device. Light emitted from the sample is transmitted through collection optics into an optical fiber, which transmits the light to a detector (not shown).

FIG. 70 is a schematic illustration of a detection system in which a laser positioned below a microfluidics device transmits a beam at an angle to an interrogation volume having a detection point within a microfluidic device. Light emitted from the sample is transmitted through collection optics into an optical fiber, which transmits the light to a detector (not shown).

FIG. 71 is a schematic illustration of a collection optics configuration including an aspherical lens pair configured to transmit light from a sample in an interrogation volume to an optical fiber.

FIG. 72 is a schematic illustration of a collection optics configuration including an aspherical lens pair configured to transmit light from a sample in an interrogation volume to an optical detector.

FIG. 73 is a schematic illustration of a collection optics configuration including an aspherical lens pair configured to transmit light from a sample in an interrogation volume to two optical detectors. In this configuration, a dichroic mirror selectively passes light to an optical detector and/or reflects all or a portion of the light to a second optical detector.

FIGS. 74A-74C are schematic representations of a microfluidic device including electrodes for measuring electrical properties, e.g., impedance. FIG. 74A shows a top view of a detection channel including electrodes. FIG. 74B shows a cross-sectional view of a detection channel including coplanar electrodes. FIG. 74C shows a cross-sectional view of a detection channel including parallel facing electrodes. The droplet contains a gel bead (GB) and a cell.

FIGS. 75A-75B are representations of equivalent circuit models for the measurement of impedance by a device of the invention. FIG. 75A shows a representation of one electrode measuring the impedance of a cell, which includes contributions from membrane capacitance (Cₘₑₘ) and the interior of the cell (R_{cell}). The second electrode measures the impedance of the continuous phase (R_{aq}). FIG. 75B shows a representation of measuring cell viability in a detection channel. A viable cell, having an impedance with contributions from intact cell membrane capacitance (Cₘₑₘ) and cell interior (R_{cell}) is measured. This measurement differs from that of a second cell with contributions from a permeable cell membrane capacitance (Cₚₘₑₘ) and cell interior (R_{pcell}).

FIG. 76 is a representation of equivalent circuit models for the measurement of impedance by the device. One electrode measures the impedance of a droplet, which contains a cell, having a cell membrane (Cₘₑₘ), a cell interior (R_{cell}), a gel bead (GB) (R_{GB}), and the aqueous phase of the droplet (R_{daq}). A second electrode measures the impedance of the continuous phase (R_{aq}).

FIG. 77 is a representation of equivalent circuit models showing the differences in impedance of a droplet containing a gel bead (R_{gb}) and a cell having a cell membrane capacitance (Cₘₑₘ) and cell interior (R_{cell}) and a droplet containing only a gel bead (R_{gb}).

### DETAILED DESCRIPTION OF THE INVENTION

The invention provides devices, kits, and systems for detecting and/or sorting droplets or particles and methods of their use. Devices may include droplet or particle forming devices. The devices of the invention can be integrated with any of the droplet or particle forming devices described herein or known in the art, or a population of droplets or particles produced by any method may be provided to a device or system of the invention. Droplets or particles may be first formed in a larger volume, such as in a reservoir, and then reentrained into a channel, e.g., for sorting and/or detection.

Detection and/or sorting of droplets or particles is desirable in order to characterize and/or sort them in their final state or detect a number of droplets or particles. The characteristic of the droplet may be the viability of the cell (e.g., alive or dead), the size of the droplet or particle, the location of a particle in the droplet, the composition of the droplet or particle, the occupancy of the droplet (e.g., an empty droplet or a droplet having a particle (e.g., a bead, a cell, a bead and a cell)) or the velocity of the droplet or particle.

In addition to the features described below, any of the devices, systems, methods and kits described in US 2019/0060890, US 2019/0060905, US 2019/0060904, US 2019/0060906, and US 2019/0064173 are contemplated for adaptation in the present systems and methods. Exemplary fluidic configurations are described herein and shown in the devices of Examples 1-22. Each of these devices may include or be modified to include a sorting and/or detection region (e.g., channel) as described herein.

The devices may be used to sort droplets or particles of a desired property and/or size suitable for utilization as microscale chemical reactors, e.g., for genetic sequencing. In general, droplets or particles are provided by a droplet or particle source. The droplets or particles may be first formed by flowing a first liquid through a channel and into a droplet or particle formation region including a second liquid, i.e., the continuous phase, which may or may not be externally driven. Thus, droplets or particles can be formed without the need for externally driving the second liquid.

Sorting droplets or particles includes allowing the droplets or particles to enter into a sorting region. The sorting region is in fluid communication with (e.g., fluidically connected to) the droplet or particle source. In some embodiments, the droplet or particle source includes a droplet or particle formation region. The droplets or particles are then sorted by a sorter to sort one or more of the droplets or particles into different partitions. The sorter can employ active or passive sorting mechanisms. Furthermore, the sorter can employ two-way sorting (e.g., sorting the droplets or particles into one of two different partitions) or multi-way sorting (e.g., sorting the droplets or particles into one of three or more (e.g., 4, 5, 6, 7, 8, 9, 10, or more) partitions). The droplet or particle sorter can function either in a microfluidic channel or in a larger volume, such as in a reservoir. The droplet or particle sorter can sort premade droplets or particles, or it can sort droplets or particles as they are generated. As a result of the sorting process, the invention can provide populations of droplets or particles enriched in a certain characteristic.

Accumulation in a droplet or particle collection region can obstruct detection of single droplets or particles due to optical obstruction or other interference (e.g., light reflection or refraction) caused by surrounding droplets or particles. Furthermore, low signal strength and low signal to noise ratio of detectable light can hinder accurate detection. The devices, systems, and methods described herein may feature a droplet or particle source and a detection region includes a surface that laterally deflects droplets or particles before accumulation in a collection region, thereby allowing detection of the droplet or particle as it passes through a detection region. The deflecting surface can be part of any one of various device designs, such as those described herein. In addition, the devices of the present invention can be configured for various means of detection described herein and known in the art.

The invention also provides devices, systems and methods for detecting light scattered from a droplet and/or a particle in a channel.

The detection may be used to determine a characteristic of a droplet and/or a particle (e.g., a bead, a cell, or a bead and a cell) by illuminating the droplet or particle in an emulsion (or mixture) and measuring the light as the droplet or particle passes.

These devices, systems, and methods may include one or more detectors and/or light sources on one or more sides of the device (e.g., microfluidic device) for imaging droplets and/or particles therein. The detectors gather scattered light at various angles and attenuated light from droplets and/or particles. By using a plurality of detectors on multiple sides of the device, scattered and attenuated light can be collected simultaneously. This provides increased sensitivity for detection and the ability to tune illumination sources and detectors. This also provides the ability to detect the contents of a droplet.

The present invention also provides devices, systems, and methods for improved imaging of droplets or particles. The content of droplets or particles, e.g., cells or particles, can be distributed in different planes that may be difficult to image at one time. The invention allows imaging of droplets or particles simultaneously at multiple focal planes, sequential imaging of multiple cross sections of a droplet as the droplet passes through a focal plane, or imaging of droplets or particles in a single plane where the contents of the droplet have been constricted.

The invention also provides systems and methods for simultaneous detection of multiple detection points in a microfluidic device. In particular, systems described herein combining signals from multiple points into a single image or video without overlapping. Such systems and methods are useful in the detection of droplets or particles at multiple points in a microfluidic device, e.g., from multiple droplet or particle sources.

The invention also provides systems and methods for improved detection of contents within a sample (e.g., droplets formed in microfluidic devices). In particular, the invention may employ light that is transmitted through a sample at an angle, such as Brewster's angle, which eliminates the need for conventional objectives and/or bulky configurations to detect small volumes. Such systems and methods are useful in the detection of droplets made using microfluidic devices.

The invention also provides devices and methods for detecting the electrical properties, e.g., impedance, of a droplet or particle in a channel.

In one aspect, the electrical property of a cell (e.g., permeability) may be used to determine the viability of a cell. Although a cell is naturally semi-permeable (via its cell membrane), a non-viable cell may have an abnormal permeability (e.g., abnormally increased or decreased permeability) relative to a viable cell. The methods described herein may be used for detecting differences in cell permeability, thereby detecting differences in cell viability. Impedance may also be measured at various frequencies, allowing for the determination of multiple characteristics in a high-throughput fashion.

These devices and methods described herein include two or more electrodes on one or more portions (e.g. a top portion or a bottom portion) of a channel of the device. The electrodes measure an electrical property, e.g., impedance of the droplets and/or particles as they pass through the detection channel. By using a plurality of electrodes measuring at one or more frequencies the impedance of a plurality of droplets rapidly be measured at an increased sensitivity. A plurality of detection and sorting mechanisms are described herein, and the skilled artisan would understand that droplets or particles can be detected and/or sorted with any combination of detection and/or sorting mechanisms described herein. For example, sorting mechanisms described below include, e.g., electrode(s), a dielectrophoretic actuator, an acoustic actuator, a fluidic (e.g., pneumatic) actuator, a mechanical actuator, a bubble generator, an optical tweezer, a magnet, and an electrostatic charger. Meanwhile, detection mechanisms described below may include, e.g., deflection, reflection (e.g., with a quarter wave plate), a plurality of detectors and/or light guides or light sources, slanted channels or reservoirs, multiple point detection, Brewster angle detection, angle of incidence detection, and electrical impedance. The devices, systems, and methods described herein may employ any combination of sorting and detection mechanisms, or a plurality of sorting and/or detection mechanisms.

### Devices

A device of the invention may include a first channel, region, or reservoir having a depth, a width, a proximal end, and a distal end. The proximal end is or is configured to be in fluid communication with a source of liquid, e.g., a reservoir integral to the device or coupled to the device, e.g., by tubing. The distal end may be in fluid communication with, e.g., fluidically connected to, a droplet or particle source (e.g., a droplet or particle formation region). A droplet or particle formation region may allow liquid from the first channel to expand in at least one dimension, leading to droplet formation under appropriate conditions as described herein. A droplet or particle formation region can be of any suitable geometry. The droplet or particle source (e.g., droplet or particle formation region) is in fluid communication with, e.g., fluidically connected to, a sorting region. A device for sorting droplets or particles includes a sorting region that allows the droplets or particles from the droplet or particle source, e.g., the droplets or particles that are formed in a droplet or particle formation region, to be sorted according to a particular property or characteristic. A device for detecting droplets or particles includes a detection region, e.g., a channel. A device may include both a sorting region and a detection region. The sorting and detection regions may be in fluid communication with, e.g., fluidically connected to, each other. The sorting region may be upstream of the detection region or vice versa. The detection region may be configured to provide feedback to the sorting region, e.g., by actuating the sorter. The detection region may include a detector (e.g., a sensor) for detection. If coupled to a sorter, the detector may provide a stimulus to the actuator, thereby directing the actuator to sort the droplets or particles in a particular manner.

### Sorting Region

The invention features devices that may include a droplet or particle sorting region. A droplet or particle sorting region includes a sorter to sort one or more of the droplets or particles into one or more partitions. The sorter can be part of the device or may be provided as a separate component, e.g., operably coupled to the sorting region, to form a system that includes the device. The sorting region can be of any suitable geometry and may be, for example, a well, a channel, a reservoir, a portion thereof, or the like. The sorting region may be enclosed or not enclosed (e.g., open ended). The sorter may include any mechanism suitable for sorting droplets or particles based on a particular characteristic or parameter (e.g., size, charge, composition, mass, material properties (e.g. magnetic properties, dielectric properties, acoustic properties, electrical properties), or presence/absence of a particle). The sorter may provide an active or passive force to sort the droplets or particles to a partition in the collection region, e.g., by laterally deflecting the sorted droplet or particle into a collection region. The sorter can employ two-way sorting (e.g., sorting the droplets or particles into one of two different partitions) or multi-way sorting (e.g., sorting the droplets or particles into one or three or more (e.g., 4, 5, 6, 7, 8, 9, 10, or more) partitions). The sorting region can be open-ended (e.g., connected to subsequent partitions, e.g., channels or reservoirs) or enclosed. The sorting region can have any length, width, and/or height suitable for sorting one or more droplets or particles. For example, the length, width, and/or height may be at least, independently, e.g., 1 μm-10 mm (e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm). The sorting region may have a volume of e.g., 1 nL-10 mL (e.g., 1 nL, 2 nL, 3 nL, 4 nL, 5 nL, 6 nL, 7 nL, 8 nL, 9 nL, 10 nL, e.g., 10 nL-100 nL, e.g., 20 nL, 30 nL, 40 nL, 50 nL, 60 nL, 70 nL, 80 nL, 90 nL, 100 nL, e.g., 100 nL-1 μL, e.g., 200 nL, 300 nL, 400 nL, 500 nL, 600 nL, 700 nL, 800 nL, 900 nL, 1 μL, e.g., 1 μL-10 μL, e.g., 2 μL, 3 μL, 4 μL, 5 μL, 6 μL, 7 μL, 8 μL, 9 μL, 10 μL, e.g., 10-100 μL, e.g., 20 μL, 30 μL, 40 μL, 50 μL, 60 μL, 70 μL, 80 μL, 90 μL, 100 μL, e.g., 100 μL-1 mL, e.g., 200 μL, 300 μL, 400 μL, 500 μL, 600 μL, 700 μL, 800 μL, 900 μL, 1 mL, e.g., 1 mL-10 mL, e.g., 2 mL, 3 mL, 4 mL, 5 mL, 6 mL, 7 mL, 8 mL, 9 mL, 10 mL). In some embodiments, the sorting region has no cross-sectional dimension of less than 1 mm. For example, each cross-sectional dimension of the sorting region may have a length of at least 1 mm (e.g., 2 mm, 3 mm, 4 mm, 5 mm, 6 mm, 7 mm, 8 mm, 9 mm, 10 mm, or more). In some embodiments, the device includes a sorting region in at least one, e.g., each, cross-sectional dimension is greater than at least 1 mm in fluid communication with (e.g., fluidically coupled to) a second region (e.g., a channel) in which each cross-section dimension is less than at least 1 mm. The sorter may include a surface configured to laterally deflect the droplets or particles to the one or more partitions in the collection region. The surface may include one or more openings through which the deflected droplets or particles pass. Mechanisms or actuators that may be used to sort droplets or particles include, for example, a dielectrophoretic actuator, an acoustic actuator, a fluidic (e.g., pneumatic) actuator, a mechanical actuator, a bubble generator, an optical tweezer, a magnet, or an electrostatic charger. In one embodiment, the sorter provides a surface that actively or passively sorts the droplets or particles, e.g., based on size or content. The mechanisms and actuators that may be used for sorting are described in more detail below.

### Dielectrophoretic Actuator

Dielectrophorersis (DEP) is a phenomenon in which a force is exerted on a dielectric particle when it is subjected to a non-uniform electric field. The basis for generating a DEP force is the interaction between the particle's dipole and the spatial gradient of the electric field. A DEP actuator includes one or more (e.g., 2, 3, 4, 5, 6, 7, 8, 9, 10, 20, 30, 40, 50, 60, 70, 80, 90, or 100, or more) patterned electrodes that generate the DEP force. The electrodes may be arranged, in a grid pattern, as microwells, DEP wells, extruded patterns, a sidewall pattern, or a top-bottom pattern. The pattern of the DEP electrodes may be determined, e.g., empirically, based on the size of the particle or the type of sorting to be performed. The one or more electrodes may be spaced at a predetermined width of, e.g., 1 μm-10 mm (e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1 nm, e.g., 200 μm, 300 μm, 400 μm, 500 μm, 600 μm, 700 μm, 800 μm, 900 μm, 1 nm, e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1 μm-10 μm, e.g., 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm). Exemplary DEP electrodes are described, e.g., in Yafouz et al. Sensors 14: 6336-6369, 2014, which is hereby incorporated by reference. Electrodes may be solid, semisolid (e.g., gel), or liquid conducting paths. For example, metal or other conducting materials may be incorporated into a device as electrodes. Alternatively, channels containing a liquid, gel, or combination thereof can be incorporated into a device and used as electrodes. Combinations of different types of electrodes may also be employed.

A DEP actuator generates a DEP force that interacts with the droplet or particle being sorted. For example, if the droplet contains a particle, the DEP force moves the droplet or particle in a certain way, thereby directing the droplet or particle into a particular partition. Alternatively, if the droplet does not contain a particle, the DEP force moves the droplet in a different manner or not at all, thereby directing the droplet into a different partition (e.g., waste channel). The reverse of this sorting regime can also be employed. In one embodiment, the DEP actuator is triggered to sort a droplet or particle by applying a DEP force upon receiving an input stimulus (e.g., feedback), e.g., from a sensor in the detection region. In some embodiments, the DEP electrodes are patterned or spaced at a distance that permits the passage of the droplet or particle during sorting. The DEP electrodes may be used for either active or passive DEP sorting.

### Acoustic Actuator

Acoustic force is a physical phenomenon resulting from the interaction of an acoustic wave with an obstacle placed along its path. Acoustic force is proportional to the volume of the particle, acoustic pressure, acoustic wavelength, the density of the sphere and medium, and the compressibility of the sphere and medium. An acoustic actuator generates an acoustic wave that provides a force that interacts with the droplet or particle being sorted. Depending on the particle and medium properties, acoustic force can push a particle towards a pressure or anti-pressure node, consequently changing the direction of the particle and sorting it. For example, if the droplet contains a particle, the acoustic force moves the droplet or particle in a certain way, thereby directing the droplet or particle into a particular partition. Alternatively, if the droplet does not contain a particle, the acoustic force moves the droplet in a different manner or not at all, thereby directing the droplet into a different partition (e.g., waste channel). The reverse of this sorting regime can also be employed. In one embodiment, the acoustic actuator is triggered to sort a droplet by applying an acoustic force upon receiving an input stimulus (e.g., feedback), e.g., from a sensor in the detection region.

### Fluidic Actuator

Fluidic (e.g., pneumatic) force is generated by movement of a flow in a microfluidic channel or deformation of the microfluidic channel by, for example, controlling hydrodynamic pressure by hydrodynamic gating or actuating using fluid (e.g., air) pressure; or using a valve to change the hydrodynamic resistance. This mechanical motion may be employed to sort droplets or particles, e.g., into a collection region. A fluidic (e.g., pneumatic) actuator generates pressure that provides a force that interacts with the droplet or particle being sorted. For example, if the droplet contains a particle, the fluidic (e.g., pneumatic) force moves the droplet or particle in a certain way, thereby directing the droplet or particle into a particular partition. Alternatively, if the droplet does not contain a particle, the fluidic (e.g., pneumatic) force moves the droplet in a different manner or not at all, thereby directing the droplet into a different partition (e.g., waste channel). The reverse of this sorting regime can also be employed. In one embodiment, the fluidic (e.g., pneumatic) actuator is triggered to sort a droplet or particle by applying a fluidic (e.g., pneumatic) force upon receiving an input stimulus (e.g., feedback), e.g., from a sensor in the detection region.

### Mechanical Actuator

A mechanical actuator generates motion (e.g., with a lever, arm, valve, rod, protrusion, pump or the like) and may or may not physically interact with the droplet or particle being sorted. Suitable mechanical actuators are known in the art. For example, the mechanical actuator may be a valve, a mechanical arm, or a divider. Valves useful for a device of the present invention include diaphragm valves, solenoid valves, pinch valves, or a combination thereof. Valves can be controlled manually, electrically, magnetically, hydraulically, fluidically (e.g., pneumatically), or by a combination thereof. In some embodiments, the actuator is a mechanical arm that physically moves droplets or particles into a desired partition. In some instances, the mechanical actuator includes a valve whereby the droplets or particles push up against the valve. The valve may open to allow passage of the droplets or particles through a barrier and into a partition. In certain embodiments, if the droplet contains a particle, the mechanical actuator moves the droplet or particle in a certain way, thereby directing the droplet or particle into a particular partition. Alternatively, if the droplet does not contain a particle, the mechanical actuator moves the droplet in a different manner or not at all, thereby directing the droplet into a different partition (e.g., waste channel). The reverse of this sorting regime can also be employed. In one embodiment, the mechanical actuator is triggered to sort a droplet or particle by applying a mechanical force upon receiving an input stimulus (e.g., feedback), e.g., from a sensor in the detection region.

The sorting mechanism may include a divider (e.g., a surface, e.g., angled surface), to sort one or more droplets or particles. A divider may be any feature that at least, in part, protrudes into or recedes from the sorting region. A divider may sort droplets or particles by redirecting or deflecting the directionality of one or more droplets or particles depending on a certain property (e.g., density, mass, or velocity). A divider may laterally deflect a droplet or particle by an angle from 1°-180° (e.g., 5°, 10°, 15°, 20°, 25°, 30°, 40°, 50°, 60°, 70°, 80°, 90°, 100°, 110°, 120°, 130°, 140°, 150°, 160°, 170°, 180°) into a partition in the collection region. The sorting region may optionally include multiple dividers arranged consecutively (e.g., in parallel or in series) for more complex or serial sorting mechanisms. A divider may also sort by moving laterally to determine the partition a particular droplet or particle enters as it traverses the sorting region, e.g., floating or sinking.

### Bubble Generator

A bubble generator generates bubbles composed of gas (e.g., air) that are released into the emulsion. The bubble may be generated, e.g., by releasing air or via thermal bubble actuation into a fluid medium. The bubble may physically interact (directly or indirectly) with the droplet or particle being sorted. For example, if the droplet contains a particle, the bubble will push the droplet or particle in a certain way, thereby directing the droplet or particle into a particular partition. Alternatively, if the droplet does not contain a particle, the bubble pushes the droplet in a different manner or not at all, thereby directing the droplet into a different partition (e.g., waste channel). The reverse of this sorting regime can also be employed. In one embodiment, the bubble generator is triggered to sort a droplet or particle by generating a bubble upon receiving an input stimulus (e.g., feedback), e.g., from a sensor in the detection region.

### Optical Tweezer

An optical tweezer can be used to manipulate a droplet or a particle. The optical tweezer may physically interact (directly or indirectly) with the droplet or particle being sorted. For example, if the droplet contains a particle, the tweezer moves the droplet or particle in a certain way, thereby directing the droplet or particle into a particular partition. Alternatively, if the droplet does not contain a particle, the optical tweezer moves the droplet in a different manner or not at all, thereby directing the droplet into a different partition (e.g., waste channel). The reverse of this sorting regime can also be employed. In one embodiment, the tweezer is triggered to sort a droplet or particle by activating the tweezer upon receiving an input stimulus (e.g., feedback), e.g., from a sensor in the detection region.

### Magnetic Actuator

A magnet or magnetic field can be used to manipulate a droplet or a particle (e.g., a droplet with a magnetic particle). The magnet imparts a magnetic field (e.g. electromagnetic field or a permanent magnet) that affects the motion of fluid due to its magnetic properties (e.g. ferrofluid), the motion of the droplet or a particle within the droplet based on their magnetic properties (magnetic susceptibility) or magnetic particles inside the droplet. For example, if the droplet contains a particle, the magnetic field moves the droplet or particle in a certain way, thereby directing the droplet or particle into a particular partition. Alternatively, if the droplet does not contain a particle, the magnetic field moves the droplet in a different manner, thereby directing the droplet into a different partition (e.g., waste channel) or not directing the droplet into any partition. In one embodiment, the magnetic field is triggered to actively sort a droplet or particle by activating the field upon receiving an input stimulus (e.g., feedback), e.g., from a sensor in the detection region. Alternatively, droplets or particles can be sorted passively by passing over or through a magnetic field. In one embodiment, the droplet may contain a neutral ferromagnetic particle that is magnetized and can be sorted by a global magnetic field.

### Thermal Actuator

A thermal actuator can be used to manipulate a droplet or a particle. For example, a thermal actuator can change the viscosity or other fluidic properties of the medium to deflect the droplet. Moreover, the thermal actuator can generate a vortex to deflect the droplet. Thermal actuation can be generated by a laser, an electrode, or any other method of generating heat in a fluid. For example, if the droplet contains a particle, the thermal actuator moves the droplet or particle in a certain way, thereby directing the droplet or particle into a particular partition. Alternatively, if the droplet does not contain a particle, the thermal actuator moves the droplet in a different manner or not at all, thereby directing the droplet into a different partition (e.g., waste channel). The reverse of this sorting regime can also be employed. In one embodiment, the thermal actuator is triggered to sort a droplet or particle by activating the thermal actuator upon receiving an input stimulus (e.g., feedback), e.g., from a sensor in the detection region.

### Electrostatic Charger

Electrostatic forces are generated by the interaction of electric charges. During formation, the droplets or particles may pass through a channel where the flow is electrostatically charged, e.g., right before the target droplet forms. Based on the force exerted on the charged or uncharged droplets or particles, the droplets or particles may be sorted to a particular partition. In some embodiments, the electrostatic charger includes one or more (e.g., 2, 3, 4, 5, 6, 7, 8, 9, 10, 20, 30, 40, 50, 60, 70, 80, 90, or 100, or more) patterned electrodes that generate the electrostatic charge. The electrodes may be arranged, in a grid pattern, as microwells, extruded patterns, a sidewall pattern, or a top-bottom pattern. The pattern of the electrodes may be determined, e.g., empirically, based on the size of the particle or the type of sorting to be performed. The one or more electrodes may be spaced at a predetermined width of, e.g., 1-10 mm (e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1 nm, e.g., 200 μm, 300 μm, 400 μm, 500 μm, 600 μm, 700 μm, 800 μm, 900 μm, 1 nm, e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1-10 μm, e.g., 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm). Electrodes may be solid, semisolid (e.g., gel), or liquid conducting paths. For example, metal or other conducting materials may be incorporated into a device as electrodes. Alternatively, channels containing a liquid, gel, or combination thereof can be incorporated into a device and used as electrodes. Combinations of different types of electrodes may also be employed.

In some embodiments, for example, droplets or particles with a negative charge are sorted into one partition, droplets or particles with a positive charge are sorted into a different partition, and droplets or particles with no charge are sorted into yet a different partition. In some embodiments, only, e.g., negatively charged droplets or particles are sorted into one partition while the positively charged droplets or particles or uncharged droplets or particles are directed into a different partition (e.g., waste channel). In some embodiments, only, e.g., positively charged droplets or particles are partitioned into one partition while the negatively charged droplets or particles or uncharged droplets or particles are directed into a different partition (e.g., waste channel). In some embodiments, only, e.g., uncharged droplets or particles are sorted into one partition while the positively charged droplets or particles or negatively charged droplets or particles are directed into a different partition (e.g., waste channel).

The charge of the droplet or particle can be employed by the sorter to perform multi-way sorting of droplets or particles in addition to two-way sorting. For example, droplets or particles can be charged with a positive or negative charge, or multiple positive or negative charges. In some embodiments, e.g., negatively charged droplets or particles are sorted into one partition while the multiple-negative charged droplets or particles are sorted into a different partition (e.g., waste channel), positive charged droplets or particles are sorted into yet another different partition, and multiple-positive charged droplets or particles are sorted into yet another different partition. Electrostatic charging systems are described, e.g., in McCarty and Whitesides, Angew. Chem. 47: 2188-2207, 2008, which is hereby incorporated by reference in its entirety.

### Electrodes

An electrode is an electrical conductor that can be used to generate and exert an electromagnetic force on a droplet or particle. The electromagnetic field exerts a force on a droplet or particle that has an electric or magnetic dipole moment, thereby causing the droplet or particle to move. Electrodes may be used for sorting or detection of droplets or particles. Droplets or particles may be sorted or detected by employing a force (e.g., electromagnetic force) generated by an electrode on a droplet or particle. A barrier and one or more electrodes in operative contact with the barrier may be disposed adjacent to the sorting or detection region (e.g., channel). The one or more electrodes may not be present on the device and instead provided as a separate component. The barrier operatively connects the one or more electrodes with the sorting or detection region (or one or more channels within the sorting or detection region) and the contents thereof. The barrier may be of a suitable thickness to separate the sorting region from the one or more electrodes. The sorting mechanism may employ a force to sort the droplets or particles to a partition in the collection region, e.g., by generating a force from the electrode to move the sorted droplet or particle into a collection region.

When sorting or detecting with an electrode, the device may include one or more (e.g., 2, 3, 4, 5, 6, 7, 8, 9, 10 or more) cavities configured to receive the one or more electrodes to position them adjacent to and in operative contact with the barrier. The barrier is configured to operatively connect the electrode with the sorting region. A cavity is a recessed feature of the device that accommodates the electrode. The cavity may be any suitable geometry sufficient to receive the electrode. For example, a cylindrical or square-shaped tip of an electrode may fit within a cylindrical or square-shaped recessed cavity in the device to fit snugly. This arrangement ensures proper alignment between the electrode and the sorting or detection region (e.g., channel). The cavity may have a cross-sectional width of from about 1 μm to about 100 mm (e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 10 μm-1000 μm, 10 μm-100 μm, 100 μm-1000 μm e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm, e.g., 10 mm-100 mm, e.g., 20 mm, 30 mm, 40 mm, 50 mm, 60 mm, 70 mm, 80 mm, 90 mm, 100 mm).

An electrode may be solid, semisolid (e.g., gel), or a liquid conducting path. For example, metal or other conducting materials may be used in an electrode. Alternatively, a channel containing a liquid, gel, or combination thereof can be used as an electrode. The devices and systems described herein employ one or more (e.g., 2, 3, 4, 5, 6, 7, 8, 9, 10, or more) electrodes to generate a force on a droplet or particle, e.g., a droplet with a particle. Combinations of different types of electrodes may also be employed. The one or more electrodes may contact the device via a barrier disposed between device channel and the electrode. The device may have one or more cavities configured to receive the one or more electrodes and position them adjacent to the barrier. A conductive fluid (e.g., gel or salt buffer) can be disposed between, e.g., the tip of the electrode and the surface of the barrier to establish a robust electrical connection, if necessary.

The electrode may be, for example, an electrical discharge machining (EDM) electrode, anode, cathode, cathode emitter or filament, furnace electrode, or an electrical contact. The electrode may be a dielectrophorersis (DEP) electrode or an electrostatic charger. An electrode may have any suitable geometry in order to operatively connect to a device (e.g., microfluidic device), e.g., via a surface of the device or a barrier disposed between the device and the electrode. The electrode may be composed of any suitable material known to one of skill in the art, such as copper, carbon, graphite, titanium, copper, zinc, brass (e.g., alloy of copper and zinc), silver, platinum, palladium, indium alloy, or a mixed metal oxide (MMO). The material properties can be chosen or adjusted in order to suit the specific purpose of the electrode or the sorting mechanism. For example, properties such as conductivity, corrosion resistance, hardness, form (e.g., shape), and size can vary according to the desired type of sorting.

The one or more electrodes may be patterned or spaced along the barrier or surface of the device at a predetermined width of, e.g., 1 μm-10 mm (e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1 nm, e.g., 200 μm, 300 μm, 400 μm, 500 μm, 600 μm, 700 μm, 800 μm, 900 μm, 1 nm, e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1 μm-10 μm, e.g., 2 μm, 3 μm, 4 μm, 5 μm, 6 μm, 7 μm, 8 μm, 9 μm, 10 μm, e.g., 10 μm-1000 μm, 10 μm-100 μm, 100 μm-1000 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm).

The one or more electrodes may optionally be held in place by a housing or a plurality of housings (e.g., one housing per electrode). The housing positions the electrode adjacent to the barrier, thereby allowing operative contact between the electrode and the barrier. The housing may include any suitable geometric or structural features to physically position the electrode adjacent to the barrier. This assists in maintaining an electrical connection of the electrode in order to optimize the connection between the electrode, the barrier, and the sorting region (e.g., channel). The housing optionally may have a controller, such as a piezoelectric controller, to adjust the position of the housing and/or the electrode.

In some embodiments, the width of the electrodes is between 0.5 μm and 1000 μm. In some embodiments, the width is from 0.5 μm to 10 μm (e.g., 1 μm, 2 μm, 3 μm, 4 μm, 5 μm, 6 μm, 7 μm, 8 μm, or 9 μm), 10 μm to 100 μm (e.g., 20 μm, 30 μm, 40 μm, 50 μm, 60 μm, 70 μm, 80 μm, or 90 μm), 100 μm to 400 μm (e.g., 150 μm, 200 μm, 250 μm, 300 μm, or 350 μm), 400 μm to 700 μm (e.g., 450 μm, 500 μm, 550 μm, 600 μm, or 650 μm), or 700 μm to 1000 μm (e.g., 750 μm, 800 μm, 850 μm, 900 μm, or 950 μm). In some embodiments, the width is from 0.5 μm to 900 μm, 0.5 μm to 800 μm, 0.5 μm to 600 μm, 0.5 μm to 500 μm, 0.5 μm to 400 μm, 0.5 μm to 300 μm, 0.5 μm to 200 μm, 0.5 μm to 100 μm, 1 μm to 80 μm, 2 μm to 60 μm, 3 μm to 50 μm, 4 μm to 40 μm, or 5 μm to 30 μm. In some embodiments, the width is from 3 μm to 50 μm e.g., 3 μm to 5 μm, 5 μm to 10 μm, 10 μm to 20 μm, 20 μm to 30 μm, 30 μm to 40 μm, or 40 μm to 50 μm (e.g., 10 μm, 11 μm, 12 μm, 13 μm, 14 μm, 15 μm, 20 μm, 25 μm, 30 μm, 35 μm, 40 μm, or 45 μm). In general, a smaller width can provide for greater sensitivity, e.g., for detection.

In some embodiments, the width of the electrodes is between 0.5% (i.e., one 200th) and 1000% (i.e., ten times) of the width or depth of the channel. In some embodiments, the width is from 0.5% to 10% (e.g., 1%, 2%, 3%, 4%, 5%, 6%, 7%, 8%, or 9%), 10% to 100% (e.g., 20%, 30%, 40%, 50%, 60%, 70%, 80%, or 90%), 100% to 400% (e.g., 150%, 200%, 250%, 300%, or 350%), 400% to 700% (e.g., 450%, 500%, 550%, 600%, or 650%), or 700% to 1000% (e.g., 750%, 800%, 850%, 900%, or 950%) of the width or depth of the channel. In some embodiments, the width is from 0.5% to 900%, 0.5% to 800%, 0.5% to 600%, 0.5% to 500%, 0.5% to 400%, 0.5% to 300%, 0.5% to 200%, 0.5% to 100%, 1% to 80%, 2% to 60%, 3% to 50%, 4% to 40%, or 5% to 30% of the width or depth of the channel. In some embodiments, the width is from 3% to 200% e.g., 3% to 5%, 5% to 10%, 10% to 20%, 20% to 30%, 30% to 40%, 40% to 50%, 50% to 60%, 60% to 70%, 70% to 80%, 80% to 90%, 90% to 100%, 100% to 110%, 110% to 120%, 120% to 130%, 130% to 140%, 140% to 150%, 150% to 160%, 160% to 170%, 170% to 180%, 180% to 190% (e.g., 10%, 11%, 12%, 13%, 14%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 195%) of the width or depth of the channel.

In some embodiments, the distance between the electrodes can be less than or equal to the width of the electrode. In certain embodiments, the distance between the electrodes can be greater than the width of the electrodes. In some embodiments, the distance between the electrodes is between 0.5 μm and 1000 μm. In some embodiments, the width is from 0.5 μm to 10 μm (e.g., 1 μm, 2 μm, 3 μm, 4 μm, 5 μm, 6 μm, 7 μm, 8 μm, or 9 μm), 10 μm to 100 μm (e.g., 20 μm, 30 μm, 40 μm, 50 μm, 60 μm, 70 μm, 80 μm, or 90 μm), 100 μm to 400 μm (e.g., 150 μm, 200 μm, 250 μm, 300 μm, or 350 μm), 400 μm to 700 μm (e.g., 450 μm, 500 μm, 550 μm, 600 μm, or 650 μm), or 700 μm to 1000 μm (e.g., 750 μm, 800 μm, 850 μm, 900 μm, or 950 μm). In some embodiments, the width is from 0.5 μm to 900 μm, 0.5 μm to 800 μm, 0.5 μm to 600 μm, 0.5 μm to 500 μm, 0.5 μm to 400 μm, 0.5 μm to 300 μm, 0.5 μm to 200 μm, 0.5 μm to 100 μm, 1 μm to 80 μm, 2 μm to 60 μm, 3 μm to 50 μm, 4 μm to 40 μm, or 5 μm to 30 μm. In some embodiments, the width is from 3 μm to 50 μm e.g., 3 μm to 5 μm, 5 μm to 10 μm, 10 μm to 20 μm, 20 μm to 30 μm, 30 μm to 40 μm, or 40 μm to 50 μm (e.g., 4 μm, 5 μm, 6 μm, 7 μm, 8 μm, 9 μm, 10 μm, 11 μm, 12 μm, 13 μm, 14 μm, 15 μm, 20 μm, 25 μm, 30 μm, 35 μm, 40 μm, or 45 μm).

In some embodiments, the distance between the electrodes can be less than or equal to the width of the electrode. In certain embodiments, the distance between the electrodes can be greater than the width of the electrodes. In some embodiments, the distance between the electrodes is between 0.5% and 1000% of the width or depth of the channel. In some embodiments, the distance between the electrodes is from 0.5% to 10% (e.g., 1%, 2%, 3%, 4%, 5%, 6%, 7%, 8%, or 9%), 10% to 100% (e.g., 20%, 30%, 40%, 50%, 60%, 70%, 80%, or 90%), 100% to 400% (e.g., 150%, 200%, 250%, 300%, or 350%), 400% to 700% (e.g., 450%, 500%, 550%, 600%, or 650%), or 700% to 1000% (e.g., 750%, 800%, 850%, 900%, or 950%) of the width or depth of the channel. In some embodiments, the distance between the electrodes is from 0.5% to 900%, 0.5% to 800%, 0.5% to 600%, 0.5% to 500%, 0.5% to 400%, 0.5% to 300%, 0.5% to 200%, 0.5% to 100%, 1% to 80%, 2% to 60%, 3% to 50%, 4% to 40%, or 5% to 30% of the width or depth of the channel. In some embodiments, the distance between the electrodes is from 3% to 200% e.g., 3% to 5%, 5% to 10%, 10% to 20%, 20% to 30%, 30% to 40%, 40% to 50%, 50% to 60%, 60% to 70%, 70% to 80%, 80% to 90%, 90% to 100%, 100% to 110%, 110% to 120%, 120% to 130%, 130% to 140%, 140% to 150%, 150% to 160%, 160% to 170%, 170% to 180%, 180% to 190% (e.g., 10%, 11%, 12%, 13%, 14%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, or 195%) of the width or depth of the channel.

In some embodiments, the electrode can be any micro-electro-mechanical systems (MEMS) material well known in the art. Electrode materials can include, but are not limited to, titanium, gold, silver, platinum, aluminum, copper, lithium, carbon (e.g., polyacrylonitrile (PAN)), and nickel.

While the foregoing sections describe electrodes for sorting, one of skill in the art would appreciate that the same electrodes and/or features may also be used for detection as described herein.

The devices and systems for electrode-based sorting described herein may include a barrier disposed between the one or more electrodes and the device (e.g., the sorting region of the device or one or more channels in the sorting region). The barrier is configured to operatively connect the electrode with the channel, thereby establishing an electrical connection between the electrode and the channel. The barrier may have any suitable thickness to separate the electrode and the device while still maintaining an operative connection. For example, the barrier may have a thickness of from about 1 μm to about 1 mm (e.g., 1-500 μm, 1-200 μm, 1-100 μm, 5 μm-100 μm, 5 μm-50 μm, and 10 μm-50 μm) or a thickness of at least 1 μm (e.g., at least 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, or 1000 μm).

The barrier can be made of any suitable material sufficient to separate the electrode and the device and maintain an electrical connect. Suitable materials include, for example, polymers, such as acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof.

The barrier may be positioned on the top, bottom, side of the device. The barrier may be positioned inside of or external to the device. The electrode can be positioned from the top, bottom, or side to contact the barrier adjacent a sorting region (e.g., channel). If using, for example, a microfluidic device, the electrode may contact the device from, e.g., the top or bottom, and the barrier may include a layer (e.g., laminate layer, e.g., plastic) that covers the top or bottom of the microfluidic device to operatively connect the electrode and the sorting region (e.g., channel).

### Collection Region

The invention provides devices that may include a collection region. A collection region includes one or more partitions to receive droplets or particles from the detection and/or sorting region and may be in fluid communication with, e.g., fluidically connected to, the detection and/or sorting region. A collection region or the one or more partitions within a collection region can be of any suitable geometry and may be or include, for example, a well, channel, reservoir, or portion thereof, or the like. The collection region can be open-ended (e.g., connected to subsequent partitions, e.g., channels or reservoirs) or enclosed. The collection region may include one or more (e.g., 2, 3, 4, 5, 6, 7, 8, 9, 10, 20, 30, 40, 50, 60, 70, 80, 90, 100, 200, 300, 400, 500, 600, 700, 800, 900, 1000, or more) partitions (e.g., channels or reservoirs) configured to receive the droplets or particles after sorting or detection. The one or more partitions in the collection region can have any length, width, and height suitable for receiving one or more droplets or particles. For example, the length, width, and height may be independently, e.g., 1 μm-10 mm (e.g., 1 μm, 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1 nm, e.g., 200 μm, 300 μm, 400 μm, 500 μm, 600 μm, 700 μm, 800 μm, 900 μm, 1 nm, e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1 μm-10 μm, e.g., 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm). In some embodiments, the collection region has no cross-sectional dimension of less than 1 mm. For example, each cross-sectional dimension of the collection region has a length of at least 1 mm (e.g., 2 mm, 3 mm, 4 mm, 5 mm, 6 mm, 7 mm, 8 mm, 9 mm, 10 mm, 20 mm, 30 mm, 40 mm, 50 mm, 60 mm, 70 mm, 80 mm, 90 mm, 100 mm, or more). The one or more partitions may have one or more dividers between them to physically separate the sorted droplets or particles. A divider may be any feature that can obstruct or prevent the droplets or particles from moving into a different partition, thereby unsorting the sorted droplets or particles. A divider may be an insert in or between partitions or may be, e.g., a hollow cylindrical or partially cylindrical insert configured to fit within a cylindrical well. For example, a collection region may include multiple adjacent partitions, with each partition separated from its neighboring partition by a divider. This provides separation between the partitions so that the droplets or particles within each partition cannot mix with the droplets or particles in the neighboring partition, and the sorted populations of droplets or particles are maintained as separate populations.

### Detection Region

The devices of the invention may include a detection region. A detection region may be used to detect one or more droplets or particles. In devices for sorting droplets or particles, droplets or particles may be detected, for example, prior to, or following, sorting. The detection region may optionally include one or more sensors that are used to detect one or more features or characteristics of a droplet. Upon sensing the presence or absence of the feature or characteristic, the one or more sensors may provide feedback to the sorting mechanism (e.g., electrode), if present, in the sorting region to actuate a particular mode of sorting. The source, which may be positioned distal to (e.g., downstream of) a droplet or particle formation region, may be configured to provide droplets or particles within a continuous phase to the detection region.

The detection region may be, for example, in a channel or a reservoir. In some embodiments, the depth and/or width of the channel or reservoir is between about 0.1 μm and 1000 μm. In some embodiments, the depth and/or width is from 1 to 750 μm, 1 to 500 μm, 1 to 250 μm, 1 to 100 μm, 1 to 50 μm, or 3 to 40 μm. The width and depths of the channel or reservoir may or may not be constant over its length. In particular, the width may increase or decrease adjacent the distal end. In general, the channel or reservoir may be of any suitable cross section, such as a rectangular, triangular, or circular, or a combination thereof. In particular embodiments, a channel or reservoir may include a groove along the bottom surface. The width or depth of the channel or reservoir may also increase or decrease, e.g., in discrete portions, to alter the rate of flow of liquid or particles or the alignment of particles. The detection channel or reservoir can have any length, width, and height suitable for detecting one or more droplets or particles. For example, the length, width, and height may be independently, e.g., 1 nm-10 mm (e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1-10 μm, e.g., 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm).

Droplets or particles can be optically detectable, e.g., using a conventional optical microscope or with bright-field microscopy, as described herein. In some embodiments, droplets or particles are detectable by light absorbance, scatter, and/or transmission. Additionally or alternatively, optical detection can include fluorescent detection, e.g., by fluorescence microscopy. In still further embodiments, devices can be configured for detection of cells or particulate components thereof having electrical, optical, or magnetic labels. Other modes of detection are known in the art, and include, e.g., light scatter (e.g., side, forward, and/or back scatter), obscuration, imaging, or impedance. The detector may include a sensor, such as an optical, electrical, magnetic, impedance, or fluorescent sensor. The sensor may sense a particular feature (e.g., fluorescence, charge) or characteristic (e.g., diameter or volume) and provide feedback to the sorter to actuate sorting of droplets or particles thereof in a certain manner.

The detection region may include a detector. The detector may be any detector capable of measuring light (e.g., the scattered or attenuated light) from the droplet or particle, e.g., a spectrometer, a light meter, a photometer, a photodiode, a photomultiplier tube, a CCD array, a CMOS sensor, or a photovoltaic device.

In one embodiment, the detection region includes a wave plate (e.g., quarter wave plate). The detection region may further include a reflector configured to reflect light back through the wave plate.

In some embodiments, the droplet or particle source is the point at which the droplet or particle exits a channel and enters a reservoir, such as a well. In this embodiment, the droplet or particle source may be any suitable distance from a droplet or particle formation region. In some instances, the droplet or particle may pass through the droplet or particle source soon or immediately after it has been formed. For example, the droplet or particle source can be at or downstream of the droplet formation region at a distance from 1 μm to 1000 mm (e.g., from 1 μm to 100 mm, e.g., from 1 μm to 10 mm, from 5 μm to 5 mm, from 10 μm to 1 mm, or from 50 μm to 500 μm, e.g., from 1 μm to 2 μm, from 2 μm to 4 μm, from 4 μm to 6 μm, from 6 μm to 8 μm, from 8 μm to 10 μm, from 10 μm to 20 μm, from 20 μm to 40 μm, from 40 μm to 60 μm, from 60 μm to 80 μm, from 80 μm to 100 μm, from 100 μm to 200 μm, from 200 μm to 400 μm, from 400 μm to 600 μm, from 600 μm to 800 μm, from 800 μm to 1 mm, from 1 mm to 2 mm, from 2 mm to 4 mm, from 4 mm to 6 mm, from 6 mm to 8 mm from 8 mm to 10 mm, from 10 mm to 50 mm, from 50 mm to 100 mm, from 100 mm to 200 mm, from 200 mm to 300 mm, from 300 mm to 400 mm, from 400 mm to 500 mm, from 500 mm to 600 mm, from 600 mm to 700 mm, from 700 mm to 800 mm, from 800 mm to 900 mm, or from 900 mm to 1000 mm, e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1 mm, about 10 mm, about 20 mm, about 30 mm, about 40 mm, about 50 mm, about 60 mm, about 70 mm, about 80 mm, about 90 mm, about 100 mm, or more). Alternatively, droplets or particles may be provided to the source from a reservoir holding previously formed droplets or particles.

Upon emerging from the droplet or particle source (e.g., a droplet or particle formation region), a droplet or particle may float or sink, depending on the geometry of the device and whether its density is less than or greater than the continuous phase. A surface (i.e., deflecting surface) in fluid communication with the droplet or particle source may deflect the droplet or particle laterally, e.g., in the same lateral direction of egress from the droplet or particle source. For example, as a droplet or particle having a lower density than the continuous phase flows from the droplet or particle source into an open volume, it rises, until the top of the droplet or particle contacts the deflecting surface. The droplet or particle then flows laterally along the surface until reaching the end of the surface.

The deflecting surface can position the droplets or particles for detection by deflecting a stream of droplets or particles to allow detection of individual droplets or particles. For example, a detector (e.g., a microscope objective) may be substantially beneath a stream of droplets or particles as they emerge from the droplet or particle source. In the absence of a deflecting surface, the droplets or particles align with the detector and overlap in the detection region, thereby obstructing a view of any single droplet or particle. In the presence of a deflecting surface, the droplets or particles are deflected such that individual droplets or particles are unobstructed by the adjacent droplets or particles. In some embodiments, the droplets or particles flow through the detection region one-by-one. A detector may be, for example, a photoelectric, photoemission, thermal energy, semiconductor, photovoltaic, polarization, photochemical, graphene/silicone, or weak interaction effect detector. Specific examples include CCD and CMOS sensors, photodiodes, and photomultiplier tubes. Other types of light detectors are well known to the skilled artisan.

The deflecting surface can be at any suitable angle to achieve particle detection described herein. In embodiments in which the droplets or particles float in the continuous phase, the surface can be at an angle from 10° to 80° above a horizontal plane (e.g., from 10° to 70°, from 15° to 60°, from 20° to 50°, from 25° to 45°, or from 30° to 40° above a horizontal plane, e.g., from 10° to 15°, from 15° to 20°, from 20° to 25°, from 25° to 30°, from 30° to 35°, from 35° to 40°, from 40° to 45°, from 45° to 50°, from 50° to 55°, from 55° to 60°, from 60° to 65°, from 65° to 70°, from 70° to 75°, or from 75° to 80° above a horizontal plane, e.g., about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 31°, about 32°, about 33°, about 34°, about 35°, about 36°, about 37°, about 38°, about 39°, about 40°, about 41°, about 42°, about 43°, about 44°, about 45°, about 46°, about 47°, about 48°, about 49°, about 50°, about 51°, about 52°, about 53°, about 54°, about 55°, about 56°, about 57°, about 58°, about 59°, about 60°, about 61°, about 62°, about 63°, about 64°, about 65°, about 66°, about 67°, about 68°, about 69°, about 70°, about 71°, about 72°, about 73°, about 74°, about 75°, about 76°, about 77°, about 78°, about 79°, or about 80° above a horizontal plane). In embodiments in which the droplets or particles sink in the continuous phase, the deflecting surface can be at an angle from 10° to 80° below a horizontal plane (e.g., from 10° to 70°, from 15° to 60°, from 20° to 50°, from 25° to 45°, or from 30° to 40° below a horizontal plane, e.g., from 10° to 15°, from 15° to 20°, from 20° to 25°, from 25° to 30°, from 30° to 35°, from 35° to 40°, from 40° to 45°, from 45° to 50°, from 50° to 55°, from 55° to 60°, from 60° to 65°, from 65° to 70°, from 70° to 75°, or from 75° to 80° below a horizontal plane, e.g., about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 31°, about 32°, about 33°, about 34°, about 35°, about 36°, about 37°, about 38°, about 39°, about 40°, about 41°, about 42°, about 43°, about 44°, about 45°, about 46°, about 47°, about 48°, about 49°, about 50°, about 51°, about 52°, about 53°, about 54°, about 55°, about 56°, about 57°, about 58°, about 59°, about 60°, about 61°, about 62°, about 63°, about 64°, about 65°, about 66°, about 67°, about 68°, about 69°, about 70°, about 71°, about 72°, about 73°, about 74°, about 75°, about 76°, about 77°, about 78°, about 79°, or about 80° below a horizontal plane).

Additionally, or alternatively, the deflecting surface can have more than one angle or a variable angle (e.g., a curve, e.g., a concave or convex surface). The angle or curvature of the deflecting surface can be selected to provide a suitable speed and/or position of a floating or sinking droplet or particle, e.g., at the detection region, which can be adapted for a particular means of detection (e.g., based on frame-rate of image acquisition or video).

To facilitate detection (e.g., optical detection), the deflecting surface can be made, wholly or partially, from a transparent material, e.g., to allow light to pass through the surface (e.g., to a reflective surface above, e.g., at the top of the well). Such a transparent material can have a refractive index that about matches the refractive index of the continuous phase. For example, the refractive index can be within 10%, within 9%, within 8%, within 7%, within 6%, within 5%, within 4%, within 3%, within 2%, within 1%, within 0.5%, within 0.1%, within 0.05%, or within 0.01% of the refractive index of the continuous phase.

The refractive index of the deflecting surface can be from 1.3 to 1.6 (e.g., from 1.4 to 1.55 or from 1.45 to 1.50, e.g., from 1.3 to 1.35, from 1.35 to 1.40, from 1.40 to 1.45, from 1.45 to 1.50, from 1.50 to 1.55, or from 1.55 to 1.60, e.g., about 1.30, about 1.31, about 1.32, about 1.33, about 1.333, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, or about 1.60). In some instances, the refractive indexes of the deflecting surface and the continuous phase are both from 1.3 to 1.6 (e.g., from 1.4 to 1.55 or from 1.45 to 1.50, e.g., from 1.3 to 1.35, from 1.35 to 1.40, from 1.40 to 1.45, from 1.45 to 1.50, from 1.50 to 1.55, or from 1.55 to 1.60, e.g., about 1.30, about 1.31, about 1.32, about 1.33, about 1.333, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, or about 1.60).

In other embodiments, the deflecting surface can be black or opaque (e.g., wholly or partially opaque, or translucent) to facilitate detection in instances that do not require light transmission through the surface, e.g., fluorescent detection.

The deflecting surface can be made of any suitable materials, such as polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof.

A droplet or particle enters the sorting region or collection region upon traversing the deflecting surface. In some embodiments, the collection region is defined by a volume in a reservoir (e.g., a well) that is unoccupied by the surface and its supporting structures. For example, in a device configured to detect floating droplets or particles in a well, a deflecting surface may be disposed on a downward-facing surface of a structure that can be inserted into the well (i.e., an insert), occupying a portion of its volume. After emerging from a droplet or particle source at or near the bottom of the well, droplets or particles are deflected by the downward facing surface and, after passing the edge of the deflecting surface, continue to rise into a collection region to the side of the insert.

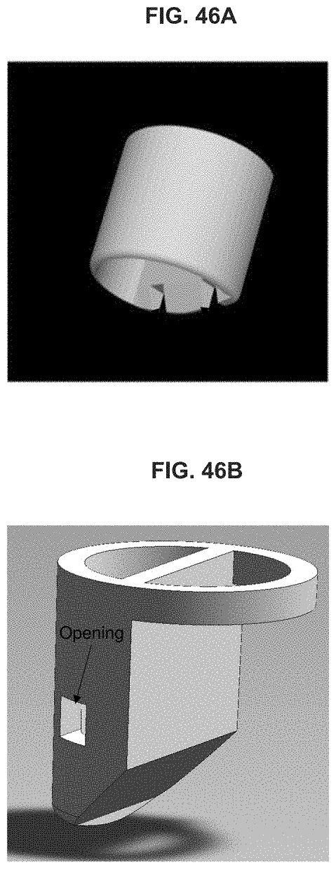

Thus, an insert can define one or more boundaries of the collection region. In some instances, an insert can define all lateral boundaries of the collection region, e.g., as a hollow cylindrical or partially cylindrical insert configured to fit within a cylindrical well. In some embodiments, an insert defines all lateral boundaries of a continuous phase reservoir (except at one or more openings configured to shunt the continuous phase) and does not define all lateral boundaries of the collection region (e.g., as in the device shown in FIG. 46).

The insert can have a size and shape suitable to occupy a low volume of the reservoir in order to provide a suitable collection region volume. For example, the collection region can occupy from 10% to 99% of the lateral area of the reservoir (e.g., from 15% to 98%, from 20% to 97%, from 25% to 96%, from 30% to 95%, from 35% to 90%, from 40% to 85% from 45% to 80%, or from 50% to 75% of the lateral area of the reservoir, e.g., from 10% to 15%, from 15% to 20%, from 20% to 25%, from 25% to 30%, from 30% to 35%, from 35% to 40%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 99% of the lateral area of the reservoir, e.g., about 40%, about 45%, about 50%, about 55%, about 60%, about 65%, about 70%, about 75%, about 80%, about 85%, about 90%, or about 95% of the lateral area of the reservoir). Alternatively, the device can be made so that the deflecting surface and collection region are not separate.

The invention further provides elements that enhance the capacity of the collection region to collect droplets or particles. For example, the device can be configured to shunt the continuous phase from the collection region to a separate reservoir (i.e., a continuous phase reservoir) as droplets or particles accumulate in the droplet or particle collection region. A structure, such as that on which the deflecting surface is disposed (e.g., an insert), can feature one or more openings (e.g., one, two, three, four, or more openings) that render the droplet or particle detection region and the droplet or particle collection region in fluid communication with a continuous phase reservoir. The one or more openings can be positioned to prevent droplets or particles from flowing into the continuous phase reservoir while allowing the continuous phase to freely pass in and out. For example, the one or more openings can be disposed near the bottom of a device configured for detecting floating droplets or particles. Additionally, or alternatively, the one or more openings can be positioned to either side of the stream of droplets or particles as they emerge from the droplet or particle source.

The continuous phase reservoir can occupy from 5% to 50% of the lateral area of the reservoir (e.g., from 10% to 45%, from 15%, to 40%, or from 20% to 30% of the lateral area of the reservoir, e.g., from 5% to 10%, from 10% to 15%, from 15% to 20%, from 20% to 25%, from 25% to 30%, from 30% to 35%, from 35% to 40%, or from 45% to 50% of the lateral area of the reservoir, e.g., about 5%, about 10%, about 15%, about 20%, about 25%, about 30%, about 35%, about 40%, about 45%, or about 50% of the lateral area of the reservoir).

In one embodiment, the detection region includes a wave plate (e.g., quarter wave plate). The detection region may further include a reflector configured to reflect light back through the wave plate. In some embodiments, the wave plate includes a reflective surface or coating. In some embodiments, the device includes a polarizer (e.g., a polarization beam splitter) to provide polarized light for droplet or particle detection. The polarizer is configured to polarize light from a light source in a first polarization and direct it to the detection region. The polarizer may also be configured to direct light polarized in a second polarization (e.g., orthogonal to the first polarization) to a detector, e.g., when the light is reflected back through the wave plate by a reflector or a reflective coating on the wave plate.

The devices described herein provide enhanced imaging by reducing background noise during imaging. A wave plate rotates the polarization of light, thereby providing the ability to differentiate signal and noise. Without a wave plate (e.g., with a reflective plate), light detected contains both background noise and useful image signal that were reflected from the device. Since both the signal and noise are unpolarized or have the same polarization, the detector cannot differentiate between the two. In the invention, the illumination signal is directed towards a wave plate, which then rotates the polarization of the light. Thus, the useful image signal has an altered polarization signal from the noise. Therefore, the signal and the noise can be more effectively differentiated during imaging. The devices described herein may also include a polarizer to polarize the light before directed to the wave plate. Various embodiments of the devices described herein are described in more detail below (Example 42).

In some instances, the detection region includes one or more (e.g., 1, 2, 3, 4, 5, 6, 7, 8, 9, 10, or more) reflectors. For example, the deflecting surface can feature a reflective portion across which droplets or particles can flow. The one or more reflectors can be used in devices configured for optical detection, e.g., by bright-field imaging, e.g., bright-field microscopy. In some instances, a reflector can be within a portion on the deflecting surface, e.g., as a flat surface in an angled deflecting surface. Such a configuration can provide a perpendicular surface to align reflected light toward the detector, while providing a suitably angled surface for lateral deflection of droplets or particles. All or a portion of the deflecting surface can be adapted as a reflector by coating the surface with a reflective material, such as a reflective paint or tape (e.g., chrome paint or aluminum tape, etc.).

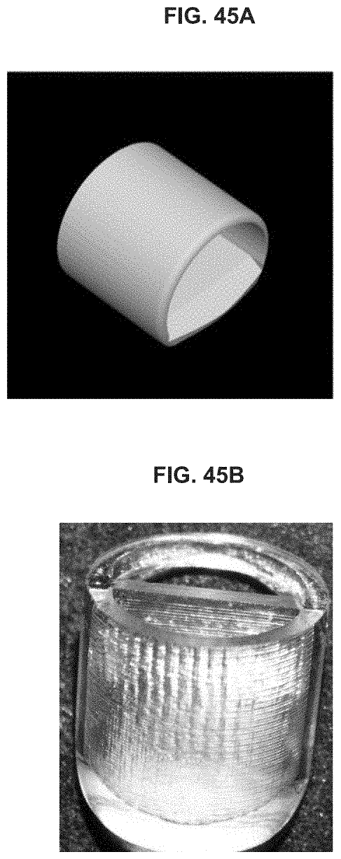

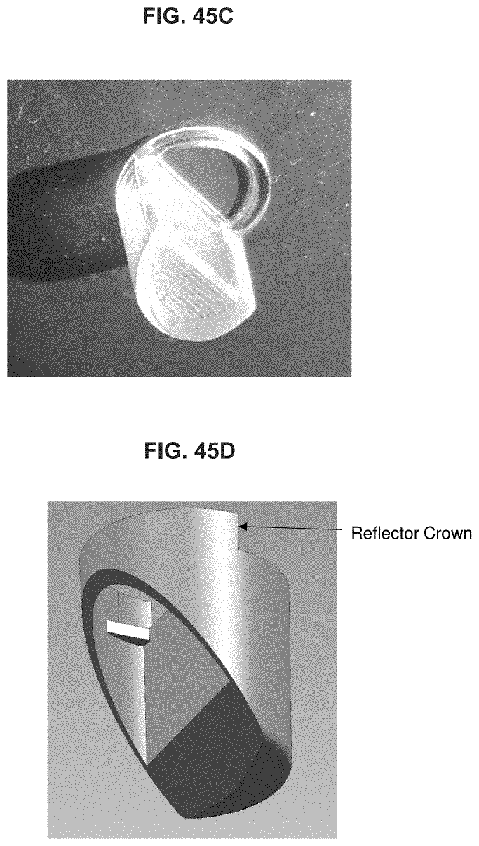

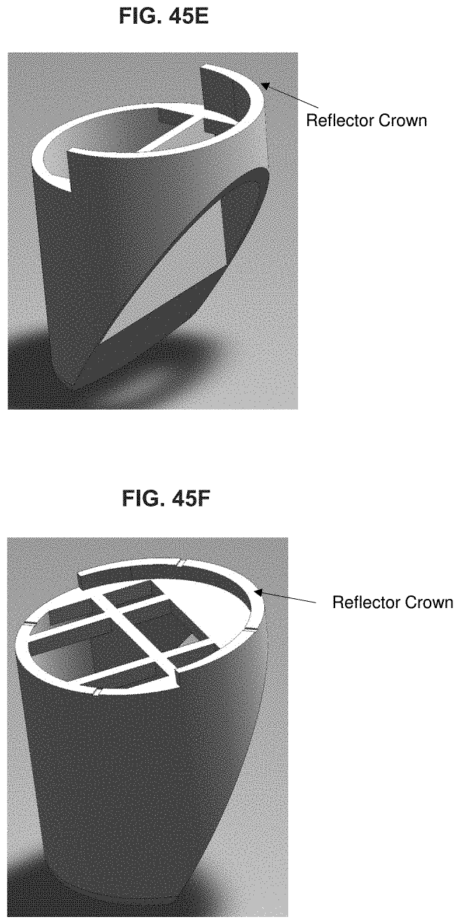

Alternatively, a reflector can be disposed beyond the deflecting surface (e.g., at or near the top of a device having a low droplet or particle source for floating droplets or particles, or vice-versa). For example, in some instances, a reflector (e.g., a mirror), is at the top of the well to reflect light downward toward a detector positioned below the detection region. In such embodiments, a device (e.g., an insert) can include one or more structural features configured to hold or stabilize the reflector. For example, a device can include a lip or crown (e.g., a portion that protrudes beyond another edge of the device, such as that shown in FIGS. 45D and 45E). In some embodiments, the crown extends about halfway around the top of the device, as shown in FIGS. 45D and 45E. In other embodiments, the crown extends completely around the top of the device to surround all edges of the reflector.

Droplets or particles can be optically detectable, e.g., using a conventional optical microscope or with bright-field microscopy, as described herein. In some embodiments, droplets or particles are detectable by light absorbance, scatter, and/or transmission, e.g., by dark-field microscopy, phase-contrast microscopy, or cross-polarized light microscopy. Additionally, or alternatively, optical detection can include fluorescent detection, e.g., by fluorescent microscopy. In still further embodiments, devices can be configured for detection of droplets or particles having electrical or magnetic labels.

Detection may employ a piezoelectric element. The piezoelectric element can have various shapes and sizes. The piezoelectric element may have a shape or cross-section that is circular, triangular, square, rectangular, or partial shapes or combination of shapes thereof. The piezoelectric element can have a thickness from about 100 femtometers (fm) to about 100 millimeters (mm). The piezoelectric element can have a dimension (e.g., cross-section) of at least about 1 mm. The piezoelectric element can be formed of, for example, lead zirconate titanate, zinc oxide, barium titanate, potassium niobate, sodium tungstate, Ba₂NaNb₅O₅, and Pb₂KNb₅O₁₅. The piezoelectric element, for example, can be a piezo crystal. The piezoelectric element may contract when a voltage is applied and return to its original state when the voltage is unapplied. Alternatively, the piezoelectric element may expand when a voltage is applied and return to its original state when the voltage is unapplied. Alternatively, or in addition, application of a voltage to the piezoelectric element can cause mechanical stress, vibration, bending, deformation, compression, decompression, expansion, and/or a combination thereof in its structure, and vice versa (e.g., applying some form of mechanical stress or pressure on the piezoelectric element may produce a voltage). In some instances, the piezoelectric element may include a composite of both piezoelectric material and non-piezoelectric material. Interdigital transducers (IDTs) can be also patterned on top of piezoelectric element to generate an acoustic wave at a certain frequency, depending on the size and distance of the fingers on the IDTs.

In some instances, the piezoelectric element may be in a first state when no electrical charge is applied, e.g., an equilibrium state. When an electrical charge is applied to the piezoelectric element, the piezoelectric element may bend backwards, pulling a part of the moving (e.g., oscillating) detector or lens outwards. When the electrical charge is altered, the piezoelectric element may bend in another direction (e.g., inwards towards the contents of the oscillating detector or lens), pushing a part of the detector or lens inwards. After the detector or lens is moved (e.g., oscillated), the piezoelectric element may return to the first state. The cycle can be repeated (e.g., 1, 2, 3, 4, 5, 6, 7, 8, 9, 10, 11, 12, 13, 14, 15, 16, 17, 18, 19, 20, or more times) to move (e.g., oscillate) the detector or lens multiple (e.g., more than 1, such as 2, 3, 4, 5, 6, 7, 8, 9, 10, 11, 12, 13, 14, 15, 16, 17, 18, 19, 20, or more) times.

While the above non-limiting example describes bending of the piezoelectric element in response to application of an electrical charge, the piezoelectric element may undergo or experience vibration, bending, deformation, compression, decompression, expansion, other mechanical stress and/or a combination thereof upon application of an electrical charge, which movement may be translated to the oscillating detector or lens.

The frequency of application of electrical charge to the piezoelectric element may be adjusted to control the speed. For example, the frequency of oscillation may increase with the frequency of alternating electrical charge. Additionally, the material of the piezoelectric element, number of piezoelectric elements in the oscillating detector or lens, the location of the piezoelectric elements, strength of the electrical charge applied, and other factors may be adjusted to control oscillation of the oscillating detector or lens. For example, without wishing to be bound by a particular theory, if the strength of the electrical charge applied is increased, the mechanical stress experienced by the piezoelectric element may be increased, which can increase the impact on the structural deformation of the oscillating detector or lens, resulting in increased oscillation.

Alternatively, or in addition, one or more piezoelectric elements may be used to control oscillation acoustically (e.g., for acoustic oscillation of the oscillating detector or lens).

The piezoelectric element can be in electrical communication with a controller. The piezoelectric element can be responsive to (e.g., excited by) an electric voltage driven at radio frequency (RF). In some embodiments, the piezoelectric element can be made from zinc oxide (ZnO).

Droplet or particle detection devices may include a droplet or particle source and a detection channel having a light source or light guide and one or more (e.g., 2, 3, 4, 5, 6, 7, 8, 9, 10, 20, 30, 40, 50, 60, 70, 80, 90, or 100, or more) detectors or light guides. The detection channel is downstream of the droplet or particle source, e.g., at a distance from 1 μm to 1000 mm (e.g., from 1 μm to 10 mm, from 5 μm to 5 mm, from 10 μm to 1 mm, or from 50 μm to 500 μm, e.g., from 1 μm to 2 μm, from 2 μm to 4 μm, from 4 μm to 6 μm, from 6 μm to 8 μm, from 8 μm to 10 μm, from 10 μm to 20 μm, from 20 μm to 40 μm, from 40 μm to 60 μm, from 60 μm to 80 μm, from 80 μm to 100 μm, from 100 μm to 200 μm, from 200 μm to 400 μm, from 400 μm to 600 μm, from 600 μm to 800 μm, from 800 μm to 1 mm, from 1 mm to 2 mm, from 2 mm to 4 mm, from 4 mm to 6 mm, from 6 mm to 8 mm from 8 mm to 10 mm, from 10 mm to 50 mm, from 50 mm to 100 mm, from 100 mm to 200 mm, from 200 mm to 300 mm, from 300 mm to 400 mm, from 400 mm to 500 mm, from 500 mm to 600 mm, from 600 mm to 700 mm, from 700 mm to 800 mm, from 800 mm to 900 mm, or from 900 mm to 1000 mm, e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1 mm, about 10 mm, about 20 mm, about 30 mm, about 40 mm, about 50 mm, about 60 mm, about 70 mm, about 80 mm, about 90 mm, about 100 mm, or more). The droplet or particle source may be a droplet formation region or a reservoir (e.g., a reservoir holding previously formed droplets and/or particles).

As described herein, reference to a light source or detector may also be a reference to a light guide that mates with a light source or detector to conduct light to or from the source or detector.

A device of the invention may include a detection channel having a proximal end and a distal end and a light source and one or more detectors. The channel, the light source, and the one or more detectors are positioned to measure the light from a particle as it passes through the detection channel. The light source may be positioned above, below, or to the side of the channel, and the one or more detectors may be placed on the same side of the light source, a different side (e.g., the opposite side) of the light source or light guide, or any combination thereof.

Without wishing to be bound by theory, droplets or particles can pass through the channel at a rate determined by the flow of the continuous phase from the proximal end to the distal end. As the continuous phase continues to flow through the channel, it allows the droplets or particles to pass the light source, wherein the scattered or attenuated light from the droplet and/or particle is measured by the one or more detectors. It will be understood that the continuous phase may be externally driven, e.g., by gently stirring or vibration but such motion is not necessary where the droplets and/or particles move relative to the continuous phase, e.g., by differences in density. The light source may be any element that produces photons, such as a laser or a light emitting diode (LED).

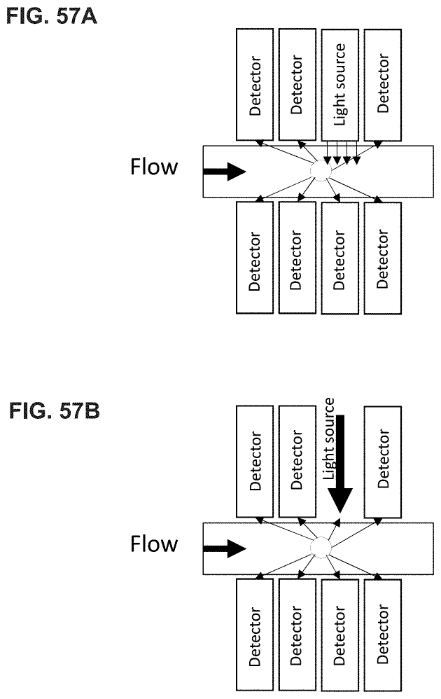

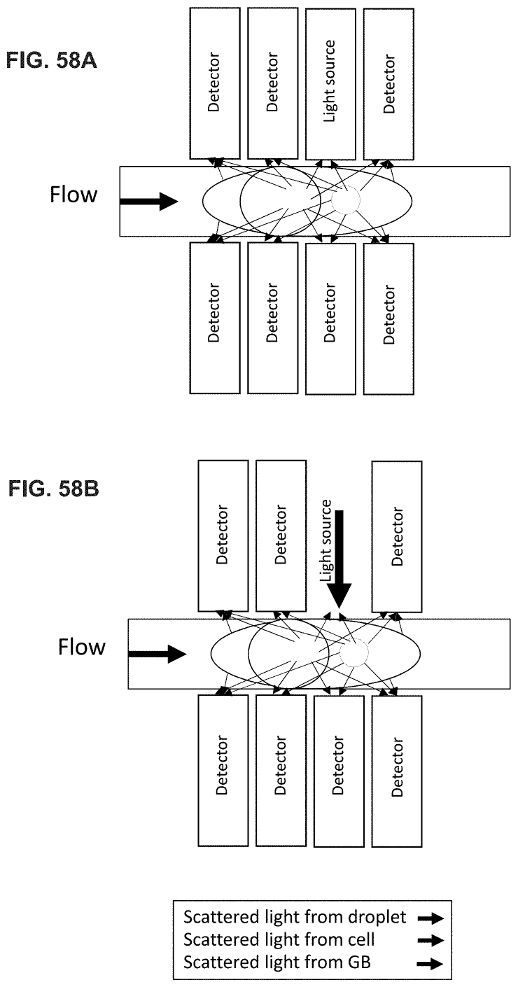

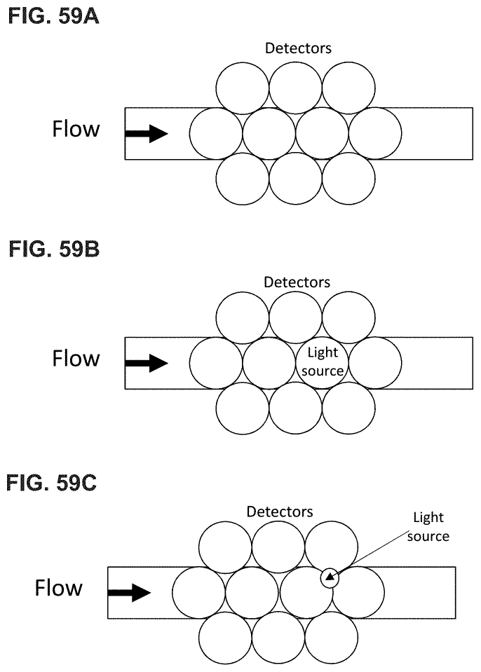

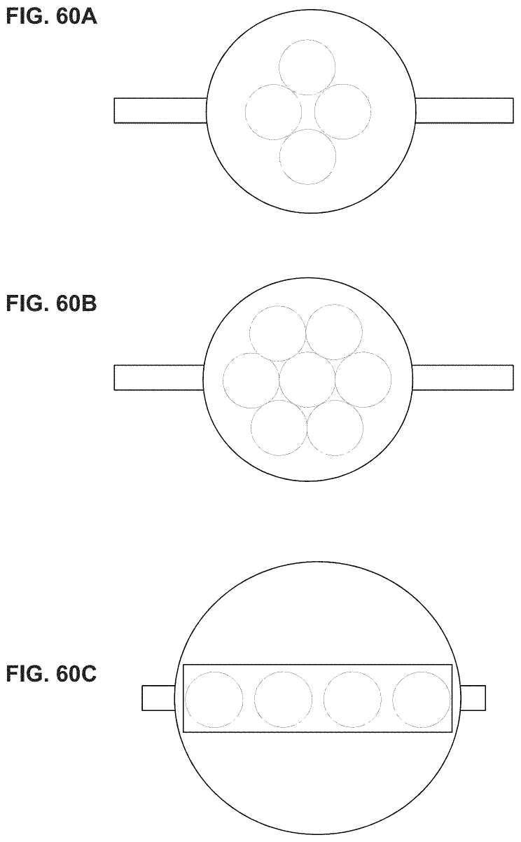

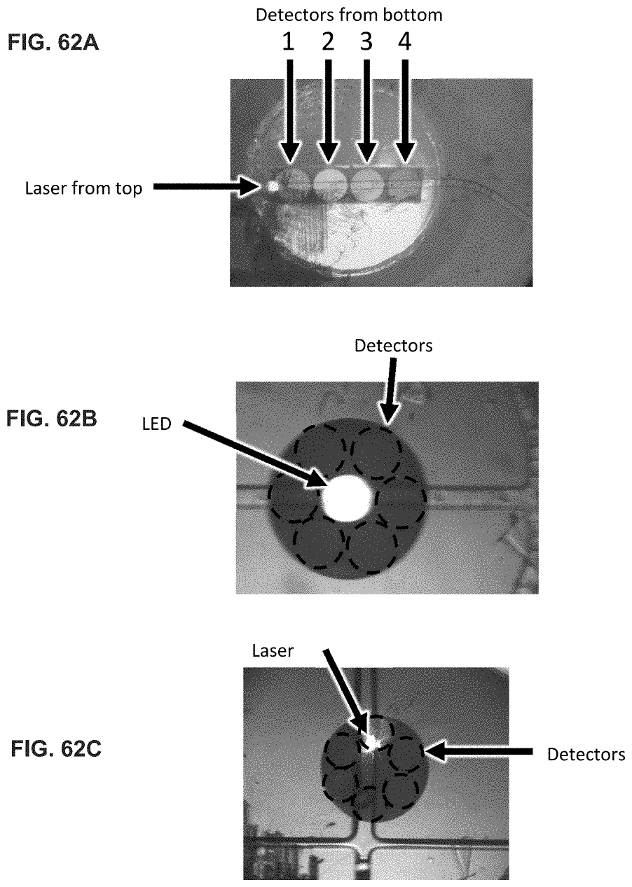

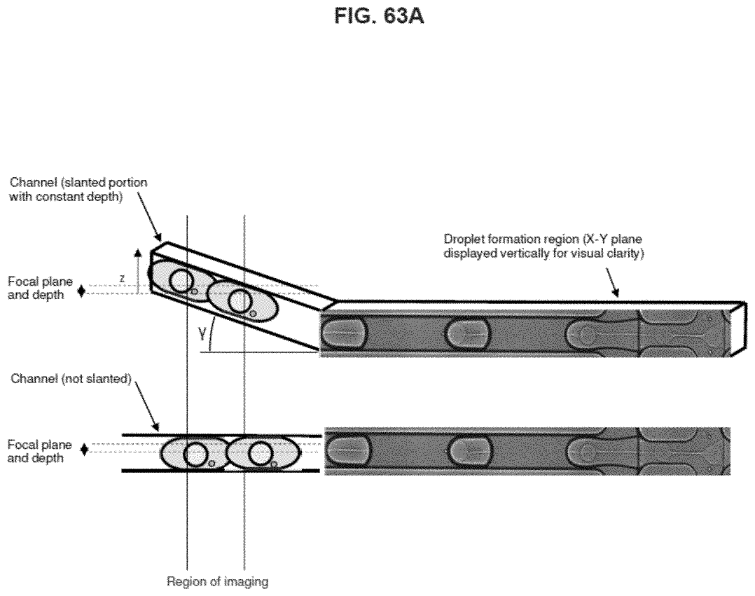

The devices described herein may include one or more detectors positioned on one or more sides of the detection channel. The detectors may be positioned in an orientation to detect a sufficient amount of scattered and/or attenuated light. For example, the detectors may be arranged in a geometrical pattern that minimizes gaps between the detectors (see, e.g., FIGS. 59A-59C and 60A-60C). The detectors may be arranged in a circular, polygonal, or linear pattern. The detectors may have any suitable shape, such as a square, rectangle, circle, or ellipse. In some embodiments, the detectors are positioned along one side of the detection channel. In other embodiments, the light source is positioned between two or more detectors (FIGS. 57A-57B, 58A-58B, and 62A-62C).

The detectors can have any length, width, and height suitable for detecting one or more droplets or particles. For example, the length, width, and height may be independently, e.g., 1 nm-10 mm (e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1-10 μm, e.g., 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm).

In some embodiments, the light source or detectors is positioned to deliver light to the detection channel or detect light from the detection channel. In another embodiment the light source or detector is connected to a light guide (e.g., a waveguide, fiber optic, or liquid light guide) positioned to transmit light between the channel and the source or detector.

Devices may include multiple droplet or particle sources, e.g., to increase the rate at which a droplet or particle is detected. In general, throughput may significantly increase by increasing the number of droplet or particle sources in a device. For example, a device having five droplet or particle sources may measure the light of five times as many droplets or particles simultaneously relative to a device having one droplet or particle source, provided that the liquid flow rate is about the same. A device may have as many channels as is practical and allowed for the size of the source of liquid, e.g., reservoir. For example, the device may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 2000 or more channels. Inclusion of multiple channels may require the inclusion of channels that traverse but do not intersect, e.g., the flow path is in a different plane. Multiple detection channels may be in fluid communication with, e.g., fluidically connected to, a separate source. In other embodiments, two or more detection channels are in fluid communication with, e.g., fluidically connected to, the source, e.g., where the multiple detection channels branch from a single, upstream channel. The number channels in fluid communication with an upstream channel may be between 3 and 10 (e.g., 3, 4, 5, 6, 7, 8, 9, or 10). Alternatively or in addition, the throughput of detection (e.g., via impedance) can be increased by increasing the flow rate of the continuous phase.

Devices of the invention may include a droplet or particle source (e.g., a droplet formation region) configured for providing droplets, and a channel aligned with one or more detectors or lenses for optical detection of the droplets or particles (e.g., droplets that are formed in and/or provided by the droplet source). In some embodiments, the channel has a slanted portion (e.g., a slanted portion with constant depth or a slanted portion with variable depth). As droplets or particles flow along the slanted portion of the channel, the droplets or particles pass through a focal plane of a detector or lens with which the channel is aligned, allowing the detection of multiple cross sections of the droplets or particles in sequence. In other embodiments, the channel is aligned with multiple detectors or lenses having different focal planes, allowing detection of droplets or particles at the different focal planes as the droplets or particles flow along the channel. Alternatively, the channel is aligned with a moving detector or lens. Movement (e.g., piezoelectric or acoustic oscillation) allows detection of droplets or particles at multiple focal planes of the oscillating detector or lens, as the droplets or particles flow along the channel. In further embodiments, the channel is constricted to position droplets or particles and/or contents of droplets (e.g., particles, such as cells and/or one or more beads within the droplets) in a single plane, such as a focal plane for detection.

The channel of the device may be aligned with one or more (e.g., 1, 2, 3, 4, 5, 6, 7, 8, 9, 10, 11, 12, 13, 14, 15, 16, 17, 18, 19, 20, or more) detectors or lenses for optical detection of droplets or particles as the droplets or particles flow along the channel. In some instances, the detector may be operatively connected to one or more (e.g., 1, 2, 3, 4, 5, 6, 7, 8, 9, 10, 11, 12, 13, 14, 15, 16, 17, 18, 19, 20, or more) lenses or other light gathering component (e.g., light guide). The channel of the device is aligned with focal plane(s) of the one or more detectors or lenses for optical detection of droplets or particles as the droplets or particles flow along the channel. Each of the one or more detectors or lenses may be configured to detect a distinct focal plane in the channel. As droplets or particles (e.g., droplets or particles formed in and/or provided by the droplet or particle source) flow along the channel, the droplets or particles may be detected at the focal plane(s) of the one or more detectors or lenses.

In some embodiments, the channel has a slanted portion. As droplets or particles flow along the slanted portion of the channel, the droplets or particles pass through the focal plane, allowing for the detection of multiple cross sections of each droplet or particle (e.g., more than 1, such as 2, 3, 4, 5, 6, 7, 8, 9, 10, 11, 12, 13, 14, 15, 16, 17, 18, 19, 20, or more cross sections). In other embodiments, the channel (slanted or not) is aligned with multiple detectors or lenses, allowing detection of droplets or particles at multiple focal planes (e.g., more than 1, such as 2, 3, 4, 5, 6, 7, 8, 9, 10, 11, 12, 13, 14, 15, 16, 17, 18, 19, 20, or more focal planes) as the droplets or particles flow along the channel. Alternatively, the channel may be aligned with a moving (e.g., oscillating) detector or lens, and movement of the detector or lens allows detection of droplets or particles at multiple focal planes (e.g., more than 1, such as 2, 3, 4, 5, 6, 7, 8, 9, 10, 11, 12, 13, 14, 15, 16, 17, 18, 19, 20, or more focal planes) as the droplets or particles flow along the channel. In other embodiments, the channel is constricted and positions droplets or particles and their contents in a single plane, such as a focal plane of a detector or lens for detection. Various embodiments of the channel are described herein.

A slanted portion of the channel, if present, may be aligned with one or more detectors or lenses. Each of the one or more detectors or lenses may be configured for detection of distinct focal planes in the slanted portion. Upon entering the slanted portion of the channel (e.g., from a droplet or particle source), the droplets or particles flow along the slanted portion of the channel. As the droplets or particles flow along the slanted portion of the channel, the droplets or particles pass through the focal plane(s) of the one or more detectors or lenses with which the channel is aligned. This allows for detection of multiple cross sections (e.g., 2, 3, 4, 5, 6, 7, 8, 9, 10, 11, 12, 13, 14, 15, 16, 17, 18, 19, 20, or more cross sections) of each droplet or particle as the droplets or particles flow along the slanted portion of the channel. By detecting multiple cross sections of a droplet or particle, material within the droplet (e.g., one or more particles) can be identified, e.g., to determine the presence, type (e.g., synthetic or biological), and/or number of particles in the droplet.

Thus, the slanted portion may be configured for detection (e.g., optical detection) of multiple cross sections of each droplet or particle (e.g., 2, 3, 4, 5, 6, 7, 8, 9, 10, 11, 12, 13, 14, 15, 16, 17, 18, 19, 20, or more cross sections) as the droplets or particles flow along the slanted portion. In some embodiments, the slanted portion of the channel is slanted in the Z direction (i.e., at a non-zero angle relative to the horizontal plane). The slanted portion of the channel may constitute from 5% to 100% of the length of the channel (e.g., from 10% to 99%, from 15% to 98%, from 20% to 97%, from 25% to 96%, from 30% to 95%, from 35% to 90%, from 40% to 85% from 45% to 80%, or from 50% to 75% of the length of the channel, e.g., from 10% to 15%, from 15% to 20%, from 20% to 25%, from 25% to 30%, from 30% to 35%, from 35% to 40%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100% of the length of the channel, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the length of the channel).

The slanted portion of the channel may be at an angle from 1° to 90° to (e.g., above or below) the focal plane of the one or more detectors or lenses with which the channel is aligned (e.g., from 5° to 70°, from 10° to 60°, from 15° to 50°, or from 20° to 40° to (e.g., above or below) the focal plane, e.g., from 1° to 5°, from 5° to 10°, from 10° to 15°, from 15° to 20°, from 20° to 25°, from 25° to 30°, from 30° to 35°, from 35° to 40°, from 40° to 45°, from 45° to 50°, from 50° to 55°, from 55° to 60°, from 60° to 65°, from 65° to 70°, from 70° to 75°, or from 75° to 80° to (e.g., above or below) the focal plane, e.g., about 1°, about 2°, about 3°, about 4°, about 5°, about 6°, about 7°, about 8°, about 9°, about 10°, about 11°, about 12°, about 13°, about 14°, about 15°, about 16°, about 17°, about 18°, about 19°, about 20°, about 21°, about 22°, about 23°, about 24°, about 25°, about 26°, about 27°, about 28°, about 29°, about 30°, about 31°, about 32°, about 33°, about 34°, about 35°, about 36°, about 37°, about 38°, about 39°, about 40°, about 41°, about 42°, about 43°, about 44°, about 45°, about 46°, about 47°, about 48°, about 49°, about 50°, about 51°, about 52°, about 53°, about 54°, about 55°, about 56°, about 57°, about 58°, about 59°, about 60°, about 61°, about 62°, about 63°, about 64°, about 65°, about 66°, about 67°, about 68°, about 69°, about 70°, about 71°, about 72°, about 73°, about 74°, about 75°, about 76°, about 77°, about 78°, about 79°, about 80°, about 81°, about 82°, about 83°, about 84°, about 85°, about 86°, about 87°, about 88°, about 89°, or about 90° to (e.g., above or below) the focal plane). The focal plane of the one or more detectors or lenses can be horizontal. In such embodiments, the slanted portion is slanted relative to the horizontal plane at any of the angles described.

In addition to a slanted portion, the channel may include a non-slanted, e.g., horizontal portion. The non-slanted portion of the channel may constitute from 5% to 95% of the length of the channel (e.g., from 10% to 94%, from 15% to 93%, from 20% to 92%, from 25% to 91%, from 30% to 90%, from 35% to 85%, from 40% to 80% from 45% to 75%, or from 50% to 70% of the length of the channel, e.g., from 10% to 15%, from 15% to 20%, from 20% to 25%, from 25% to 30%, from 30% to 35%, from 35% to 40%, from 45% to 50%, from 50% to 55%, from 55% to 60%, from 60% to 65%, from 65% to 70%, from 70% to 75%, from 75% to 80%, from 80% to 85%, from 85% to 90%, or from 90% to 95% of the length of the channel, e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of the length of the channel).

As such, the channel may have a slanted portion and a non-slanted, e.g., horizontal portion. The non-slanted portion of the channel may be positioned between the droplet or particle source (e.g., droplet or particle formation region) and the slanted portion of the channel. In such instances, the slanted portion of the channel may be in fluid communication with the droplet or particle source via the non-slanted portion of the channel (e.g., droplets or particles (e.g., droplets or particles formed in the droplet or particle source) may flow from the droplet or particle source to the non-slanted portion of the channel, and then to the slanted portion of the channel).

A slanted portion may have a constant (e.g., linear) or a non-linear (e.g., curving) slant (e.g., vertical change in position, such as rise or fall). The slant may be configured to induce a desired droplet or particle velocity (e.g., a constant or variable velocity), according to known principles.

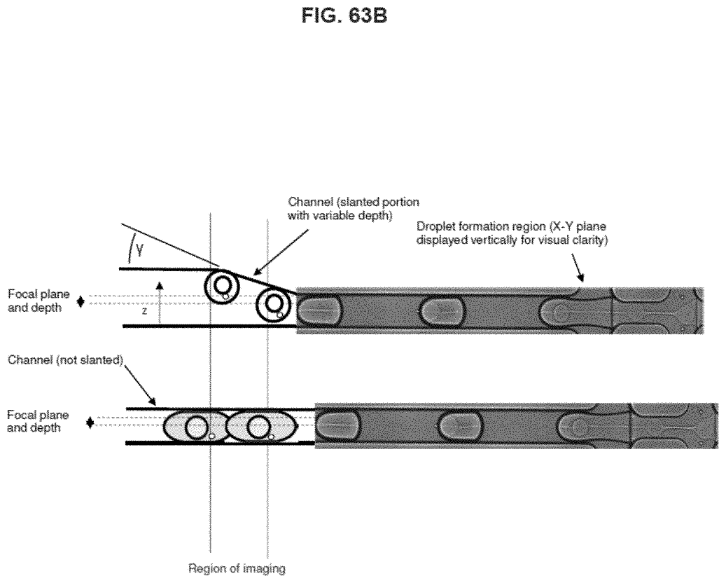

The slanted portion of the channel may have a constant depth (e.g., a constant distance is maintained between the upper and lower walls of the channel). In some instances, at least a portion (e.g., at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more) of the slanted portion of the channel has a constant depth (e.g., a constant distance is maintained between the upper and lower walls of the channel). In other instances, the entire slanted portion of the channel has a constant depth (e.g., a constant distance is maintained between the upper and lower walls of the channel), as shown, for example, in FIG. 63A.

Alternatively, the slanted portion of the channel may have a variable depth (e.g., a constant distance is not maintained between the upper and lower walls of the channel). In some instances, at least a portion (e.g., at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more) of the slanted portion of the channel has a variable depth (e.g., a constant distance is not maintained between the upper and lower walls of the channel). For example, the distance between the upper and lower walls of the channel may increase (e.g., increase by 1% to 100% (e.g., from 5% to 99%, from 10% to 98%, from 15% to 97%, from 20% to 96%, from 25% to 95%, from 30% to 90%, from 35% to 85%, from 40% to 80%, from 45% to 75%, or from 50% to 70%, e.g., from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100%, e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%), or more) in at least a portion (e.g., in at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more) of the slanted portion of the channel, as shown, for example, in FIG. 63B. Alternatively or additionally, the distance between the upper and lower walls of the channel may decrease (e.g., decrease by 1% to 100% (e.g., from 5% to 99%, from 10% to 98%, from 15% to 97%, from 20% to 96%, from 25% to 95%, from 30% to 90%, from 35% to 85%, from 40% to 80%, from 45% to 75%, or from 50% to 70%, e.g., from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100%, e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%), or more) in at least a portion (e.g., in at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more) of the slanted portion of the channel.

Variation in depth of the slanted portion of the channel (e.g., the increase or decrease in distance between the upper and lower walls of the channel) may correlate with the number of cross sections of droplets or particles that are detected via the one or more detectors or lenses as the droplets or particles pass through their focal plane(s) while flowing along the channel. For example, a greater variation in depth may provide for detection of a greater number of droplet or particle cross sections, as droplets or particles flow along the slanted portion of the channel.

Additionally or alternatively, vertical change in position of droplets or particles (e.g., in embodiments having a horizontal focal plane) along the slanted portion of the channel may correlate with the number of cross sections of droplets or particles that are detected by the one or more detectors or lenses as the droplets or particles pass through their focal plane(s) while flowing along the slanted portion of the channel. Vertical change in position (e.g., rise or fall) of droplets or particles along the slanted portion of the channel with variable depth may be regulated by a buoyant force acting on the droplets or particles. For example, the magnitude of the buoyant force acting on the droplets or particles may correlate with the rate of vertical change in position (e.g., rise or fall) of droplets or particles along the slanted portion of the channel with variable depth, and hence correlate with the number of cross sections of the droplets or particles that are detected by the one or more detectors or lenses as the droplets or particles pass through their focal plane(s) while flowing along the slanted portion of the channel.

### Multiple Detectors and Focal Planes

In some embodiments, the channel of the device may be aligned with multiple (e.g., more than 1, such as 2, 3, 4, 5, 6, 7, 8, 9, 10, 11, 12, 13, 14, 15, 16, 17, 18, 19, 20, or more) detectors or lenses for detection of droplets or particles at multiple (e.g., more than 1, such as 2, 3, 4, 5, 6, 7, 8, 9, 10, 11, 12, 13, 14, 15, 16, 17, 18, 19, 20, or more) focal planes. In some instances, the detector is operatively connected with a lens (so that light from the channel is focused on the sensor element). Alternatively, the detector may be operatively connected with multiple (e.g., more than 1, such as 2, 3, 4, 5, 6, 7, 8, 9, 10, 11, 12, 13, 14, 15, 16, 17, 18, 19, 20, or more) lenses. In particular, the channel of the device may be aligned with multiple (e.g., more than 1, such as 2, 3, 4, 5, 6, 7, 8, 9, 10, 11, 12, 13, 14, 15, 16, 17, 18, 19, 20, or more) lenses for detection of droplets or particles at multiple focal planes. In some instances, the lens is a micro-lens. For example, the channel of the device may be aligned with multiple (e.g., more than 1, such as 2, 3, 4, 5, 6, 7, 8, 9, 10, 11, 12, 13, 14, 15, 16, 17, 18, 19, 20, or more) micro-lenses for detection of droplets or particles at multiple focal planes. The multiple detectors or lenses (e.g., micro-lenses) may have different focal planes. For example, the channel of the featured device may be aligned with multiple (e.g., more than 1, such as 2, 3, 4, 5, 6, 7, 8, 9, 10, 11, 12, 13, 14, 15, 16, 17, 18, 19, 20, or more) detectors or lenses (e.g., micro-lenses) with different focal planes for optical detection of droplets or particles at multiple focal planes. Each of the multiple detectors or lenses may be configured to detect distinct focal planes, such as focal planes aligned with the channel of the device. In some embodiments, the droplets or particles may be detected at each of the different focal planes of the multiple detectors or lenses (e.g., micro-lenses) as the droplets or particles flow along the channel. Alternatively, the droplets or particles may be detected at some (e.g., 2 or more, such as 2, 3, 4, 5, 6, 7, 8, 9, 10, 11, 12, 13, 14, 15, 16, 17, 18, 19, 20, or more) of the different focal planes of the multiple detectors or lenses (e.g., micro-lenses) as the droplets or particles flow along the channel. Detection of droplets or particles at each of the different focal planes or at some of the different focal planes of the multiple detectors or lenses (e.g., micro-lenses) may result in imaging of droplets or particles at multiple (e.g., more than 1, such as 2, 3, 4, 5, 6, 7, 8, 9, 10, 11, 12, 13, 14, 15, 16, 17, 18, 19, 20, or more) focal planes as the droplets or particles flow along the channel.

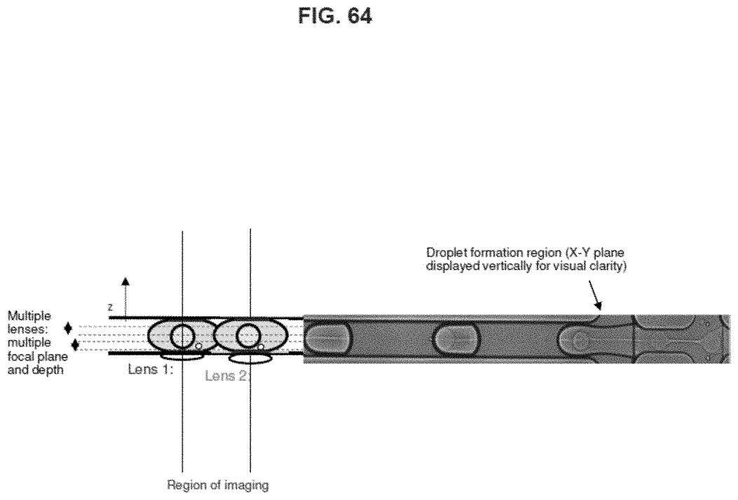

In some embodiments, the multiple detectors or lenses (e.g., micro-lenses), if present, may have different focal distances. Multiple detectors or lenses (e.g., micro-lenses) with different focal distances may be positioned at the same distance from the channel and/or the flowing droplets or particles but may be employed in detecting droplets or particles at different horizontal planes along the channel, as shown, for example, in FIG. 64. In some embodiments, the multiple detectors or lenses (e.g., micro-lenses) may have identical magnification. Alternatively, the multiple detectors or lenses (e.g., micro-lenses) may have different magnification.

Additionally, or alternatively, the multiple detectors or lenses (e.g., micro-lenses) can have the same or similar focal distances but can detect distinct focal planes. For example, detectors or lenses positioned at different distances (in the Z direction) from the channel and/or the flowing droplets or particles may be used in detecting different focal planes if their focal distances are the same.

### Moving Detectors and Lenses

In some embodiments, the channel of the device is aligned with one or more moving detectors or lenses for detection of droplets or particles at multiple (e.g., more than 1, such as 2, 3, 4, 5, 6, 7, 8, 9, 10, 11, 12, 13, 14, 15, 16, 17, 18, 19, 20, or more) focal planes. The moving detector or lens may be moved (e.g., oscillated) piezoelectrically and/or acoustically for detection of droplets or particles at multiple (e.g., more than 1, such as 2, 3, 4, 5, 6, 7, 8, 9, 10, 11, 12, 13, 14, 15, 16, 17, 18, 19, 20, or more) focal planes. In some instances, the detector is operatively connected with a lens. Alternatively, the detector may be operatively connected with multiple (e.g., more than 1, such as 2, 3, 4, 5, 6, 7, 8, 9, 10, 11, 12, 13, 14, 15, 16, 17, 18, 19, 20, or more) lenses. In some embodiments, the droplets or particles are detected at each of the multiple focal planes of the detector or lens (e.g., oscillating detector or lens that is oscillated piezoelectrically and/or acoustically) as the droplets or particles flow along the channel. Alternatively, the droplets or particles may be detected at some (e.g., 2 or more, such as 2, 3, 4, 5, 6, 7, 8, 9, 10, 11, 12, 13, 14, 15, 16, 17, 18, 19, 20, or more) of the multiple focal planes of the detector or lens (e.g., oscillating detector or lens that is oscillated piezoelectrically and/or acoustically) as the droplets or particles flow along the channel. Detection of droplets or particles at the multiple focal planes (e.g., at each of the multiple focal planes or at some of the multiple focal planes) of the detector or lens (e.g., oscillating detector or lens that is oscillated piezoelectrically and/or acoustically) may result in imaging of droplets or particles at multiple (e.g., more than 1, such as 2, 3, 4, 5, 6, 7, 8, 9, 10, 11, 12, 13, 14, 15, 16, 17, 18, 19, 20, or more) focal planes as the droplets or particles flow along the channel. The channel of the device may be aligned with one detector or lens (e.g., oscillating detector or lens that is oscillated piezoelectrically and/or acoustically) for optical detection of droplets or particles at multiple (e.g., more than 1, such as 2, 3, 4, 5, 6, 7, 8, 9, 10, 11, 12, 13, 14, 15, 16, 17, 18, 19, 20, or more) focal planes. Alternatively, the channel of the device may be aligned with more than one (e.g., 2, 3, 4, 5, 6, 7, 8, 9, 10, 11, 12, 13, 14, 15, 16, 17, 18, 19, 20, or more) detectors or lenses (e.g., oscillating detectors or lenses that are oscillated piezoelectrically and/or acoustically) for optical detection of droplets or particles at multiple (e.g., more than 1, such as 2, 3, 4, 5, 6, 7, 8, 9, 10, 11, 12, 13, 14, 15, 16, 17, 18, 19, 20, or more) focal planes.

The frequency that drives the electric voltage applied to the piezoelectric element may be from about 5 to about 300 megahertz (MHz). e.g., about 5 MHz, about 6 MHz, about 7 MHz, about 8 MHz, about 9 MHz, about 10 MHz, about 20 MHz, about 30 MHz, about 40 MHz, about 50 MHz, about 60 MHz, about 70 MHz, about 80 MHz, about 90 MHz, about 100 MHz, about 110 MHz, about 120 MHz, about 130 MHz, about 140 MHz, about 150 MHz, about 160 MHz, about 170 MHz, about 180 MHz, about 190 MHz, about 200 MHz, about 210 MHz, about 220 MHz, about 230 MHz, about 240 MHz, about 250 MHz, about 260 MHz, about 270 MHz, about 280 MHz, about 290 MHz, or about 300 MHz. Alternatively, the RF energy may have a frequency range of less than about 5 MHz or greater than about 300 MHz. As will be appreciated, the necessary voltage and/or the RF frequency driving the electric voltage may change with the properties of the piezoelectric element (e.g., efficiency). In some embodiments, the frequency of oscillation is sufficient to detect focal planes spanning the full depth of the droplet or particle being detected or, e.g., the full depth of the channel.

The number and duration of electric voltage pulses applied to the piezoelectric element may be adjusted to control the frequency, intensity and/or speed of oscillation. For example, the frequency of oscillation may increase with the number of electric voltage pulses. Additionally, the material and size of the piezoelectric element, material and size of the buffer substrate, material, size, and shape of the acoustic lens, number of piezoelectric elements, number of buffer substrates, number of acoustic lenses, respective locations of the one or more piezoelectric elements, respective locations of the one or more buffer substrates, respective locations of the one or more acoustic lenses, dimensions (e.g., length, width, height, expansion angle) of the respective oscillating detector or lens, level of electric voltage applied to the piezoelectric element, and other factors may be adjusted to control frequency, intensity and/or speed of oscillation.

Alternatively or additionally, for imaging objects (e.g., droplets or particles) at multiple focal planes, detectors or lenses may be oscillated (e.g., acoustically) by using a tunable acoustic gradient (TAG), following the methods of Duocastella et al. (“Simultaneous acquisition of multiple focal planes for real time 3-D microscopy using ultra-high speed adaptive optics,” 2012 Conference on Lasers and Electro-Optics (CLEO), San Jose, Calif., 2012, pp. 1-2. doi: 10.1364/CLEO_SI.2012.CTu3J.3), which is incorporated by reference in its entirety. For example, the channel of the featured device may be aligned with one or more TAG detectors or lenses that are oscillated acoustically (e.g., at a frequency from about 5 kHz to about 300 MHz, such as about 5 kHz, about 6 kHz, about 7 kHz, about 8 kHz, about 9 kHz, about 10 kHz, about 20 kHz, about 30 kHz, about 40 kHz, about 50 kHz, about 60 kHz, about 70 kHz, about 80 kHz, about 90 kHz, about 100 kHz, about 150 kHz, about 200 kHz, about 250 kHz, about 300 kHz, about 350 kHz, about 400 kHz, about 450 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, about 20 MHz, about 30 MHz, about 40 MHz, about 50 MHz, about 60 MHz, about 70 MHz, about 80 MHz, about 90 MHz, about 100 MHz, about 110 MHz, about 120 MHz, about 130 MHz, about 140 MHz, about 150 MHz, about 160 MHz, about 170 MHz, about 180 MHz, about 190 MHz, about 200 MHz, about 210 MHz, about 220 MHz, about 230 MHz, about 240 MHz, about 250 MHz, about 260 MHz, about 270 MHz, about 280 MHz, about 290 MHz, or about 300 MHz) for optical detection of droplets or particles at multiple focal planes. In particular, the channel of the featured device may be aligned with one or more TAG detectors or lenses that are oscillated acoustically (e.g., at a frequency of about 72 kHz) for optical detection of droplets or particles at multiple focal planes.

### Constricted Channel

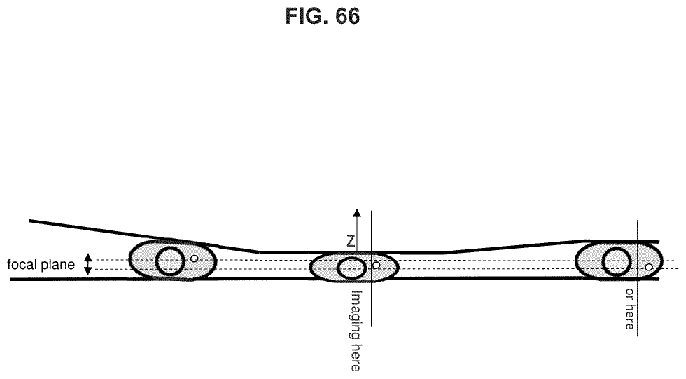

In some embodiments, the channel of the device may be constricted (e.g., depth of the channel or distance between the opposing walls of the channel is reduced) for positioning and imaging of droplets or particles or contents of droplets or particles (e.g., particles, such as cells and/or one or more beads, within the droplets) in a plane, such as the focal plane of a detector or lens (e.g., a detector or lens with which the channel is aligned). Such an embodiment is exemplified in FIG. 66. The channel may be constricted by decreasing the depth of the channel or the distance between the upper and lower walls of the channel (e.g., by 1% to 99% (e.g., from 5% to 99%, from 10% to 98%, from 15% to 97%, from 20% to 96%, from 25% to 95%, from 30% to 90%, from 35% to 85%, from 40% to 80%, from 45% to 75%, or from 50% to 70%, e.g., from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100%, e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%). In some instances, at least a portion (e.g., at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or more) of the channel may be constricted. In particular, in the constricted portion of the channel, the depth of the channel or distance between the opposing walls of the channel may be decreased by about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 99%, or more. As droplets or particles (e.g., droplets provided by the droplet or particle source) flow along the constricted portion of the channel (e.g., a portion of the channel with decreased depth or decreased distance between the upper and lower walls), the depth of the droplets or particles may be reduced. For example, depth of the droplets or particles may be reduced (e.g., by 1% to 99% (e.g., from 5% to 99%, from 10% to 98%, from 15% to 97%, from 20% to 96%, from 25% to 95%, from 30% to 90%, from 35% to 85%, from 40% to 80%, from 45% to 75%, or from 50% to 70%, e.g., from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 95%, or from 95% to 100%, e.g., about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or more). In some instances, reduction in depth of droplets or particles results in change in position of particles (e.g., cells and/or one or more beads) within the droplets or particles as the droplets or particles flow along the constricted portion of the channel. For example, reduction in depth of droplets or particles may change the position of cells with respect to beads within the droplets or particles as the droplets or particles flow along the constricted portion of the channel. Also, reduction in depth of droplets or particles may change the position of cells and/or beads within the droplets or particles and bring the cells and/or beads to the center of the droplets or particles as the droplets or particles flow along the constricted portion of the channel. Additionally, or alternatively, the device can be configured to center the cell(s) in the droplets or particles using shear-induced centering and/or methods known in the art, e.g., those described in Kamperman et al., small 2017, 13(22): 1603711; which is incorporated herein by reference.

The constricted channel (or constricted portion of the channel) may be aligned with one or more (e.g., 1, 2, 3, 4, 5, 6, 7, 8, 9, 10, 11, 12, 13, 14, 15, 16, 17, 18, 19, 20, or more) detectors or lenses. The constricted channel (or constricted portion of the channel) may also be aligned with the focal plane(s) of one or more detectors or lenses. Each of the one or more detectors or lenses may be configured to detect distinct focal planes. Accordingly, each of the one or more detectors or lenses may be configured to detect the constricted portion of the channel (e.g., constricted portion of the channel that is aligned with the one or more detectors or lenses and/or constricted portion of the channel that is aligned with the focal plane(s) of the one or more detectors or lenses) and also detect droplets or particles as the droplets or particles flow along the constricted portion of the channel.

### Detectors

Any of the devices described herein may be aligned with one or more (e.g., 1, 2, 3, 4, 5, 6, 7, 8, 9, 10, 11, 12, 13, 14, 15, 16, 17, 18, 19, 20, or more) cameras (e.g., area scan cameras or line scan cameras, such as time delay and integration (TDI) line scan cameras) for optical detection of droplets or particles. In some embodiments, the devices described herein are aligned with one or more (e.g., 1, 2, 3, 4, 5, 6, 7, 8, 9, 10, 11, 12, 13, 14, 15, 16, 17, 18, 19, 20, or more) area scan cameras for optical detection of droplets or particles. Devices of the present invention may be aligned with one or more area scan cameras for optical detection (e.g., imaging, such as imaging at a fixed resolution) of droplets or particles (e.g., a defined area of a droplet or particle in the channel of a device). In some embodiments, the devices described herein are aligned with one or more (e.g., 1, 2, 3, 4, 5, 6, 7, 8, 9, 10, 11, 12, 13, 14, 15, 16, 17, 18, 19, 20, or more) line scan cameras (e.g., TDI line scan cameras) for optical detection of droplets or particles. Devices of the present invention may be aligned with one or more line scan cameras for high-speed optical detection (e.g., high-speed imaging) of droplets or particles (e.g., droplets or particles flowing at a high speed along the channel of a device). For example, the slanted portion of the channel of a device featured herein may be aligned with one or more (e.g., 1, 2, 3, 4, 5, 6, 7, 8, 9, 10, 11, 12, 13, 14, 15, 16, 17, 18, 19, 20, or more) cameras (e.g., area scan cameras or line scan cameras, such as TDI line scan cameras) for optical detection of droplets or particles as the droplets or particles flow along the slanted portion of the channel of the device.

Any of the devices described herein may be configured for optical detection of droplets or particles, e.g., by using a conventional optical microscope, e.g., for bright-field microscopy. In some embodiments, droplets or particles are detectable by light absorbance, scatter, and/or transmission, e.g., by dark-field microscopy, phase-contrast microscopy, or cross-polarized light microscopy. Additionally, or alternatively, optical detection can include fluorescent detection, e.g., by fluorescent microscopy. In still further embodiments, devices can be configured for detection of droplets or particles having electrical or magnetic labels.

Other exemplary detectors include array detectors, such as CCD or CMOS that produce an image of the focal plane. Other detectors include photodiodes, photovoltaics, and photomultiplier tubes.

Images or other data obtained from detectors (e.g., impedance detectors), or electrodes may be stored on a computer or other memory. The images or data may be used to reconstruct models, e.g., 2D or 3D models of the droplets or particles.

### Electrode Detection Channel

The invention provides devices for improved detection of droplets by detection of particles within droplets by impedance. In some embodiments, the device may include a droplet or particle source (e.g., droplet formation region) and a channel including two or more electrodes. The detection channel is downstream of the droplet or particle source, e.g., at a distance from 1 μm to 1000 mm (e.g., from 1 μm to 10 mm, from 5 μm to 5 mm, from 10 μm to 1 mm, or from 50 μm to 500 μm, e.g., from 1 μm to 2 μm, from 2 μm to 4 μm, from 4 μm to 6 μm, from 6 μm to 8 μm, from 8 μm to 10 μm, from 10 μm to 20 μm, from 20 μm to 40 μm, from 40 μm to 60 μm, from 60 μm to 80 μm, from 80 μm to 100 μm, from 100 μm to 200 μm, from 200 μm to 400 μm, from 400 μm to 600 μm, from 600 μm to 800 μm, from 800 μm to 1 mm, from 1 mm to 2 mm, from 2 mm to 4 mm, from 4 mm to 6 mm, from 6 mm to 8 mm from 8 mm to 10 mm, from 10 mm to 50 mm, from 50 mm to 100 mm, from 100 mm to 200 mm, from 200 mm to 300 mm, from 300 mm to 400 mm, from 400 mm to 500 mm, from 500 mm to 600 mm, from 600 mm to 700 mm, from 700 mm to 800 mm, from 800 mm to 900 mm, or from 900 mm to 1000 mm, e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 150 μm, about 200 μm, about 250 μm, about 300 μm, about 350 μm, about 400 μm, about 450 μm, about 500 μm, about 600 μm, about 700 μm, about 800 μm, about 900 μm, about 1 mm, about 10 mm, about 20 mm, about 30 mm, about 40 mm, about 50 mm, about 60 mm, about 70 mm, about 80 mm, about 90 mm, about 100 mm, or more). The droplet or particle source may be a droplet formation region or a reservoir (e.g., a reservoir holding previously formed droplets and/or particles).

While dimensions of the devices may be described as width or depths, the channels may be disposed in any plane. For example, the width of the electrode may be in the x-y plane, the x-z plane, the y-z plane or any plane there between. In addition, the electrodes may be laterally spaced in the x-y plane relative to the first electrode or located above or below the detection channel. The spatial displacement of electrodes may be oriented in any plane suitable to allow the impedance measurement of a droplet or particle passing through the channel. The fluidic components may also be in different planes so long as connectivity and other dimensional requirements are met.

A device of the invention includes a channel having a proximal end and a distal end and two or more electrodes (e.g., 3, 4, 5, 6, 7, 8, 9, 10, 20, 30, 40, 50, 60, 70, 80, 90, or 100, or more). The channel and two or more electrodes may be used to measure the impedance of a droplet or particle as it passes from the proximal end to the distal end of the channel. The electrodes may be positioned on one side (e.g., the bottom or top portion) of the channel. In another aspect, electrodes are positioned on opposing sides of the channel. In another aspect, two electrodes are located directly across from one another on opposing sides of the channel.

Without wishing to be bound by theory, droplets or particles can pass through the channel at a rate determined by the flow of the continuous phase from the proximal end to the distal end. As the continuous phase continues to flow through the channel, it allows the droplets or particles to pass the electrodes, where an electrical property, e.g., impedance, is measured. It will be understood that the continuous phase may be externally driven, e.g., by gently stirring or vibration but such motion is not necessary, e.g., where the droplet or particle moves relative to the continuous phase by differences in density.

In some embodiments, the depth and/or width of the channel is between about 0.1 μm and 1000 μm. In some embodiments, the depth and/or width is from 1 to 750 μm, 1 to 500 μm, 1 to 250 μm, 1 to 100 μm, 1 to 50 μm, or 3 to 40 μm. The width and depths of the channel may or may not be constant over its length. In particular, the width may increase or decrease adjacent the distal end. In general, the channel may be of any suitable cross section, such as a rectangular, triangular, or circular, or a combination thereof. In particular embodiments, a channel may include a groove along the bottom surface. The width or depth of the channel may also increase or decrease, e.g., in discrete portions, to alter the rate of flow of liquid or particles or the alignment of particles. The detection channel can have any length, width, and height suitable for detecting one or more droplets or particles. For example, the length, width, and height may be independently, e.g., 1 nm-10 mm (e.g., 1 nm-10 nm, e.g., 2 nm, 3 nm, 4 nm, 5 nm, 6 nm, 7 nm, 8 nm, 9 nm, 10 nm, e.g., 10 nm-100 nm, e.g., 20 nm, 30 nm, 40 nm, 50 nm, 60 nm, 70 nm, 80 nm, 90 nm, 100 nm, e.g., 100 nm-1000 nm, e.g., 200 nm, 300 nm, 400 nm, 500 nm, 600 nm, 700 nm, 800 nm, 900 nm, 1000 nm, e.g., 1-10 μm, e.g., 2 μm, 3 μm, 4 μm, 5 μm, 6 μm, 7 μm, 8 μm, 9 μm, 10 μm, e.g., 10-100 μm, e.g., 20 μm, 30 μm, 40 μm, 50 μm, 60 μm, 70 μm, 80 μm, 90 μm, 100 μm, e.g., 100 μm-1000 μm, e.g., 200 μm, 300 μm, 400 μm, 500 μm, 600 μm, 700 μm, 800 μm, 900 μm, 1000 μm, e.g., 1 mm-10 mm, e.g., 2 mm, 3 mm, 4 mm, 5 mm, 6 mm, 7 mm, 8 mm, 9 mm, 10 mm). Additional details on electrodes are described in more detail above in the section on electrodes.

### Droplet or Particle Source

The devices, systems, and methods described herein may include a droplet or particle source. The droplet or particle source may provide droplets or particles to the detection and/or sorting region of a device described herein. The droplet or particle source may include a droplet or particle formation region. A droplet or particle source may include a sample of one or more droplets or particles that is in fluid communication with, e.g., fluidically connected to, the detection and/or sorting region. Exemplary devices and systems that may be used for forming droplets or particles are shown in Examples 1-22. (See also U.S. 2019/0060890, U.S. 2019/0060905, U.S. 2019/0060904, U.S. 2019/0060906, U.S. 2019/0064173, and WO 2019/040637, the disclosures of which are hereby incorporated by reference in their entirety).

The droplets can be a size suitable for utilization as microscale chemical reactors, e.g., for genetic sequencing. Droplets may be formed in a device by flowing a first liquid through a channel and into a droplet source (e.g., a droplet formation region) including a second liquid, i.e., the continuous phase, which may or may not be externally driven. Thus, droplets can be formed without the need for externally driving the second liquid. The size of the generated droplets is significantly less sensitive to changes in liquid properties. For example, the size of the generated droplets is less sensitive to the dispersed phase flow rate. Adding multiple formation regions is also significantly easier from a layout and manufacturing standpoint. The addition of further formation regions allows for formation of droplets even in the event that one droplet source (e.g., one droplet formation region) becomes blocked. Droplet formation can be controlled by adjusting one or more geometric features of fluidic channel architecture, such as a width, height, and/or expansion angle of one or more fluidic channels. For example, droplet size and speed of droplet formation may be controlled. In some instances, the number of regions of formation at a driven pressure can be increased to increase the throughput of droplet formation.

The droplet or particle source may include a droplet or particle formation region. Droplets or particles may be formed by any suitable method known in the art. In general, droplet formation includes two liquid phases. The two phases may be, for example, the sample phase (e.g., an aqueous phase) and an oil phase. During formation, a plurality of discrete volume droplets or particles are formed.

The droplets may be formed by shaking or stirring a liquid to form individual droplets, creating a suspension or an emulsion containing individual droplets, or forming the droplets through pipetting techniques, e.g., with needles, or the like. The droplets may be formed made using a micro-, or nanofluidic droplet maker. Examples of such droplet makers include, e.g., a T-junction droplet maker, a Y-junction droplet maker, a channel-within-a-channel junction droplet maker, a cross (or “X”) junction droplet maker, a flow-focusing junction droplet maker, a micro-capillary droplet maker (e.g., co-flow or flow-focus), and a three-dimensional droplet maker. The droplets may be produced using a flow-focusing device, or with emulsification systems, such as homogenization, membrane emulsification, shear cell emulsification, and fluidic emulsification.

Discrete liquid droplets may be encapsulated by a carrier fluid that wets the microchannel. These droplets, sometimes known as plugs, form the dispersed phase in which the reactions occur. Systems that use plugs differ from segmented-flow injection analysis in that reagents in plugs do not come into contact with the microchannel. In T junctions, the disperse phase and the continuous phase are injected from two branches of the “T”. Droplets of the disperse phase are produced as a result of the shear force and interfacial tension at the fluid-fluid interface. The phase that has lower interfacial tension with the channel wall is the continuous phase. To generate droplets in a flow-focusing configuration, the continuous phase is injected through two outside channels and the disperse phase is injected through a central channel into a narrow orifice. Other geometric designs to create droplets would be known to one of skill in the art. Methods of producing droplets are disclosed in Song et al. Angew. Chem. 45: 7336-7356, 2006, Mazutis et al. Nat. Protoc. 8(5):870-891, 2013, U.S. Pat. No. 9,839,911; U.S. Pub. Nos. 2005/0172476, 2006/0163385, and 2007/0003442, PCT Pub. Nos. WO 2009/005680 and WO 2018/009766. In some embodiments, electric fields or acoustic waves may be used to produce droplets, e.g., as described in PCT Pub. No. WO 2018/009766.

In particular embodiments, droplet forming devices can have a first channel having a depth, a width, a proximal end, and a distal end. The proximal end is or is configured to be in fluid communication with a source of liquid, e.g., a reservoir integral to the device or coupled to the device, e.g., by tubing. The distal end is in fluid communication with, e.g., fluidically connected to, a droplet formation region. A droplet formation region allows liquid from the first channel to expand in at least one dimension, leading to droplet formation under appropriate conditions as described herein. A droplet formation region can be of any suitable geometry.

In one embodiment, the droplet formation region includes a shelf region that allows liquid to expand substantially in one dimension, e.g., perpendicular to the direction of flow. The width of the shelf region is greater than the width of the first channel at its distal end. In certain embodiments, the first channel is a channel distinct from a shelf region, e.g., the shelf region widens or widens at a steeper slope or curvature than the distal end of the first channel. In other embodiments, the first channel and shelf region are merged into a continuous flow path, e.g., one that widens linearly or non-linearly from its proximal end to its distal end; in these embodiments, the distal end of the first channel can be considered to be an arbitrary point along the merged first channel and shelf region. In another embodiment, the droplet formation region includes a step region, which provides a spatial displacement and allows the liquid to expand in more than one dimension. The spatial displacement may be upward or downward or both relative to the channel. The choice of direction may be made based on the relative density of the dispersed and continuous phases, with an upward step employed when the dispersed phase is less dense than the continuous phase and a downward step employed when the dispersed phase is denser than the continuous phase. Droplet formation regions may also include combinations of a shelf and a step region, e.g., with the shelf region disposed between the channel and the step region.

Without wishing to be bound by theory, droplets of a first liquid can be formed in a second liquid in the devices of the invention by flow of the first liquid from the distal end into the droplet formation region. In embodiments with a shelf region and a step region, the stream of first liquid expands laterally into a disk-like shape in the shelf region. As the stream of first liquid continues to flow across the shelf region, the stream passes into the step region wherein the droplet assumes a more spherical shape and eventually detaches from the liquid stream. As the droplet is forming, passive flow of the continuous phase around the nascent droplet occurs, e.g., into the shelf region, where it reforms the continuous phase as the droplet separates from its liquid stream. Droplet formation by this mechanism can occur without externally driving the continuous phase, unlike in other systems. It will be understood that the continuous phase may be externally driven during droplet formation, e.g., by gently stirring or vibration but such motion is not necessary for droplet formation.

In these embodiments, the size of the generated droplets is significantly less sensitive to changes in liquid properties. For example, the size of the generated droplets is less sensitive to the dispersed phase flow rate. Adding multiple formation regions is also significantly easier from a layout and manufacturing standpoint. The addition of further formation regions allows for formation of droplets even in the event that one droplet formation region becomes blocked. Droplet formation can be controlled by adjusting one or more geometric features of fluidic channel architecture, such as a width, height, and/or expansion angle of one or more fluidic channels. For example, droplet size and speed of droplet formation may be controlled. In some instances, the number of regions of formation at a driven pressure can be increased to increase the throughput of droplet formation.

Passive flow of the continuous phase may occur simply around the nascent droplet. The droplet or particle formation region may also include one or more channels that allow for flow of the continuous phase to a location between the distal end of the first channel and the bulk of the nascent droplet. These channels allow for the continuous phase to flow behind a nascent droplet, which modifies (e.g., increase or decreases) the rate of droplet formation. Such channels may be fluidically connected to a reservoir of the droplet or particle formation region or to different reservoirs of the continuous phase. Although externally driving the continuous phase is not necessary, external driving may be employed, e.g., to pump continuous phase into the droplet or particle formation region via additional channels. Such additional channels may be to one or both lateral sides of the nascent droplet or above or below the plane of the nascent droplet.

In general, the components of a device, e.g., channels, may have certain geometric features that at least partly determine the sizes of the droplets. For example, any of the channels described herein have a depth, a height, h₀, and width, w. The droplet or particle formation region may have an expansion angle, α. Droplet size may decrease with increasing expansion angle. The resulting droplet radius, R_{d}, may be predicted by the following equation for the aforementioned geometric parameters of h₀, w, and α:

$${R}_{a} \approx 0.44 ( 1 + 2.2 \sqrt{tan α} \frac{w}{{h}_{0}} ) \frac{{h}_{0}}{\sqrt{tan α}}$$

As a non-limiting example, for a channel with w=21 μm, h=21 μm, and α=3°, the predicted droplet size is 121 μm. In another example, for a channel with w=25 μm, h=25 μm, and α=5°, the predicted droplet size is 123 μm. In yet another example, for a channel with w=28 μm, h=28 μm, and α=7°, the predicted droplet size is 124 μm. In some instances, the expansion angle may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

The depth and width of the first channel may be the same, or one may be larger than the other, e.g., the width is larger than the depth, or first depth is larger than the width. In some embodiments, the depth and/or width is between about 0.1 μm and 1000 μm. In some embodiments, the depth and/or width of the first channel is from 1 to 750 μm, 1 to 500 μm, 1 to 250 μm, 1 to 100 μm, 1 to 50 μm, or 3 to 40 μm. In some cases, when the width and length differ, the ratio of the width to depth is, e.g., from 0.1 to 10, e.g., 0.5 to 2 or greater than 3, such as 3 to 10, 3 to 7, or 3 to 5. The width and depths of the first channel may or may not be constant over its length. In particular, the width may increase or decrease adjacent the distal end. In general, channels may be of any suitable cross section, such as a rectangular, triangular, or circular, or a combination thereof. In particular embodiments, a channel may include a groove along the bottom surface. The width or depth of the channel may also increase or decrease, e.g., in discrete portions, to alter the rate of flow of liquid or particles or the alignment of particles.

Devices of the invention may also include additional channels that intersect the first channel between its proximal and distal ends, e.g., one or more second channels having a second depth, a second width, a second proximal end, and a second distal end. Each of the first proximal end and second proximal ends are or are configured to be in fluid communication with, e.g., fluidically connected to, a source of liquid, e.g., a reservoir integral to the device or coupled to the device, e.g., by tubing. The inclusion of one or more intersection channels allows for splitting liquid from the first channel or introduction of liquids into the first channel, e.g., that combine with the liquid in the first channel or do not combine with the liquid in the first channel, e.g., to form a sheath flow. Channels can intersect the first channel at any suitable angle, e.g., between 5° and 135° relative to the centerline of the first channel, such as between 75° and 115° or 85° and 95°. Additional channels may similarly be present to allow introduction of further liquids or additional flows of the same liquid. Multiple channels can intersect the first channel on the same side or different sides of the first channel. When multiple channels intersect on different sides, the channels may intersect along the length of the first channel to allow liquid introduction at the same point. Alternatively, channels may intersect at different points along the length of the first channel. In some instances, a channel configured to direct a liquid comprising a plurality of particles may comprise one or more grooves in one or more surface of the channel to direct the plurality of particles towards the droplet or particle formation fluidic connection. For example, such guidance may increase single occupancy rates of the generated droplets or particles. These additional channels may have any of the structural features discussed above for the first channel.

Devices may include multiple first channels, e.g., to increase the rate of droplet or particle formation. In general, throughput may significantly increase by increasing the number of droplet or particle formation regions of a device. For example, a device having five droplet or particle formation regions may generate five times as many droplets or particles than a device having one droplet or particle formation region, provided that the liquid flow rate is about the same. A device may have as many droplet or particle formation regions as is practical and allowed for the size of the source of liquid, e.g., reservoir. For example, the device may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 2000 or more droplet or particle formation regions. Inclusion of multiple droplet or particle formation regions may require the inclusion of channels that traverse but do not intersect, e.g., the flow path is in a different plane. Multiple first channel may be in fluid communication with, e.g., fluidically connected to, a separate source reservoir and/or a separate droplet or particle formation region. In other embodiments, two or more first channels are in fluid communication with, e.g., fluidically connected to, the same fluid source, e.g., where the multiple first channels branch from a single, upstream channel. The droplet or particle formation region may include a plurality of inlets in fluid communication with the first proximal end and a plurality of outlets (e.g., plurality of outlets in fluid communication with a collection region) (e.g., fluidically connected to the first proximal end and in fluid communication with a plurality of outlets). The number of inlets and the number of outlets in the droplet or particle formation region may be the same (e.g., there may be 3-10 inlets and/or 3-10 outlets). Alternatively, or in addition, the throughput of droplet or particle formation can be increased by increasing the flow rate of the first liquid. In some cases, the throughput of droplet or particle formation can be increased by having a plurality of single droplet or particle forming devices, e.g., devices with a first channel and a droplet or particle formation region, in a single device, e.g., parallel droplet or particle formation.

In certain embodiments, the droplet formation region is a multiplexed droplet formation region having a width that is at least five times greater (e.g., at least 6 times greater, at least 7 times greater, at least 8 times greater, at least 9 times greater, at least 10 times greater, at least 15 times greater, at least 20 times greater, at least 25 times greater, at least 30 times greater, or at least 40 time greater; e.g., 5 to 50 times greater, 10 to 50 times greater, or 15 to 50 times greater) than the combined widths of the channel outlets fluidically connected to the droplet formation region. The length of the shelf region may be greater than the width of a single first channel outlet by at least 100% (e.g., at least 200%, at least 300%, at least 400%, at least 500%, at least 600%, at least 700%, at least 800%, at least 900%, at least 1000%, at least 1400%, at least 1500%, at least 1900%, or at least 2000%). The length of the shelf region may be greater than the width of a single first channel outlet by 2000% or less (e.g., by 1500% or less, 1000% or less, 900% or less, 800% or less, 700% or less, or 600% or less). For example, the shelf region length may be 100% to 2000% (e.g., 100% to 200%, 100% to 300%, 100% to 400%, 100% to 500%, 100% to 600%, 100% to 700%, 100% to 800%, 100% to 900%, 100% to 1000%, 100% to 1500%, 100% to 2000%, 200% to 300%, 200% to 400%, 200% to 500%, 200% to 600%, 200% to 700%, 200% to 800%, 200% to 900%, 200% to 1000%, 200% to 1500%, 200% to 2000%, 300% to 400%, 300% to 500%, 300% to 600%, 300% to 700%, 300% to 800%, 300% to 900%, 300% to 1000%, 300% to 1500%, 300% to 2000%, 400% to 500%, 400% to 600%, 400% to 700%, 400% to 800%, 400% to 900%, 400% to 1000%, 400% to 1500%, 400% to 2000%, 500% to 600%, 500% to 700%, 500% to 800%, 500% to 900%, 500% to 1000%, 500% to 1500%, 500% to 2000%, 600% to 700%, 600% to 800%, 600% to 900%, 600% to 1000%, 600% to 1500%, 600% to 2000%, 700% to 500%, 700% to 600%, 700% to 700%, 700% to 800%, 700% to 900%, 700% to 1000%, 700% to 1500%, or 700% to 2000%) of the width of a single first channel outlet. The droplet formation region may occupy at least 5% (e.g., at least 10%, at least 15%, at least 20%, at least 25%, or at least 30%) of the perimeter of the droplet collection region. The droplet formation region may occupy 75% or less (e.g., 70% or less, 60% or less, 50% or less, or 40% or less) of the perimeter of the droplet collection region. For example, the droplet formation region may occupy 5% to 75% (e.g., 5% to 70%, 5% to 60%, 5% to 50%, 5% to 40%, 10% to 70%, 10% to 60%, 10% to 50%, 10% to 40%, 15% to 70%, 15% to 60%, 15% to 50%, 15% to 40%, 20% to 70%, 20% to 60%, 20% to 50%, 20% to 40%, 25% to 70%, 25% to 60%, 25% to 50%, 25% to 40%, 30% to 70%, 30% to 60%, 30% to 50%, or 30% to 40%) of the perimeter of the droplet collection region.

In some preferred embodiments, the droplet formation region includes a shelf region protruding from the first channel outlet towards the droplet collection region. For example, the shelf region may be protruding into the step region. In these embodiments, the shelf region width may be twice the width of the first channel outlet or less.

The width of a shelf region may be from 0.1 μm to 1000 μm. In particular embodiments, the width of the shelf is from 1 to 750 μm, 10 to 500 μm, 10 to 250 μm, or 10 to 150 μm. The width of the shelf region may be constant along its length, e.g., forming a rectangular shape. Alternatively, the width of the shelf region may increase along its length away from the distal end of the first channel. This increase may be linear, nonlinear, or a combination thereof. In certain embodiments, the shelf widens 5% to 10,000%, e.g., at least 300%, (e.g., 10% to 500%, 100% to 750%, 300% to 1000%, or 500% to 1000%) relative to the width of the distal end of the first channel. The depth of the shelf can be the same as or different from the first channel. For example, the bottom of the first channel at its distal end and the bottom of the shelf region may be coplanar. Alternatively, a step or ramp may be present where the distal end meets the shelf region. The depth of the distal end may also be greater than the shelf region, such that the first channel forms a notch in the shelf region. The depth of the shelf may be from 0.1 to 1000 μm, e.g., 1 to 750 μm, 1 to 500 μm, 1 to 250 μm, 1 to 100 μm, 1 to 50 μm, or 3 to 40 μm. In some embodiments, the depth is about constant along the length of the shelf (e.g., the depth may vary by about +/−10%). Alternatively, the depth of the shelf slopes, e.g., downward or upward, from the distal end of the liquid channel to the step region. The final depth of the sloped shelf may be, for example, from 5% to 1000% greater than the shortest depth, e.g., 10 to 750%, 10 to 500%, 50 to 500%, 60 to 250%, 70 to 200%, or 100 to 150%. The overall length of the shelf region may be from at least about 0.1 μm to about 1000 μm, e.g., 0.1 to 750 μm, 0.1 to 500 μm, 0.1 to 250 μm, 0.1 to 150 μm, 1 to 150 μm, 10 to 150 μm, 50 to 150 μm, 100 to 150 μm, 10 to 80 μm, or 10 to 50 μm. In certain embodiments, the lateral walls of the shelf region, i.e., those defining the width, may be not parallel to one another. In other embodiments, the walls of the shelf region may narrower from the distal end of the first channel towards the step region. For example, the width of the shelf region adjacent the distal end of the first channel may be sufficiently large to support droplet formation. In other embodiments, the shelf region is not substantially rectangular, e.g., not rectangular or not rectangular with rounded or chamfered corners.

A step region includes a spatial displacement (e.g., depth). Typically, this displacement occurs at an angle of approximately 90°, e.g., between 85° and 95°. Other angles are possible, e.g., 10-90°, e.g., 20 to 90°, 45 to 90°, or 70 to 90°. The spatial displacement of the step region may be any suitable size to be accommodated on a device, as the ultimate extent of displacement does not affect performance of the device. Preferably the displacement is several times the diameter of the droplet being formed. In certain embodiments, the displacement is from about 1 μm to about 10 cm, e.g., at least 10 μm, at least 40 μm, at least 100 μm, or at least 500 μm, e.g., 40 μm to 600 μm. In some embodiments, the displacement is at least 40 μm, at least 45 μm, at least 50 μm, at least 55 μm, at least 60 μm, at least 65 μm, at least 70 μm, at least 75 μm, at least 80 μm, at least 85 μm, at least 90 μm, at least 95 μm, at least 100 μm, at least 110 μm, at least 120 μm, at least 130 μm, at least 140 μm, at least 150 μm, at least 160 μm, at least 170 μm, at least 180 μm, at least 190 μm, at least 200 μm, at least 220 μm, at least 240 μm, at least 260 μm, at least 280 μm, at least 300 μm, at least 320 μm, at least 340 μm, at least 360 μm, at least 380 μm, at least 400 μm, at least 420 μm, at least 440 μm, at least 460 μm, at least 480 μm, at least 500 μm, at least 520 μm, at least 540 μm, at least 560 μm, at least 580 μm, or at least 600 μm. In some cases, the depth of the step region is about constant. Alternatively, the depth of the step region may increase away from the shelf region, e.g., to allow droplets that sink or float to roll away from the spatial displacement as they are formed. The step region may also increase in depth in two dimensions relative to the shelf region, e.g., both above and below the plane of the shelf region. The reservoir may have an inlet and/or an outlet for the addition of continuous phase, flow of continuous phase, or removal of the continuous phase and/or droplets. The step may be part of a wall of a collection reservoir. The depth of the step may be greater than that of the channel and the shelf.

While dimension of the devices may be described as width or depths, the channels, shelf regions, and step regions may be disposed in any plane. For example, the width of the shelf may be in the x-y plane, the x-z plane, the y-z plane or any plane therebetween. In addition, a droplet or particle formation region, e.g., including a shelf region, may be laterally spaced in the x-y plane relative to the first channel or located above or below the first channel. Similarly, a droplet formation region, e.g., including a step region, may be laterally spaced in the x-y plane, e.g., relative to a shelf region or located above or below a shelf region. The spatial displacement in a step region may be oriented in any plane suitable to allow the nascent droplet to form a spherical shape. The fluidic components may also be in different planes so long as connectivity and other dimensional requirements are met.

The device may also include reservoirs for liquid reagents. For example, the device may include a reservoir for the liquid to flow in the first channel and/or a reservoir for the liquid into which droplets or particles are formed. In some cases, devices of the invention include a collection region, e.g., a volume for collecting formed droplets or particles. A droplet or particle collection region may be a reservoir that houses continuous phase or can be any other suitable structure, e.g., a channel, a shelf, a chamber, or a cavity, on or in the device. For reservoirs or other elements used in collection, the walls may be smooth and not include an orthogonal element that would impede droplet or particle movement. For example, the walls may not include any feature that at least in part protrudes or recedes from the surface. It will be understood, however, that such elements may have a ceiling or floor. The droplets or particles that are formed may be moved out of the path of the next droplet or particle being formed by gravity (either upward or downward depending on the relative density of the droplet or particle and continuous phase). Alternatively, or in addition, formed droplets or particles may be moved out of the path of the next droplet or particle being formed by an external force applied to the liquid in the collection region, e.g., gentle stirring, flowing continuous phase, or vibration. Similarly, a reservoir for liquids to flow in additional channels, such as those intersecting the first channel may be present. A single reservoir may also be connected to multiple channels in a device, e.g., when the same liquid is to be introduced at two or more different locations in the device. Waste reservoirs or overflow reservoirs may also be included to collect waste or overflow when droplets or particles are formed. Alternatively, the device may be configured to mate with sources of the liquids, which may be external reservoirs such as vials, tubes, or pouches. Similarly, the device may be configured to mate with a separate component that houses the reservoirs. Reservoirs may be of any appropriate size, e.g., to hold 10 μL to 500 mL, e.g., 10 μL to 300 mL, 25 μL to 10 mL, 100 μL to 1 mL, 40 μL to 300 μL, 1 mL to 10 mL, or 10 mL to 50 mL. When multiple reservoirs are present, each reservoir may have the same or a different size.

In addition to the components discussed above, devices of the invention can include additional components. For example, channels may include filters to prevent introduction of debris into the device. In some cases, the microfluidic systems described herein may comprise one or more liquid flow units to direct the flow of one or more liquids, such as the aqueous liquid and/or the second liquid immiscible with the aqueous liquid. In some instances, the liquid flow unit may comprise a compressor to provide positive pressure at an upstream location to direct the liquid from the upstream location to flow to a downstream location. In some instances, the liquid flow unit may comprise a pump to provide negative pressure at a downstream location to direct the liquid from an upstream location to flow to the downstream location. In some instances, the liquid flow unit may comprise both a compressor and a pump, each at different locations. In some instances, the liquid flow unit may comprise different devices at different locations. The liquid flow unit may comprise an actuator. In some instances, where the second liquid is substantially stationary, the reservoir may maintain a constant pressure field at or near each droplet or particle formation region. Devices may also include various valves to control the flow of liquids along a channel or to allow introduction or removal of liquids or droplets or particles from the device. Suitable valves are known in the art. Valves useful for a device of the present invention include diaphragm valves, solenoid valves, pinch valves, or a combination thereof. Valves can be controlled manually, electrically, magnetically, hydraulically, fluidically (e.g., pneumatically), or by a combination thereof. The device may also include integral liquid pumps or be connectable to a pump to allow for pumping in the first channels and any other channels requiring flow. Examples of pressure pumps include syringe, peristaltic, diaphragm pumps, and sources of vacuum. Other pumps can employ centrifugal or electrokinetic forces. Alternatively, liquid movement may be controlled by gravity, capillarity, or surface treatments. Multiple pumps and mechanisms for liquid movement may be employed in a single device. The device may also include one or more vents to allow pressure equalization, and one or more filters to remove particulates or other undesirable components from a liquid. The device may also include one or more inlets and or outlets, e.g., to introduce liquids and/or remove droplets or particles. Such additional components may be actuated or monitored by one or more controllers or computers operatively coupled to the device, e.g., by being integrated with, physically connected to (mechanically or electrically), or by wired or wireless connection.

Alternatively, or in addition to controlling droplet or particle formation via microfluidic channel geometry, droplet or particle formation may be controlled using one or more piezoelectric elements. Piezoelectric elements may be positioned inside a channel (i.e., in contact with a fluid in the channel), outside the channel (i.e., isolated from the fluid), or a combination thereof. In some cases, the piezoelectric element may be at the exit of a channel, e.g., where the channel connects to a reservoir or other channel that serves as a droplet or particle generation point. For example, the piezoelectric element may be integrated with the channel or coupled or otherwise fastened to the channel. Examples of fastenings include, but are not limited to, complementary threading, form-fitting pairs, hooks and loops, latches, threads, screws, staples, clips, clamps, prongs, rings, brads, rubber bands, rivets, grommets, pins, ties, snaps, adhesives (e.g., glue), tapes, vacuum, seals, magnets, soldering, or a combination thereof. In some instances, piezoelectric material can be deposited on the chip. In some instances, the piezoelectric element can be built into the channel. Alternatively, or in addition, the piezoelectric element may be connected to a reservoir or channel or may be a component of a reservoir or channel, such as a wall. In some cases, the piezoelectric element may further include an aperture therethrough such that liquids can pass upon actuation of the piezoelectric element, or the device may include an aperture operatively coupled to the piezoelectric element.

The piezoelectric element can have various shapes and sizes. The piezoelectric element may have a shape or cross-section that is circular, triangular, square, rectangular, or partial shapes or combination of shapes thereof. The piezoelectric element can have a thickness from about 100 femtometers (fm) to about 100 millimeters (mm) (e.g., about 100 micrometers (μm) to about 100 mm). The piezoelectric element can have a dimension (e.g., cross-section) of at least about 1 mm. The piezoelectric element can be formed of, for example, lead zirconate titanate, zinc oxide, barium titanate, potassium niobate, sodium tungstate, Ba₂NaNb₅O₅, and Pb₂KNb₅O₁₅. The piezoelectric element, for example, can be a piezo crystal. The piezoelectric element may contract when a voltage is applied and return to its original state when the voltage is unapplied. Alternatively, the piezoelectric element may expand when a voltage is applied and return to its original state when the voltage is unapplied. Alternatively, or in addition, application of a voltage to the piezoelectric element can cause mechanical stress, vibration, bending, deformation, compression, decompression, expansion, and/or a combination thereof in its structure, and vice versa (e.g., applying some form of mechanical stress or pressure on the piezoelectric element may produce a voltage). In some instances, the piezoelectric element may include a composite of both piezoelectric material and non-piezoelectric material. Interdigital transducers (IDTs) can be also patterned on top of piezoelectric element to generate an acoustic wave at a certain frequency, depending on the size and distance of the fingers on the IDTs.

In some instances, the piezoelectric element may be in a first state when no electrical charge is applied, e.g., an equilibrium state. When an electrical charge is applied to the piezoelectric element, the piezoelectric element may bend backwards, pulling a part of the first channel outwards, and drawing in more of the first fluid into the first channel via negative pressure, such as from a reservoir of the first fluid. When the electrical charge is altered, the piezoelectric element may bend in another direction (e.g., inwards towards the contents of the channel), pushing a part of the first channel inwards, and propelling (e.g., at least partly via displacement) a volume of the first fluid, thereby generating a droplet of the first fluid in a second fluid. After the droplet or particle is propelled, the piezoelectric element may return to the first state. The cycle can be repeated to generate more droplets or particles. In some instances, each cycle may generate a plurality of droplets or particles (e.g., a volume of the first fluid propelled breaks off as it enters the second fluid to form a plurality of discrete droplets). A plurality of droplets or particles can be collected in a second channel for continued transportation to a different location (e.g., reservoir), direct harvesting, and/or storage.

While the above non-limiting example describes bending of the piezoelectric element in response to application of an electrical charge, the piezoelectric may undergo or experience vibration, bending, deformation, compression, decompression, expansion, other mechanical stress and/or a combination thereof upon application of an electrical charge, which movement may be translated to the first channel.

In some cases, a channel may include a plurality of piezoelectric elements working independently or cooperatively to achieve the desired formation (e.g., propelling) of droplets or particles. For example, a first channel of a device can be coupled to at least 1, 2, 3, 4, 5, 6, 7, 8, 9, 10, 20, 30, 40, 50, 60, 70, 80, 90, 100, 200, 300, 400, or 500 piezoelectric elements. In an example, a separate piezoelectric element may be operatively coupled to (or be integrally part of) each side wall of a channel. In another example, multiple piezoelectric elements may be positioned adjacent to one another along an axis parallel to the direction of flow in the first channel. Alternatively, or in addition, multiple piezoelectric elements may circumscribe the first channel. For example, a plurality of piezoelectric elements may each be in electrical communication with the same controller or one or more different controllers. The throughput of droplet or particle generation can be increased by increasing the points of generation, such as increasing the number of junctions between first fluid channels and the second fluid channel. For example, each of the first fluid channels may comprise a piezoelectric element for controlled droplet or particle generation at each point of generation. The piezoelectric element may be actuated to facilitate droplet or particle formation and/or flow of the droplets or particles.

The frequency of application of electrical charge to the piezoelectric element may be adjusted to control the speed of droplet or particle generation. For example, the frequency of droplet or particle generation may increase with the frequency of alternating electrical charge. Additionally, the material of the piezoelectric element, number of piezoelectric elements in the channel, the location of the piezoelectric elements, strength of the electrical charge applied, hydrodynamic forces of the respective fluids, and other factors may be adjusted to control droplet or particle generation and/or size of the droplets or particles generated. For example, without wishing to be bound by a particular theory, if the strength of the electrical charge applied is increased, the mechanical stress experienced by the piezoelectric element may be increased, which can increase the impact on the structural deformation of the first channel, increasing the volume of the first fluid propelled, resulting in an increased droplet or particle size.

In a non-limiting example, the first channel can carry a first fluid (e.g., aqueous) and the second channel can carry a second fluid (e.g., oil) that is immiscible with the first fluid. The two fluids can communicate at a junction. In some instances, the first fluid in the first channel may include suspended particles. The particles may be beads, biological particles, cells, cell beads, or any combination thereof (e.g., a combination of beads and cells or a combination of beads and cell beads, etc.). A discrete droplet generated may include a particle, such as when one or more particles are suspended in the volume of the first fluid that is propelled into the second fluid. Alternatively, a discrete droplet generated may include more than one particle. Alternatively, a discrete droplet generated may not include any particles. For example, in some instances, a discrete droplet generated may contain one or more biological particles where the first fluid in the first channel includes a plurality of biological particles.

Alternatively, or in addition, one or more piezoelectric elements may be used to control droplet or particle formation acoustically.

The piezoelectric element may be operatively coupled to a first end of a buffer substrate (e.g., glass). A second end of the buffer substrate, opposite the first end, may include an acoustic lens. In some instances, the acoustic lens can have a spherical, e.g., hemispherical, cavity. In other instances, the acoustic lens can be a different shape and/or include one or more other objects for focusing acoustic waves. The second end of the buffer substrate and/or the acoustic lens can be in contact with the first fluid in the first channel. Alternatively, the piezoelectric element may be operatively coupled to a part (e.g., wall) of the first channel without an intermediary substrate. The piezoelectric element can be in electrical communication with a controller. The piezoelectric element can be responsive to (e.g., excited by) an electric voltage driven at RF frequency. In some embodiments, the piezoelectric element can be made from zinc oxide (ZnO).

The frequency that drives the electric voltage applied to the piezoelectric element may be from about 5 to about 300 megahertz (MHz). e.g., about 5 MHz, about 6 MHz, about 7 MHz, about MHz, about 9 MHz, about 10 MHz, about 20 MHz, about 30 MHz, about 40 MHz, about 50 MHz, about 60 MHz, about 70 MHz, about 80 MHz, about 90 MHz, about 100 MHz, about 110 MHz, about 120 MHz, about 130 MHz, about 140 MHz, about 150 MHz, about 160 MHz, about 170 MHz, about 180 MHz, about 190 MHz, about 200 MHz, about 210 MHz, about 220 MHz, about 230 MHz, about 240 MHz, about 250 MHz, about 260 MHz, about 270 MHz, about 280 MHz, about 290 MHz, or about 300 MHz. Alternatively, the RF energy may have a frequency range of less than about 5 MHz or greater than about 300 MHz. As will be appreciated, the necessary voltage and/or the RF frequency driving the electric voltage may change with the properties of the piezoelectric element (e.g., efficiency).

Before an electric voltage is applied to a piezoelectric element, the first fluid and the second fluid may remain separated at or near the junction via an immiscible barrier. When the electric voltage is applied to the piezoelectric element, it can generate acoustic waves (e.g., sound waves) that propagate in the buffer substrate. The buffer substrate, such as glass, can be any material that can transfer acoustic waves. The acoustic lens of the buffer substrate can focus the acoustic waves towards the immiscible interface between the two immiscible fluids. The acoustic lens may be located such that the interface is located at the focal plane of the converging beam of the acoustic waves. Upon impact of the sound burst on the barrier, the pressure of the acoustic waves may cause a volume of the first fluid to be propelled into the second fluid, thereby generating a droplet or particle of the volume of the first fluid in the second fluid. In some instances, each propelling may generate a plurality of droplets or particles (e.g., a volume of the first fluid propelled breaks off as it enters the second fluid to form a plurality of discrete droplets or particles). After ejection of the droplet or particle, the immiscible interface can return to its original state. Subsequent applications of electric voltage to the piezoelectric element can be repeated to subsequently generate more droplets or particles. A plurality of droplets or particles can be collected in the second channel for continued transportation to a different location (e.g., reservoir), direct harvesting, and/or storage. Beneficially, the droplets or particles generated can have about uniform size, velocity (when ejected), and/or directionality, any of which can be detected using methods of detection provided herein.

In some cases, a device may include a plurality of piezoelectric elements working independently or cooperatively to achieve the desired formation (e.g., propelling) of droplets or particles. For example, the first channel can be coupled to at least 1, 2, 3, 4, 5, 6, 7, 8, 9, 10, 20, 30, 40, 50, 60, 70, 80, 90, 100, 200, 300, 400, or 500 piezoelectric elements. In an example, multiple piezoelectric elements may be positioned adjacent to one another along an axis parallel of the first channel. Alternatively, or in addition, multiple piezoelectric elements may circumscribe the first channel. In some instances, the plurality of piezoelectric elements may each be in electrical communication with the same controller or one or more different controllers. The plurality of piezoelectric elements may each transmit acoustic waves from the same buffer substrate or one or more different buffer substrates. In some instances, a single buffer substrate may comprise a plurality of acoustic lenses at different locations.

In some instances, the first channel may be in communication with a third channel. The third channel may carry the first fluid to the first channel such as from a reservoir of the first fluid. The third channel may include one or more piezoelectric elements, for example, as described herein in the described devices. As described elsewhere herein, the third channel may carry first fluid with one or more particles (e.g., beads, biological particles, etc.) and/or one or more reagents suspended in the fluid. Alternatively, or in addition, the device may include one or more other channels communicating with the first channel and/or the second channel.

The number and duration of electric voltage pulses applied to the piezoelectric element may be adjusted to control the speed of droplet or particle generation. For example, the frequency of droplet or particle generation may increase with the number of electric voltage pulses. Additionally, the material and size of the piezoelectric element, material and size of the buffer substrate, material, size, and shape of the acoustic lens, number of piezoelectric elements, number of buffer substrates, number of acoustic lenses, respective locations of the one or more piezoelectric elements, respective locations of the one or more buffer substrates, respective locations of the one or more acoustic lenses, dimensions (e.g., length, width, height, expansion angle) of the respective channels, level of electric voltage applied to the piezoelectric element, hydrodynamic forces of the respective fluids, and other factors may be adjusted to control droplet or particle generation speed and/or size of the droplets or particles generated.

A discrete droplet generated may include a particle, such as when one or more beads are suspended in the volume of the first fluid that is propelled into the second fluid. Alternatively, a discrete droplet generated may include more than one particle. Alternatively, a discrete droplet generated may not include any particles. For example, in some instances, a discrete droplet generated may contain one or more biological particles where the first fluid in the first channel further includes a suspension of a plurality of biological particles.

In some cases, the droplets or particles formed using a piezoelectric element may be collected in a collection reservoir that is disposed below the droplet or particle generation point. The collection reservoir may be configured to hold a source of fluid to keep the formed droplets or particles isolated from one another. The collection reservoir used after piezoelectric or acoustic element-assisted droplet or particle formation may contain an oil that is continuously circulated, e.g., using a paddle mixer, conveyor system, or a magnetic stir bar. Alternatively, the collection reservoir may contain one or more reagents for chemical reactions that can provide a coating on the droplets or particles to ensure isolation, e.g., polymerization, e.g., thermal- or photo-initiated polymerization.

### Surface Properties

A surface of the device may include a material, coating, or surface texture that determines the physical properties of the device. In particular, the flow of liquids through a device of the invention may be controlled by the device surface properties (e.g., wettability of a liquid-contacting surface). In some cases, a device portion (e.g., a region, channel, or sorter) may have a surface having a wettability suitable for facilitating liquid flow (e.g., in a channel) or assisting droplet formation of a first liquid in a second liquid (e.g., in a channel), e.g., if droplet formation is performed.

Wetting, which is the ability of a liquid to maintain contact with a solid surface, may be measured as a function of a water contact angle. A water contact angle of a material can be measured by any suitable method known in the art, such as the static sessile drop method, pendant drop method, dynamic sessile drop method, dynamic Wilhelmy method, single-fiber Wilhelmy method, single-fiber meniscus method, and Washburn's equation capillary rise method. The wettability of each surface may be suited to sorting cells or particulate components thereof or, if coupled to a droplet formation device, producing droplets of a first liquid in a second liquid.

For example, portions of the device carrying aqueous phases (e.g., a channel) may have a surface material or coating that is hydrophilic or more hydrophilic than the exterior of the device, e.g., include a material or coating having a water contact angle of less than or equal to about 90°, and/or the exterior of the device around the channel may have a surface material or coating that is hydrophobic or more hydrophobic than the channel, e.g., include a material or coating having a water contact angle of greater than 70° (e.g., greater than 90°, greater than 95°, greater than 100° (e.g., 95°-120° or)) 100°-10°. In certain embodiments, the exterior of the device may include a material or surface coating that reduces or prevents wetting by aqueous phases. The device can be designed to have a single type of material or coating throughout. Alternatively, the device may have separate regions having different materials or coatings.

The device surface properties may be those of a native surface (i.e., the surface properties of the bulk material used for the device fabrication) or of a surface treatment. Non-limiting examples of surface treatments include, e.g., surface coatings and surface textures. In one approach, the device surface properties are attributable to one or more surface coatings present in a device portion. Hydrophobic coatings may include fluoropolymers (e.g., AQUAPEL® glass treatment), silanes, siloxanes, silicones, or other coatings known in the art. Other coatings include those vapor deposited from a precursor such as henicosyl-1,1,2,2-tetrahydrododecyldimethyltris(dimethylaminosilane); henicosyl-1,1,2,2-tetrahydrododecyltrichlorosilane (C12); heptadecafluoro-1,1,2,2-tetrahydrodecyltrichlorosilane (C10); nonafluoro-1,1,2,2-tetrahydrohexyltris(dimethylamino)silane; 3,3,3,4,4,5,5,6,6-nonafluorohexyltrichlorosilane; tridecafluoro-1,1,2,2-tetrahydrooctyltrichlorosilane (C8); bis(tridecafluoro-1,1,2,2-tetrahydrooctyl)dimethylsiloxymethylchlorosilane; nonafluorohexyltriethoxysilane (C6); dodecyltrichlorosilane (DTS); dimethyldichlorosilane (DDMS); or 10-undecenyltrichlorosilane (V11); pentafluorophenylpropyltrichlorosilane (C5). Hydrophilic coatings include polymers such as polysaccharides, polyethylene glycol, polyamines, and polycarboxylic acids. Hydrophilic surfaces may also be created by oxygen plasma treatment of certain materials.

A coated surface may be formed by depositing a metal oxide onto a surface of the device. Example metal oxides useful for coating surfaces include, but are not limited to, Al₂O₃, TiO₂, SiO₂, or a combination thereof. Other metal oxides useful for surface modifications are known in the art. The metal oxide can be deposited onto a surface by standard deposition techniques, including, but not limited to, atomic layer deposition (ALD), physical vapor deposition (PVD), e.g., sputtering, chemical vapor deposition (CVD), or laser deposition. Other deposition techniques for coating surfaces, e.g., liquid-based deposition, are known in the art. For example, an atomic layer of Al₂O₃ can be deposited on a surface by contacting it with trimethylaluminum (TMA) and water.

In another approach, the device surface properties may be attributable to surface texture. For example, a surface may have a nanotexture, e.g., have a surface with nanometer surface features, such as cones or columns, that alters the wettability of the surface. Nanotextured surface may be hydrophilic, hydrophobic, or superhydrophobic, e.g., have a water contact angle greater than 150°. Exemplary superhydrophobic materials include Manganese Oxide Polystyrene (MnO₂/PS) nano-composite, Zinc Oxide Polystyrene (ZnO/PS) nano-composite, Precipitated Calcium Carbonate, Carbon nano-tube structures, and a silica nano-coating. Superhydrophobic coatings may also include a low surface energy material (e.g., an inherently hydrophobic material) and a surface roughness (e.g., using laser ablation techniques, plasma etching techniques, or lithographic techniques in which a material is etched through apertures in a patterned mask). Examples of low surface energy materials include fluorocarbon materials, e.g., polytetrafluoroethylene (PTFE), fluorinated ethylene propylene (FEP), ethylene tetrafluoroethylene (ETFE), ethylene chloro-trifluoroethylene (ECTFE), perfluoro-alkoxyalkane (PFA), poly(chloro-trifluoro-ethylene) (CTFE), perfluoro-alkoxyalkane (PFA), and poly(vinylidene fluoride) (PVDF). Other superhydrophobic surfaces are known in the art.

In some cases, the water contact angle of a hydrophilic or more hydrophilic material or coating is less than or equal to about 90°, e.g., less than 80°, 70°, 60°, 50°, 40°, 30°, 20°, or 10°, e.g., 90°, 85°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, or 0°. In some cases, the water contact angle of a hydrophobic or more hydrophobic material or coating is at least 70°, e.g., at least 80°, at least 85°, at least 90°, at least 95°, or at least 100° (e.g., about 100°, 101°, 102°, 103°, 104°, 105°, 106°, 107°, 108°, 109°, 110°, 115°, 120°, 125°, 130°, 135°, 140°, 145°, or about 150°).

The difference in water contact angles between that of a hydrophilic or more hydrophilic material or coating and a hydrophobic or more hydrophobic material or coating may be 5° to 100°, e.g., 5° to 80°, 5° to 60°, 5° to 50°, 5° to 40°, 5° to 30°, 5° to 20°, 10° to 75°, 15° to 70°, 20° to 65°, 25° to 60°, 30 to 50°, 35° to 45°, e.g., 5°, 6°, 7°, 8°, 9°, 10°, 15°, 20°, 25°, 30°, 35°, 40°, 45°, 50°, 55°, 60, 65°, 70°, 75°, 80°, 85°, 90°, 95°, or 100°.

The above discussion centers on the water contact angle. It will be understood that liquids employed in the devices and methods of the invention may not be water, or even aqueous. Accordingly, the actual contact angle of a liquid on a surface of the device may differ from the water contact angle. Furthermore, the determination of a water contact angle of a material or coating can be made on that material or coating when not incorporated into a device of the invention.

### Particles

The invention includes devices, systems, and kits having particles, e.g., for use in detection (e.g., analyte detection). For example, particles configured with analyte detection moieties (e.g., barcodes, nucleic acids, binding molecules (e.g., proteins, peptides, aptamers, antibodies, or antibody fragments), enzymes, substrates, etc.) can be included in a droplet containing an analyte to modify the analyte and/or detect the presence or concentration of the analyte. In some embodiments, particles are synthetic particles (e.g., beads, e.g., gel beads).

For example, a droplet may include one or more analyte-detection moieties, e.g., unique identifiers, such as barcodes. Analyte-detection moieties, e.g., barcodes, may be introduced into droplets previous to, subsequent to, or concurrently with droplet formation. The delivery of the analyte-detection moieties, e.g., barcodes, to a particular droplet allows for the later attribution of the characteristics of an individual sample (e.g., biological particle) to the particular droplet. Analyte-detection moieties, e.g., barcodes, may be delivered, for example on a nucleic acid (e.g., an oligonucleotide), to a droplet via any suitable mechanism. Analyte-detection moieties, e.g., barcoded nucleic acids (e.g., oligonucleotides), can be introduced into a droplet via a particle, such as a microcapsule. In some cases, analyte-detection moieties, e.g., barcoded nucleic acids (e.g., oligonucleotides), can be initially associated with the particle (e.g., microcapsule) and then released upon application of a stimulus which allows the analyte-detection moieties, e.g., nucleic acids (e.g., oligonucleotides), to dissociate or to be released from the particle.

A particle, e.g., a bead, may be porous, non-porous, hollow (e.g., a microcapsule), solid, semi-solid, semi-fluidic, fluidic, and/or a combination thereof. In some instances, a particle, e.g., a bead, may be dissolvable, disruptable, and/or degradable. In some cases, a particle, e.g., a bead, may not be degradable. In some cases, the particle, e.g., a bead, may be a gel bead. A gel bead may be a hydrogel bead. A gel bead may be formed from molecular precursors, such as a polymeric or monomeric species. A semi-solid particle, e.g., a bead, may be a liposomal bead. Solid particles, e.g., beads, may comprise metals including iron oxide, gold, and silver. In some cases, the particle, e.g., the bead, may be a silica bead. In some cases, the particle, e.g., a bead, can be rigid. In other cases, the particle, e.g., a bead, may be flexible and/or compressible.

A particle, e.g., a bead, may comprise natural and/or synthetic materials. For example, a particle, e.g., a bead, can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the particle, e.g., the bead, may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the particle, e.g., the bead, may comprise prepolymers, which are oligomers capable of further polymerization. For example, polyurethane beads may be prepared using prepolymers. In some cases, the particle, e.g., the bead, may contain individual polymers that may be further polymerized together. In some cases, particles, e.g., beads, may be generated via polymerization of different precursors, such that they comprise mixed polymers, co-polymers, and/or block co-polymers. In some cases, the particle, e.g., the bead, may comprise covalent or ionic bonds between polymeric precursors (e.g., monomers, oligomers, linear polymers), oligonucleotides, primers, and other entities. In some cases, the covalent bonds can be carbon-carbon bonds or thioether bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

Particles, e.g., beads, may be of uniform size or heterogeneous size. In some cases, the diameter of a particle, e.g., a bead, may be at least about 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a particle, e.g., a bead, may have a diameter of less than about 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a particle, e.g., a bead, may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm. The size of a particle, e.g., a bead, e.g., a gel bead, used to produce droplets is typically on the order of a cross section of the first channel (width or depth). In some cases, the gel beads are larger than the width and/or depth of the first channel and/or shelf, e.g., at least 1.5×, 2×, 3×, or 4× larger than the width and/or depth of the first channel and/or shelf.

In certain embodiments, particles, e.g., beads, can be provided as a population or plurality of particles, e.g., beads, having a relatively monodisperse size distribution. Where it may be desirable to provide relatively consistent amounts of reagents within droplets or particles, maintaining relatively consistent particle, e.g., bead, characteristics, such as size, can contribute to the overall consistency. In particular, the particles, e.g., beads, described herein may have size distributions that have a coefficient of variation in their cross-sectional dimensions of less than 50%, less than 40%, less than 30%, less than 20%, and in some cases less than 15%, less than 10%, less than 5%, or less.

Particles may be of any suitable shape. Examples of particles, e.g., beads, shapes include, but are not limited to, spherical, non-spherical, oval, oblong, amorphous, circular, cylindrical, and variations thereof.

A particle, e.g., bead, injected or otherwise introduced into a droplet may comprise releasably, cleavably, or reversibly attached analyte detection moieties (e.g., barcodes). A particle, e.g., bead, injected or otherwise introduced into a droplet may comprise activatable analyte detection moieties (e.g., barcodes). A particle, e.g., bead, injected or otherwise introduced into a droplet may be a degradable, disruptable, or dissolvable particle, e.g., dissolvable bead.

Particles, e.g., beads, within a channel may flow at a regular flow profile (e.g., at a regular flow rate or about +/−10% of the flow rate). Such regular flow profiles can permit a droplet, when formed, to include a single particle (e.g., bead) and a single cell or other biological particle. Such regular flow profiles may permit the droplets to have an dual occupancy (e.g., droplets having at least one bead and at least one cell or other biological particle) greater than 5%, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97% 98%, or 99% of the population. In some embodiments, the droplets have a 1:1 dual occupancy (i.e., droplets having exactly one particle (e.g., bead) and exactly one cell or other biological particle) greater than 5%, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97% 98%, or 99% of the population. Such regular flow profiles and devices that may be used to provide such regular flow profiles are provided, for example, in U.S. Patent Publication No. 2015/0292988, which is entirely incorporated herein by reference.

As discussed above, analyte-detection moieties (e.g., barcodes) can be releasably, cleavably or reversibly attached to the particles, e.g., beads, such that analyte detection moieties (e.g., barcodes) can be released or be releasable through cleavage of a linkage between the barcode molecule and the particle, e.g., bead, or released through degradation of the particle (e.g., bead) itself, allowing the barcodes to be accessed or be accessible by other reagents, or both. Releasable analyte-detection moieties (e.g., barcodes) may sometimes be referred to as activatable analyte-detection moieties (e.g., activatable barcodes), in that they are available for reaction once released. Thus, for example, an activatable analyte detection-moiety (e.g., activatable barcode) may be activated by releasing the analyte detection moiety (e.g., barcode) from a particle, e.g., bead (or other suitable type of droplet described herein). Other activatable configurations are also envisioned in the context of the described methods and systems.

In addition to, or as an alternative to the cleavable linkages between the particles, e.g., beads, and the associated antigen detection moieties, such as barcode containing nucleic acids (e.g., oligonucleotides), the particles, e.g., beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a particle, e.g., bead, may be dissolvable, such that material components of the particle, e.g., bead, are degraded or solubilized when exposed to a particular chemical species or an environmental change, such as a change temperature or a change in pH. In some cases, a gel bead can be degraded or dissolved at elevated temperature and/or in basic conditions. In some cases, a particle, e.g., bead, may be thermally degradable such that when the particle, e.g., bead, is exposed to an appropriate change in temperature (e.g., heat), the particle, e.g., bead, degrades. Degradation or dissolution of a particle (e.g., bead) bound to a species (e.g., a nucleic acid, e.g., an oligonucleotide, e.g., barcoded oligonucleotide) may result in release of the species from the particle, e.g., bead. As will be appreciated from the above disclosure, the degradation of a particle, e.g., bead, may refer to the disassociation of a bound or entrained species from a particle, e.g., bead, both with and without structurally degrading the physical particle, e.g., bead, itself. For example, entrained species may be released from particles, e.g., beads, through osmotic pressure differences due to, for example, changing chemical environments. By way of example, alteration of particle, e.g., bead, pore sizes due to osmotic pressure differences can generally occur without structural degradation of the particle, e.g., bead, itself. In some cases, an increase in pore size due to osmotic swelling of a particle, e.g., bead or microcapsule (e.g., liposome), can permit the release of entrained species within the particle. In other cases, osmotic shrinking of a particle may cause the particle, e.g., bead, to better retain an entrained species due to pore size contraction.

A degradable particle, e.g., bead, may be introduced into a droplet, such as a droplet of an emulsion or a well, such that the particle, e.g., bead, degrades within the droplet and any associated species (e.g., nucleic acids, oligonucleotides, or fragments thereof) are released within the droplet when the appropriate stimulus is applied. The free species (e.g., nucleic acid, oligonucleotide, or fragment thereof) may interact with other reagents contained in the droplet. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in particle, e.g., bead, degradation and release of the barcode sequence into the aqueous, inner environment of the droplet. In another example, heating of a droplet comprising a particle-, e.g., bead-, bound analyte-detection moiety (e.g., barcode) in basic solution may also result in particle, e.g., bead, degradation and release of the attached barcode sequence into the aqueous, inner environment of the droplet.

Any suitable number of analyte-detection moieties (e.g., molecular tag molecules (e.g., primer, barcoded oligonucleotide, etc.)) can be associated with a particle, e.g., bead, such that, upon release from the particle, the analyte detection moieties (e.g., molecular tag molecules (e.g., primer, e.g., barcoded oligonucleotide, etc.)) are present in the droplet at a pre-defined concentration. Such pre-defined concentration may be selected to facilitate certain reactions for generating a sequencing library, e.g., amplification, within the droplet. In some cases, the pre-defined concentration of a primer can be limited by the process of producing oligonucleotide-bearing particles, e.g., beads.

Additional reagents may be included as part of the particles (e.g., analyte-detection moieties) and/or in solution or dispersed in the droplet, for example, to activate, mediate, or otherwise participate in a reaction, e.g., between the analyte and analyte-detection moiety.

### Biological Samples

A droplet of the present invention may include biological particles (e.g., cells) and/or macromolecular constituents thereof (e.g., components of cells (e.g., intracellular or extracellular proteins, nucleic acids, glycans, or lipids) or products of cells (e.g., secretion products)). An analyte from a biological particle, e.g., component or product thereof, may be considered to be a bioanalyte. In some embodiments, a biological particle, e.g., cell, or product thereof is included in a droplet, e.g., with one or more particles (e.g., beads), such as particles having an analyte detection moiety. A biological particle, e.g., cell, and/or components or products thereof can, in some embodiments, be encased inside a gel, such as via polymerization of a droplet containing the biological particle and precursors capable of being polymerized or gelled.

In the case of encapsulated biological particles (e.g., cells), a biological particle may be included in a droplet that contains lysis reagents in order to release the contents (e.g., contents containing one or more analytes (e.g., bioanalytes)) of the biological particles within the droplet. In such cases, the lysis agents can be contacted with the biological particle suspension concurrently with, or immediately prior to the introduction of the biological particles into the droplet or particle formation region, for example, through an additional channel or channels upstream or proximal to a second channel or a third channel that is upstream or proximal to a second droplet or particle formation region. Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally, or alternatively, be contained in a droplet with the biological particles (e.g., cells) to cause the release of the biological particles' contents into the droplets or particles. For example, in some cases, surfactant based lysis solutions may be used to lyse cells, although these may be less desirable for emulsion based systems where the surfactants can interfere with stable emulsions. In some cases, lysis solutions may include non-ionic surfactants such as, for example, TRITON X-100 and TWEEN 20. In some cases, lysis solutions may include ionic surfactants such as, for example, sarcosyl and sodium dodecyl sulfate (SDS). In some embodiments, lysis solutions are hypotonic, thereby lysing cells by osmotic shock. Electroporation, thermal, acoustic or mechanical cellular disruption may also be used in certain cases, e.g., non-emulsion based droplet formation such as encapsulation of biological particles that may be in addition to or in place of droplet formation, where any pore size of the encapsulate is sufficiently small to retain nucleic acid fragments of a desired size, following cellular disruption.

In addition to the lysis agents, other reagents can also be included in droplets with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., cells), the biological particles may be exposed to an appropriate stimulus to release the biological particles or their contents from a microcapsule within a droplet. For example, in some cases, a chemical stimulus may be included in a droplet along with an encapsulated biological particle to allow for degradation of the encapsulating matrix and release of the cell or its contents into the larger droplet. In some cases, this stimulus may be the same as the stimulus described elsewhere herein for release of analyte detection moieties (e.g., oligonucleotides) from their respective particle (e.g., bead). In alternative aspects, this may be a different and non-overlapping stimulus, in order to allow an encapsulated biological particle to be released into a droplet at a different time from the release of analyte detection moieties (e.g., oligonucleotides) into the same droplet.

Additional reagents may also be included in droplets with the biological particles, such as endonucleases to fragment a biological particle's DNA, DNA polymerase enzymes and dNTPs used to amplify the biological particle's nucleic acid fragments and to attach the barcode molecular tags to the amplified fragments. Other reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyinosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 nucleotides or longer.

In some cases, the length of a switch oligo may be at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective droplets, the macromolecular components (e.g., macromolecular constituents of biological particles, such as RNA, DNA, or proteins) contained therein may be further processed within the droplets.

As described above, the macromolecular components (e.g., bioanalytes) of individual biological particles (e.g., cells) can be provided with unique identifiers (e.g., barcodes) such that upon characterization of those macromolecular components, at which point components from a heterogeneous population of cells may have been mixed and are interspersed or solubilized in a common liquid, any given component (e.g., bioanalyte) may be traced to the biological particle (e.g., cell) from which it was obtained. The ability to attribute characteristics to individual biological particles or groups of biological particles is provided by the assignment of unique identifiers specifically to an individual biological particle or groups of biological particles. Unique identifiers, for example, in the form of nucleic acid barcodes, can be assigned or associated with individual biological particles (e.g., cells) or populations of biological particles (e.g., cells), in order to tag or label the biological particle's macromolecular components (and as a result, its characteristics) with the unique identifiers. These unique identifiers can then be used to attribute the biological particle's components and characteristics to an individual biological particle or group of biological particles. This can be performed by forming droplets including the individual biological particle or groups of biological particles with the unique identifiers (via particles, e.g., beads), as described in the systems and methods herein.

In some aspects, the unique identifiers are provided in the form of oligonucleotides that comprise nucleic acid barcode sequences that may be attached to or otherwise associated with the nucleic acid contents of individual biological particle, or to other components of the biological particle, and particularly to fragments of those nucleic acids. The oligonucleotides are partitioned such that as between oligonucleotides in a given droplet, the nucleic acid barcode sequences contained therein are the same, but as between different droplets, the oligonucleotides can, and do have differing barcode sequences, or at least represent a large number of different barcode sequences across all of the droplets in a given analysis. In some aspects, only one nucleic acid barcode sequence can be associated with a given droplet, although in some cases, two or more different barcode sequences may be present.

The nucleic acid barcode sequences can include from 6 to about 20 or more nucleotides within the sequence of the oligonucleotides. In some cases, the length of a barcode sequence may be 6, 7, 8, 9, 10, 11, 12, 13, 14, 15, 16, 17, 18, 19, 20 nucleotides or longer. In some cases, the length of a barcode sequence may be at least 6, 7, 8, 9, 10, 11, 12, 13, 14, 15, 16, 17, 18, 19, 20 nucleotides or longer. In some cases, the length of a barcode sequence may be at most 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be 4, 5, 6, 7, 8, 9, 10, 11, 12, 13, 14, 15, 16 nucleotides or longer. In some cases, the barcode subsequence may be at least 4, 5, 6, 7, 8, 9, 10, 11, 12, 13, 14, 15, 16 nucleotides or longer. In some cases, the barcode subsequence may be at most 4, 5, 6, 7, 8, 9, 10, 11, 12, 13, 14, 15, 16 nucleotides or shorter.

Analyte-detection moieties (e.g., oligonucleotides) in droplets can also include other functional sequences useful in processing of nucleic acids from biological particles contained in the droplet. These sequences include, for example, targeted or random/universal amplification primer sequences for amplifying the genomic DNA from the individual biological particles within the droplets while attaching the associated barcode sequences, sequencing primers or primer recognition sites, hybridization or probing sequences, e.g., for identification of presence of the sequences or for pulling down barcoded nucleic acids, or any of a number of other potential functional sequences.

The droplets or particles described herein may include molecular labels. The molecular labels may include barcodes (e.g., nucleic acid barcodes). The molecular labels can be provided to the cells or particulate components based on a number of different methods including, without limitation, microinjection, electroporation, liposome-based methods, nanoparticle-based methods, and lipophilic moiety-barcode conjugate methods. For instance, a lipophilic moiety conjugated to a nucleic acid barcode may be contacted with cells or particulate components of interest. The lipophilic moiety may insert into the plasma membrane of a cell thereby labeling the cell with the barcode. The devices and methods of the present invention may result in molecular labels being present on (i) the interior of a cell or particulate component and/or (ii) the exterior of a cell or particulate component (e.g., on or within the cell membrane). These and other suitable methods will be appreciated by those skilled in the art (see U.S. Published Patent App. No. 2019-0177800, and U.S. patent application Ser. Nos. 16/439,568, 16/426,762, and 16/439,675, each of which is incorporated herein by reference in its entirety).

Other mechanisms of forming droplets containing oligonucleotides may also be employed, including, e.g., coalescence of two or more droplets, where one droplet contains oligonucleotides, or microdispensing of oligonucleotides into droplets, e.g., droplets within microfluidic systems.

In an example, particles (e.g., beads) are provided that each include large numbers of the above described barcoded oligonucleotides releasably attached to the beads, where all of the oligonucleotides attached to a particular bead will include the same nucleic acid barcode sequence, but where a large number of diverse barcode sequences are represented across the population of beads used. In some embodiments, hydrogel beads, e.g., beads having polyacrylamide polymer matrices, are used as a solid support and delivery vehicle for the oligonucleotides into the droplets, as they are capable of carrying large numbers of oligonucleotide molecules, and may be configured to release those oligonucleotides upon exposure to a particular stimulus, as described elsewhere herein. In some cases, the population of beads will provide a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of oligonucleotide molecules attached. In particular, the number of molecules of oligonucleotides including the barcode sequence on an individual bead can be at least about 1,000 oligonucleotide molecules, at least about 5,000 oligonucleotide molecules, at least about 10,000 oligonucleotide molecules, at least about 50,000 oligonucleotide molecules, at least about 100,000 oligonucleotide molecules, at least about 500,000 oligonucleotides, at least about 1,000,000 oligonucleotide molecules, at least about 5,000,000 oligonucleotide molecules, at least about 10,000,000 oligonucleotide molecules, at least about 50,000,000 oligonucleotide molecules, at least about 100,000,000 oligonucleotide molecules, and in some cases at least about 1 billion oligonucleotide molecules, or more.

Moreover, when the population of beads are included in droplets, the resulting population of droplets can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each droplet of the population can include at least about 1,000 oligonucleotide molecules, at least about 5,000 oligonucleotide molecules, at least about 10,000 oligonucleotide molecules, at least about 50,000 oligonucleotide molecules, at least about 100,000 oligonucleotide molecules, at least about 500,000 oligonucleotides, at least about 1,000,000 oligonucleotide molecules, at least about 5,000,000 oligonucleotide molecules, at least about 10,000,000 oligonucleotide molecules, at least about 50,000,000 oligonucleotide molecules, at least about 100,000,000 oligonucleotide molecules, and in some cases at least about 1 billion oligonucleotide molecules.

In some cases, it may be desirable to incorporate multiple different barcodes within a given droplet, either attached to a single or multiple particles, e.g., beads, within the droplet. For example, in some cases, mixed, but known barcode sequences set may provide greater assurance of identification in the subsequent processing, for example, by providing a stronger address or attribution of the barcodes to a given droplet, as a duplicate or independent confirmation of the output from a given droplet.

Oligonucleotides may be releasable from the particles (e.g., beads) upon the application of a particular stimulus. In some cases, the stimulus may be a photo-stimulus, e.g., through cleavage of a photo-labile linkage that releases the oligonucleotides. In other cases, a thermal stimulus may be used, where increase in temperature of the particle, e.g., bead, environment will result in cleavage of a linkage or other release of the oligonucleotides form the particles, e.g., beads. In still other cases, a chemical stimulus is used that cleaves a linkage of the oligonucleotides to the beads, or otherwise results in release of the oligonucleotides from the particles, e.g., beads. In one case, such compositions include the polyacrylamide matrices described above for encapsulation of biological particles, and may be degraded for release of the attached oligonucleotides through exposure to a reducing agent, such as dithiothreitol (DTT).

The droplets described herein may contain either one or more biological particles (e.g., cells), either one or more barcode carrying particles, e.g., beads, or both at least a biological particle and at least a barcode carrying particle, e.g., bead. In some instances, a droplet may be unoccupied and contain neither biological particles nor barcode-carrying particles, e.g., beads. As noted previously, by controlling the flow characteristics of each of the liquids combining at the droplet formation region(s), as well as controlling the geometry of the droplet formation region(s), droplet formation can be optimized to achieve a desired occupancy level of particles, e.g., beads, biological particles, or both, within the droplets that are generated.

### Kits and Systems

Devices of the invention may be combined with various external components, e.g., sorters (e.g., actuators), detectors, electrodes, pumps, reservoirs, or controllers, one or more detectors (e.g., one or more lenses (e.g., tube lens), microscope objectives, lasers, spectrometers, charge sensors, magnetometers, etc.), reagents (e.g., detectable labels, such as fluorochromes, metal ions, etc., e.g., analyte detection moieties, liquids, particles (e.g., beads), and/or sample) in the form of kits and systems.

In some embodiments, one or more of the foregoing components are fabricated as a single device.

In some particular embodiments, a system or kit as described herein for detecting droplets or particles may include one or more of a device, a detector, a wave plate (e.g., quarter wave plate), a reflector, a polarizer (e.g., polarization beam splitter), and a light source. A system may further include any suitable optics system for detecting light. The optics system may be positioned at any suitable location configured to detect light passing through the device.

In particular, the invention features systems that may include a plurality of any of the devices described herein. For example, a system can includes a plurality of devices that are connected (e.g., as a chip or plate having an array of reservoirs or wells, e.g., for high-throughput droplet or particle formation and/or detection). A system of the invention can include one or more devices and reagents (e.g., reagents necessary for droplet or particle formation as described herein). In some embodiments, the reagents include the continuous phase, which, in some embodiments, is contained in the detection region and all or a portion of the collection region. The continuous phase may be in fluid communication with the droplet or particle source and the deflecting surface.

The invention features systems that may include a device for detecting light as described herein having a light source or first light guide and/or one or more detectors or second light guides and a fluidic device having a droplet or particle source and a detection channel in fluid communication with the droplet or particle source, the detection channel having a proximal end and a distal end. The fluidic device mates with the device for detecting light so that the light source or first light guide and the one or more detectors or second lights guides are configured to detect light as a droplet or particle passes from the proximal end to the distal end of the detection channel. The light source or first light guide may be positioned above, below, or to the side of the channel, and the one or more detectors or second light guides may be placed on the same side of the light source or first light guide, a different side (e.g., the opposite side) of the light source or first light guide, or any combination thereof. For example, the detectors or second light guides may be arranged in a geometrical pattern that minimizes gaps between the detectors (see, e.g., FIGS. 59A-59C and 60A-60C). The detectors or second light guides may be arranged in a circular or linear pattern next to the detection channel. The detectors or second light guides may be arranged in a circular or linear pattern next to the detection channel. In some embodiments, the detectors or second light guides are positioned along an entire side of the detection channel or along both sides of the detection channel. In other embodiments, the lights source or first light guide is positioned between two or more detectors or second light guides (FIGS. 57A-57B, 58A-58B, and 62A-62C). The light source may be any element that produces photons, such as a laser or a light emitting diode (LED).

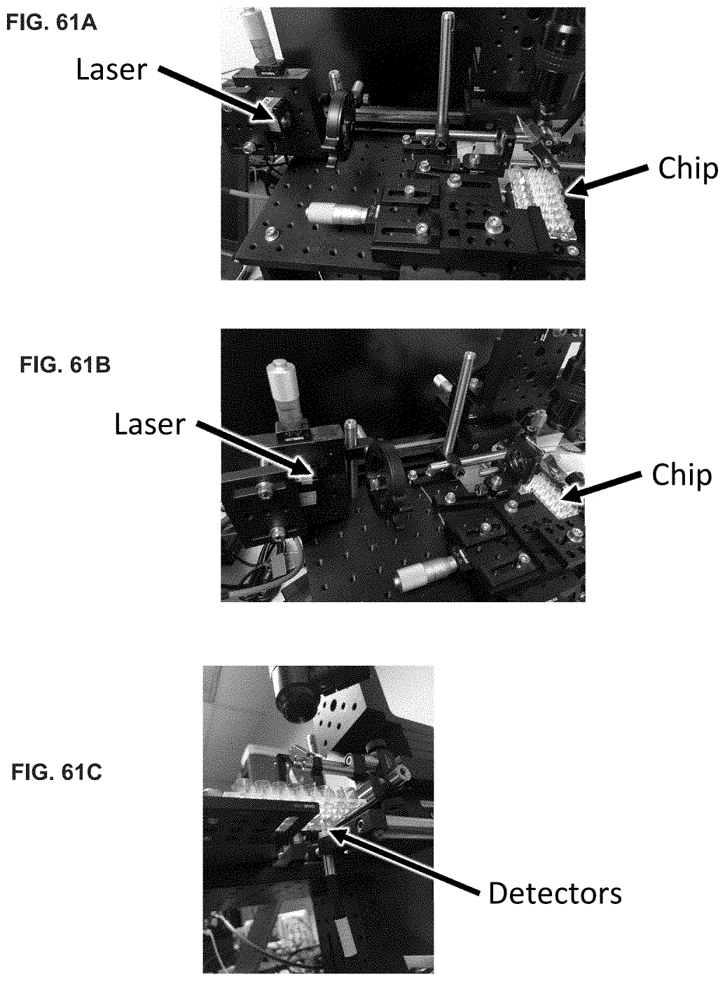

Systems described herein may further include a housing to position the optical components, such as the light source or first light guide and the one or more detectors or second light guides adjacent the device (FIGS. 61A-61C).

In certain embodiments, neither the light source, first light guide, one or more detectors or one or more second light guides is included in the fluidic device. In these embodiments, the device may include voids or other structural features to allow mating of the detection channel with the light source or first light guide and one or more detectors or second light guides to allow detection. In such embodiments, the device for detecting light includes the light source and the one or more detectors. The device for detecting light may further include a first light guide or one or more second light guides for mating with the fluidic device.

In other embodiments, the fluidic device may include one or more (but not all) of the light source, first light guide, one or more detectors or second light guides. For example, the fluidic device may include the first light guide and/or the one or more second light guides, with the device for detecting light including the light source and one or more detectors. In another example, the fluidic device includes the light source or one or more detectors, with the device for detecting light including the other component. In these embodiments, the fluidic device may include a light guide for the component in the device for detecting light.

In embodiments where the device for detecting light includes the light source, the light may be transmitted to the detection channel through the fluidic device without the use of a light guide. For example, a laser or other focused light source can be arranged to illuminate the detection channel, e.g., from a side where no detectors or second light guides are present or in a gap between detectors or second light guides.

A computer as is known in the art may be used to control and/or gather data from the light source or one or more detectors and to conduct analysis of the light detected.

### Systems for Simultaneous Detection

Systems described herein may be configured to simultaneously detect multiple (e.g., more than 1, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or more) detection points. For example, a detection system of the invention simultaneously detects multiple regions, multiple wells, multiple reservoirs, and/or multiple channels of a microfluidic device. A light guide may be provided for each channel or well of the device (e.g., 2-32). For example, the system can include a plurality of light guides and a detector with an array sensor having a plurality of regions. The receiving end of each of the plurality of light guides is configured to receive light from one detection points, and each region is configured to receive light transmitted from one of the transmitting ends of the light guides. The regions are spatially distinct in the array to allow for detection of signal without overlapping. Alternatively, a system can include a linear detector aligned with a plurality of locations, e.g., channels or reservoirs, in a microfluidic device. The linear detector is configured to detect light from the plurality of locations, e.g., channels or reservoirs, simultaneously, and the locations are disposed to allow detection of signal without overlapping. Each of the plurality of locations may be aligned with a droplet or particle formation region to detect droplets or particles formed therein. These systems allow for the detection of multiple detection points simultaneously with a single sensor, where traditional detection would employ a different sensor, e.g., camera, for each point. Systems of the invention can also include a stage configured to hold a microfluidic device.

The microfluidic device can be a part of the system, or it can be a separate component. A detection system described herein may be configured to detect droplets or particles at multiple detection points of a microfluidic device. In some embodiments, the droplets or particles are formed in the microfluidic device. For example, droplets or particles formed in a plurality of droplet or particle formation region can be simultaneously detected at multiple detection points. Alternatively, the droplets or particles may be formed elsewhere and delivered to the microfluidic device for detection by the detection systems described herein.

The detection systems provided herein may include one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32) light guides to transmit light from the detection point to the detector. A light guide may be provided for each channel or well of the device (e.g., 2-32). In some embodiments, the light guide is an image conduit. The image conduit may include multiple (e.g., more than 1, such as 2, 3, 4, 5, 6, 7, 8, 9, 10, 11, 12, 13, 14, 15, 16, 17, 18, 19, 20, or more) optical fibers, such as multi-mode optical fibers. In some embodiments, the image conduit has a diameter from about 0.1 mm to about 25 mm (e.g., about 0.1-25 mm, 0.5-25 mm, 1-25 mm, 1-20 mm, 1-15 mm, 1-10 mm, or 1-5 mm (e.g., about 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, or 25 mm)). In some embodiments, the image conduit has a length from about 1 mm to 1000 mm (e.g., about 1-1000 mm, 1-900 mm, 1-800 mm, 1-700 mm, 1-600 mm, 1-500 mm, 5-500 mm, 5-400 mm, 5-300 mm, 5-200 mm, 5-190 mm, 5-180 mm, 5-170 mm, 5-160 mm, 5-150 mm, 10-150 mm, 10-140 mm, 10-130 mm, 10-120 mm, 10-110 mm, or 10-100 mm (e.g., about 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 150 mm, 160 mm, 170 mm, 180 mm, 190 mm, 200 mm, 250 mm, 300 mm, 350 mm, 400 mm, 450 mm, 500 mm, 550 mm, 600 mm, 650 mm, 700 mm, 750 mm, 800 mm, 850 mm, 900 mm, 950 mm, or 1000 mm). In some embodiments, the transmitting end of the image conduit is at an angle from about 1° to 180° (e.g., at an angle of about 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, 90°, 95°, 100°, 105°, 110°, 115°, 120°, 125°, 130°, 135°, 140°, 145°, 150°, 155°, 160°, 165°, 170°, 175°, or 180°) to the receiving end. Light guides may also include lenses, e.g., micro lenses, alone or in combination with another component, e.g., a light conduit. Other light guides are known in the art.

Systems of the invention may include detectors. Detectors useable with the systems of the invention may include array sensors. Linear arrays may be one dimensional, i.e., include a single row/column of detecting elements, or 2D. An array sensor has multiple (e.g., more than 1, such as 2, 3, 4, 5, 6, 7, 8, 9, 10, 11, 12, 13, 14, 15, 16, 17, 18, 19, 20, or more) spatially distinct regions. In some embodiments, the array sensor is a CCD or CMOS sensor. Other sensors are known in the art, e.g., photomultiplier tubes, photodiodes, e.g., avalanche photodiodes, and solid state photomultipliers. Array sensors (linear or 2D) may be divided into spatially distinct regions. The orientation of the detector with respect to the microfluidic device and associated optics, e.g., light guides, determines which region of the sensor detects a particular sensor. In one embodiment, a linear sensor is used to detected spatially separated channels in a microfluidic device. The channels are spaced so that signal can be detected without overlapping. In another embodiment, light guides deliver signal to different regions in the 2D array. For example, if four detection points are being used, each light guide can direct signal to one quarter of the 2D array. It will also be understood that the combined signals reaching the array may not impinge on every pixel element of the sensor. Furthermore, some overlap at the edges of the regions is acceptable if the region of interest, e.g., the specific element being detected, is spaced from the edges.

In some embodiments, the array sensor may be configured to combine light signals received by the multiple regions to form a single image or video. In other embodiments, the sensor provides an output that is not an image or video, e.g., an integrated sum. In one embodiment, the array may produce an image, video, or other output of the microfluidic device as it exists, e.g., in embodiments in which channels are aligned for simultaneous linear detection. In another embodiment, the array may produce a composite image, video, or other output of spatially distinct areas of the microfluidic that would otherwise not be simultaneously imaged by the array.

In some embodiments, the detection systems described herein may include a linear detector that is aligned with multiple (e.g., more than 1, such as at least 2, 3, 4, 5, 6, 7, 8, 9, 10, 11, 12, 13, 14, 15, 16, 17, 18, 19, 20 or more) detection points (e.g., multiple wells and/or multiple channels) of a microfluidic device and is configured to simultaneously detect light transmitted from the multiple detection points. In particular embodiments, the linear detector is a line scan camera. For example, a detection system described herein may include a line scan camera that is configured to simultaneously detect light (e.g., non-overlapping light signal) transmitted from multiple (e.g., more than 1, such as at least 2, 3, 4, 5, 6, 7, 8, 9, 10, 11, 12, 13, 14, 15, 16, 17, 18, 19, 20 or more) channels of a microfluidic device. In some embodiments, the distance between subsequent channels is between 50 μm to 2 cm (e.g., 50 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 mm, 5 mm, 10 mm, 50 mm, 100 mm, 150 mm, 200 mm, 250 mm, 300 mm, 350 mm, 400 mm, 450 mm, 500 mm, 550 mm, 600 mm, 650 mm, 700 mm, 750 mm, 800 mm, 850 mm, 900 mm, 950 mm, 1 cm, or 2 cm). In some embodiments, the array, such as in a line scan camera, has at least 12,000 pixels (e.g., at least 12,000 pixels, 13,000 pixels, 14,000 pixels, 15,000 pixels, 16,000 pixels, 17,000 pixels, 18,000 pixels, 19,000 pixels, 20,000 pixels, 21,000 pixels, 22,000 pixels, 23,000 pixels, 24,000 pixels, 25,000 pixels, 26,000 pixels, 27,000 pixels, 28,000 pixels, 29,000 pixels, or 30,000 pixels, or more).

In some embodiments, the one or more detectors is a camera (e.g., a video camera). In systems having a video camera, the camera can record 2-1,000,000 frames per second (e.g., 5-1500, 10-1000, 20-800, 40-500, or 60-200 frames per second, e.g., 2-5, 5-10, 10-15, 15-20, 20-25, 25-30, 30-35, 35-40, 40-45, 45-50, 50-60, 60-70, 70-80, 80-90, 90-100, 100-110, 110-120, 120-130, 130-140, 140-150, 150-160, 160-170, 170-180, 180-190, 200-250, 250-300, 300-350, 350-400, 400-450, 450-500, 500-600, 600-700, 700-800, 800-900, 900-1000, 1000-1500, or 1500-2000 frames per second, e.g., 2, 3, 4, 5, 6, 7, 8, 9, 10, 15, 20, 25, 30, 35, 40, 45, 50, 60, 70, 80, 90, 100, 200, 250, 300, 400, 500, 600, 700, 800, 900, 1000, 1100, 1200, 1300, 1400, 1500, 1600, 1700, 1800, 1900, or 2000 frames per second).

In some embodiments, a filter, e.g., emission or neutral density filter, is disposed between the array sensor and the plurality of detection points. Other optical components, e.g., relay or other lens, may be employed to transmit the light to the sensor. Images or other data obtained from detectors may be stored on a computer or other memory. The images or data may be used to reconstruct models, e.g., 2D or 3D models of the droplets.

One or more light sources may be disposed to transmit a beam of light toward one or more (e.g., 1, 2, 3, 4, 5, 6, 7, 8, 9, 10, 11, 12, 13, 14, 15, 16, 17, 18, 19, 20, or more) detection points. In some instances, the light source is a laser or source of white light.

Systems of the invention may be combined with various additional components, e.g., an emission filter (e.g., disposed between the microfluidic device and the detector, or as part of the detector), reagents (e.g., liquids, particles (e.g., beads), and/or sample), pumps, reservoirs, or controllers. For example, the system may include a computer to received data, e.g., images, video, or other output, from the detector for display, storage, or analysis. A computer may also be employed to control the system.

### Systems for Improved Detection Using Angles Beams

Systems for detecting light in a microfluidic device generally include a light source configured to transmit a beam of light at an angle toward a sample, which can be a sample in a microfluidic device (e.g., any suitable device known in the art or described herein). The microfluidic device can be part of the system, or it can be a separate component. For example, systems of the invention can include a stage configured to hold a microfluidic device. Such a system can also include collection optics (e.g., an aspherical lens pair) configured to receive and transmit the light to one or more detectors, for example, through one or more fiber optic cables. In some instances, the one or more detectors and/or one or more fiber optic cables are also part of the system. One advantage of the systems of the invention is reduction of the size/footprint of optical components relative to those conventionally used for detection of a sample, such as standard microscope components such as bulky objectives and/or filters. Such reduced size/footprint is achieved by the angled light and the transmission of light from the sample through collection optics (e.g., through one or more optical fibers). Another advantage of the systems provided herein, when used for detection of droplet formation in the microfluidic devices described herein, is the ability to detect individual droplets as they are made and flow vertically into a collection region. This feature is enabled by the non-vertical angle of the beam of light through the detection point, which can illuminate each droplet as it floats or sinks.

A light source is disposed to transmit a beam of light at an angle toward a detection point, which can be occupied by a sample (e.g., by placing a microfluidic device on the stage of the system). This angle α can be 25° to 40° to the stage (corresponding to an angle of incidence of 50° to 65°). In certain instances, the angle of incidence is Brewster's angle, which will be understood to rely on the material properties (e.g., refractive index) of the microfluidic device in which the sample is to be detected. For example, the angle of incidence can be 50° to 60°, which can be Brewster's angle. In some instances, the angle of incidence is 50° (angle α is 40°) and Brewster's angle is 50°; the angle of incidence is 51° (angle α is 39°) and Brewster's angle is 51°; the angle of incidence is 52° (angle α is 38°) and Brewster's angle is 52°; the angle of incidence is 53° (angle α is 37°) and Brewster's angle is 53°; the angle of incidence is 54° (angle α is 36°) and Brewster's angle is 54°; the angle of incidence is 55° (angle α is 35°) and Brewster's angle is 55°; the angle of incidence is 56° (angle α is) 34° and Brewster's angle is 56°; the angle of incidence is 57° (angle α is 33°) and Brewster's angle is 57°; the angle of incidence is 58° (angle α is 32°) and Brewster's angle is 58°; the angle of incidence is 59° (angle α is 31°) and Brewster's angle is 59°; or the angle of incidence is 60° (angle α is 30°) and Brewster's angle is 60°.

Accordingly, the refractive index of the surface of the device can be from 1.3 to 1.6 (e.g., from 1.4 to 1.55 or from 1.45 to 1.50, e.g., from 1.3 to 1.35, from 1.35 to 1.40, from 1.40 to 1.45, from 1.45 to 1.50, from 1.50 to 1.55, or from 1.55 to 1.60, e.g., about 1.30, about 1.31, about 1.32, about 1.33, about 1.333, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, or about 1.60). In some instances, the refractive indexes of the deflecting surface and the continuous phase are both from 1.3 to 1.6 (e.g., from 1.4 to 1.55 or from 1.45 to 1.50, e.g., from 1.3 to 1.35, from 1.35 to 1.40, from 1.40 to 1.45, from 1.45 to 1.50, from 1.50 to 1.55, or from 1.55 to 1.60, e.g., about 1.30, about 1.31, about 1.32, about 1.33, about 1.333, about 1.34, about 1.35, about 1.36, about 1.37, about 1.38, about 1.39, about 1.40, about 1.41, about 1.42, about 1.43, about 1.44, about 1.45, about 1.46, about 1.47, about 1.48, about 1.49, about 1.50, about 1.51, about 1.52, about 1.53, about 1.54, about 1.55, about 1.56, about 1.57, about 1.58, about 1.59, or about 1.60). The surface (and, optionally, more or all of the device) can be made of any suitable materials, such as polymeric materials, such as polyethylene or polyethylene derivatives, such as cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, polystyrene, or the like, or they may be fabricated in whole or in part from inorganic materials, such as silicon, or other silica based materials, e.g., glass, quartz, fused silica, borosilicate glass, metals, ceramics, and combinations thereof.

In some instances, the light source is a laser. Additionally, or alternatively, the system can include a beam-shaping optical element (e.g., a polarizer) between the light source (e.g., laser) and the detection point.

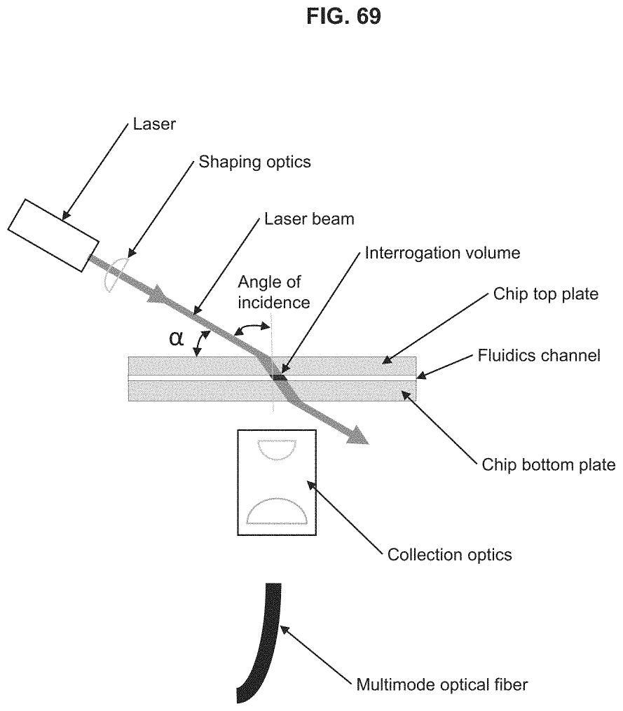

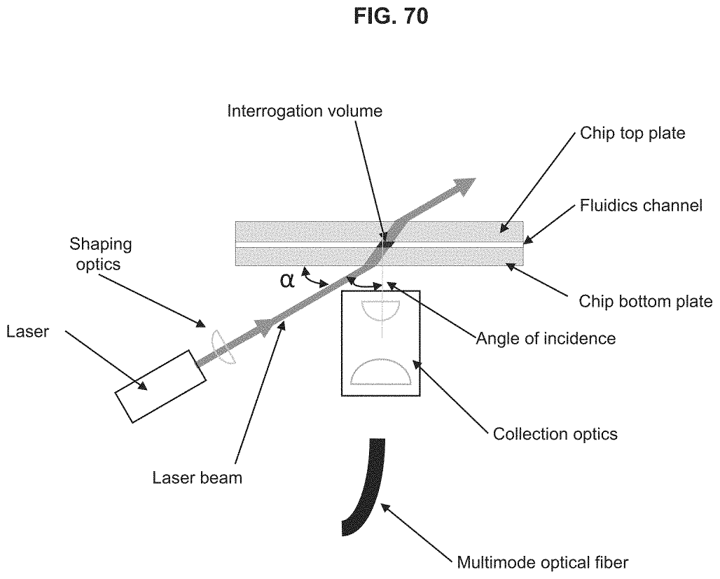

In embodiments in which the stage and/or microfluidic device are positioned substantially horizontally (in the X-Y plane), the light source (e.g., laser) may be above the plane. In such instances, the collection optics can be below the plane, in a configuration such as that shown in FIG. 69. Alternatively, the light source can be below the plane, and the collection optics can be above the plane. Alternatively, the light source and the collection optics can be on the same side of the plane, as depicted in FIG. 70. For example, both the light source and the collection optics can be above the plane or below the plane.

The systems provided herein may include collection optics (e.g., an aspherical lens pair) configured to transmit light from the detection point within a sample to one or more detectors. In some embodiments, the collection optics (e.g., aspherical lens pair) collect the light from the sample and transmit it through one or more optical fibers (e.g., multi-modal optical fibers) to one or more detectors.

An aspherical lens pair has an input focal point and an output focal point. The aspherical lens pair is positioned relative to the microfluidic device and/or stage such that the input focal point is co-localized with the detection point within an interrogation volume in the sample to effectively receive the light transmitted from a component of the sample (e.g., a fluorescently labeled component).

Any of the systems of the invention can further include an optical fiber (e.g., a multi-mode optical fiber) to transmit the light to one or more detectors (e.g., from an aspherical lens pair). The optical fiber has a receiving end and a transmitting end, wherein the receiving end is disposed to co-localize with the output focal point of the aspherical lens pair, and wherein the detector detects the light transmitted from the transmitting end of the optical fiber.

The input focal point and the output focal point of the aspherical lens pair delineate the axis of the aspherical lens pair, which can be normal to the stage and/or microfluidic device. Alternatively, the axis of the aspherical lens pair is not normal to the stage and/or microfluidic device. For example, the axis can be non-normal to the stage and/or microfluidic device and be substantially perpendicular to the angle of the beam of light at the detection point.

A system of the invention may further include one or more dichroic mirrors, e.g., between the aspherical lens pair and the output focal point, or between the aspherical lens pair and a detector. In such instances, an additional detector (e.g., a reflected light detector) can be configured to detect the reflected light from the dichroic mirror.

Detectors of the systems of the invention may include any of the detectors described herein or any suitable means of detection known in the art. In certain instances, the one or more detectors of the present systems may include a camera (e.g., a video camera). In systems having a video camera, the camera can record 2-2000 frames per second (e.g., 5-1500, 10-1000, 20-800, 40-500, or 60-200 frames per second, e.g., 2-5, 5-10, 10-15, 15-20, 20-25, 25-30, 30-35, 35-40, 40-45, 45-50, 50-60, 60-70, 70-80, 80-90, 90-100, 100-110, 110-120, 120-130, 130-140, 140-150, 150-160, 160-170, 170-180, 180-190, 200-250, 250-300, 300-350, 350-400, 400-450, 450-500, 500-600, 600-700, 700-800, 800-900, 900-1000, 1000-1500, or 1500-2000 frames per second, e.g., 2, 3, 4, 5, 6, 7, 8, 9, 10, 15, 20, 25, 30, 35, 40, 45, 50, 60, 70, 80, 90, 100, 200, 250, 300, 400, 500, 600, 700, 800, 900, 1000, 1100, 1200, 1300, 1400, 1500, 1600, 1700, 1800, 1900, or 2000 frames per second).

Systems of the invention may be combined with various additional components, e.g., an emission filter (e.g., disposed between the microfluidic device and the detector, or as part of the detector), one or more lenses, objectives, spectrometers, charge sensors, magnetometers, reagents (e.g., detectable labels, such as fluorochromes, metal ions, etc., analyte detection moieties, liquids, particles (e.g., beads), and/or sample), pumps, reservoirs, or controllers in the form of kits and systems. In certain instances, the system of the invention is multiplexed, e.g., for high-throughput droplet formation and/or detection.

A system may further include additional components, such as detectors or lenses, configured to detect droplets within the device. For example, a system for detection of multiple cross sections of droplets can include a device having droplet source (e.g., a droplet formation region) for providing droplets and a channel with a slanted portion, in combination with one or more detectors and/or lenses, which may be part of a housing configured to engage with (e.g., attach to or mate with) the device. A system for detection of droplets at multiple focal planes can include a droplet source (e.g., a droplet formation region) for providing droplets, a channel for detecting droplets, and multiple detectors or lenses with different focal planes. Alternatively, a system for detection of droplets at multiple focal planes can include a droplet source (e.g., a droplet formation region) for providing droplets, a channel for detecting droplets, and one or more detectors or lenses (e.g., moving or oscillating detectors or lenses). A system for focused imaging of droplets and/or contents of droplets (e.g., imaging of droplets and/or contents of droplets at a single plane) may include a droplet source (e.g., a droplet formation region) for providing droplets, a constricted channel for positioning droplets and/or contents of droplets in a focal plane, and one or more detectors or lenses. Systems described herein may include a plurality of devices that are connected (e.g., as a chip or plate having an array of reservoirs or wells, e.g., for high-throughput droplet formation and/or detection). A system of the invention can include one or more devices and reagents (e.g., reagents necessary for droplet formation as described herein). In some embodiments, the reagents include the continuous phase, which, in some embodiments, is contained in the detection region and all or a portion of the collection region. The continuous phase may be in contact with the droplet source and the deflecting surface.

### Methods for Sorting Droplets or Particles

The invention provides methods for sorting droplets or particles. Droplets or particles are in general sorted by providing a device of the invention and allowing the droplets or particles from a droplet or particle source (e.g., droplet or particle formation region) to move into a sorting region. The sorting region includes a sorter that provides an active or passive force that sorts the droplets or particles into a partition in the collection region. For example, the sorter includes a mechanism (e.g., as described herein) that sorts one or more droplets or particles into one of two or more partitions in the collection region. The sorting mechanism may include a dielectrophoretic actuator, an acoustic actuator, a fluidic (e.g., pneumatic) actuator, a mechanical actuator, a bubble generator, an optical tweezer, a magnet, or an electrostatic charger. The sorting mechanism may provide electromagnetic or mechanical force to sort the droplets or particles. The sorting mechanism may include a divider (e.g., a surface, e.g., angled surface). In some embodiments, the sorting region may employ more than one sorting mechanism (e.g., in parallel or in series).

Droplets or particles may be first formed or provided in a larger volume, such as in a reservoir, and then reentrained into a channel, e.g., for sorting and/or detection. A device may include a first region in fluid communication with (e.g., fluidically coupled to) a second region, e.g., with at least one (e.g., each) cross-sectional dimension smaller than the corresponding cross-sectional dimension of the first region. For example, the droplets or particles may be formed or provided in a region in which each cross-sectional dimension of the sorting region may have a length of at least 1 mm (e.g., 2 mm, 3 mm, 4 mm, 5 mm, 6 mm, 7 mm, 8 mm, 9 mm, 10 mm, or more). Following formation or provision, the droplets or particles may be reentrained into a second region (e.g., a channel) in which each cross-section dimension is less than about 1 mm (e.g., less than about 900 nm, 800 nm, 700 nm, 600 nm, 500 nm, 400 nm, 300 nm, 200 nm, 100 nm, 90 nm, 80 nm, 70 nm, 60 nm, 50 nm, 40 nm, 30 nm, 20 nm, 10 nm, 5 nm, 1 nm, 900 μm, 800 μm, 700 μm, 600 μm, 500 μm, 400 μm, 300 μm, 200 μm, 100 μm, 50 μm, 10 μm, 5 μm, 2 μm, 1 μm, or less). Sorting may be employed in the first region and/or the second region or a subsequent region downstream. This method may include detecting the droplets, e.g., as they are formed or provided in the first region, reentrained in the second region, or while traversing a subsequent region downstream. The detection may provide feedback to a sorter, e.g., to actuate the sorter.

In electrode-based sorting, the electromagnetic field generated by the one or more electrodes is exerted on the sorting region or a channel within the sorting region and provides an active or passive force that sorts the droplets or particles into a partition in the collection region. For example, the electrode-based sorting mechanism sorts one or more droplets or particles into one of two or more partitions in the collection region. The electrode may include a dielectrophoretic electrode or an electrostatic charging electrode. The electrode-based sorting mechanism may provide electromagnetic force to sort the droplets or particles.

The device can be employed to sort droplets or particles that are premade, or it can be employed to sort droplets or particles as they are generated. For example, if the rate of droplet or particle formation does not match rate of sorting, it may be desirable to separate the droplet or particle formation and sorting operations. In some embodiments, one may perform one or more assays on the droplets or particles after generation but before sorting is desired (e.g., incubation of droplets or particles to perform a molecular transformation).

A variety of droplet or particle characteristics can be used to sort droplets or particles. For example, characteristics such as droplet or particle size (e.g., diameter) and shape, the number of particles (e.g., beads) and/or cells within a droplet, optical characteristics, magnetism, electrical properties (e.g. conductivity and permittivity), acoustic properties (e.g. compressibility and density), and electric charge. In some embodiments, methods of the invention include sorting droplets or particles based on the presence and/or intensity of a label, e.g., a fluorescently or ionically tagged antigen-binding molecule bound to a biological antigen (e.g., a protein or nucleic acid, e.g., associated with an intact cell). Such sorting mechanisms can therefore be used to separate droplets or particles containing biological material within a droplet or particle as it passes through the detection region. In some embodiments, the droplets or particles are sorted based on cell viability. The sorting mechanisms described herein may also be used to maintain cell viability, e.g., without disturbing the droplets or particles or the contents therein.

In some embodiments, the methods of sorting allow a user to produce a population of droplets or particles having desired characteristics. For example, in some embodiments, droplet or particle sorting generates populations of droplets or particles that include a suitable fraction of usable droplets or particles (e.g., from 50% to 100%, from 60% to 100%, from 70% to 100%, from 80% to 100%, from 90% to 100%, or from 95% to 100% of droplets). In some embodiments, the droplet or particle sorting generates at least 10% e.g., at least 20%, 30%, 40%, 50%, 60%, 70%, 80%, 85%, 90%, 95%, 97%, 99%, or 100%, of the droplets or particles are usable for a desired purpose.

### Methods for Detecting Droplets or Particles

Provided herein are methods for detecting one or more droplets or particles. A method of detecting a droplet or particle includes, for example, providing a device of the invention (e.g., a droplet or particle detection device or system as described herein), allowing a droplet or particle to enter the detection region, and detecting the droplet or particle in the detection region. After detection, droplets or particles can be collected in a collection region and further analyzed according to any of the analyses described herein (e.g., for genetic analysis).

A variety of droplet or particle characteristics can be observed and/or quantified. For example, characteristics such as droplet or particle size (e.g., diameter) and shape can be readily observed visually and recorded by image or video acquisition software known in the art. In addition, the number of particles (e.g., beads) and/or cells within a droplet or particle can similarly be observed visually, by using detectable labels, or by other optical characteristics (e.g., scatter, absorbance, transmission, fluorescence, etc.). In some embodiments, methods of the invention include observing the presence and/or intensity of a fluorescently or ionically tagged antigen-binding molecule bound to a biological antigen (e.g., a protein or nucleic acid, e.g., associated with an intact cell). Such detection can therefore identify biological material with a droplet as it passes through the detection region. Methods of detection of the invention include detection of any of the various elements described herein, such as analyte-detection moieties or other properties of particles (e.g. beads) or sample within the droplet.

When a droplet or particle enters the detection region, the droplet or particle may be illuminated with light polarized in a first polarization via a polarizer. The light passes through the detection region and through the wave plate (e.g., quarter wave plate) and to a detector. The light may be reflected by a reflector back through the wave plate. This step rotates the polarization to a second polarization orthogonal to the first polarization. This light may further pass through the polarizer to the detector. As described in more detail, the use of polarization reduces background signal from reflections from device surfaces.

The methods described herein allow detection of one or more droplets or particles, e.g., within a droplet. For example, a droplet may contain a particle (e.g., a biological particle, e.g., a cell or a fragment thereof). As the droplet or particle passes through the detection channel, the light source or light guide illuminates it. The scattered and attenuated light from the droplet or particle is detected and measured by the one or more detectors. In some embodiments, the droplet or particle moves to a collection region that includes two or more (e.g., 3, 4, 5, 6, 7, 8, 9, 10, or more) partitions configured for collection after detection. A controller may be used to direct the transport of the droplet or particle through the detection channel.

Methods of detection include optical detection, e.g., by visual observation, e.g., using a conventional optical microscope or with bright-field microscopy. In some embodiments, droplets are detectable by light absorbance, scatter, and/or transmission. Additionally or alternatively, optical detection can include fluorescent detection, e.g., by fluorescent microscopy.

A variety of droplet or particle characteristics can be observed and/or quantified. For example, characteristics such as droplet or particle size (e.g., diameter) and shape can be readily observed visually and recorded by image or video acquisition software known in the art. In addition, the number of particles (e.g., cells and/or beads) within a droplet or particle can similarly be observed visually, by using detectable labels, or by other optical characteristics (e.g., scatter, absorbance, transmission, fluorescence, etc.). In some embodiments, methods of the invention include observing the presence and/or intensity of a fluorescently tagged or otherwise labeled antigen-binding molecule bound to a biological antigen (e.g., a protein or nucleic acid, e.g., associated with an intact cell). Such detection can therefore identify biological material within a droplet or particle as it passes through the detection region. Methods of detection of the invention include detection of any of the various elements described herein, such as analyte-detection moieties or other properties of particles (e.g. cells and/or beads) or sample within the droplet or particle.

In some embodiments, the methods of detection allow a user to monitor the number, frequency, or rate of droplets or particles having desired characteristics. For example, in some embodiments, droplet or particle detection serves as a quality control measure to ensure a suitable fraction of droplets or particles (e.g., from 50% to 100%, from 60% to 100%, from 70% to 100%, from 80% to 100%, from 90% to 100%, or from 95% to 100% of droplets or particles, e.g., at least 80% to 85%, at least 85% to 90%, or at least 90% to 95% of droplets or particles, e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of droplets or particles) pass a given criteria (e.g., contain one bead and one cell, contain at least one bead and at least one cell, contain at least one bead and no more than one cell, contain at least one cell and no more than one bead, contain one bead, contain one cell, contain at least one bead, contain at least one cell, contain no more than one cell, contain no more than one bead, contain one bead and one live cell, contain at least one bead and at least one live cell, contain at least one bead and no more than one live cell, contain at least one live cell and no more than one bead, contain one live cell, contain at least one live cell, contain no more than one live cell, etc.).

The methods described herein provide enhanced imaging capabilities due to the ability to reduce background noise during imaging. When imaging a droplet or particle using a wave plate, the illumination signal is directed towards a wave plate, which then rotates the polarization of the light. The useful image signal has an altered polarization signal from the noise. Therefore, the signal and the noise can be more effectively differentiated to reduce noise during imaging. The methods described herein may also include polarizing the light before directed it to the wave plate, thereby rotating the polarization of the light, e.g., from s-polarized light to p-polarized light. The useful image signal will be p-polarized and the noise will be s-polarized. When the light reflects back through the polarizer, only the image signal will be directed to the detector. A detector configured to detect light in a plane orthogonal to the noise can detect an enhanced useful image and reduce the amount of noise detected.

Methods of droplet or particle detection include optical detection, e.g., by visual observation, e.g., using a conventional optical microscope or with bright-field microscopy. In some embodiments, droplets or particles are detectable by light absorbance, scatter, and/or transmission. Additionally, or alternatively, optical detection can include fluorescent detection, e.g., by fluorescent microscopy. In still further embodiments, methods of the invention include detection of droplets or particles having electrical or magnetic labels.

Light scattered from a particle or droplet can be a function of its size and composition. Thus, various cell types (e.g., lymphocytes, monocytes, granulocytes) can be distinguished based on their scattering properties (e.g., forward and side scatter). This may be used to distinguish droplets that contain a cell, as compared to droplets that don't contain a cell. Furthermore, it can be used to distinguish droplets that contain different types of cells. The detection of a characteristic of the droplet or particle may be used as a trigger to actuate a sorter to send a droplet to a particular partition.

Provided herein are methods for detecting one or more droplets, such as detecting multiple cross sections of droplets, detecting droplets at multiple focal planes, or detecting droplets and/or contents of droplets in the focal plane of a detector. A method for detecting a droplet includes, for example, providing a device of the invention (e.g., a droplet detection device or system as described herein), allowing a droplet to enter the channel of the device for detection, and detecting the droplet in the channel by the one or more detectors or lenses with which the channel is aligned. After detection, droplets can be collected in a collection region and further analyzed according to any of the analyses described herein (e.g., genetic analysis).

In some embodiments are described methods for detecting multiple cross sections of droplets. A channel with a slanted portion can be used to detect multiple cross sections of droplets (e.g., droplets provided by the droplet source). The slanted portion of the channel may be aligned with one or more detectors or lenses. The slanted portion of the channel may also be aligned with the focal plane(s) of the one or more detectors or lenses. While flowing along the slanted portion of the channel, droplets pass through the focal plane(s) of the one or more detectors or lenses that are aligned with the channel. Accordingly, the one or more detectors or lenses may detect multiple (e.g., at least 2, 3, 4, 5, 6, 7, 8, 9, 10, 11, 12, 13, 14, 15, 16, 17, 18, 19, 20, or more) cross sections of the droplets as the droplets flow along the slanted portion of the channel. Additionally or alternatively, the droplets may be detected multiple (e.g., at least 2, 3, 4, 5, 6, 7, 8, 9, 10, 11, 12, 13, 14, 15, 16, 17, 18, 19, 20, or more) times, as the droplets flow along the slanted portion of the channel.

In other embodiments, a channel aligned with multiple detectors or lenses (e.g., micro-lenses) can be used in the detection of droplets (e.g., droplets provided by the droplet source) at multiple focal planes. The channel may be aligned with multiple detectors or lenses having different focal planes, and droplets can be detected at the different focal planes while flowing along the channel.

Alternatively, a channel aligned with a detector or lens (e.g., detector or lens that is oscillated piezoelectrically and/or acoustically) can be used in the detection of droplets (e.g., droplets provided by the droplet source) at multiple focal planes. As described, a constricted channel or a channel with a constricted portion can be used to position droplets (e.g., droplets provided by the droplet source) in a single plane, such as the focal plane of a detector or lens with which the channel is aligned. Constriction (e.g., decrease in depth or distance between the opposing walls of the channel) may also position contents of the droplets (e.g., particles, such as cells and/or one or more beads, within the droplets) in the focal plane of the one or more detectors or lenses (e.g., the one or more detectors or lenses with which the channel is aligned) for detection of the contents of the droplets as the droplets flow along the constricted portion of the channel.

Methods of detecting light in a microfluidic device and/or any of the systems described herein are also provided by the present invention using angle of incidence. A characteristic of a sample is detected by transmitting light from the light source to the detection point within the microfluidic device at angle α. A component of the sample (e.g., a droplet, or a component within the droplet (e.g., a labeled particle)) redirects the light at the detection point, and the redirected light is transmitted through the collection optics (e.g., an aspherical lens pair) to the detector (e.g., from the aspherical lens pair to through an optical fiber, to the detector). In some embodiments, the microfluidic device is a droplet-forming device, and a droplet or characteristic thereof is detected.

In certain instances, the detection is fluorescent detection, and the light is redirected by fluorescent excitation/emission (e.g., fluorescent excitation/emission of a fluorescently labeled component). Additionally, or alternatively, detection is by scattered light.

The electrical property, e.g., impedance, may be used to detect a certain characteristic of the droplet or particle. For example, the characteristic may be the size of the droplet or particle, the velocity of the droplet or particle, the presence or absence of one or more particles in the droplet, and/or the location of one or more particles in the droplet. A droplet may contain more than one (e.g., 2, 3, 4, 5, 6, 7, 8, 9, 10, or more) particles. The particle may include a cell, a bead, or a combination thereof. The characteristic of the droplet may be a characteristic of the particle in the droplet. For example, the method may be used to detect the type of cell, the composition of a cell, viability of the cell, or the size of a cell, e.g., in the droplet.

The electrical property, e.g., impedance, of a particle or droplet can be a function of its size and composition. Thus, various cell types (e.g., lymphocytes, monocytes, granulocytes) can be distinguished based on their impedance. This may be used to distinguish droplets that contain a cell, as compared to droplets that do not contain a cell. Furthermore, it can be used to distinguish droplets that contain different types of cells. The detection of a characteristic of the droplet or particle may be used as a trigger to actuate a sorter to send a droplet to a particular partition.

In some embodiments, methods for detection include detecting light simultaneously from a plurality of detection points in a microfluidic device. The device or a system including the device may include a plurality of light guides with a receiving end and a transmitting end, and the receiving end of each of the plurality of light guides receive light from one of the plurality of detection points. A detector may include an array sensor (e.g., a charge-coupled device (CCD) or a complementary metal oxide semiconductor (CMOS) sensor) having a plurality of regions, and each region may receive light transmitted from one of the transmitting ends of the plurality of light guides. The regions may be spatially distinct to allow simultaneous detection of each region without overlapping signal.

Other methods include simultaneously detecting a plurality of droplets or particles in a microfluidic device with a plurality of droplet or particle sources. The method may include passing a liquid containing the plurality of droplets or particles to the plurality of detection points and simultaneously detecting light from the plurality of detection points using the array sensor, thereby simultaneously detecting the plurality of droplets or particles. In some embodiments, the simultaneous detection includes producing a digital image or a video of light detected from the plurality of detection points, e.g., a single digital image with each region spatially distinct to allow simultaneous detection of each region without overlapping signal.

### Methods for Generating Droplets or Particles

The methods described herein may be used to generate, detect, and/or sort droplets or particles, e.g., of uniform and predictable sizes, and with high throughput. This may be used to greatly increase the efficiency of single cell applications and/or other applications receiving droplet- or particle-based input. Such single cell applications and other applications may often be capable of processing a certain range of droplet or particle sizes. The methods may be employed to generate, sort, and/or detect droplets or particles for use as microscale chemical reactors, where the volumes of the chemical reactants are small (˜pLs).

The methods disclosed herein may produce emulsions, generally, i.e., droplet of a dispersed phases in a continuous phase. For example, droplets may include a first liquid, and the other liquid may be a second liquid. The first liquid may be substantially immiscible with the second liquid. In some instances, the first liquid may be an aqueous liquid or may be substantially miscible with water. Droplets produced according to the methods disclosed herein may combine multiple liquids. For example, a droplet may combine a first and third liquids. The first liquid may be substantially miscible with the third liquid. The second liquid may be an oil, as described herein.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

The methods described herein may allow for the sorting and/or production of one or more droplets containing a single particle, e.g., bead, and/or single biological particle (e.g., cell) with uniform and predictable droplet size. The methods also allow for the sorting and/or production of one or more droplets comprising a single biological particle (e.g., cell) and more than one particle, e.g., bead, one or more droplets comprising more than one biological particle (e.g., cell) and a single particle, e.g., bead, and/or one or more droplets comprising more than one biological particle (e.g., cell) and more than one particle, e.g., beads. The methods may also allow for increased throughput of droplet sorting and/or formation.

Droplets are in general formed by allowing a first liquid to flow into a second liquid in a droplet formation region, where droplets spontaneously form as described herein. The droplets may comprise an aqueous liquid dispersed phase within a non-aqueous continuous phase, such as an oil phase. In some cases, droplet formation may occur in the absence of externally driven movement of the continuous phase, e.g., a second liquid, e.g., an oil. As discussed above, the continuous phase may nonetheless be externally driven, even though it is not required for droplet formation. Emulsion systems for creating stable droplets in non-aqueous (e.g., oil) continuous phases are described in detail in, for example, U.S. Pat. No. 9,012,390, which is entirely incorporated herein by reference for all purposes. Alternatively, or in addition, the droplets may comprise, for example, micro-vesicles that have an outer barrier surrounding an inner liquid center or core. In some cases, the droplets may comprise a porous matrix that is capable of entraining and/or retaining materials within its matrix. A variety of different vessels are described in, for example, U.S. Patent Application Publication No. 2014/0155295, which is entirely incorporated herein by reference for all purposes. The droplets can be collected in a substantially stationary volume of liquid, e.g., with the buoyancy of the formed droplets moving them out of the path of nascent droplets (up or down depending on the relative density of the droplets and continuous phase). Alternatively, or in addition, the formed droplets can be moved out of the path of nascent droplets actively, e.g., using a gentle flow of the continuous phase, e.g., a liquid stream or gently stirred liquid.

Allocating particles, e.g., beads (e.g., microcapsules carrying barcoded oligonucleotides) or biological particles (e.g., cells) to discrete droplets may generally be accomplished by introducing a flowing stream of particles, e.g., beads, in an aqueous liquid into a flowing stream or non-flowing reservoir of a non-aqueous liquid, such that droplets are generated. In some instances, the occupancy of the resulting droplets (e.g., number of particles, e.g., beads, per droplet) can be controlled by providing the aqueous stream at a certain concentration or frequency of particles, e.g., beads and sorting the droplets in a suitable manner. In some instances, the occupancy of the resulting droplets can also be controlled by adjusting one or more geometric features at the point of droplet formation, such as a width of a fluidic channel carrying the particles, e.g., beads, relative to a diameter of a given particles, e.g., beads and subsequently sorting the droplets to provide uniform populations within the partitions of the collection region.

Where single particle-, e.g., bead-, containing droplets are desired, the relative flow rates of the liquids can be selected such that, on average, the droplets contain fewer than one particle, e.g., bead, per droplet in order to ensure that those droplets that are occupied are primarily singly occupied. In some embodiments, the relative flow rates of the liquids can be selected such that a majority of droplets are occupied, for example, allowing for only a small percentage of unoccupied droplets. The flows and channel architectures can be controlled as to ensure a desired number of singly occupied droplets, less than a certain level of unoccupied droplets and/or less than a certain level of multiply occupied droplets.

The methods described herein can be operated such that a majority of occupied droplets include no more than one biological particle per occupied droplet. In some cases, the droplet sorting and/or formation processes are conducted such that fewer than 25% of the occupied droplets contain more than one biological particle (e.g., multiply occupied droplets), and in many cases, fewer than 20% of the occupied droplets have more than one biological particle. In some cases, fewer than 10% or even fewer than 5% of the occupied droplets include more than one biological particle per droplet.

It may be desirable to avoid the creation of excessive numbers of empty droplets, for example, from a cost perspective and/or efficiency perspective. However, while this may be accomplished by providing sufficient numbers of particles, e.g., beads, into the droplet or particle formation region, the Poisson distribution may expectedly increase the number of droplets that may include multiple biological particles. As such, at most about 95%, 90%, 85%, 80%, 75%, 70%, 65%, 60%, 55%, 50%, 45%, 40%, 35%, 30%, 25%, 20%, 15%, 10%, 5% or less of the generated droplets can be unoccupied. In some cases, the flow of one or more of the particles, or liquids directed into the droplet or particle formation region can be conducted such that, in many cases, no more than about 50% of the generated droplets, no more than about 25% of the generated droplets, or no more than about 10% of the generated droplets are unoccupied. Furthermore, in the instance where too many empty droplets are formed, it is desirable to sort out the droplets that are not empty for subsequent use. Furthermore, in the instance where too many droplets are formed that do not contain the desired material but are not empty, it is desirable to sort out the droplets that do not contain the desired material for subsequent use. The flows can be controlled so as to present non-Poisson distribution of singly occupied droplets while providing lower levels of unoccupied droplets. The above noted ranges of unoccupied droplets can be achieved while still providing any of the single occupancy rates described above. For example, in many cases, the use of the systems and methods described herein creates resulting droplets that have multiple occupancy rates of less than about 25%, less than about 20%, less than about 15%, less than about 10%, and in many cases, less than about 5%, while having unoccupied droplets of less than about 50%, less than about 40%, less than about 30%, less than about 20%, less than about 10%, less than about 5%, or less.

The flow of the first fluid may be such that the droplets contain a single particle, e.g., bead. In certain embodiments, the yield of droplets containing a single particle is at least 80%, e.g., at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%.

As will be appreciated, the above-described occupancy rates are also applicable to droplets that include both biological particles (e.g., cells) and beads. The occupied droplets (e.g., at least about 10%, 20%, 30%, 40%, 50%, 60%, 70%, 80%, 90%, 95%, or 99% of the occupied droplets) can include both a bead and a biological particle. Particles, e.g., beads, within a channel (e.g., a particle channel) may flow at a substantially regular flow profile (e.g., at a regular flow rate+/−10%) to provide a droplet, when formed and/or sorted, with a single particle (e.g., bead) and a single cell or other biological particle. Such regular flow profiles may permit the droplets to have a dual occupancy (e.g., droplets having at least one bead and at least one cell or biological particle) greater than 5%, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97% 98%, or 99%. In some embodiments, the droplets have a 1:1 dual occupancy (i.e., droplets having exactly one particle (e.g., bead) and exactly one cell or biological particle) greater than 5%, 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97% 98%, or 99%. Such regular flow profiles and devices that may be used to provide such regular flow profiles are provided, for example, in U.S. Patent Publication No. 2015/0292988, which is entirely incorporated herein by reference. Any of the aforementioned occupancy characteristics can be readily quantified by the methods of detection described herein

In some cases, additional particles may be used to deliver additional reagents to a droplet. In such cases, it may be advantageous to introduce different particles (e.g., beads) into a common channel (e.g., proximal to or upstream from a droplet or particle formation region) or droplet formation intersection from different bead sources (e.g., containing different associated reagents) through different channel inlets into such common channel or droplet or particle formation region. In such cases, the flow and/or frequency of each of the different particle, e.g., bead, sources into the channel or fluidic connections may be controlled to provide for the desired ratio of particles, e.g., beads, from each source, while optionally ensuring the desired pairing or combination of such particles, e.g., beads, are formed into a droplet with the desired number of biological particles.

The droplets or particles described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less. For example, the droplets or particles may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where the droplets further comprise particles (e.g., beads or microcapsules), it will be appreciated that the sample liquid volume within the droplets or particles may be less than about 90% of the above described volumes, less than about 80%, less than about 70%, less than about 60%, less than about 50%, less than about 40%, less than about 30%, less than about 20%, or less than about 10% the above described volumes (e.g., of a partitioning liquid), e.g., from 1% to 99%, from 5% to 95%, from 10% to 90%, from 20% to 80%, from 30% to 70%, or from 40% to 60%, e.g., from 1% to 5%, 5% to 10%, 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%, 85% to 90%, 90% to 95%, or 95% to 100% of the above described volumes.

Any suitable number of droplets or particles can be generated, detected, and/or sorted. For example, in a method described herein, a plurality of droplets or particles may be generated, detected, and/or sorted that comprises at least about 1,000 droplets or particles, at least about 5,000 droplets or particles, at least about 10,000 droplets or particles, at least about 50,000 droplets or particles, at least about 100,000 droplets or particles, at least about 500,000 droplets or particles, at least about 1,000,000 droplets or particles, at least about 5,000,000 droplets or particles at least about 10,000,000 droplets or particles, at least about 50,000,000 droplets or particles, at least about 100,000,000 droplets or particles, at least about 500,000,000 droplets or particles, at least about 1,000,000,000 droplets or particles, or more. Moreover, the plurality of droplets or particles may comprise both unoccupied droplets or particles (e.g., empty droplets) and occupied droplets.

The droplets or particles may be polydisperse or monodisperse (e.g., having a homogenous distribution of diameters+/−10%). A plurality of droplets or particles is monodisperse in instances where the droplets or particles have a distribution of diameters such that no more than about 10%, about 5%, about 4%, about 3%, about 2%, about 1%, or less, of the droplets or particles have a diameter greater than or less than about 20%, about 30%, about 50%, about 75%, about 80%, about 90%, about 95%, about 99%, or more, of the average diameter of all of the droplets or particles.

The fluid to be dispersed into droplets or particles may be transported from a reservoir to the droplet or particle formation region. Alternatively, the fluid to be dispersed into droplets is formed in situ by combining two or more fluids in the device. For example, the fluid to be dispersed may be formed by combining one fluid containing one or more reagents with one or more other fluids containing one or more reagents. In these embodiments, the mixing of the fluid streams may result in a chemical reaction. For example, when a particle is employed, a fluid having reagents that disintegrates the particle may be combined with the particle, e.g., immediately upstream of the droplet generating region. In these embodiments, the particles may be cells, which can be combined with lysing reagents, such as surfactants. When particles, e.g., beads, are employed, the particles, e.g., beads, may be dissolved or chemically degraded, e.g., by a change in pH (acid or base), redox potential (e.g., addition of an oxidizing or reducing agent), enzymatic activity, change in salt or ion concentration, or other mechanism.

The first fluid is transported through the first channel at a flow rate sufficient to produce droplets in the droplet formation region. Faster flow rates of the first fluid generally increase the rate of droplet production; however, at a high enough rate, the first fluid will form a jet, which may not break up into droplets. Typically, the flow rate of the first fluid though the first channel may be between about 0.01 μL/min to about 100 μL/min, e.g., 0.1 to 50 μL/min, 0.1 to 10 μL/min, or 1 to 5 μL/min. In some instances, the flow rate of the first liquid may be between about 0.04 μL/min and about 40 μL/min. In some instances, the flow rate of the first liquid may be between about 0.01 μL/min and about 100 μL/min. Alternatively, the flow rate of the first liquid may be less than about 0.01 μL/min. Alternatively, the flow rate of the first liquid may be greater than about 40 μL/min, e.g., 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 μL/min, the droplet radius may not be dependent on the flow rate of first liquid. Alternatively, or in addition, for any of the abovementioned flow rates, the droplet radius may be independent of the flow rate of the first liquid.

The typical droplet or particle formation rate for a single channel in a device of the invention is between 0.1 Hz to 10,000 Hz, e.g., 1 to 1000 Hz or 1 to 500 Hz. The use of multiple first channels can increase the rate of droplet or particle formation by increasing the number of locations of formation.

As discussed above, droplet or particle sorting and/or formation may occur in the absence of externally driven movement of the continuous phase. In such embodiments, the continuous phase flows in response to displacement by the advancing stream of the first fluid or other forces. Channels may be present in the droplet or particle formation region, e.g., including a shelf region, to allow more rapid transport of the continuous phase around the first fluid. Partitions (e.g., channels or reservoirs) may also be present in the sorting region, detection region, and/or collection region. This increase in transport of the continuous phase can increase the rate of droplet or particle sorting and/or formation. Alternatively, the continuous phase may be actively transported. For example, the continuous phase may be actively transported into the droplet or particle formation region, e.g., including a shelf region, to increase the rate of droplet or particle sorting and/or formation; continuous phase may be actively transported to form a sheath flow around the first fluid as it exits the distal end; or the continuous phase may be actively transported to move droplets or particles away from the point of formation.

Additional factors that affect the rate of droplet or particle sorting and/or formation include the viscosity of the first fluid and of the continuous phase, where increasing the viscosity of either fluid reduces the rate of droplet or particle formation. In certain embodiments, the viscosity of the first fluid and/or continuous is between 0.5 cP to 10 cP. Furthermore, lower interfacial tension results in slower droplet or particle formation. In certain embodiments, the interfacial tension is between 0.1 and 100 mN/m, e.g., 1 to 100 mN/m or 2 mN/m to 60 mN/m. The depth of the shelf region can also be used to control the rate of droplet or particle formation, with a shallower depth resulting in a faster rate of formation.

The methods may be used to produce and/or sort droplets or particles in range of 1 μm to 500 μm in diameter, e.g., 1 to 250 μm, 5 to 200 μm, 5 to 150 μm, or 12 to 125 μm. Factors that affect the size of the droplets or particles include the rate of formation, the cross-sectional dimension of the distal end of the first channel, the depth of the shelf, and fluid properties and dynamic effects, such as the interfacial tension, viscosity, and flow rate.

The first liquid may be aqueous, and the second liquid may be an oil (or vice versa). Examples of oils include perfluorinated oils, mineral oil, and silicone oils. For example, a fluorinated oil may include a fluorosurfactant for stabilizing the resulting droplets or particles, for example, inhibiting subsequent coalescence of the resulting droplets or particles. Examples of particularly useful liquids and fluorosurfactants are described, for example, in U.S. Pat. No. 9,012,390, which is entirely incorporated herein by reference for all purposes. Specific examples include hydrofluoroethers, such as HFE 7500, 7300, 7200, or 7100. Suitable liquids are those described in US 2015/0224466 and US 62/522,292, the liquids of which are hereby incorporated by reference. In some cases, liquids include additional components such as a particle, e.g., a cell or a gel bead. As discussed above, the first fluid or continuous phase may include reagents for carrying out various reactions, such as nucleic acid amplification, lysis, or bead dissolution. The first liquid or continuous phase may include additional components that stabilize or otherwise affect the droplets or particles or a component inside the droplet. Such additional components include surfactants, antioxidants, preservatives, buffering agents, antibiotic agents, salts, chaotropic agents, enzymes, nanoparticles, and sugars.

Devices, systems, compositions, and methods of the present disclosure may be used for various applications, such as, for example, processing a single analyte (e.g., bioanalytes, e.g., RNA, DNA, or protein) or multiple analytes (e.g., bioanalytes, e.g., DNA and RNA, DNA and protein, RNA and protein, or RNA, DNA and protein) from a single cell. For example, a biological particle (e.g., a cell or virus) can be formed in a droplet, and one or more analytes (e.g., bioanalytes) from the biological particle (e.g., cell) can be modified or detected (e.g., bound, labeled, or otherwise modified by an analyte detection moiety) for subsequent processing. The multiple analytes may be from the single cell. This process may enable, for example, proteomic, transcriptomic, and/or genomic analysis of the cell or population thereof (e.g., simultaneous proteomic, transcriptomic, and/or genomic analysis of the cell or population thereof).

Methods of modifying analytes include providing a plurality of particles (e.g., beads) in a liquid carrier (e.g., an aqueous carrier); providing a sample containing an analyte (e.g., as part of a cell, or component or product thereof) in a sample liquid; and using the device to combine the liquids and form an analyte detection droplet containing one or more particles and one or more analytes (e.g., as part of one or more cells, or components or products thereof). Such sequestration of one or more particles with analyte (e.g., bioanalyte associated with a cell) in a droplet enables labeling of discrete portions of large, heterologous samples (e.g., single cells within a heterologous population). Once labeled or otherwise modified, droplets or particles can be subsequently sorted or combined (e.g., by breaking an emulsion), and the resulting liquid can be analyzed to determine a variety of properties associated with each of numerous single cells.

In particular embodiments, the invention features methods of sorting and/or producing analyte detection droplets using a device having a particle channel and a sample channel that intersect proximal to a droplet or particle formation region. Particles having an analyte-detection moiety in a liquid carrier flow proximal-to-distal (e.g., towards the droplet or particle formation region) through the particle channel and a sample liquid containing an analyte flows proximal-to-distal (e.g., towards the droplet or particle formation region) through the sample channel until the two liquids meet and combine at the intersection of the sample channel and the particle channel, upstream (and/or proximal to) the droplet or particle formation region. The combination of the liquid carrier with the sample liquid results in an analyte detection liquid. In some embodiments, the two liquids are miscible (e.g., they both contain solutes in water or aqueous buffer). The combination of the two liquids can occur at a controlled relative rate, such that the analyte detection liquid has a desired volumetric ratio of particle liquid to sample liquid, a desired numeric ratio of particles to cells, or a combination thereof (e.g., one particle per cell per 50 pL). As the analyte detection liquid flows through the droplet or particle formation region into a partitioning liquid (e.g., a liquid which is immiscible with the analyte detection liquid, such as an oil), analyte detection droplets form. These analyte detection droplets may continue to flow through a sorting region and into a collection region following sorting. The sorting region or collection region may contain one or more partitions (e.g., channels). In some embodiments, the analyte detection droplets may flow through one or more channels. Alternatively, or in addition, the analyte detection droplets may accumulate (e.g., as a substantially stationary population) in a collection region. In some cases, the accumulation of a population of droplets may occur by a gentle flow of a fluid within the collection region, e.g., to move the formed droplets out of the path of the nascent droplets.

Devices useful for analyte detection may feature any combination of elements described herein. For example, various droplet or particle formation regions can be employed in the design of a device for analyte detection. In some embodiments, analyte detection droplets are formed at a droplet or particle formation region having a shelf region, where the analyte detection liquid expands in at least one dimension as it passes through the droplet or particle formation region. Any shelf region described herein can be useful in the methods of analyte detection droplet sorting and/or formation provided herein. Additionally, or alternatively, the droplet or particle formation region may have a step at or distal to an inlet of the droplet or particle formation region (e.g., within the droplet or particle formation region or distal to the droplet or particle formation region). In some embodiments, analyte detection droplets are formed without externally driven flow of a continuous phase (e.g., by one or more crossing flows of liquid at the droplet or particle formation region). Alternatively, analyte detection droplets are formed in the presence of an externally driven flow of a continuous phase.

A device useful for droplet formation, e.g., analyte detection, may feature multiple droplet formation, sorting, and/or collection regions (e.g., in or out of (e.g., as independent, parallel circuits) fluid communication with one another. For example, such a device may have 2-100, 3-50, 4-40, 5-30, 6-24, 8-18, or 9-12, e.g., 2-6, 6-12, 12-18, 18-24, 24-36, 36-48, or 48-96, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more droplet formation, sorting, and/or collection regions configured to produce analyte detection droplets).

Source reservoirs can store liquids prior to and during droplet or particle formation. In some embodiments, a device useful in analyte detection droplet or particle formation includes one or more particle reservoirs connected proximally to one or more particle channels. Particle suspensions can be stored in particle reservoirs prior to analyte detection droplet formation. Particle reservoirs can be configured to store particles containing an analyte detection moiety. For example, particle reservoirs can include, e.g., a coating to prevent adsorption or binding (e.g., specific or non-specific binding) of particles or analyte-detection moieties. Additionally, or alternatively, particle reservoirs can be configured to minimize degradation of analyte detection moieties (e.g., by containing nuclease, e.g., DNAse or RNAse) or the particle matrix itself, accordingly.

Additionally, or alternatively, a device includes one or more sample reservoirs connected proximally to one or more sample channels. Samples containing cells and/or other reagents useful in analyte detection and/or droplet sorting and/or formation can be stored in sample reservoirs prior to analyte detection droplet sorting and/or formation. Sample reservoirs can be configured to reduce degradation of sample components, e.g., by including nuclease (e.g., DNAse or RNAse).

Methods of the invention include administering a sample and/or particles to the device, for example, (a) by pipetting a sample liquid, or a component or concentrate thereof, into a sample reservoir and/or (b) by pipetting a liquid carrier (e.g., an aqueous carrier) and/or particles into a particle reservoir. In some embodiments, the method involves first pipetting the liquid carrier (e.g., an aqueous carrier) and/or particles into the particle reservoir prior to pipetting the sample liquid, or a component or concentrate thereof, into the sample reservoir.

The sample reservoir and/or particle reservoir may be incubated in conditions suitable to preserve or promote activity of their contents until the initiation or commencement of droplet or particle formation and sorting.

Sorting and/or formation of bioanalyte detection droplets or particles, as provided herein, can be used for various applications. In particular, by sorting and/or forming bioanalyte detection droplets using the methods, devices, systems, and kits herein, a user can perform standard downstream processing methods to barcode heterogeneous populations of cells or perform single-cell nucleic acid sequencing.

In methods of barcoding a population of cells, an aqueous sample having a population of cells is combined with bioanalyte detection particles having a nucleic acid primer sequence and a barcode in an aqueous carrier at an intersection of the sample channel and the particle channel to form a reaction liquid. Upon passing through the droplet or particle formation region, the reaction liquid meets a partitioning liquid (e.g., a partitioning oil) under droplet-forming conditions to form a plurality of reaction droplets, each reaction droplet having one or more of the particles and one or more cells in the reaction liquid. The reaction droplets are incubated under conditions sufficient to allow for barcoding of the nucleic acid of the cells in the reaction droplets or particles. In some embodiments, the conditions sufficient for barcoding are thermally optimized for nucleic acid replication, transcription, and/or amplification. For example, reaction droplets can be incubated at temperatures configured to enable reverse transcription of RNA produced by a cell in a droplet into DNA, using reverse transcriptase. Additionally, or alternatively, reaction droplets may be cycled through a series of temperatures to promote amplification, e.g., as in a polymerase chain reaction (PCR). Accordingly, in some embodiments, one or more nucleotide amplification reagents (e.g., PCR reagents) are included in the reaction droplets (e.g., primers, nucleotides, and/or polymerase). Any one or more reagents for nucleic acid replication, transcription, and/or amplification can be provided to the reaction droplet by the aqueous sample, the liquid carrier, or both. In some embodiments, one or more of the reagents for nucleic acid replication, transcription, and/or amplification are in the aqueous sample.

The invention also provides methods of single-cell nucleic acid sequencing, in which a heterologous population of cells can be characterized by their individual gene expression, e.g., relative to other cells of the population. Methods of barcoding cells discussed above and known in the art can be part of the methods of single-cell nucleic acid sequencing provided herein. After barcoding, nucleic acid transcripts that have been barcoded are sequenced, and sequences can be processed, analyzed, and stored according to known methods. In some embodiments, these methods enable the generation of a genome library containing gene expression data for any single cell within a heterologous population.

Alternatively, the ability to sequester a single cell in a reaction droplet provided by methods herein enables bioanalyte detection for applications beyond genome characterization. For example, a reaction droplet containing a single cell and variety of analyte detection moieties capable of binding different proteins can allow a single cell to be detectably labeled to provide relative protein expression data. In some embodiments, analyte detection moieties are antigen-binding molecules (e.g., antibodies or fragments thereof), wherein each antibody clone is detectably labeled (e.g., with a fluorescent marker having a distinct emission wavelength). Binding of antibodies to proteins can occur within the reaction droplet, and cells can be subsequently analyzed for bound antibodies according to known methods to generate a library of protein expression. Other methods known in the art can be employed to characterize cells within heterologous populations after detecting analytes using the methods provided herein. In one example, following the sorting and/or formation of droplets, subsequent operations that can be performed can include formation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the droplet). An exemplary use for droplets formed and/or sorted using methods of the invention is in performing nucleic acid amplification, e.g., polymerase chain reaction (PCR), where the reagents necessary to carry out the amplification are contained within the first fluid. In the case where a droplet is a droplet in an emulsion, the emulsion can be broken and the contents of the droplet pooled for additional operations. Additional reagents that may be included in a droplet along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of poly-T sequence during sequencing and/or sequence the 5′ end of a polynucleotide sequence. The amplification products, for example first amplification products and/or second amplification products, may be subject to sequencing for sequence analysis. In some cases, amplification may be performed using the Partial Hairpin Amplification for Sequencing (PHASE) method.

### Methods of Device Manufacture

The devices (e.g., microfluidic devices) of the present disclosure may be fabricated in any of a variety of conventional ways. For example, in some cases the devices comprise layered structures, where a first layer includes a planar surface into which is disposed a series of channels or grooves that correspond to the channel network in the finished device. A second layer includes a planar surface on one side, and a series of reservoirs defined on the opposing surface, where the reservoirs communicate as passages through to the planar layer, such that when the planar surface of the second layer is mated with the planar surface of the first layer, the reservoirs defined in the second layer are positioned in liquid communication with the termini of the channels on the first layer. Alternatively, both the reservoirs and the connected channels may be fabricated into a single part, where the reservoirs are provided upon a first surface of the structure, with the apertures of the reservoirs extending through to the opposing surface of the structure. The channel network is fabricated as a series of grooves and features in this second surface. A thin laminating layer is then provided over the second surface to seal, and provide the final wall of the channel network, and the bottom surface of the reservoirs.

These layered structures may be fabricated in whole or in part from polymeric materials, such as polyethylene or polyethylene derivatives, such as cyclic olefin copolymers (COC), polymethylmethacrylate (PMMA), polydimethylsiloxane (PDMS), polycarbonate, polystyrene, polypropylene, polyvinyl chloride, polytetrafluoroethylene, polyoxymethylene, polyether ether ketone, polycarbonate, polystyrene, or the like, or they may be fabricated in whole or in part from inorganic materials, such as silicon, or other silica based materials, e.g., glass, quartz, fused silica, borosilicate glass, metals, ceramics, and combinations thereof. Polymeric device components may be fabricated using any of a number of processes including soft lithography, embossing techniques, micromachining, e.g., laser machining, or in some aspects injection molding of the layer components that include the defined channels as well as other structures, e.g., reservoirs, integrated functional components, etc. In some aspects, the structure comprising the reservoirs and channels may be fabricated using, e.g., injection molding techniques to produce polymeric structures. In such cases, a laminating layer may be adhered to the molded structured part through readily available methods, including thermal lamination, solvent based lamination, sonic welding, or the like.

As will be appreciated, structures made of or including inorganic materials also may be fabricated using known techniques. For example, channels and other structures may be micro-machined into surfaces or etched into the surfaces using standard photolithographic techniques. In some aspects, the microfluidic devices or components thereof may be fabricated using three-dimensional printing techniques to fabricate the channel or other structures of the devices and/or their discrete components.

### Methods for Surface Modifications

The invention features methods for producing a microfluidic device that has a surface modification, e.g., a surface with a modified water contact angle. The methods may be employed to modify the surface of a device such that a liquid can “wet” the surface by altering the contact angle the liquid makes with the surface. An exemplary use of the methods of the invention is in creating a device having differentially coated surfaces to optimize droplet or particle formation.

Devices to be modified with surface coating agents may be primed, e.g., pre-treated, before coating processes occur. In one embodiment, the device has a channel that is in fluid communication with a droplet or particle formation region. In particular, the droplet or particle formation region is configured to allow a liquid exiting the channel to expand in at least one dimension. A surface of the droplet or particle formation region is contacted by at least one reagent that has an affinity for the primed surface to produce a surface having a first water contact angle of greater than about 90°, e.g., a hydrophobic or fluorophillic surface. In certain embodiments, the first contact angle is greater than the water contact angle of the primed surface. In other embodiments, the first contact angle is greater than the water contact angle of the channel surface. Thus, the method allows for the differential coating of surfaces within the microfluidic device.

A surface may be primed by depositing a metal oxide onto it. Example metal oxides useful for priming surfaces include, but are not limited to, Al₂O₃, TiO₂, SiO₂, or a combination thereof. Other metal oxides useful for surface modifications are known in the art. The metal oxide can be applied to the surface by standard deposition techniques, including, but not limited to, atomic layer deposition (ALD), physical vapor deposition (PVD), e.g., sputtering, chemical vapor deposition (CVD), or laser deposition. Other deposition techniques for coating surfaces, e.g., liquid-based deposition, are known in the art. For example, an atomic layer of Al₂O₃ can be prepared on a surface by depositing trimethylaluminum (TMA) and water.

In some cases, the coating agent may create a surface that has a water contact angle greater than 90°, e.g., hydrophobic or fluorophillic, or may create a surface with a water contact angle of less than 90°, e.g., hydrophilic. For example, a fluorophillic surface may be created by flowing fluorosilane (e.g., H₃FSi) through a primed device surface, e.g., a surface coated in a metal oxide. The priming of the surfaces of the device enhances the adhesion of the coating agents to the surface by providing appropriate surface functional groups. In some cases, the coating agent used to coat the primed surface may be a liquid reagent. For example, when a liquid coating agent is used to coat a surface, the coating agent may be directly introduced to the droplet or particle formation region by a feed channel in fluid communication with the droplet or particle formation region. In order to keep the coating agent localized to the droplet or particle formation region, e.g., prevent ingress of the coating agent to another portion of the device, e.g., the channel, the portion of the device that is not to be coated can be substantially blocked by a substance that does not allow the coating agent to pass (e.g., no more than 10% of the device is not blocked). For example, in order to prevent ingress of a liquid coating agent into the channel, the channel may be filled with a blocking liquid that is substantially immiscible with the coating agent (e.g., no more than 10% of the blocking liquid is miscible. The blocking liquid may be actively transported through the portion of the device not to be coated, or the blocking liquid may be stationary. Alternatively, the channel may be filled with a pressurized gas such that the pressure prevents ingress of the coating agent into the channel. The coating agent may also be applied to the regions of interest external to the main device. For example, the device may incorporate an additional reservoir and at least one feed channel that connects to the region of interest such that no coating agent is passed through the device.

### EXAMPLES

The following examples describe devices and methods for sorting droplets. The devices and methods described may also be employed for sorting particles.

### Example 1

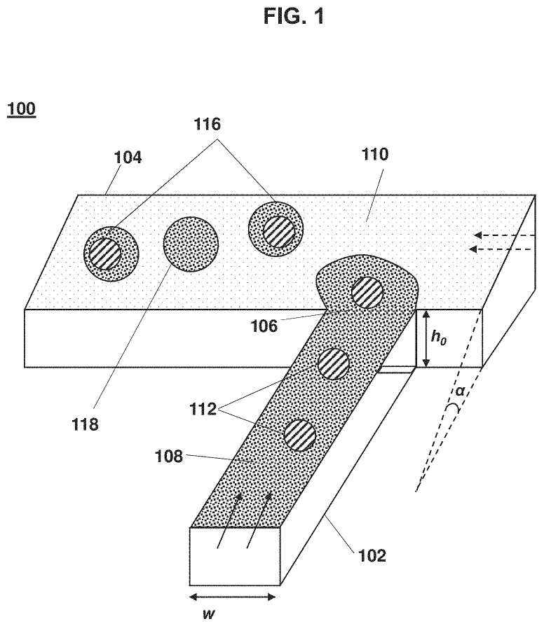

FIG. 1 shows an example of a microfluidic device for the controlled inclusion of particles, e.g., beads, into discrete droplets. A device 100 can include a channel 102 communicating at a fluidic connection 106 (or intersection) with a reservoir 104. The reservoir 104 can be a chamber. Any reference to “reservoir,” as used herein, can also refer to a “chamber.” In operation, an aqueous liquid 108 that includes suspended beads 112 may be transported along the channel 102 into the fluidic connection 106 to meet a second liquid 110 that is immiscible with the aqueous liquid 108 in the reservoir 104 to create droplets 116, 118 of the aqueous liquid 108 flowing into the reservoir 104. At the fluidic connection 106 where the aqueous liquid 108 and the second liquid 110 meet, droplets can form based on factors such as the hydrodynamic forces at the fluidic connection 106, flow rates of the two liquids 108, 110, liquid properties, and certain geometric parameters (e.g., w, h₀, α, etc.) of the device 100. A plurality of droplets can be collected in the reservoir 104 by continuously injecting the aqueous liquid 108 from the channel 102 through the fluidic connection 106.

In some instances, the second liquid 110 may not be subjected to and/or directed to any flow in or out of the reservoir 104. For example, the second liquid 110 may be substantially stationary in the reservoir 104. In some instances, the second liquid 110 may be subjected to flow within the reservoir 104, but not in or out of the reservoir 104, such as via application of pressure to the reservoir 104 and/or as affected by the incoming flow of the aqueous liquid 108 at the fluidic connection 106. Alternatively, the second liquid 110 may be subjected and/or directed to flow in or out of the reservoir 104. For example, the reservoir 104 can be a channel directing the second liquid 110 from upstream to downstream, transporting the generated droplets. Alternatively, or in addition, the second liquid 110 in reservoir 104 may be used to sweep formed droplets away from the path of the nascent droplets.

While FIG. 1 illustrates the reservoir 104 having a substantially linear inclination (e.g., creating the expansion angle, a) relative to the channel 102, the inclination may be non-linear. The expansion angle may be an angle between the immediate tangent of a sloping inclination and the channel 102. In an example, the reservoir 104 may have a dome-like (e.g., hemispherical) shape. The reservoir 104 may have any other shape.

### Example 2

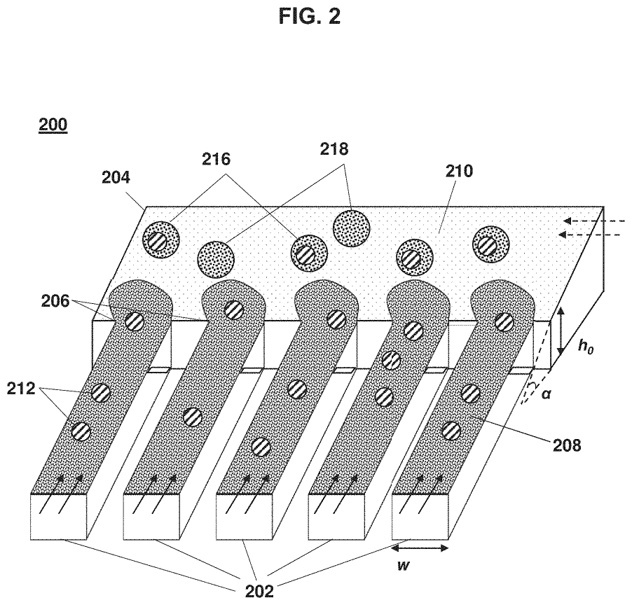

FIG. 2 shows an example of a microfluidic device for increased droplet formation throughput. A device 200 can comprise a plurality of channels 202 and a reservoir 204. Each of the plurality of channels 202 may be in fluid communication with the reservoir 204. The device 200 can comprise a plurality of fluidic connections 206 between the plurality of channels 202 and the reservoir 204. Each fluidic connection can be a point of droplet formation. The channel 102 from the device 100 in FIG. 1 and any description to the components thereof may correspond to a given channel of the plurality of channels 202 in device 200 and any description to the corresponding components thereof. The reservoir 104 from the device 100 and any description to the components thereof may correspond to the reservoir 204 from the device 200 and any description to the corresponding components thereof.

Each channel of the plurality of channels 202 may comprise an aqueous liquid 208 that includes suspended particles, e.g., beads, 212. The reservoir 204 may comprise a second liquid 210 that is immiscible with the aqueous liquid 208. In some instances, the second liquid 210 may not be subjected to and/or directed to any flow in or out of the reservoir 204. For example, the second liquid 210 may be substantially stationary in the reservoir 204. Alternatively, or in addition, the formed droplets can be moved out of the path of nascent droplets using a gentle flow of the second liquid 210 in the reservoir 204. In some instances, the second liquid 210 may be subjected to flow within the reservoir 204, but not in or out of the reservoir 204, such as via application of pressure to the reservoir 204 and/or as affected by the incoming flow of the aqueous liquid 208 at the fluidic connections. Alternatively, the second liquid 210 may be subjected and/or directed to flow in or out of the reservoir 204. For example, the reservoir 204 can be a channel directing the second liquid 210 from upstream to downstream, transporting the generated droplets. Alternatively, or in addition, the second liquid 210 in reservoir 204 may be used to sweep formed droplets away from the path of the nascent droplets.

In operation, the aqueous liquid 208 that includes suspended particles, e.g., beads, 212 may be transported along the plurality of channels 202 into the plurality of fluidic connections 206 to meet the second liquid 210 in the reservoir 204 to create droplets 216, 218. A droplet may form from each channel at each corresponding fluidic connection with the reservoir 204. At the fluidic connection where the aqueous liquid 208 and the second liquid 210 meet, droplets can form based on factors such as the hydrodynamic forces at the fluidic connection, flow rates of the two liquids 208, 210, liquid properties, and certain geometric parameters (e.g., w, h₀, α, etc.) of the device 200, as described elsewhere herein. A plurality of droplets can be collected in the reservoir 204 by continuously injecting the aqueous liquid 208 from the plurality of channels 202 through the plurality of fluidic connections 206. The geometric parameters, w, h₀, and α, may or may not be uniform for each of the channels in the plurality of channels 202. For example, each channel may have the same or different widths at or near its respective fluidic connection with the reservoir 204. For example, each channel may have the same or different height at or near its respective fluidic connection with the reservoir 204. In another example, the reservoir 204 may have the same or different expansion angle at the different fluidic connections with the plurality of channels 202. When the geometric parameters are uniform, beneficially, droplet size may also be controlled to be uniform even with the increased throughput. In some instances, when it is desirable to have a different distribution of droplet sizes, the geometric parameters for the plurality of channels 202 may be varied accordingly.

### Example 3

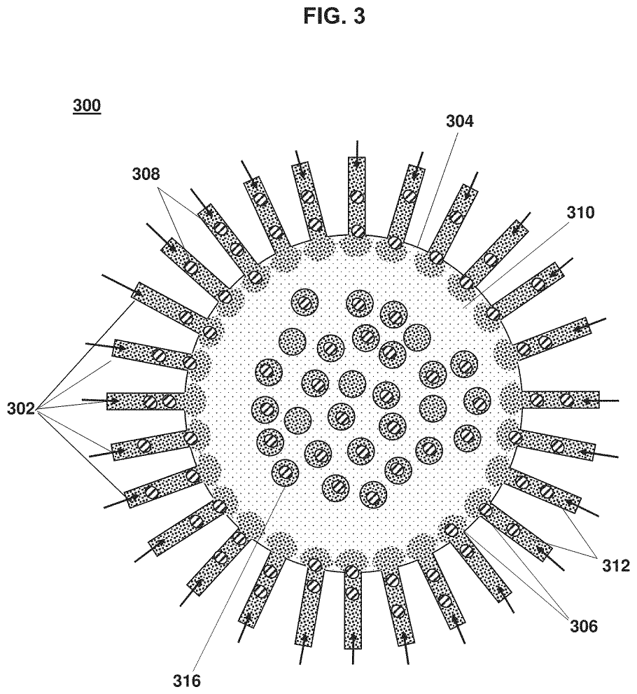

FIG. 3 shows another example of a microfluidic device for increased droplet formation throughput. A microfluidic device 300 can comprise a plurality of channels 302 arranged generally circularly around the perimeter of a reservoir 304. Each of the plurality of channels 302 may be in liquid communication with the reservoir 304. The device 300 can comprise a plurality of fluidic connections 306 between the plurality of channels 302 and the reservoir 304. Each fluidic connection can be a point of droplet formation. The channel 102 from the device 100 in FIG. 1 and any description to the components thereof may correspond to a given channel of the plurality of channels 302 in device 300 and any description to the corresponding components thereof. The reservoir 104 from the device 100 and any description to the components thereof may correspond to the reservoir 304 from the device 300 and any description to the corresponding components thereof.

Each channel of the plurality of channels 302 may comprise an aqueous liquid 308 that includes suspended particles, e.g., beads, 312. The reservoir 304 may comprise a second liquid 310 that is immiscible with the aqueous liquid 308. In some instances, the second liquid 310 may not be subjected to and/or directed to any flow in or out of the reservoir 304. For example, the second liquid 310 may be substantially stationary in the reservoir 304. In some instances, the second liquid 310 may be subjected to flow within the reservoir 304, but not in or out of the reservoir 304, such as via application of pressure to the reservoir 304 and/or as affected by the incoming flow of the aqueous liquid 308 at the fluidic connections. Alternatively, the second liquid 310 may be subjected and/or directed to flow in or out of the reservoir 304. For example, the reservoir 304 can be a channel directing the second liquid 310 from upstream to downstream, transporting the generated droplets. Alternatively, or in addition, the second liquid 310 in reservoir 304 may be used to sweep formed droplets away from the path of the nascent droplets.

In operation, the aqueous liquid 308 that includes suspended particles, e.g., beads, 312 may be transported along the plurality of channels 302 into the plurality of fluidic connections 306 to meet the second liquid 310 in the reservoir 304 to create a plurality of droplets 316. A droplet may form from each channel at each corresponding fluidic connection with the reservoir 304. At the fluidic connection where the aqueous liquid 308 and the second liquid 310 meet, droplets can form based on factors such as the hydrodynamic forces at the fluidic connection, flow rates of the two liquids 308, 310, liquid properties, and certain geometric parameters (e.g., widths and heights of the channels 302, expansion angle of the reservoir 304, etc.) of the channel 300, as described elsewhere herein. A plurality of droplets can be collected in the reservoir 304 by continuously injecting the aqueous liquid 308 from the plurality of channels 302 through the plurality of fluidic connections 306.

### Example 4

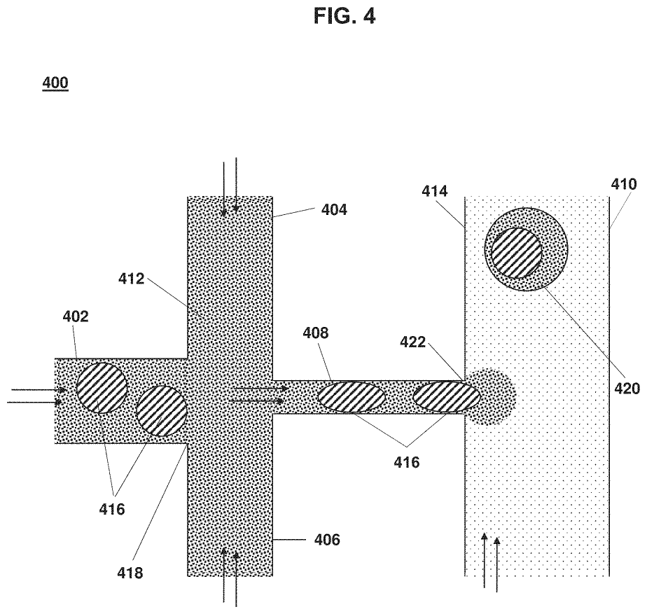

FIG. 4 shows another example of a microfluidic device for the introduction of beads into discrete droplets. A device 400 can include a first channel 402, a second channel 404, a third channel 406, a fourth channel 408, and a reservoir 410. The first channel 402, second channel 404, third channel 406, and fourth channel 408 can communicate at a first intersection 418. The fourth channel 408 and the reservoir 410 can communicate at a fluidic connection 422. In some instances, the fourth channel 408 and components thereof can correspond to the channel 102 in the device 100 in FIG. 1 and components thereof. In some instances, the reservoir 410 and components thereof can correspond to the reservoir 104 in the device 100 and components thereof.

In operation, an aqueous liquid 412 that includes suspended particles, e.g., beads, 416 may be transported along the first channel 402 into the intersection 418 at a first frequency to meet another source of the aqueous liquid 412 flowing along the second channel 404 and the third channel 406 towards the intersection 418 at a second frequency. In some instances, the aqueous liquid 412 in the second channel 404 and the third channel 406 may comprise one or more reagents. At the intersection, the combined aqueous liquid 412 carrying the suspended particles, e.g., beads, 416 (and/or the reagents) can be directed into the fourth channel 408. In some instances, a cross-section width or diameter of the fourth channel 408 can be chosen to be less than a cross-section width or diameter of the particles, e.g., beads, 416. In such cases, the particles, e.g., beads, 416 can deform and travel along the fourth channel 408 as deformed particles, e.g., beads, 416 towards the fluidic connection 422. At the fluidic connection 422, the aqueous liquid 412 can meet a second liquid 414 that is immiscible with the aqueous liquid 412 in the reservoir 410 to create droplets 420 of the aqueous liquid 412 flowing into the reservoir 410. Upon leaving the fourth channel 408, the deformed particles, e.g., beads, 416 may revert to their original shape in the droplets 420. At the fluidic connection 422 where the aqueous liquid 412 and the second liquid 414 meet, droplets can form based on factors such as the hydrodynamic forces at the fluidic connection 422, flow rates of the two liquids 412, 414, liquid properties, and certain geometric parameters (e.g., w, h₀, α, etc.) of the channel, as described elsewhere herein. A plurality of droplets can be collected in the reservoir 410 by continuously injecting the aqueous liquid 412 from the fourth channel 408 through the fluidic connection 422.

A discrete droplet generated may include a particle, e.g., a bead, (e.g., as in droplets 420). Alternatively, a discrete droplet generated may include more than one particle, e.g., bead. Alternatively, a discrete droplet generated may not include any particles, e.g., beads. In some instances, a discrete droplet generated may contain one or more biological particles, e.g., cells (not shown in FIG. 4).

In some instances, the second liquid 414 may not be subjected to and/or directed to any flow in or out of the reservoir 410. For example, the second liquid 414 may be substantially stationary in the reservoir 410. In some instances, the second liquid 414 may be subjected to flow within the reservoir 410, but not in or out of the reservoir 410, such as via application of pressure to the reservoir 410 and/or as affected by the incoming flow of the aqueous liquid 412 at the fluidic connection 422. In some instances, the second liquid 414 may be gently stirred in the reservoir 410. Alternatively, the second liquid 414 may be subjected and/or directed to flow in or out of the reservoir 410. For example, the reservoir 410 can be a channel directing the second liquid 414 from upstream to downstream, transporting the generated droplets. Alternatively, or in addition, the second liquid 414 in reservoir 410 may be used to sweep formed droplets away from the path of the nascent droplets.

### Example 5

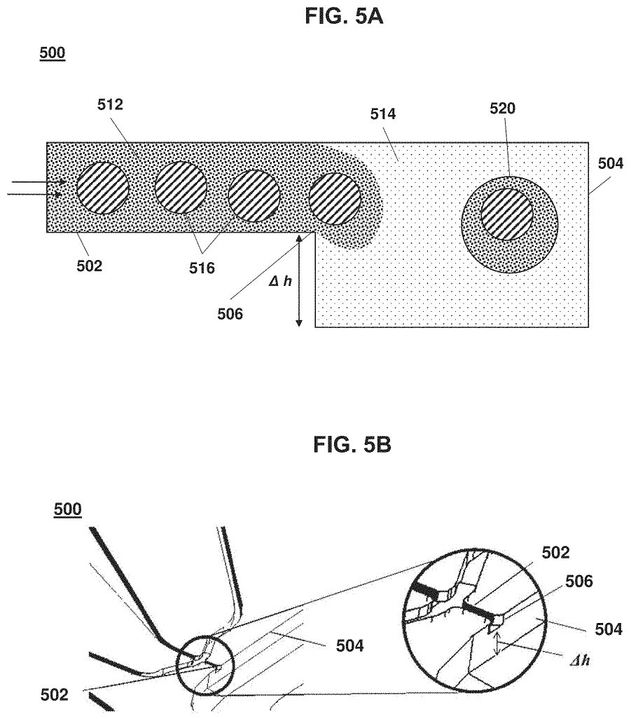

FIG. 5A shows a cross-section view of another example of a microfluidic device with a geometric feature for droplet formation. A device 500 can include a channel 502 communicating at a fluidic connection 506 (or intersection) with a reservoir 504. In some instances, the device 500 and one or more of its components can correspond to the device 100 and one or more of its components. FIG. 5B shows a perspective view of the device 500 of FIG. 5A.

An aqueous liquid 512 comprising a plurality of particles 516 may be transported along the channel 502 into the fluidic connection 506 to meet a second liquid 514 (e.g., oil, etc.) that is immiscible with the aqueous liquid 512 in the reservoir 504 to create droplets 520 of the aqueous liquid 512 flowing into the reservoir 504. At the fluidic connection 506 where the aqueous liquid 512 and the second liquid 514 meet, droplets can form based on factors such as the hydrodynamic forces at the fluidic connection 506, relative flow rates of the two liquids 512, 514, liquid properties, and certain geometric parameters (e.g., Δh, etc.) of the device 500. A plurality of droplets can be collected in the reservoir 504 by continuously injecting the aqueous liquid 512 from the channel 502 at the fluidic connection 506.

While FIGS. 5A and 5B illustrate the height difference, Δh, being abrupt at the fluidic connection 506 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, wherein an angle between any one differential segment of a height profile and an immediately adjacent differential segment of the height profile is greater than 90°. For example, at the fluidic connection 506, a bottom wall of the channel and a bottom wall of the reservoir can meet at an angle greater than 90°. Alternatively, or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively, or in addition, the height difference may variably increase and/or decrease linearly or non-linearly.

### Example 6

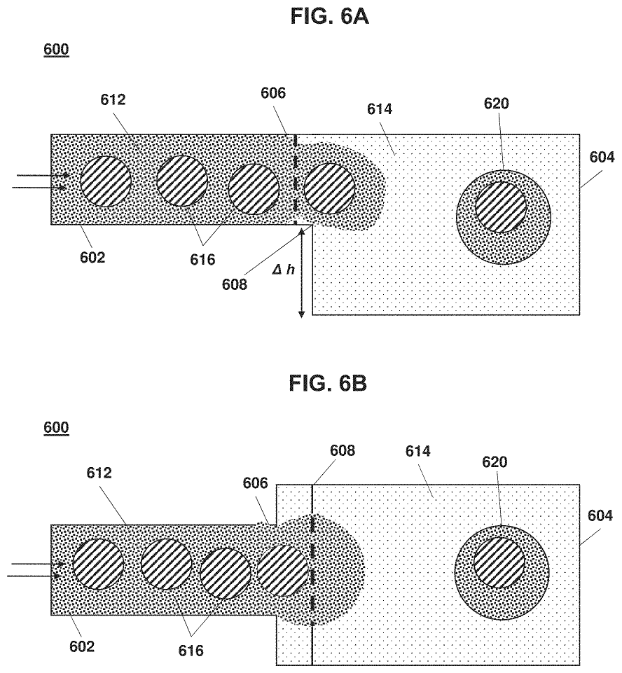

FIGS. 6A and 6B show a cross-section view and a top view, respectively, of another example of a microfluidic device with a geometric feature for droplet formation. A device 600 can include a channel 602 communicating at a fluidic connection 606 (or intersection) with a reservoir 604. In some instances, the device 600 and one or more of its components can correspond to the device 500 and one or more of its components.

An aqueous liquid 612 comprising a plurality of particles 616 may be transported along the channel 602 into the fluidic connection 606 to meet a second liquid 614 (e.g., oil, etc.) that is immiscible with the aqueous liquid 612 in the reservoir 604 to create droplets 620 of the aqueous liquid 612 flowing into the reservoir 604. At the fluidic connection 606 where the aqueous liquid 612 and the second liquid 614 meet, droplets can form based on factors such as the hydrodynamic forces at the fluidic connection 606, relative flow rates of the two liquids 612, 614, liquid properties, and certain geometric parameters (e.g., Δh, ledge, etc.) of the channel 602. A plurality of droplets can be collected in the reservoir 604 by continuously injecting the aqueous liquid 612 from the channel 602 at the fluidic connection 606.

The aqueous liquid may comprise particles. The particles 616 (e.g., beads) can be introduced into the channel 602 from a separate channel (not shown in FIG. 6). In some instances, the particles 616 can be introduced into the channel 602 from a plurality of different channels, and the frequency controlled accordingly. In some instances, different particles may be introduced via separate channels. For example, a first separate channel can introduce beads and a second separate channel can introduce biological particles into the channel 602. The first separate channel introducing the beads may be upstream or downstream of the second separate channel introducing the biological particles.

While FIGS. 6A and 6B illustrate one ledge (e.g., step) in the reservoir 604, as can be appreciated, there may be a plurality of ledges in the reservoir 604, for example, each having a different cross-section height. For example, where there is a plurality of ledges, the respective cross-section height can increase with each consecutive ledge. Alternatively, the respective cross-section height can decrease and/or increase in other patterns or profiles (e.g., increase then decrease then increase again, increase then increase then increase, etc.).

While FIGS. 6A and 6B illustrate the height difference, Δh, being abrupt at the ledge 608 (e.g., a step increase), the height difference may increase gradually (e.g., from about 0 μm to a maximum height difference). In some instances, the height difference may decrease gradually (e.g., taper) from a maximum height difference. In some instances, the height difference may variably increase and/or decrease linearly or non-linearly. The same may apply to a height difference, if any, between the first cross-section and the second cross-section.

### Example 7

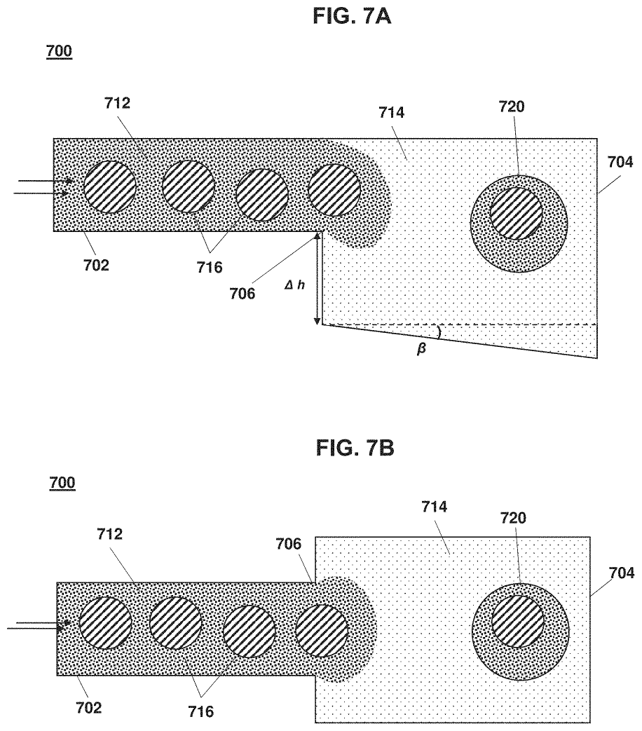

FIGS. 7A and 7B show a cross-section view and a top view, respectively, of another example of a microfluidic device with a geometric feature for droplet formation. A device 700 can include a channel 702 communicating at a fluidic connection 706 (or intersection) with a reservoir 704. In some instances, the device 700 and one or more of its components can correspond to the device 600 and one or more of its components.

An aqueous liquid 712 comprising a plurality of particles 716 may be transported along the channel 702 into the fluidic connection 706 to meet a second liquid 714 (e.g., oil, etc.) that is immiscible with the aqueous liquid 712 in the reservoir 704 to create droplets 720 of the aqueous liquid 712 flowing into the reservoir 704. At the fluidic connection 706 where the aqueous liquid 712 and the second liquid 714 meet, droplets can form based on factors such as the hydrodynamic forces at the fluidic connection 706, relative flow rates of the two liquids 712, 714, liquid properties, and certain geometric parameters (e.g., Δh, etc.) of the device 700. A plurality of droplets can be collected in the reservoir 704 by continuously injecting the aqueous liquid 712 from the channel 702 at the fluidic connection 706.

In some instances, the second liquid 714 may not be subjected to and/or directed to any flow in or out of the reservoir 704. For example, the second liquid 714 may be substantially stationary in the reservoir 704. In some instances, the second liquid 714 may be subjected to flow within the reservoir 704, but not in or out of the reservoir 704, such as via application of pressure to the reservoir 704 and/or as affected by the incoming flow of the aqueous liquid 712 at the fluidic connection 706. Alternatively, the second liquid 714 may be subjected and/or directed to flow in or out of the reservoir 704. For example, the reservoir 704 can be a channel directing the second liquid 714 from upstream to downstream, transporting the generated droplets. Alternatively, or in addition, the second liquid 714 in reservoir 704 may be used to sweep formed droplets away from the path of the nascent droplets.

The device 700 at or near the fluidic connection 706 may have certain geometric features that at least partly determine the sizes and/or shapes of the droplets formed by the device 700. The channel 702 can have a first cross-section height, h₁, and the reservoir 704 can have a second cross-section height, h₂. The first cross-section height, h₁, may be different from the second cross-section height h₂ such that at or near the fluidic connection 706, there is a height difference of Δh. The second cross-section height, h₂, may be greater than the first cross-section height, h₁. The reservoir may thereafter gradually increase in cross-section height, for example, the more distant it is from the fluidic connection 706. In some instances, the cross-section height of the reservoir may increase in accordance with expansion angle, β, at or near the fluidic connection 706. The height difference, Δh, and/or expansion angle, β, can allow the tongue (portion of the aqueous liquid 712 leaving channel 702 at fluidic connection 706 and entering the reservoir 704 before droplet formation) to increase in depth and facilitate decrease in curvature of the intermediately formed droplet. For example, droplet size may decrease with increasing height difference and/or increasing expansion angle.

While FIGS. 7A and 7B illustrate the height difference, Δh, being abrupt at the fluidic connection 706, the height difference may increase gradually (e.g., from about 0 μm to a maximum height difference). In some instances, the height difference may decrease gradually (e.g., taper) from a maximum height difference. In some instances, the height difference may variably increase and/or decrease linearly or non-linearly. While FIGS. 7A and 7B illustrate the expanding reservoir cross-section height as linear (e.g., constant expansion angle, β), the cross-section height may expand non-linearly. For example, the reservoir may be defined at least partially by a dome-like (e.g., hemispherical) shape having variable expansion angles. The cross-section height may expand in any shape.

### Example 8

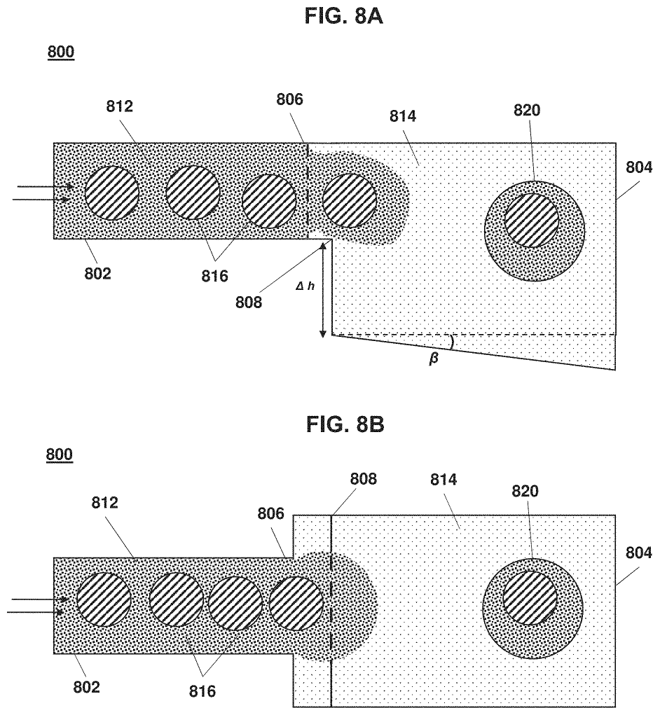

FIGS. 8A and 8B show a cross-section view and a top view, respectively, of another example of a microfluidic device with a geometric feature for droplet formation. A device 800 can include a channel 802 communicating at a fluidic connection 806 (or intersection) with a reservoir 804. In some instances, the device 800 and one or more of its components can correspond to the device 700 and one or more of its components and/or correspond to the device 600 and one or more of its components.

An aqueous liquid 812 comprising a plurality of particles 816 may be transported along the channel 802 into the fluidic connection 806 to meet a second liquid 814 (e.g., oil, etc.) that is immiscible with the aqueous liquid 812 in the reservoir 804 to create droplets 820 of the aqueous liquid 812 flowing into the reservoir 804. At the fluidic connection 806 where the aqueous liquid 812 and the second liquid 814 meet, droplets can form based on factors such as the hydrodynamic forces at the fluidic connection 806, relative flow rates of the two liquids 812, 814, liquid properties, and certain geometric parameters (e.g., Δh, etc.) of the device 800. A plurality of droplets can be collected in the reservoir 804 by continuously injecting the aqueous liquid 812 from the channel 802 at the fluidic connection 806.

A discrete droplet generated may comprise one or more particles of the plurality of particles 816. As described elsewhere herein, a particle may be any particle, such as a bead, cell bead, gel bead, biological particle, macromolecular constituents of biological particle, or other particles. Alternatively, a discrete droplet generated may not include any particles.

In some instances, the second liquid 814 may not be subjected to and/or directed to any flow in or out of the reservoir 804. For example, the second liquid 814 may be substantially stationary in the reservoir 804. In some instances, the second liquid 814 may be subjected to flow within the reservoir 804, but not in or out of the reservoir 804, such as via application of pressure to the reservoir 804 and/or as affected by the incoming flow of the aqueous liquid 812 at the fluidic connection 806. Alternatively, the second liquid 814 may be subjected and/or directed to flow in or out of the reservoir 804. For example, the reservoir 804 can be a channel directing the second liquid 814 from upstream to downstream, transporting the generated droplets. Alternatively, or in addition, the second liquid 814 in reservoir 804 may be used to sweep formed droplets away from the path of the nascent droplets.

While FIGS. 8A and 8B illustrate one ledge (e.g., step) in the reservoir 804, as can be appreciated, there may be a plurality of ledges in the reservoir 804, for example, each having a different cross-section height. For example, where there is a plurality of ledges, the respective cross-section height can increase with each consecutive ledge. Alternatively, the respective cross-section height can decrease and/or increase in other patterns or profiles (e.g., increase then decrease then increase again, increase then increase then increase, etc.).

While FIGS. 8A and 8B illustrate the height difference, Δh, being abrupt at the ledge 808, the height difference may increase gradually (e.g., from about 0 μm to a maximum height difference). In some instances, the height difference may decrease gradually (e.g., taper) from a maximum height difference. In some instances, the height difference may variably increase and/or decrease linearly or non-linearly. While FIGS. 8A and 8B illustrate the expanding reservoir cross-section height as linear (e.g., constant expansion angle), the cross-section height may expand non-linearly. For example, the reservoir may be defined at least partially by a dome-like (e.g., hemispherical) shape having variable expansion angles. The cross-section height may expand in any shape.

### Example 9

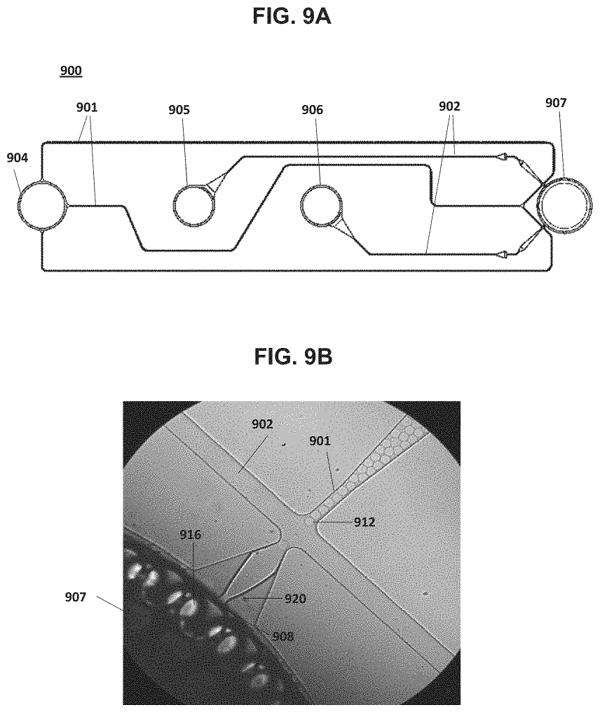

An example of a device according to the invention is shown in FIGS. 9A-9B. The device 900 includes four fluid reservoirs, 904, 905, 906, and 907, respectively. Reservoir 904 houses one liquid; reservoirs 905 and 906 house another liquid, and reservoir 907 houses continuous phase in the step region 908. This device 900 include two first channels 902 connected to reservoir 905 and reservoir 906 and connected to a shelf region 920 adjacent a step region 908. As shown, multiple channels 901 from reservoir 904 deliver additional liquid to the first channels 902. The liquids from reservoir 904 and reservoir 905 or 906 combine in the first channel 902 forming the first liquid that is dispersed into the continuous phase as droplets. In certain embodiments, the liquid in reservoir 905 and/or reservoir 906 includes a particle, such as a gel bead. FIG. 9B shows a view of the first channel 902 containing gel beads 912 intersected by a second channel 901 adjacent to a shelf region 920 leading to a step region 908, which contains multiple droplets 916.

### Example 10

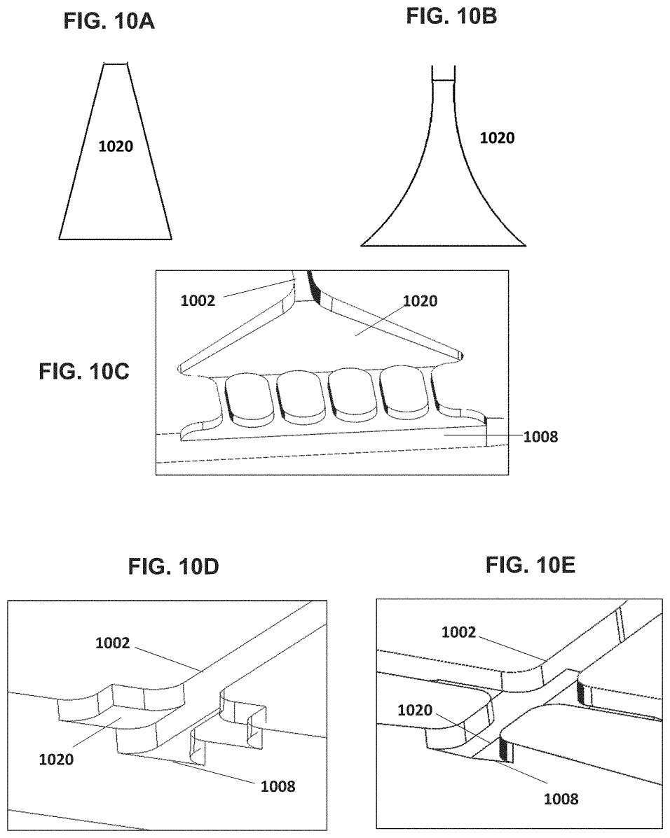

Variations on shelf regions 1020 are shown in FIGS. 10A-10E. As shown in FIGS. 10A-10B, the width of the shelf region 1020 can increase from the distal end of a first channel 1002 towards the step region 1008, linearly as in 10A or non-linearly as in 10B. As shown in FIG. 10C, multiple first channels 1002 can branch from a single feed channel 1002 and introduce fluid into interconnected shelf regions 1020. As shown in FIG. 10D, the depth of the first channel 1002 may be greater than the depth of the shelf region 1020 and cut a path through the shelf region 1020. As shown in FIG. 10E, the first channel 1002 and shelf region 1020 may contain a grooved bottom surface. This device 1000 also includes a second channel 1002 that intersects the first channel 1002 proximal to its distal end.

### Example 11

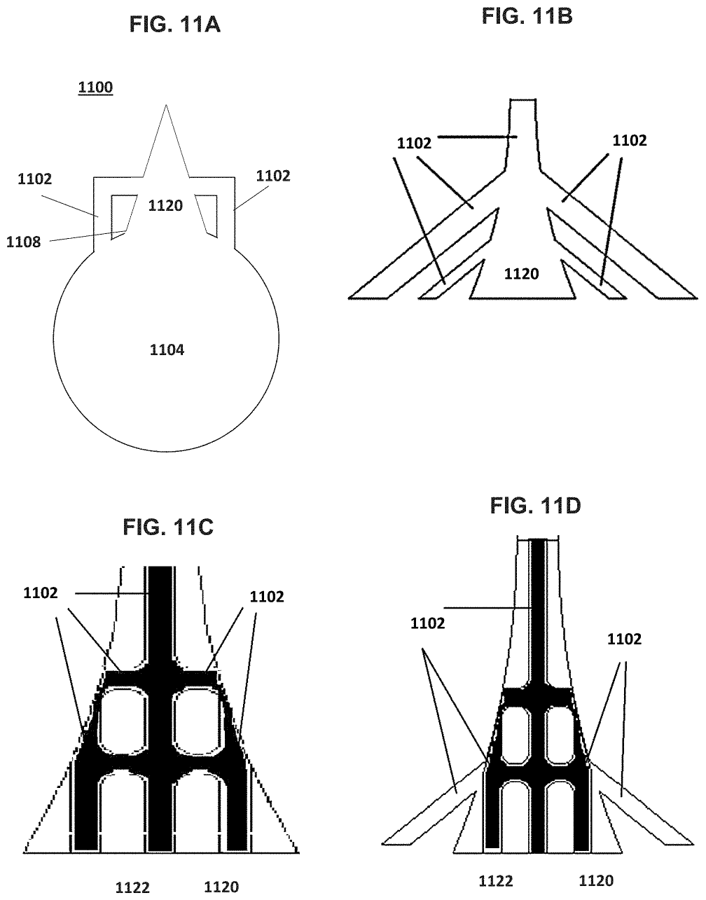

Continuous phase delivery channels 1102, shown in FIGS. 11A-11D, are variations on shelf regions 1120 including channels 1102 for delivery (passive or active) of continuous phase behind a nascent droplet. In one example in FIG. 11A, the device 1100 includes two channels 1102 that connect the reservoir 1104 of the step region 1108 to either side of the shelf region 1120. In another example in FIG. 11B, four channels 1102 provide continuous phase to the shelf region 1120. These channels 1102 can be connected to the reservoir 1104 of the step region 1108 or to a separate source of continuous phase. In a further example in FIG. 11C, the shelf region 1120 includes one or more channels 1102 (white) below the depth of the first channel 1102 (black) that connect to the reservoir 1104 of the step region 1108. The shelf region 1120 contains islands 1122 in black. In another example FIG. 11D, the shelf region 1120 of FIG. 11C includes two additional channels 1102 for delivery of continuous phase on either side of the shelf region 1120.

### Example 12

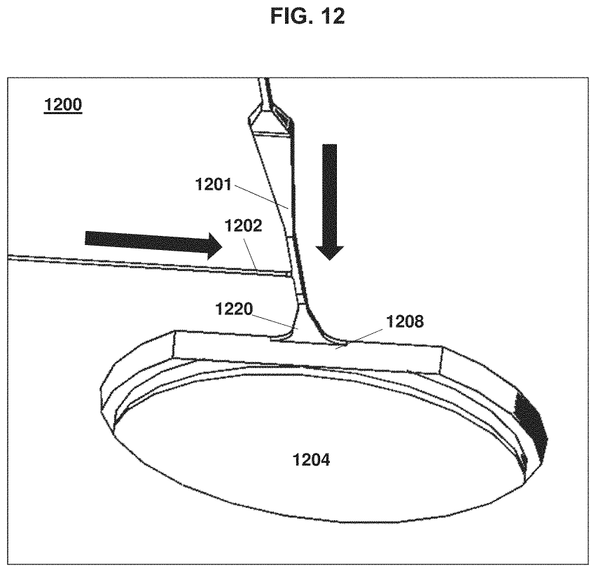

An embodiment of a device according to the invention is shown in FIG. 12. This device 1200 includes two channels 1201, 1202 that intersect upstream of a droplet formation region. The droplet formation region includes both a shelf region 1220 and a step region 1208 disposed between the distal end of the first channel 1201 and the step region 1208 that lead to a collection reservoir 1204. The black and white arrows show the flow of liquids through each of first channel 1201 and second channel 1202, respectively. In certain embodiments, the liquid flowing through the first channel 1201 or second channel 1202 includes a particle, such as a gel bead. As shown in the FIG. 12, the width of the shelf region 1220 can increase from the distal end of a first channel 1201 towards the step region 1208; in particular, the width of the shelf region 1220 in FIG. 12 increases non-linearly. In this embodiment, the shelf region extends from the edge of a reservoir to allow droplet formation away from the edge. Such a geometry allows droplets to move away from the droplet formation region due to differential density between the continuous and dispersed phase.

### Example 13

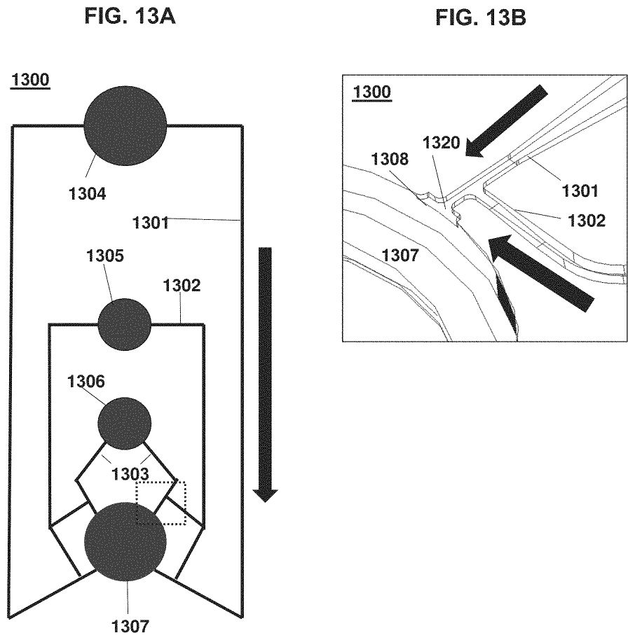

An embodiment of a device according to the invention for multiplexed droplet formation is shown in FIGS. 13A-13B. This device 1300 includes four fluid reservoirs, 1304, 1305, 1306, and 1307, and the overall direction of flow within the device 1300 is shown by the black arrow in FIG. 13A. Reservoir 1304 and reservoir 1306 house one liquid; reservoir 1305 houses another liquid, and reservoir 1307 houses continuous phase and is a collection reservoir. Fluid channels 1301, 1303 directly connect reservoir 1304 and reservoir 1306, respectively, to reservoir 1307; thus, there are four droplet formation region in this device 1300. Each droplet formation region has a shelf region 1320 and a step region 1308. This device 1300 further has two channels 1302 from the reservoir 1305 where each of these channels splits into two separate channels at their distal ends. Each of the branches of the split channel intersects the first channels 1301 or 1303 upstream of their connection to the collection reservoir 1307. As shown in the zoomed in view of the dotted line box in FIG. 13B, second channel 1302, with its flow indicated by the white arrow, has its distal end intersecting a channel 1303 from reservoir 1305, with the flow of the channel indicated by the black arrow, upstream of the droplet formation region. The liquid from reservoir 1304 and reservoir 1306, separately, are introduced into channels 1301, 1303 and flow towards the collection reservoir 1307. The liquid from the second reservoir 1305 combines with the fluid from reservoir 1304 or reservoir 1306, and the combined fluid is dispersed into the droplet formation region and to the continuous phase. In certain embodiments, the liquid flowing through the first channel 1301 or 1303 or second channel 1302 includes a particle, such as a gel bead.

### Example 14

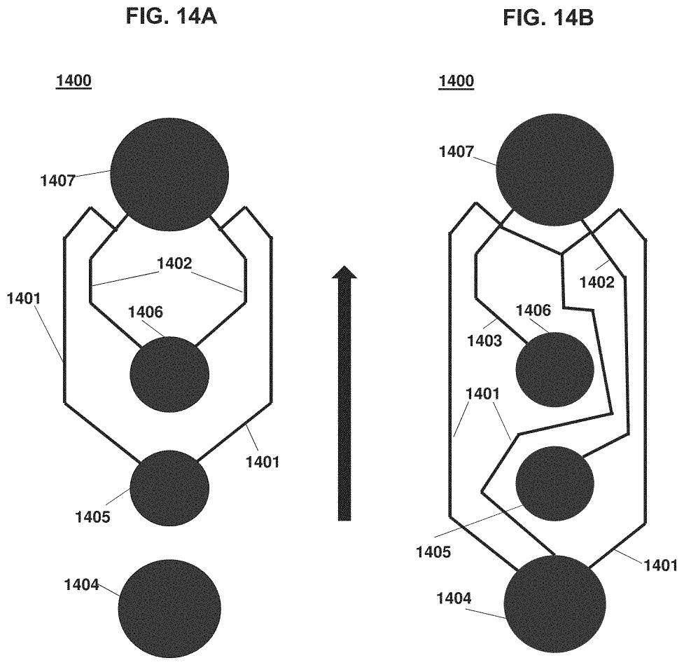

Examples of devices according to the invention that include two droplet formation regions are shown in FIGS. 14A-14B. The device 1400 of FIG. 14A includes three reservoirs, 1405, 1406, and 1407, and the device 1400 of FIG. 14B includes four reservoirs, 1404, 1405, 1406, and 1407. For the device 1400 of FIG. 14A, reservoir 1405 houses a portion of the first fluid, reservoir 1406 houses a different portion of the first fluid, and reservoir 1407 houses continuous phase and is a collection reservoir. In the device 1400 of FIG. 14B, reservoir 1404 houses a portion of the first fluid, reservoir 1405 and reservoir 1406 house different portions of the first fluid, and reservoir 1407 houses continuous phase and is a collection reservoir. In both devices 1400, there are two droplet formation regions. For the device 1400 of FIG. 14A, the connections to the collection reservoir 1407 are from the reservoir 1406, and the distal ends of the channels 1401 from reservoir 1405 intersect the channels 1402 from reservoir 1406 upstream of the droplet formation region. The liquids from reservoir 1405 and reservoir 1406 combine in the channels 1402 from reservoir 1406, forming the first liquid that is dispersed into the continuous phase in the collection reservoir 1407 as droplets. In certain embodiments, the liquid in reservoir 1405 and/or reservoir 1406 includes a particle, such as a gel bead.

In the device 1400 of FIG. 14B, each of reservoir 1405 and reservoir 1406 are connected to the collection reservoir 1407. Reservoir 1404 has three channels 1401, two of which have distal ends that intersect each of the channels 1402, 1403 from reservoir 1404 and reservoir 1406, respectively, upstream of the droplet formation region. The third channel 1401 from reservoir 1404 splits into two separate distal ends, with one end intersecting the channel 1402 from reservoir 1405 and the other distal end intersecting the channel 1403 from reservoir 1406, both upstream of droplet formation regions. The liquid from reservoir 1404 combines with the liquids from reservoir 1405 and reservoir 1406 in the channels 1402 from reservoir 1405 and reservoir 1406, forming the first liquid that is dispersed into the continuous phase in the collection reservoir 1407 as droplets. In certain embodiments, the liquid in reservoir 1404, reservoir 1405, and/or reservoir 1406 includes a particle, such as a gel bead.

### Example 15

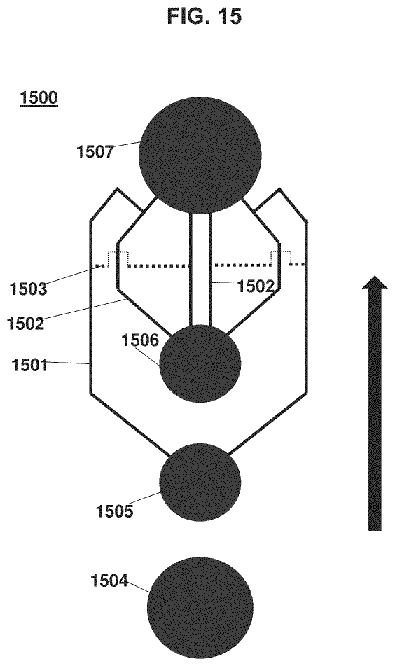

An embodiment of a device according to the invention that has four droplet formation regions is shown in FIG. 15. The device 1500 of FIG. 15 includes four reservoirs, 1504, 1505, 1506, and 1507; the reservoir labeled 1504 is unused in this embodiment. In the device 1500 of FIG. 15, reservoir 1505 houses a portion of the first fluid, reservoir 1506 houses a different portion of the first fluid, and reservoir 1507 houses continuous phase and is a collection reservoir. Reservoir 1506 has four channels 1502 that connect to the collection reservoir 1507 at four droplet formation regions. The channels 1502 from originating at reservoir 1506 include two outer channels 1502 and two inner channels 1502. Reservoir 1505 has two channels 1501 that intersect the two outer channels 1502 from reservoir 1506 upstream of the droplet formation regions. Channels 1501 and the inner channels 1502 are connected by two channels 1503 that traverse, but do not intersect, the fluid paths of the two outer channels 1502. These connecting channels 1503 from channels 1501 pass over the outer channels 1502 and intersect the inner channels 1502 upstream of the droplet formation regions. The liquids from reservoir 1505 and reservoir 1506 combine in the channels 1502, forming the first liquid that is dispersed into the continuous phase in the collection reservoir 1507 as droplets. In certain embodiments, the liquid in reservoir 1505 and/or reservoir 1506 includes a particle, such as a gel bead.

### Example 16

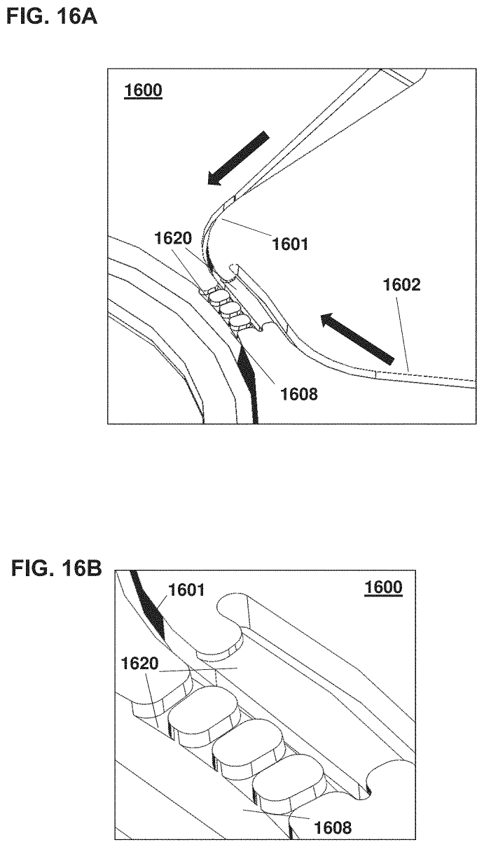

An embodiment of a device according to the invention that has a plurality of droplet formation regions is shown in FIGS. 16A-16B (FIG. 16B is a zoomed in view of FIG. 16A), with the droplet formation region including a shelf region 1620 and a step region 1608. This device 1600 includes two channels 1601, 1602 that meet at the shelf region 1620. As shown, after the two channels 1601, 1602 meet at the shelf region 1620, the combination of liquids is divided, in this example, by four shelf regions. In certain embodiments, the liquid with flow indicated by the black arrow includes a particle, such as a gel bead, and the liquid flow from the other channel, indicated by the white arrow, can move the particles into the shelf regions such that each particle can be introduced into a droplet.

### Example 17

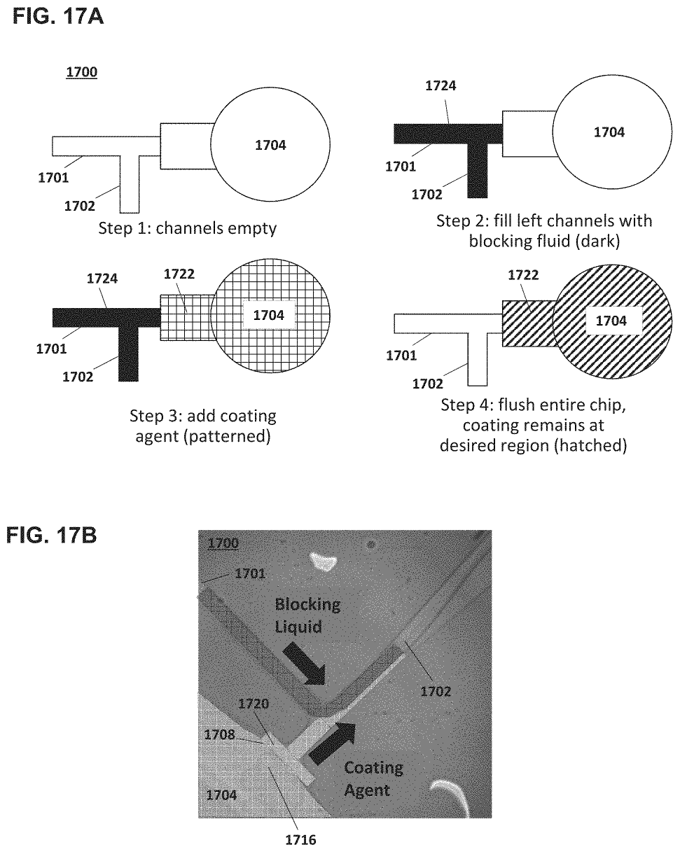

An embodiment of a method of modifying the surface of a device using a coating agent is shown in FIGS. 17A-17B. In this example, the surface of the droplet formation region of the device 1700, e.g., the rectangular area connected to the circular shaped collection reservoir 1704, is coated with a coating agent 1722 to modify its surface properties. To localize the coating agent to only the regions of interest, the first channel 1701 and second channel 1702 of the device 1700 are filled with a blocking liquid 1724 (Step 2 of FIG. 17A) such that the coating agent 1722 cannot contact the channels 1701, 1702. The device 1700 is then filled with the coating agent 1722 to fill the droplet formation region and the collection reservoir 1704 (Step 3 of FIG. 17A). After the coating process is complete, the device 1700 is flushed (Step 4 of FIG. 17A) to remove both the blocking liquid 1724 from the channels and the coating agent 1722 from the droplet formation region and the collection reservoir 1704. This leaves behind a layer of the coating agent 1722 only in the regions where it is desired. This is further exemplified in the micrograph of FIG. 17B, the blocking liquid (dark gray) fills the first channel 1701 and second channel 1702, preventing ingress of the coating agent 1722 (white) into either the first channel 1701 or the second channel 1702 while completely coating the droplet formation region and the collection reservoir 1704. In this example, the first channel 1701 is also acting as a feed channel for the blocking liquid 1724, shown by the arrow for flow direction in FIG. 17B.

### Example 18

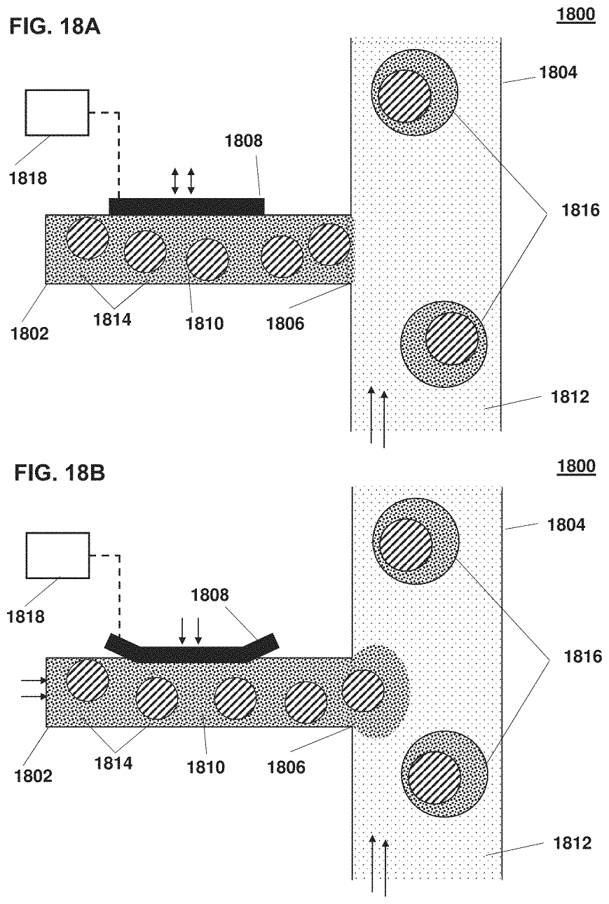

FIGS. 18A-18B show an embodiment of a device according to the invention that includes a piezoelectric element for droplet formation. A device 1800 includes a first channel 1802, a second channel 1804, and a piezoelectric element 1808. The first channel 1802 and the second channel 1804 are in fluid communication at a channel junction 1806. In some instances, the first channel 1802 and components thereof can correspond to the channel 102 in the device 100 in FIG. 1 and components thereof.

In this example, the first channel 1802 carries a first fluid 1810 (e.g., aqueous) and the second channel 1804 can carries second fluid 1812 (e.g., oil) that is immiscible with the first fluid 1810. The two fluids 1810, 1812 come in contact with one another at the junction 1806. In some instances, the first fluid 1810 in the first channel 1802 includes suspended particles 1814. The particles 1814 may be beads, biological particles, cells, cell beads, or any combination thereof (e.g., a combination of beads and cells or a combination of beads and cell beads, etc.). The piezoelectric element 1808 is operatively coupled to the first channel 1802 such that at least part of the first channel 1802 is capable of moving or deforming in response to movement of the piezoelectric element 1808. In some instances, the piezoelectric element 1808 is part of the first channel 1802, such as one or more walls of the first channel 1802. The piezoelectric element 1808 can be a piezoelectric plate. The piezoelectric element 1808 is responsive to electrical signals received from the controller 1818 and moves between at least a first state (as in FIG. 18A) and a second state (as in FIG. 18B). In the first state, the first fluid 1810 and the second fluid 1812 remain separated at or near the junction 1806 via an immiscible barrier. In the second state, the first fluid 1810 is directed towards the junction 1806 into the second fluid 1812 to create droplets 1816.

In some instances, the piezoelectric element 1808 is in the first state (shown in FIG. 18A) when no electrical charge, e.g., electric voltage, is applied. The first state can be an equilibrium state. When an electrical charge is applied to the piezoelectric element 1808, the piezoelectric element 1808 may bend backwards (not shown in FIG. 18A or 18B), pulling a part of the first channel 1802 outwards and drawing in more of the first fluid 1810 into the first channel 1802 such as from a reservoir of the first fluid 1810. When the electrical charge is altered, the piezoelectric element may bend in the other direction (e.g., inwards towards the contents of the channel 1802) (shown in FIG. 18B) pushing a part of the first channel 1802 inwards and propelling (e.g., at least partly via displacement) a volume of the first fluid 1810 into the second channel 1804, thereby generating a droplet of the first fluid 1810 in the second fluid 1812. After the droplet is propelled, the piezoelectric element 1808 may return to the first state (shown in FIG. 18A). The cycle can be repeated to generate more droplets. In some instances, each cycle may generate a plurality of droplets (e.g., a volume of the first fluid 1810 propelled breaks off as it enters the second fluid 1812 to form a plurality of discrete droplets). A plurality of droplets 1816 can be collected in the second channel 1804 for continued transportation to a different location (e.g., reservoir), direct harvesting, and/or storage.

### Example 19

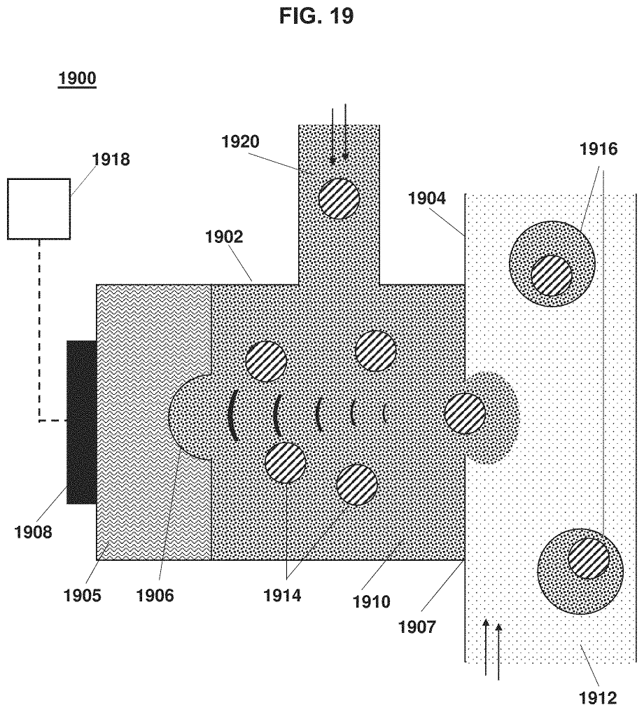

FIG. 19 shows an embodiment of a device according to the invention that uses a piezoelectric, e.g., a piezoacoustic element, for droplet formation. A device 1900 includes a first channel 1902, a second channel 1904, a piezoelectric element 1908, and a buffer substrate 1905. The first channel 1902 and the second channel 1904 communicate at a channel junction 1907. In some instances, the first channel 1902 and components thereof can correspond to the channel 102 in the channel structure 100 in FIG. 1 and components thereof.

The first channel 1902 carries a first fluid 1910 (e.g., aqueous), and the second channel 1904 carries a second fluid 1912 (e.g., oil) that is immiscible with the first fluid 1910. In some instances, the first fluid 1910 in the first channel 1902 includes suspended particles 1914. In some instances, the particles 1914, suspended in the first fluid 1910, are provided to the first channel 1902 from a third channel 1920, which is in fluid communication with the first channel 1902. The particles 1914 may be beads, biological particles, cells, cell beads, or any combination thereof (e.g., a combination of beads and cells or a combination of beads and cell beads, etc.). The piezoelectric element 1908 is operatively coupled to a buffer substrate 1905 (e.g., glass). The buffer substrate 1905 includes an acoustic lens 1906. In some instances, the acoustic lens 1906 is a substantially spherical cavity, e.g., a partially spherical cavity, e.g., hemispherical. In other instances, the acoustic lens 1906 is a different shape and/or includes one or more other objects for focusing acoustic waves. The buffer substrate 1905 and/or the acoustic lens 1906 can be in contact with the first fluid 1910 in the first channel 1902. Alternatively, the piezoelectric element 1908 is operatively coupled to a part (e.g., wall) of the first channel 1902 without an intermediary buffer substrate. The piezoelectric element 1908 is in electrical communication with a controller 1918. The piezoelectric element 1908 is responsive to a pulse of electric voltage driven at a particular frequent transmitted by the controller 1918. In some instances, the piezoelectric element 1908 and its properties can correspond to the piezoelectric element 1808 and its properties in FIGS. 18A-18B.

Before electric voltage is applied, the first fluid 1910 and the second fluid 1912 are separated at or near the junction 1907 via an immiscible barrier. When the electric voltage is applied to the piezoelectric element 1908, it generates acoustic waves that propagate in the buffer substrate 1905, from the first end to the second end. The acoustic lens 1906 at the second end of the buffer substrate 1905 focuses the acoustic waves towards the immiscible interface between the two fluids 1910, 1912. The acoustic lens 1906 may be located such that the immiscible interface is located at the focal plane of the converging beam of the acoustic waves. The pressure of the acoustic waves may cause a volume of the first fluid 1910 to be propelled into the second fluid 1912, thereby generating a droplet of the first fluid 1910 in the second fluid 1912. In some instances, each propelling may generate a plurality of droplets (e.g., a volume of the first fluid 1910 propelled breaks off as it enters the second fluid 1912 to form a plurality of discrete droplets). After ejection of the droplet, the immiscible interface can return to its original state. Subsequent bursts of electric voltage to the piezoelectric element 1908 can be repeated to generate more droplets 1916. A plurality of droplets 1916 can be collected in the second channel 1904 for continued transportation to a different location (e.g., reservoir), direct harvesting, and/or storage.

### Example 20

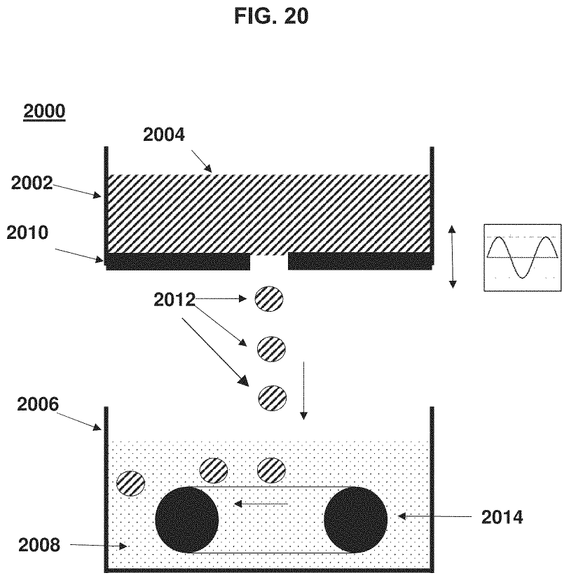

FIG. 20 shows an embodiment of a device according to the invention that includes a piezoelectric element for droplet formation. The device 2000 includes a reservoir 2002 for holding first fluid 2004 and a collection reservoir 2006 for holding second fluid 2008, such as an oil. In one wall of the reservoir 2002 is a piezoelectric element 2010 operatively coupled to an aperture.

Upon actuation of the piezoelectric element 2010, the first fluid 2004 exits the aperture and forms a droplet 2012 that is collected in collection reservoir 2006. Collection reservoir 2006 includes a mechanism 2014 for circulating second fluid 2008 and moving formed droplets 2012 through the second fluid 2008. The signal applied to the piezoelectric element 2010 may be a sinusoidal signal as indicated in the inset photo.

### Example 21

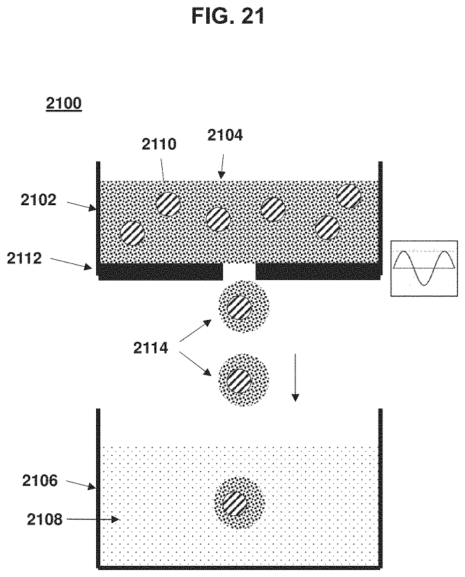

FIG. 21 shows an embodiment of a device according to the invention that includes a piezoelectric element for droplet formation. The device 2100 includes a reservoir 2102 for holding first fluid 2104 and a collection reservoir 2106 for holding second fluid 2108, such as an oil. The first fluid 2104 may contain particles 2110. In one wall of the reservoir 2102 is a piezoelectric element 2112 operatively couple to an aperture.

Upon operation of the piezoelectric element 2112 the first fluid 2104 and the particles 2110 exit the aperture and form a droplet 2114 containing the particle 2110. The droplet 2114 is collected in the second fluid 2108 held in the collection reservoir 2106. The second fluid 2108 may or may not be circulated. The signal applied to the piezoelectric element 2112 may be a sinusoidal signal as indicated in the inset photo.

### Example 22

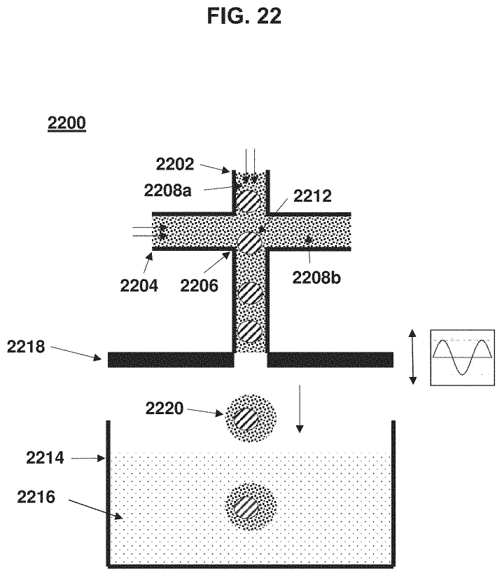

FIG. 22 shows an embodiment of a device according to the invention that includes a piezoelectric element for droplet formation. The device 2200 includes a first channel 2202 and a second channel 2204 that meet at junction 2206. The first channel 2202 carries a portion of first fluid 2208a, and the second channel 2204 carries another portion of first fluid 2208b. One of the portions of the first fluid 2208a or 2208b further includes a particle 2212. The device includes a collection reservoir 2214 for holding second fluid 2216, such as an oil. The distal end of the first channel includes a piezoelectric element 2218 operatively couple to an aperture.

The portion of first fluid 2208a flowing through the first channel 2202, e.g., carrying particles 2212, combines with the portion of the first fluid 2208b flowing through second channel 2204 to form the first fluid, and the first fluid continues to the distal end of the first channel 2202. Upon actuation of the piezoelectric element 2218 at the distal end of the first channel 2202, the first fluid and particles 2212 form a droplet 2220 containing a particle 2212. The droplet 2220 is collected in the second fluid 2216 in the collection reservoir 2214. The second fluid 2216 may or may not be circulated. The signal applied to the piezoelectric element 2218 may be a sinusoidal signal as indicated in the inset photo.

### Example 23

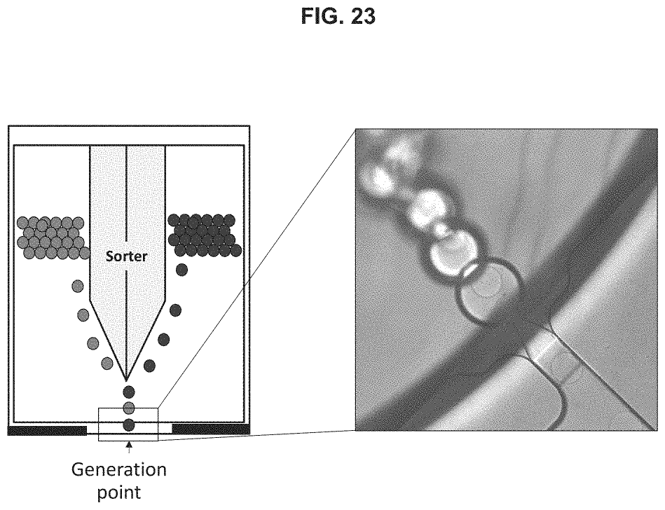

FIG. 23 shows a general embodiment of a device according to the invention that includes a sorter. The droplets are formed in the droplet formation region (generation point) and enter into the sorting region. Magnified on the right is a micrograph showing droplets being formed at the generation point in the droplet formation region as they move into the sorting region. In this embodiment, two types of droplets are formed. The first type of droplets are sorted to one partition while the second type of droplets are sorted to another partition.

### Example 24

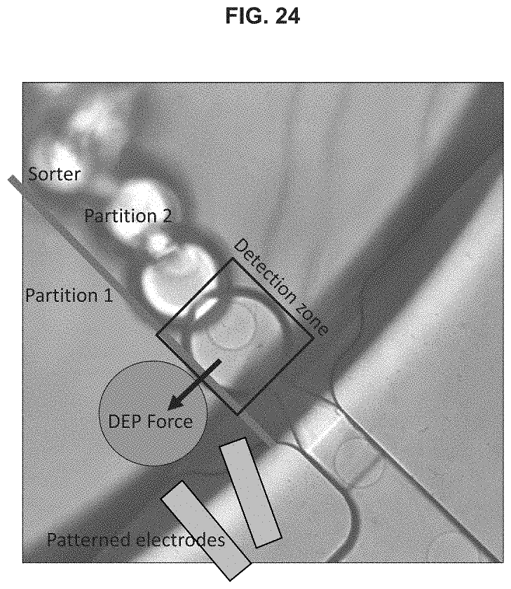

FIG. 24 shows an embodiment of a device according to the invention that includes a sorter configured to sort droplets using dielectrophoresis. The droplet passes through a detection zone and is deflected to a partition by the dielectrophoretic force generated by the patterned electrodes. Droplets that are not deflected are sorted to another partition.

### Example 25

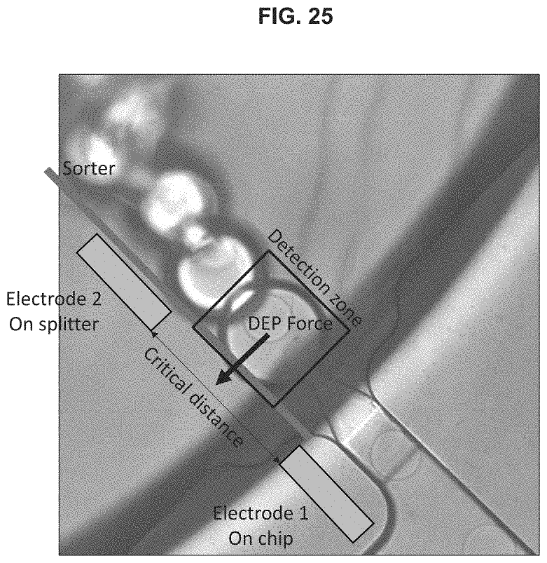

FIG. 25 shows an embodiment of a device according to the invention that includes a sorter configured to sort droplets using dielectrophoresis. The electrodes are patterned with one on a sorter and one on a chip with a distance between them to allow passage of the sorted droplet. The droplet passes through a detection zone and is deflected to a partition by the dielectrophoretic force generated by the patterned electrodes. Droplets that are not deflected are sorted to another partition.

### Example 26

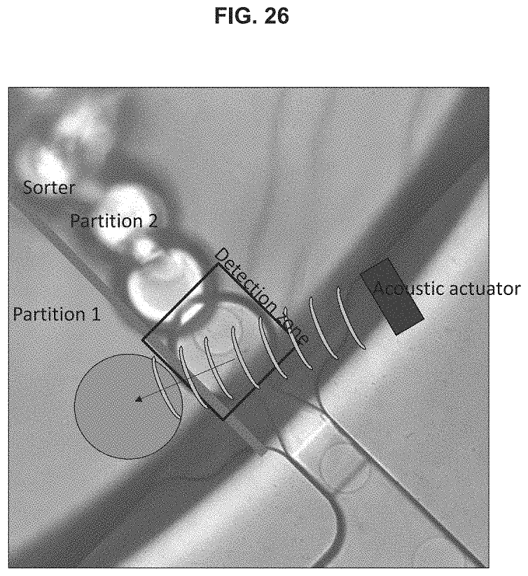

FIG. 26 shows an embodiment of a device according to the invention that includes an acoustic actuator in the sorting region. The droplet passes through a detection zone and is deflected to a partition by an acoustic force from the acoustic actuator. Droplets that are not deflected are sorted to another partition.

### Example 27

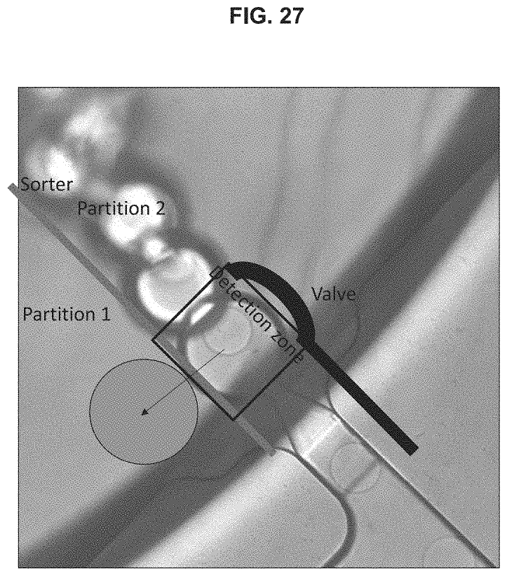

FIG. 27 shows an embodiment of a device according to the invention that includes a mechanical arm in the sorting region. The droplet passes through a detection zone and is deflected to a partition by a mechanical arm that can physically push the droplet. Droplets that are not deflected are sorted to another partition.

### Example 28

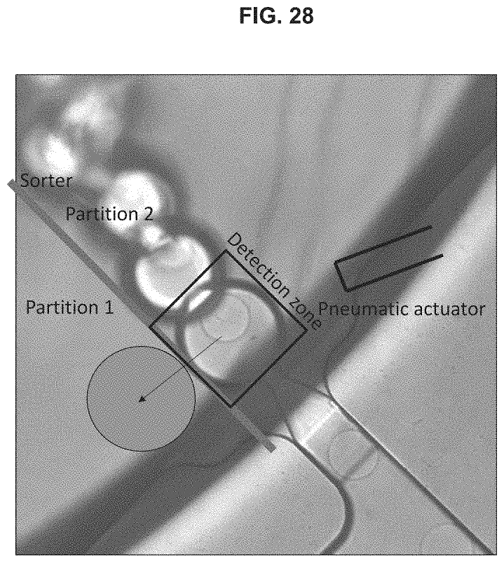

FIG. 28 shows an embodiment of a device according to the invention that includes a fluidic (e.g., pneumatic) actuator in the sorting region. The droplet passes through a detection zone and is deflected to a partition by a fluidic (e.g., pneumatic) force from the fluidic (e.g., pneumatic) actuator. Droplets that are not deflected are sorted to another partition.

### Example 29

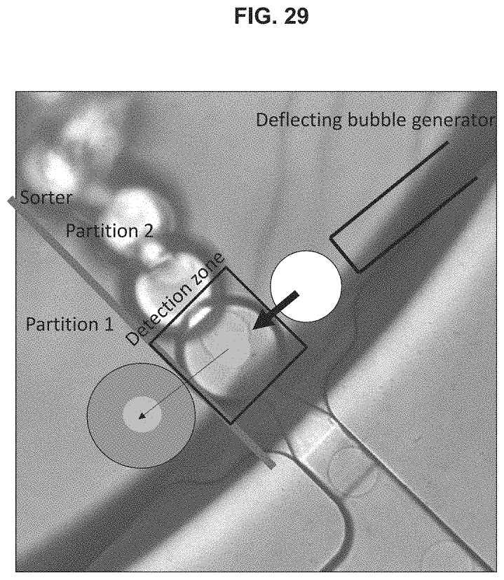

FIG. 29 shows an embodiment of a device according to the invention that includes a bubble generator in the sorting region. The droplet passes through a detection zone and is deflected to a partition by a bubble produced by the bubble generator. Droplets that are not deflected are sorted to another partition.

### Example 30

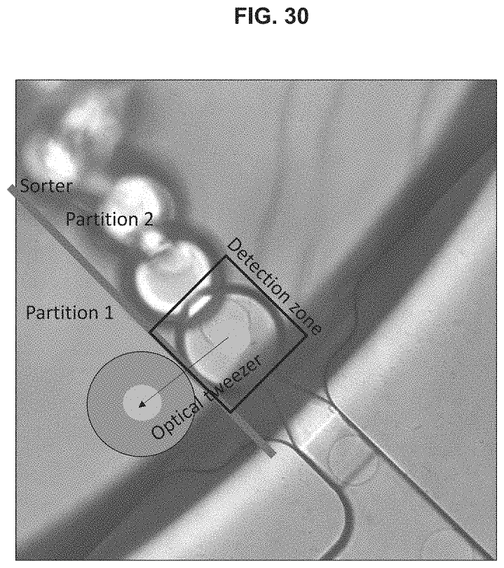

FIG. 30 shows an embodiment of a device according to the invention that includes an optical tweezer in the sorting region. The droplet passes through a detection zone and is deflected to a partition the force generated by the optical tweezer. Droplets that are not deflected are sorted to another partition.

### Example 31

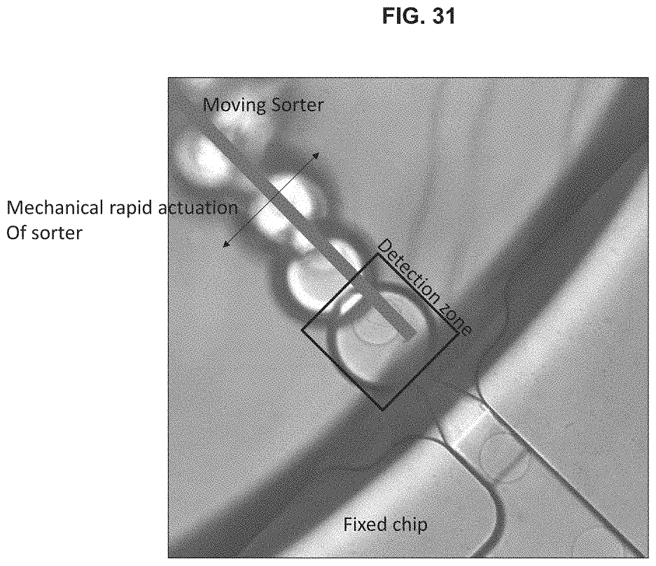

FIG. 31 shows an embodiment of a device according to the invention that includes a mechanical divider in the sorting region. The droplet passes through a detection zone and is deflected to a partition by the divider. Droplets that are not deflected are sorted to another partition.

### Example 32

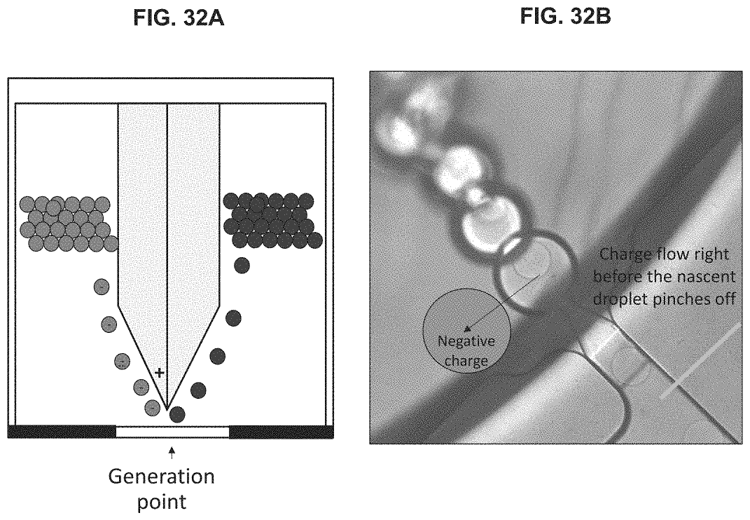

FIGS. 32A and 32B show an embodiment of a device according to the invention that includes an electrostatic charger in the sorting region. The droplets pass through a channel where the nascent droplet is electrostatically charged right before pinching off. Droplets with a negative charge are sorted into a partition. Droplets that are not negatively charged are sorted to another partition.

### Example 33

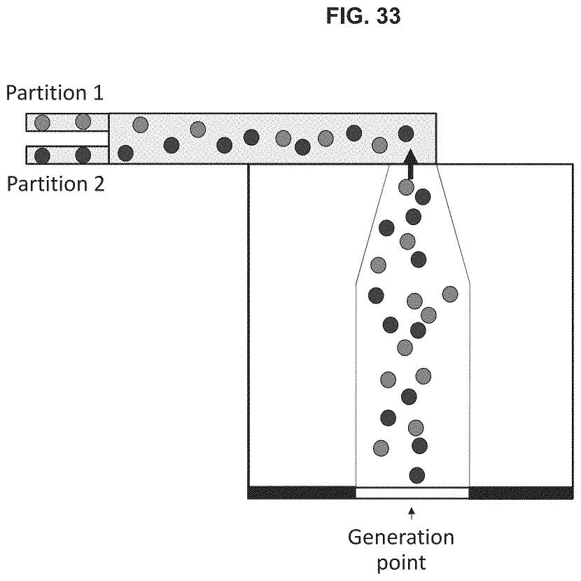

FIG. 33 shows a general embodiment of a device according to the invention that includes two partitions in the collection region. The droplets are formed in the droplet formation region (generation point) and move into a large reservoir. The droplets are then funneled into a narrower channel where the droplets line up in single file. The sorting mechanism then sorts the droplets into two channels. A first type of droplet is sorted into one partition while a second type of droplet is sorted into another partition.

### Example 34

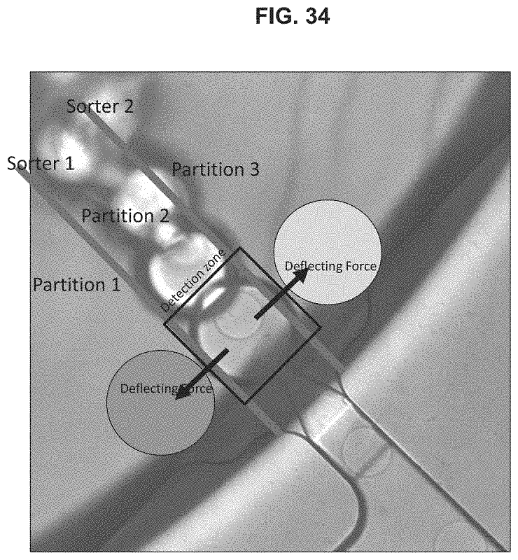

FIG. 34 shows a general embodiment of a device according to the invention that employs multi-way sorting and includes three partitions in the collection region. The droplet passes through a detection zone and is deflected to one of two partitions. Droplets that are not deflected are sorted to another partition.

### Example 35

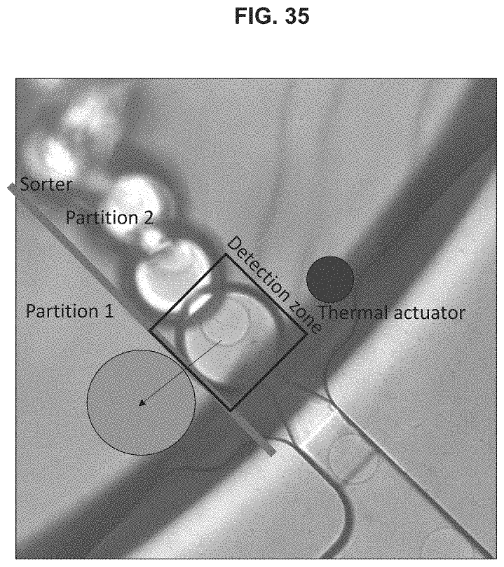

FIG. 35 shows an embodiment of a device according to the invention that includes a thermal actuator in the sorting region. The droplet passes through a detection zone and is deflected to a partition by a force generated by the thermal actuator. Droplets that are not deflected are sorted to another partition.

### Example 36

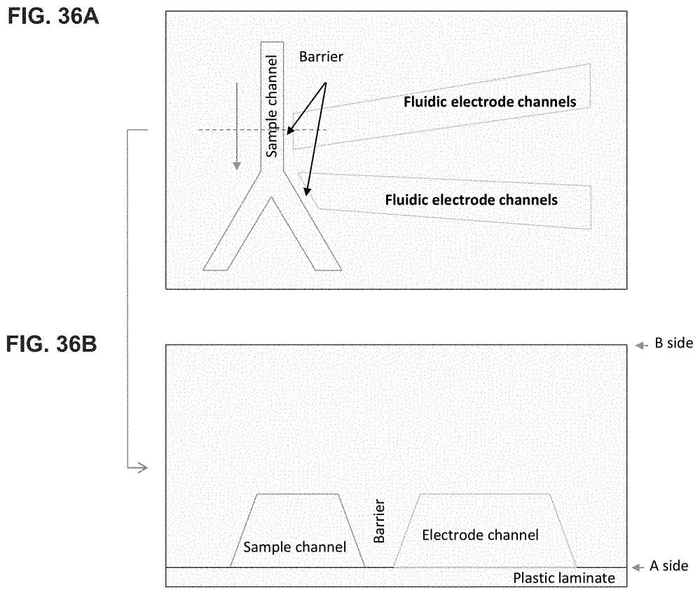

FIGS. 36A-36B show a general embodiment of a device according to the invention that includes an electrode-based sorting mechanism. The droplets or particles provided by the droplet or particle source flow through a channel. A barrier is present between the channel with the droplets or particles and two fluidic electrode channels. The barrier operatively connects the fluidic electrode channels, which generates an electric field. The force generated by the electrodes moves the droplets or particles into one of the two partitions in the collection region.

### Example 37

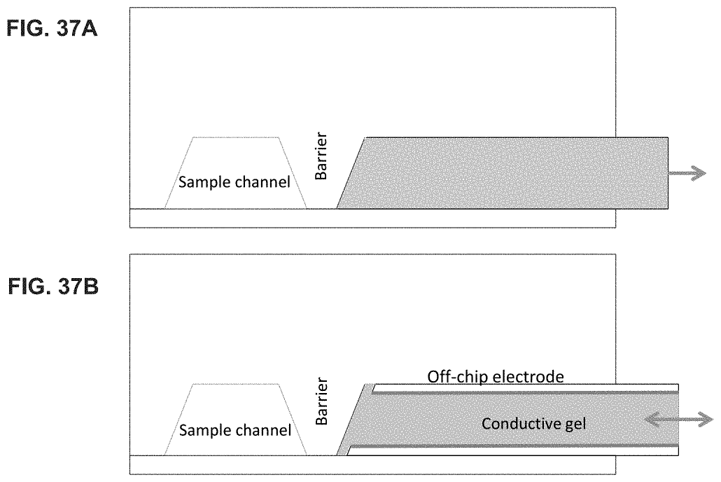

FIGS. 37A-37B show an embodiment of a device according to the invention that includes an electrode-based sorting mechanism. The electrode may be a solid conductor (FIG. 37A) or a liquid conductor (e.g., conductive gel) (FIG. 37B). The conductive gel operatively connects an off-chip electrode with the barrier, which is configured to operatively connect the electrode with the sample channel.

### Example 38

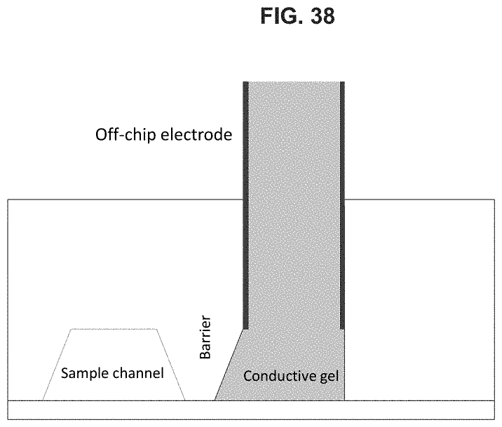

FIG. 38 shows an embodiment of a device according to the invention that includes an electrode-based sorting mechanism. The electrode is inserted into the device from the top. The electrode is a liquid conductor with a conductive gel that operatively connects an off-chip electrode with the barrier, which is configured to operatively connect the electrode with the sample channel.

### Example 39

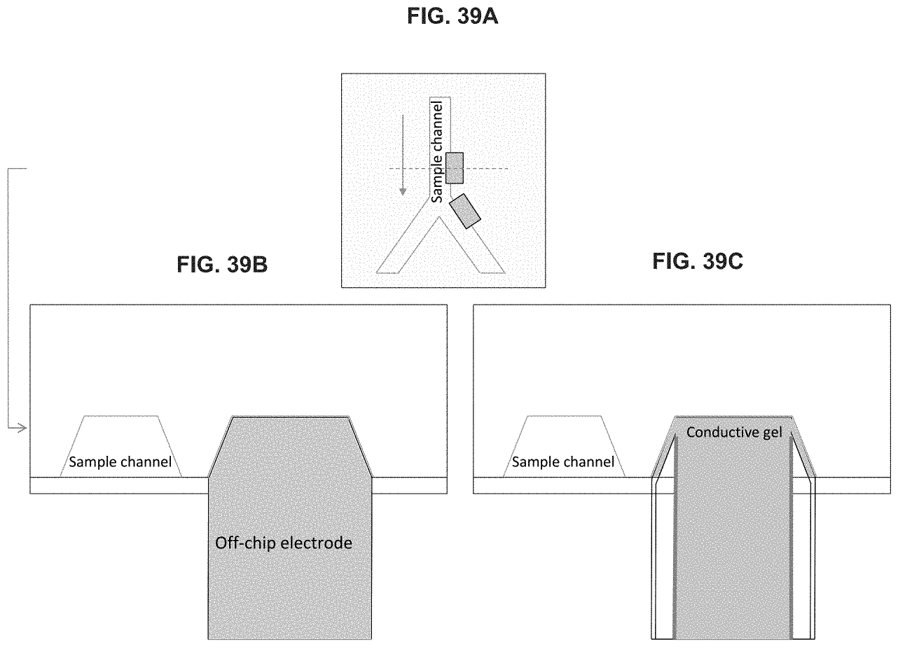

FIGS. 39A-39C show an embodiment of a device according to the invention that includes an electrode-based sorting mechanism. The droplets or particles provided by the droplet or particle source flow through a channel. A barrier is positioned between the channel with the droplets or particles and two fluidic electrode channels. The electrodes are inserted into the device from the bottom. The barrier operatively connects the electrode channels, which generate an electric field. The force generated by the electrodes move the droplets or particles into one of the two partitions in the collection region. As shown in FIGS. 39B-39C, the electrode may be a solid conductor (FIG. 39B) or a liquid conductor (e.g., conductive gel) (FIG. 39C). The conductive gel operatively connects an off-chip electrode with the barrier, which is configured to operatively connect the electrode with the sample channel.

### Example 40

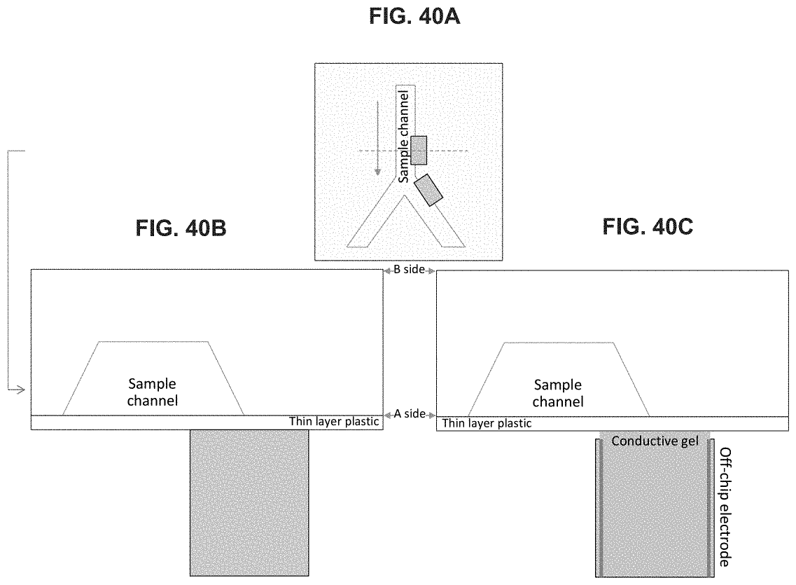

FIGS. 40A-40C show an embodiment of a device according to the invention that includes an electrode-based sorting mechanism. The droplets or particles provided by the droplet or particle source flow through a channel. The bottom of the device includes a thin layer (e.g., plastic) barrier that separates the electrode and the sample channel. The electrodes contact the barrier from the bottom of the device. The barrier operatively connects the electrode channels, which generate an electric field. The force generated by the electrodes moves the droplets or particles into one of the two partitions in the collection region. As shown in FIGS. 40B-40C, the electrode may be a solid conductor (FIG. 40B) or a liquid conductor (e.g., conductive gel) (FIG. 40C). The conductive gel operatively connects an off-chip electrode with the barrier, which is configured to operatively connect the electrode with the sample channel.

### Example 41

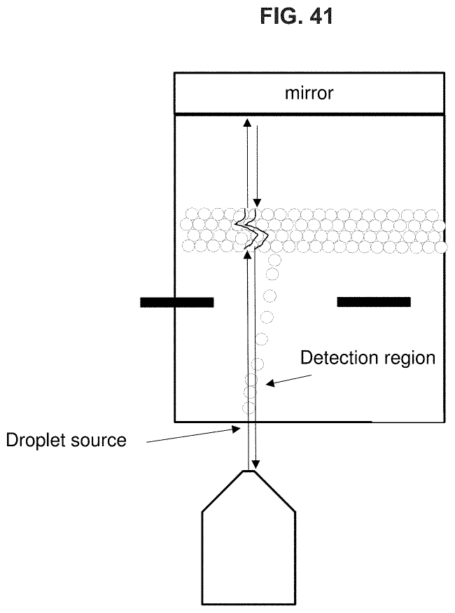

The devices of this example provide for detecting droplets after they are formed in a continuous phase and as they are released into a collection reservoir, such as a well. Such devices solve the problem depicted in FIG. 41, in which the accumulation of droplets interferes with the ability to detect the droplets using conventional means, such as bright-field microscopy.

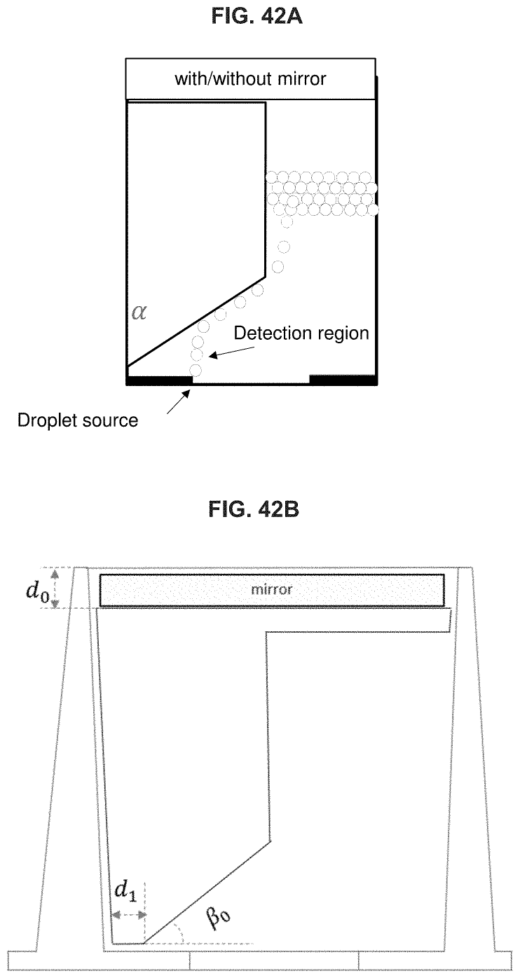

FIGS. 42A and 42B show embodiments of a device according to the invention that includes a deflecting surface having an angle α from a vertical plane (FIG. 42A) or an angle β from a horizontal plane (FIG. 42B) that laterally deflects droplets as they float from a droplet source through the continuous phase in a well. The device of FIG. 42B includes a distance d₁ between the angle of the deflecting surface and the edge of the device that abuts the bottom corner of the well in which it is inserted. The deflecting surface is transparent to provide for unobstructed detection of the droplets as they emerge from the droplet source.

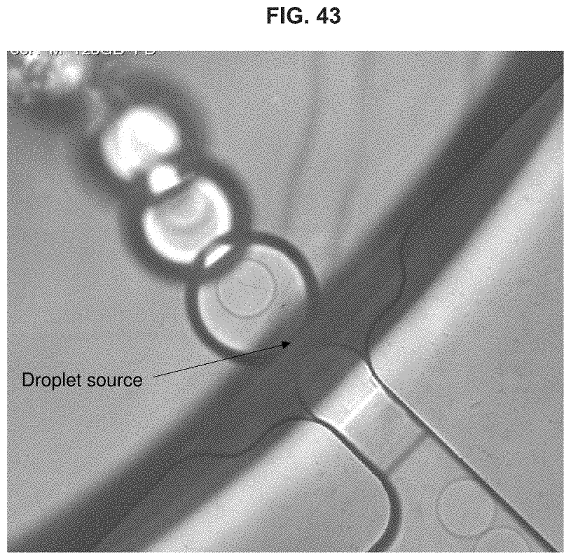

FIG. 43 is a photomicrograph obtained using a bright-field microscope with a device similar to that shown in FIGS. 42A and 42B. The stream of droplets emerges from a microfluidic device containing a droplet formation region and enters the well at the droplet source. The stream is deflected laterally (toward the upper left) by the transparent deflecting surface (invisible in FIG. 43). This deflection provides a clear view of each single droplet as it emerges from the droplet source.

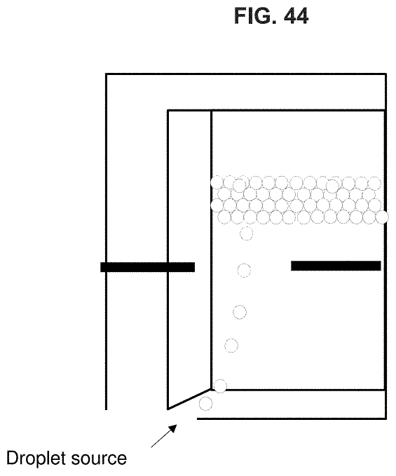

FIGS. 44 and 45A-45F show particular embodiments of a device in which the laterally deflecting surface is configured to deflect the droplets into a central collection region. In these embodiments, the deflecting surface is on the periphery of a hollow, cylindrical insert configured to fit within the walls of a cylindrical well. FIGS. 45D-45F show embodiments of a device having a top crown (e.g., a reflector crown) configured to hold a reflector in place.

FIGS. 46A and 46B show embodiments of inserts in which the deflecting surface is peripheral to a collection region bounded, in part, by the wall of the cylindrical well within which the insert is configured to fit. These embodiments allow the lateral dimension of the deflecting surface to be reduced, relative to the device shown in FIG. 42A, which can help reduce light reflection from the stream of droplets. In addition, the volume of the collection region can be increased. The devices of FIGS. 46A and 46B feature openings in the insert configured to allow the continuous phase to freely pass therethrough into a separate volume free of accumulating droplets. The ability to shunt the continuous phase into a separate continuous phase reservoir enhances the number of droplets that can be collected in the collection region, because, as droplets accumulate, the relative volume occupied by the continuous phase in the droplet collection region decreases as it flows into the continuous phase reservoir.

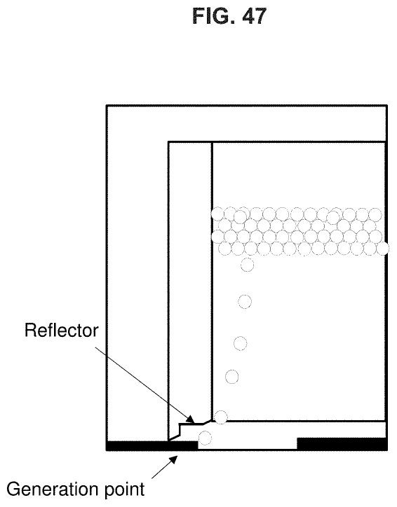

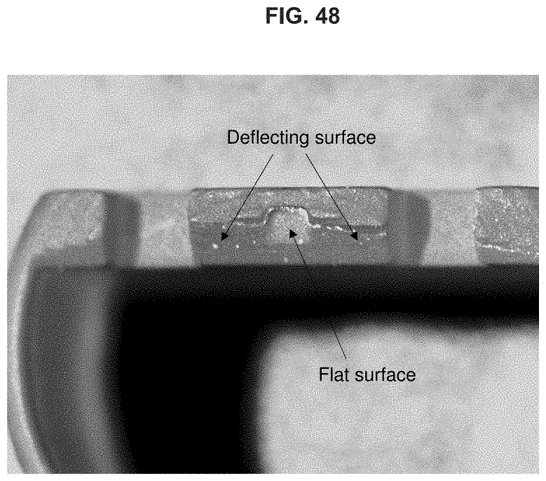

The device of FIG. 47 includes a reflector at the deflecting surface. This device allows for enhanced bright-field microscopic detection when the reflector is positioned above the objective such that the droplet detection region is positioned between the objective and reflector. FIG. 48 shows an insert having a flat surface that can be adapted as a reflector. The insert of FIG. 48 also includes openings for shunting the continuous phase, as described above and shown in FIG. 45B.

### Example 42

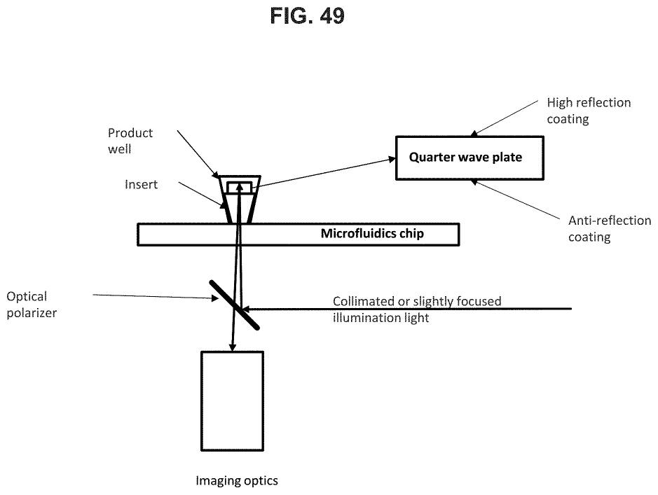

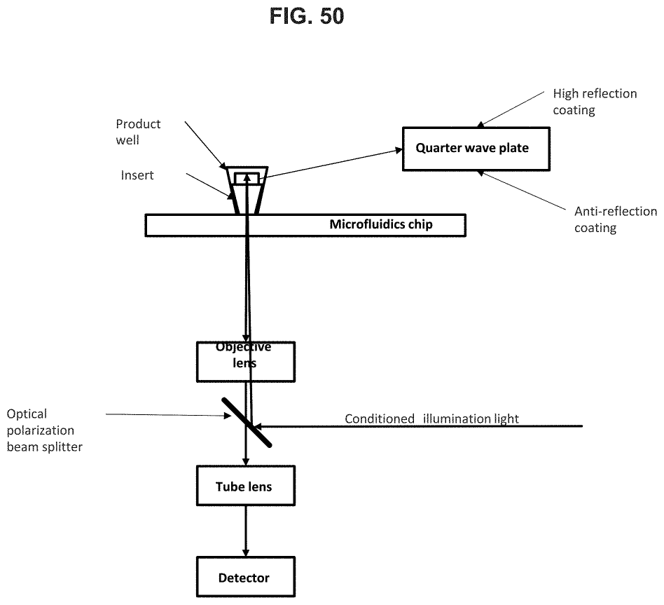

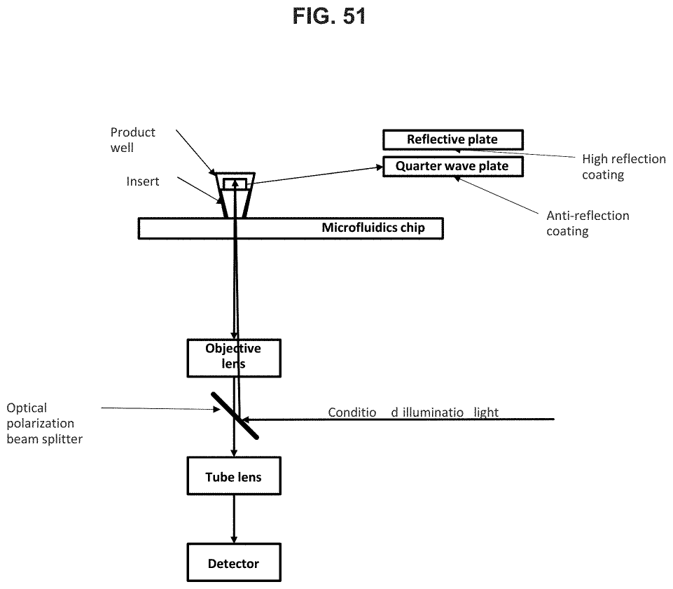

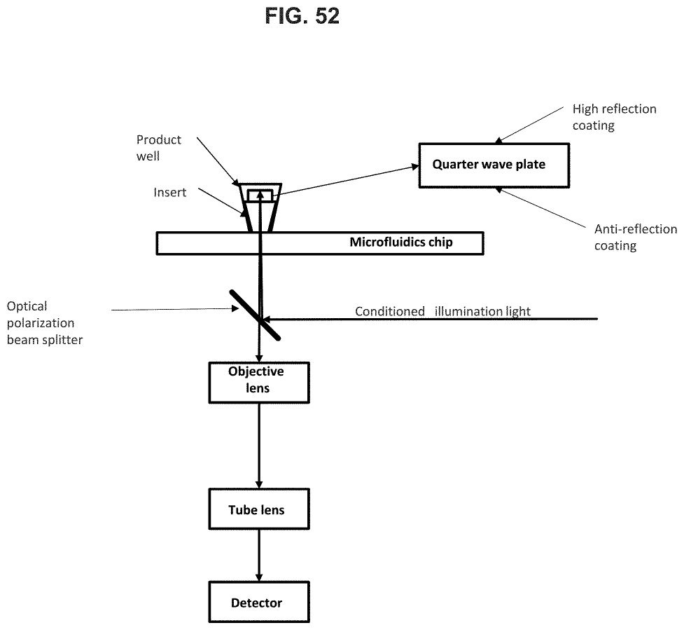

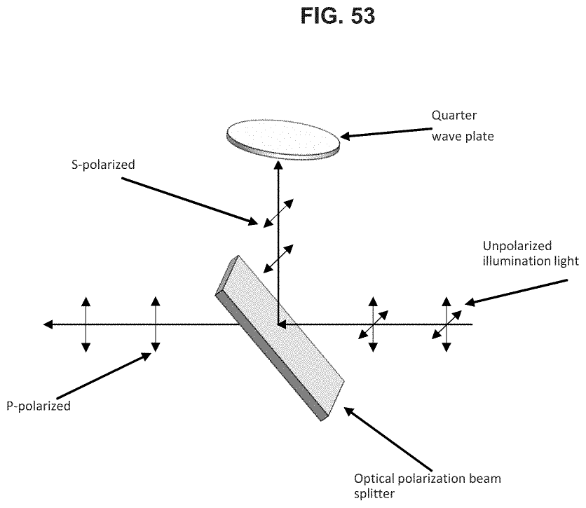

FIGS. 49-52 show various embodiments of a device including a wave plate. FIG. 49 shows a general schematic representation of a device in which collimated, or slightly focused illumination light is reflected by the optical polarizer at a first polarization, through the detection region, and a quarter wave plate rotates the polarization. The light is then reflected off a high reflective coating back through the wave plate, to a second polarization orthogonal to the first polarization. The light then passes through the detection region and the polarizer to the imaging optics system, which detects the light. FIG. 50 shows a setup that further includes an objective lens positioned between the device and the polarizer. A tube lens and a detector are positioned on the other side of the polarizer. FIG. 51 shows an embodiment similar to FIG. 50 in which the quarter wave plate does not have a reflective coating. Instead, a reflective plate with a high reflection coating is positioned above the quarter wave plate to reflect light back through the wave plate. FIG. 52 shows an embodiment similar to FIG. 50 in which the polarizer is positioned between the device and the objective lens instead of between the objective lens and the tube lens. The objective lens gathers light from the droplet or particle being observed and focuses the light to produce an image that is detected by the detector. The tube lens is used to focus the image before detection.

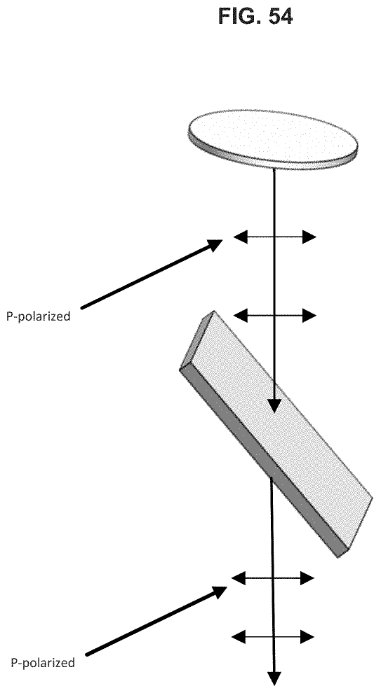

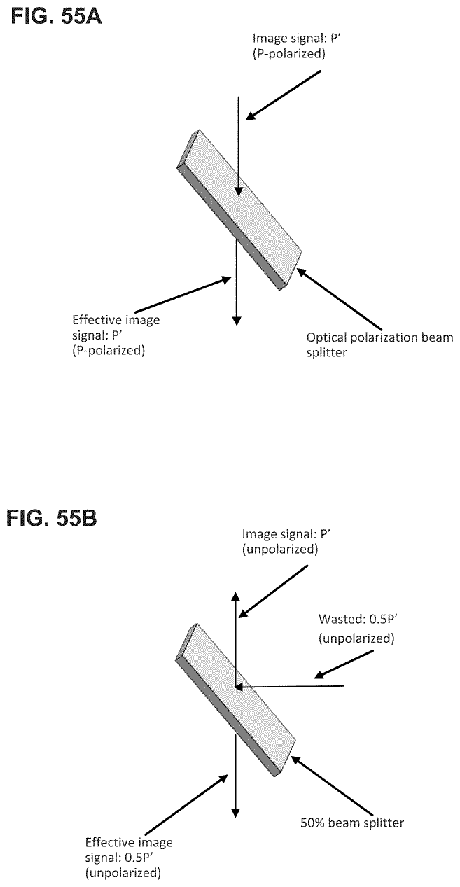

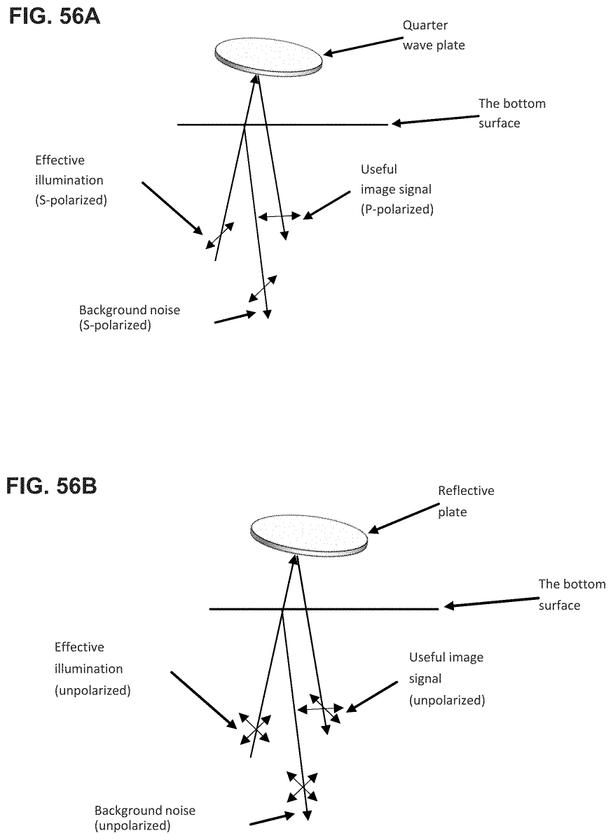

FIGS. 53, 54, 55A, 55B, 56A, and 56B illustrate the enhancement of the imaging signal when using a quarter wave plate in the detection region. FIG. 53 shows a scheme in which unpolarized illumination light is directed towards the polarizer, some of the light is reflected towards the quarter wave plate and is s-polarized, and the remaining light passes through and is p-polarized. FIG. 54 shows a scheme in which p-polarized light, resulting from s-polarized light passing through and being reflected back through a quarter wave plate, passes through the polarizer. As shown in FIG. 55B, an unpolarized image signal P′ passes through a 50% beam splitter, and the effective image signal is only 50%, whereas the remaining 50% of the image signal is reflected by the polarizer and lost. In contrast, FIG. 55A illustrates an analogous scenario in which a polarized image signal P′ passes through a polarizer, and 100% of the effective image signal is retained. FIG. 56A illustrates an embodiment with a quarter wave plate in which the useful image signal is p-polarized and the background noise is s-polarized. Only the signal passes through the polarizer, thereby providing an enhanced signal to noise ratio. In contrast, FIG. 56B illustrates an embodiment with a reflective plate in which both the useful image signal and the background noise are unpolarized. Both signal and noise are then detected.

### Example 43

The devices of this example provide for high-speed imaging of droplets in a microfluidic chip at multiple focal planes. Such devices solve a problem commonly encountered in imaging objects (e.g., droplets) using conventional means, such as bright-field microscopy. Imaging objects (e.g., droplets) at high magnification results in a relatively smaller depth of field. This fundamental tradeoff between depth of field and spatial resolution in conventional microscopy allows either imaging multiple depths of a sample (e.g., droplets) in low detail, or imaging a shallow section of the specimen with high resolution. Devices of this example circumvent this problem by simultaneously imaging multiple focal planes of a sample (e.g., droplets) at high resolution. Imaging objects (e.g., droplets) at multiple focal planes using devices of this invention allows obtaining three dimensional profiles of objects (e.g., droplets) and imaging objects (e.g., droplets) that are out of focus.

FIG. 63A and FIG. 63B show exemplary embodiments of a device according to the invention that includes a droplet formation region and a channel with a slanted portion that is slanted in Z direction. The channel of the device also has a horizontal portion positioned between the droplet formation region and the slanted portion of the channel. The slanted portion is aligned to be at angle γ to the horizontal portion of the channel. Droplets formed in the droplet formation region of the device flow from the droplet formation region to the slanted portion via the horizontal portion of the channel. While flowing along the slanted portion, the droplets change their vertical position (e.g., rise) along the slant of the slanted portion, thus flowing across one or more horizontal planes along the slanted portion of the channel. One or more detectors are aligned with the slanted portion for detection of one or more horizontal planes in the slanted portion of the channel. As the droplets flow along the slanted portion of the channel and subsequently cross the focal planes, the detector detects the droplets at one or more of those different focal planes. This results in imaging of droplets at multiple focal planes by the devices described in FIG. 63A and FIG. 63B. Specifically, FIG. 63A depicts one such device, where the slanted portion of the channel has a constant depth, i.e., a constant distance is maintained between the upper and lower walls of the channel. FIG. 63B depicts another embodiment of such devices, where the slanted portion of the channel has a variable depth, i.e., distance between the upper and lower walls is variable, at least in some portion of the channel. In the devices depicted in FIGS. 63A and 63B, change in vertical position (e.g., rise or fall) of the droplets, as the droplets flow along the slanted portion of the channel, is, at least in part, regulated by the buoyant force acting on the droplets.

### Example 44

FIG. 64 depicts an exemplary embodiment of a device according to the invention that includes a droplet formation region and a channel that is aligned with two lenses having different focal planes. The channel is aligned with the lenses in a way that droplets formed in the droplet formation region are detected at each of the different focal planes, as the droplets flow along the channel. This results in imaging of droplets at multiple focal planes by the device shown in FIG. 64. Other embodiments of such device are also possible, where the channel is aligned with more than two lenses having different focal planes.

### Example 45

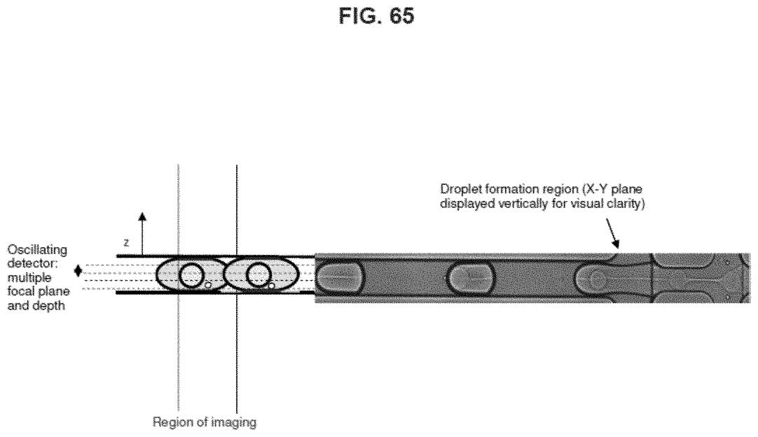

FIG. 65 depicts an exemplary embodiment of a device according to the invention that includes a droplet formation region and a channel that is aligned with an oscillating detector. Piezoelectric and/or acoustic oscillation of the oscillating detector allows the oscillating detector to detect objects (e.g., droplets) at multiple focal planes. The channel of the device runs parallel to the focal planes of the oscillating detector, and droplets formed in the droplet formation region of the device are detected at multiple focal planes of the oscillating detector, as the droplets flow along the channel. This results in imaging of droplets at multiple focal planes by the device shown in FIG. 65.

### Example 46

The device of this example provides for focused imaging of droplets in a microfluidic chip by positioning droplets in the focal plane of a detector. The device also provides focused imaging of particles (e.g., cells and/or beads) within droplets in a microfluidic chip by positioning the particles (e.g., cells and/or beads) in the focal plane of a detector. Such a device is directed to solve a problem that is encountered with random positioning (e.g., positioning in different focal planes) of particles (e.g., random positioning of cells with respect to beads) within a droplet, which may leave the particles (e.g., cells and/or beads) outside the focal plane of the detector, resulting in missed particles (i.e., false negative imaging) or poorly focused imaging of the particles. A device of this example circumvents this problem by positioning droplets (and/or particles (e.g., cells and/or beads) within such droplets) in the focal plane of a detector by constricting the droplets, ensuring highly focused imaging of droplets (and/or particles within such droplets).

FIG. 66 depicts an exemplary embodiment of a device according to the invention that includes a channel that is constricted, i.e., distance between the upper and lower walls is reduced at least in some portions of the channel. The channel is aligned with a detector for optical detection of droplets (e.g., droplets formed in the droplet formation region of the device) as the droplets flow along the channel. Droplets formed in the droplet formation region of the device are positioned in the focal plane of the detector as the droplets flow along the constricted portion of the channel, resulting in focused imaging of the droplets by the detector. Flow along the constricted portion of the channel also reduces the height of the droplets, thereby positioning the particles (e.g., cells and/or beads) within a defined focal plane. The cell can be positioned at or near the center of the droplet using known methods, such as the centering method described in Kamperman et al., small 2017, 13(22): 1603711; which is incorporated herein by reference.

### Example 47

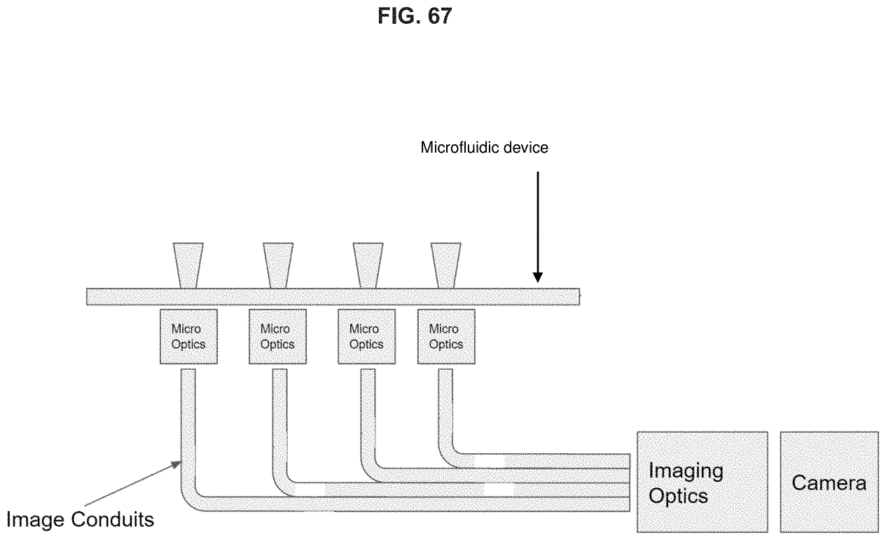

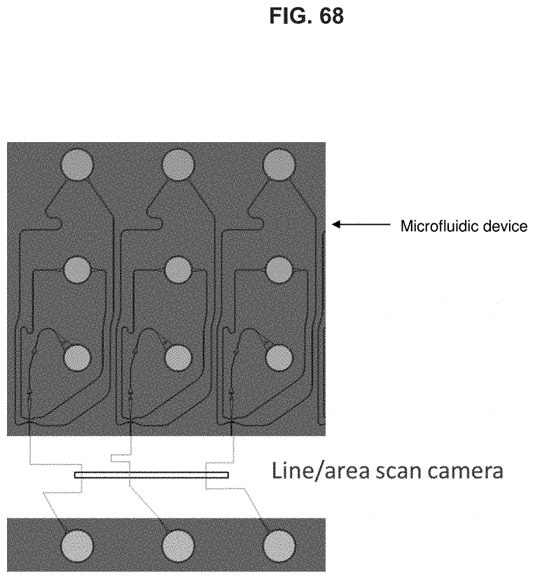

Detection systems shown in FIGS. 67 and 68 are useful for simultaneously detecting multiple detection points (e.g., wells or channels) of a microfluidic device.

In particular, the detection system shown in FIG. 67 combines images from different detection points of a microfluidic device into a single image, which is captured with a single detector. As depicted in FIG. 67, each of multiple detection points of a microfluidic device is aligned with a lens (e.g., a micro-lens). The outputs of these lenses are aligned with the receiving end of a light guide, such as an image conduit. The transmitting ends the image conduits are aligned with a detector (e.g., a camera). When the multiple detection points of the microfluidic device are illuminated by a light source (not depicted), light from each of the detection points is transmitted via the lenses and light guides to the camera. Multiple images are combined to form a single image on the sensor.

The detection system shown in FIG. 68 simultaneously detects non-overlapping signals from multiple channels of a microfluidic device with a linear detector (e.g., a line scan camera). As depicted in FIG. 68, a line scan camera is aligned with multiple channels of a microfluidic device. When the multiple channels of the microfluidic device are illuminated by a light source (not depicted), non-overlapping signals from the multiple channels are detected.

### Example 48

Detection systems, such as those shown in FIGS. 69 and 70, are useful for detecting the presence or characteristics of particular objects in a sample in a microfluidic device. In particular, the angle of the laser beam passing through the microfluidic device and the lack of bulky objectives provides a more compact and efficient configuration than traditional systems, such as fluorescent microscopy systems designed for conventional (e.g., static) culture vessels.

As shown in FIG. 69, a laser transmits a laser beam through shaping optics (such as a polarizer) at an angle α to a microfluidic device, which is shown as a fluidic channel sandwiched between a chip bottom plate and a chip top plate. The microfluidic device is placed on a stage (not depicted). The angle α is 90° minus the angle of incidence. The angle of incidence is approximately Brewster's angle, such that about all light is transmitted (e.g., at least 90%) into the microfluidic device (instead of reflected off the top surface of the device). The laser beam is thus transmitted into the fluidic channel to detect the presence or characteristics of a sample at a region to be interrogated (the interrogation volume), such as a droplet formation region. The laser beam passes through the bottom of the device and is not received by the collection optics positioned under the device. However, light directed by the sample or components thereof can be directed toward the collection optics (e.g., light that is scattered from the laser or light emitted from a fluorophore in the sample). The light directed by the sample or components thereof is detected by a detector (not shown in FIG. 69) configured to detect the light transmitted by the collection optics through a multimode optical fiber that receives light transmitted by the collection optics.

FIG. 70 shows an alternate embodiment of a detection system, in which the laser is positioned on the same side of the microfluidics device as the collection optics. Both are below the microfluidics device. As in the device of FIG. 69, light is directed by the sample or components thereof to the collection optics and optical fiber to a detector.

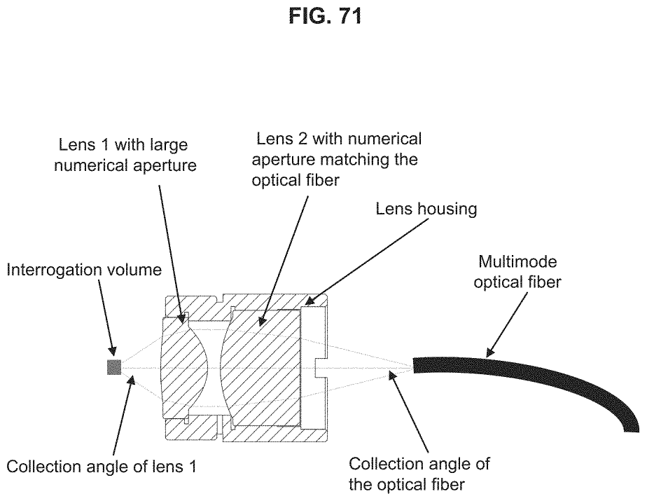

FIGS. 71-73 show various exemplary configurations of collection optics, optical fibers, and optical detectors. In general, collection optics (e.g., including an aspherical lens pair) are configured such that the input focal point is co-localized with the detection point (i.e., a point within the interrogation volume corresponding to a point of interest in the device, such as a droplet formation region). The distance between the detection point (and/or microfluidic device and/or stage) and the collection optics is thus determined by the collection angle of lens 1. Light passing through the collection optics can then be transmitted to one or more detectors in various ways.

FIG. 71 shows an embodiment in which the light transmitted through the collection optics is focused into a multimode optical fiber, which transmits the light to a detector. In this embodiment, the collection optics and optical fiber are positioned such that the output focal point of the collection optics is co-localized with the receiving end of the optical fiber. The distance between the collection optics and the receiving end of the optical fiber is determined by the collection angle of the optical fiber. Thus, the numerical aperture of lens 2 is matched with the optical fiber.

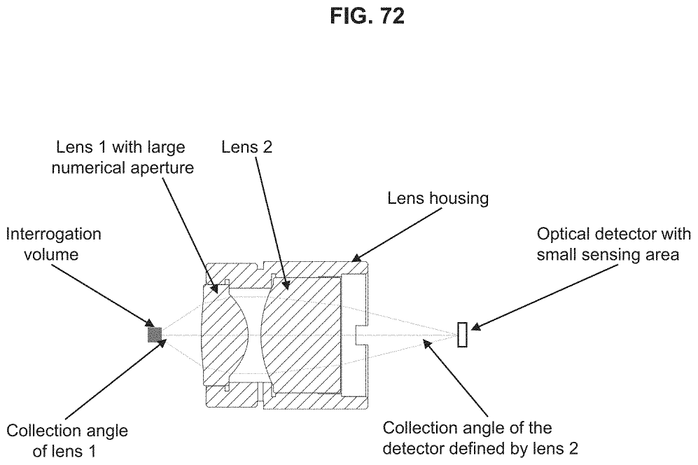

In an alternative configuration, shown in FIG. 72, optical fiber is omitted in favor of an optical detector positioned to detect light transmitted by the collection optics.

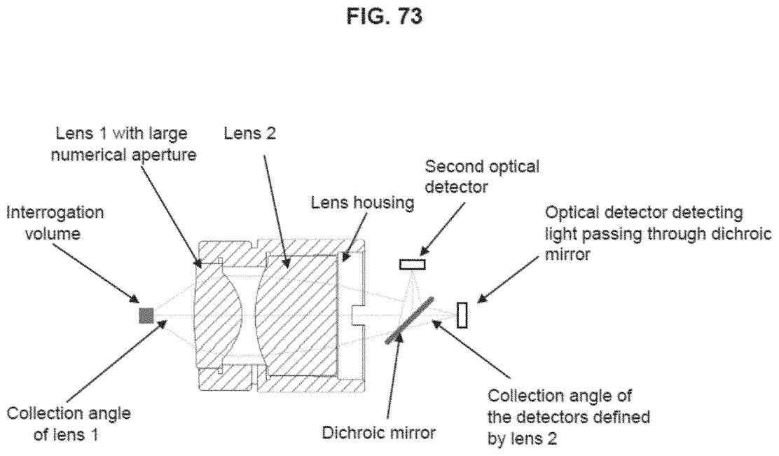

Such configurations can also be fitted with one or more dichroic mirrors, as shown in FIG. 73. By positioning a dichroic mirror to receive light transmitted by the collection optics, the light is directed to multiple detectors, e.g., by passing light of a first wavelength to a first detector and reflecting light of a second wavelength to a second detector. While FIG. 73 shows the light being directed directly from the dichroic mirror to both detectors, one or more optical fibers can be positioned to transmit the light from the dichroic mirror to the detector(s). Similarly, multiple mirrors (e.g., dichroic mirrors), optical fibers, and detectors may be configured in various ways as necessitated by the user's experimental setup.

### Example 49

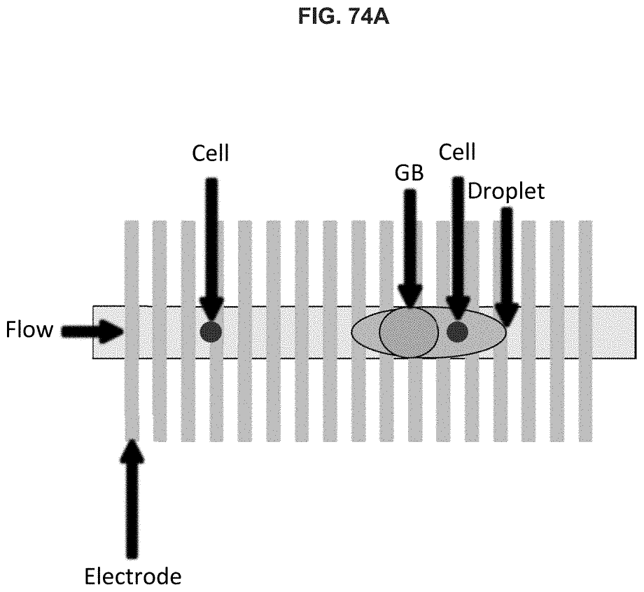

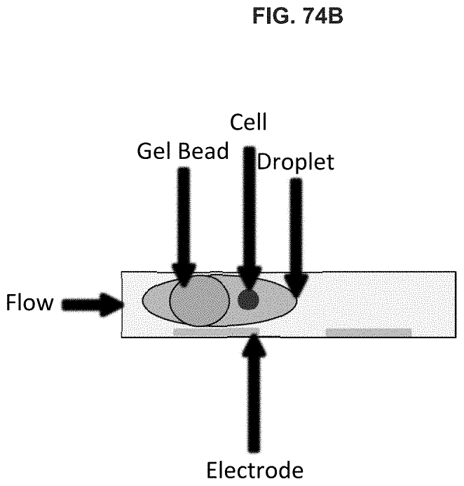

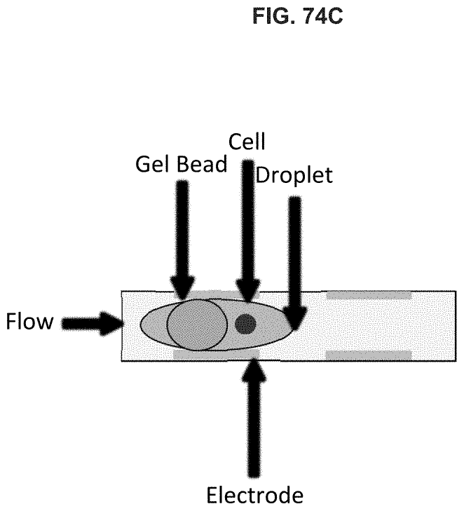

FIG. 74A shows a top view of an embodiment of a device according to the invention that includes a plurality of electrodes. The electrodes are disposed on the bottom of the channel. The impedance of the droplet and a cell not within a droplet are measured as they pass through the detection channel. The droplet contains particles (e.g., a gel bead and a cell) and a droplet aqueous phase, all of which will contribute to the impedance of the droplet. The difference in impedance between the droplet containing particles and the cell can be measured to differentiate the two. FIG. 74B shows a cross-sectional view of an embodiment of a device according to the invention detecting a droplet containing a gel bead and a cell. The view of the device shows two electrodes on the bottom of the detection channel. The electrodes are positioned to measure a droplet or particle as it passed through the channel. FIG. 74C shows a cross sectional view of an embodiment of a device according to the invention detecting a droplet containing particles. The cross-sectional view of the device shows electrodes positioned on the bottom and the top of the detection channel. The electrodes on the top are positioned directly above the electrodes positioned on the bottom. The electrodes are positioned to measure a droplet or particle as it passed through the channel. A droplet containing a gel bead, a cell, and a droplet aqueous phase is shown passing between two electrodes.

### Example 50

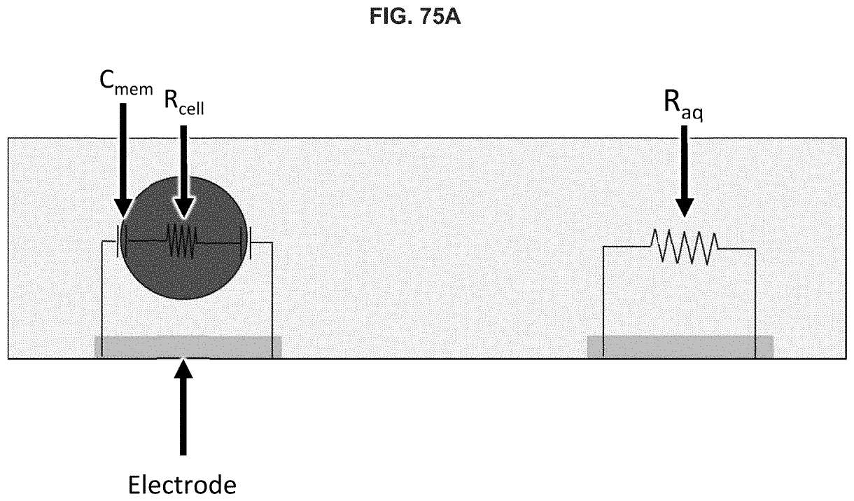

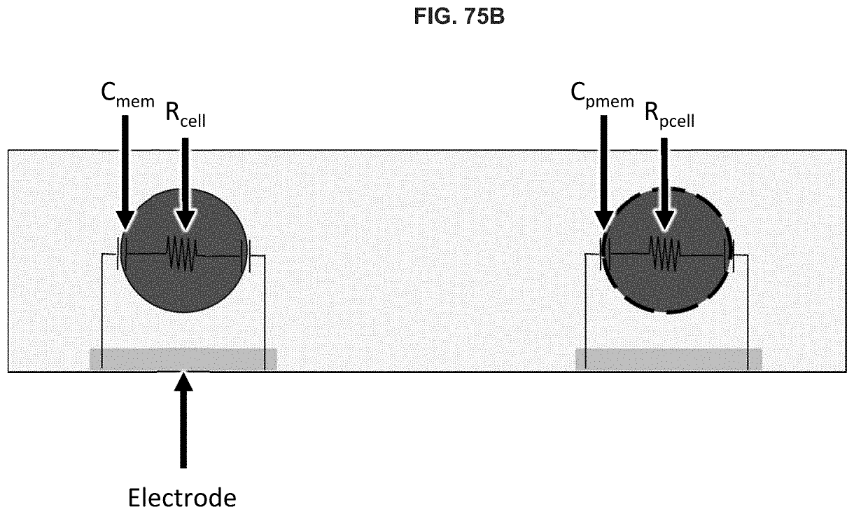

FIGS. 75A and 75B show an embodiment of a device according to the invention measuring a cell and cell viability. FIG. 75A shows a depiction of equivalent circuits of a cell and a continuous phase. FIG. 75B shows an embodiment of the device according to the invention measuring cell viability. The figure shows the equivalent circuits of a viable cell with an intact cell membrane and a non-viable cell with a cell having a cell membrane with a variable permeability (i.e., increased permeability relative to a viable cell with an intact cell membrane). Differences in impedance allow differentiation between the two.

### Example 51

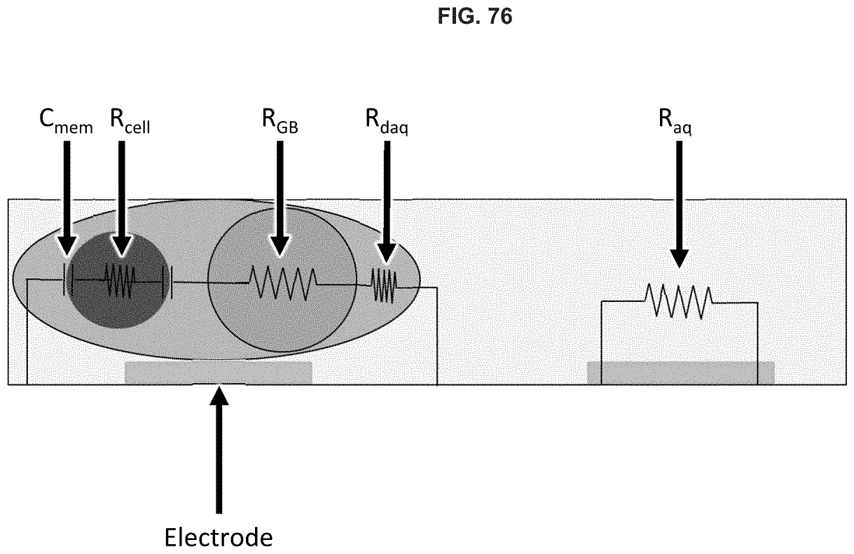

FIG. 76 shows an embodiment of a device according to the invention detecting a droplet and the particles within the droplet. The equivalent circuit for a droplet containing a cell, having a cell membrane capacitance (Cₘₑₘ) and a cell resistance (R_{cell}), a gel bead (GB) having a gel bead resistance (R_{GB}), and a droplet aqueous phase having a resistance (R_{daq}) is shown. The equivalent circuit for the continuous phase is also shown. Differences in impedance allow differentiation between the two.

### Example 52

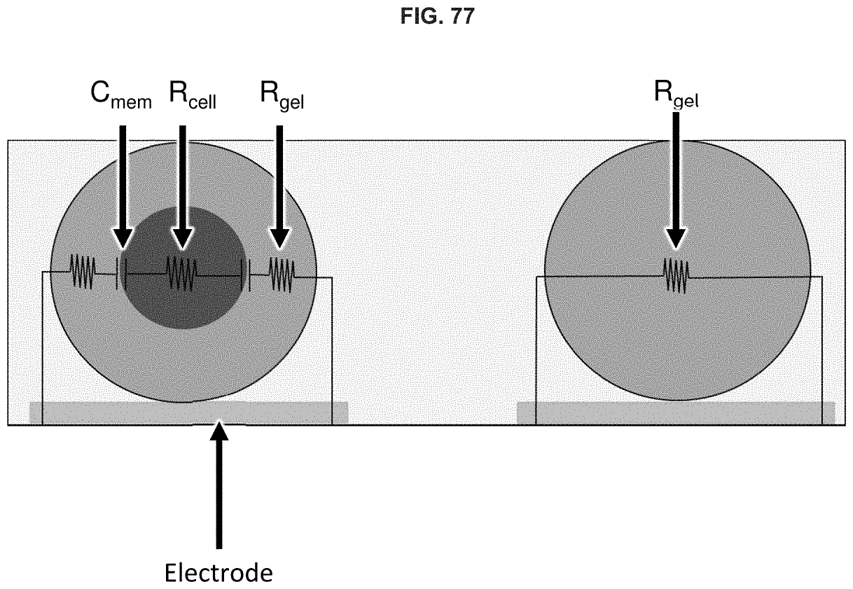

FIG. 77 shows an embodiment of the device according to the invention detecting the presence of a cell within a gel. The equivalent circuits for a cell within a gel and a gel without a cell are shown. Differences in impedance allow differentiation between the two.

### ORDERED EMBODIMENTS

The following sections describe various embodiments of the invention.

### Embodiment A

1. A device for sorting droplets or particles comprising:
(a) a droplet or particle source configured to provide droplets or particles within a continuous phase;
(b) a sorting region, in fluid communication with the droplet or particle source, comprising a sorter configured to sort one or more of the droplets or particles; and
(c) a collection region, in fluid communication with the sorting region, comprising two or more partitions configured for collection of the droplets or particles after sorting.

2. The device of embodiment 1, wherein the droplet or particle source comprises a droplet or particle formation region configured to form droplets or particles.

3. The device of embodiment 1 or 2, wherein the sorter is configured to provide mechanical or electromagnetic force to move the one or more droplets or particles into the two or more partitions.

4. The device of any one of embodiments 1-3, wherein the sorter comprises a dielectrophoretic actuator, an acoustic actuator, a fluidic (e.g., pneumatic) actuator, a mechanical actuator, a bubble generator, an optical tweezer, a magnet, a thermal actuator, or an electrostatic charger.

5. The device of embodiment 4, wherein the dielectrophoretic actuator or the electrostatic charger comprises an electrode.

6. The device of embodiment 5, wherein the electrode comprises a fluid channel or a solid conductor.

7. The device of any one of embodiments 1-6, wherein the sorter comprises a divider.

8. The device of any one of embodiments 1-7, wherein the sorting region comprises a surface configured to laterally deflect one or more of the droplets or particles to one of the two or more partitions.

9. The device of embodiment 8, wherein the surface comprises one or more openings through which the one or more deflected droplets or particles pass.

10. The device of any one of embodiments 1-9, wherein the sorting region comprises a detection region to allow detection of the droplets or particles as they pass therethrough.

11. The device of embodiment 10, wherein the detection region is configured to provide feedback to the sorting region.

12. The device of embodiment 11, wherein the feedback is configured to actuate the sorter.

13. The device of any one of embodiments 1-12, wherein the collection region comprises 3, 4, 5, 6, 7, 8 9, 10, or more partitions.

14. The device of any one of embodiments 1-13, wherein the sorting region has a volume of at least 1 μL.

15. The device of any one of embodiments 1-14, wherein each cross-sectional dimension of the sorting region has a length of at least 1 mm.

16. The device of any one of embodiments 1-15, wherein the droplet comprises a particle.

17. The device of embodiment 16, wherein the particle comprises a biological particle, a bead, or a combination thereof.

18. The device of embodiment 17, wherein the biological particle comprises a cell or one or more constituents of a cell.

19. The device of embodiment 17 or 18, wherein the biological particle comprises a matrix.

20. A method of sorting droplets or particles comprising:
(a) providing the device of any one of embodiments 1-19 comprising the continuous phase in the sorting region;
(b) allowing the droplets or particles to enter the sorting region; and
(c) sorting one or more of the droplets or particles into one or more of the two or more partitions in the collection region.

21. The method of embodiment 20, wherein the sorter provides mechanical or electromagnetic force to sort the one or more droplets or particles into the two or more partitions.

22. The method of embodiment 20 or 21, wherein the sorter laterally deflects one or more of the droplets or particles to one of the two or more partitions.

23. The method of any one of embodiments 20-22, further comprising detecting the droplets or particles prior to step (c).

24. The method of embodiment 23, wherein the detection provides feedback to the sorter.

25. The method of embodiment 24, wherein the feedback actuates the sorter to sort the one or more of the droplets or particles.

### Embodiment B

1. A device for sorting droplets or particles comprising:
(a) a droplet or particle source configured to provide droplets or particles within a continuous phase;
(b) a first channel having a first depth, a first width, a first proximal end, and a first distal end, wherein the first channel is in fluid communication with the droplet or particle source; and
(c) a barrier configured to operatively connect one or more electrodes with the first channel.

2. The device of embodiment 1, further comprising (d) a collection region comprising two or more partitions configured for collection of the droplets or particles, wherein the collection region is in fluid communication with the first channel.

3. The device of embodiment 2, wherein the collection region comprises 3, 4, 5, 6, 7, 8, 9, 10, or more partitions.

4. The device of embodiment 1, wherein the droplet or particle source comprises a droplet formation region configured to form droplets from a first liquid in a second liquid.

5. The device of embodiment 4, further comprising a second channel having a second depth, a second width, a second proximal end, and a second distal end, and a third channel having a third depth, a third width, a third proximal end, and a third distal end, wherein the third channel intersects the second channel between the second proximal and second distal ends, wherein the second distal end is in fluid communication with the first proximal end, and wherein the second channel and the droplet formation region are configured to produce droplets of the first liquid in the second liquid.

6. The device of embodiment 5, wherein the droplet formation region comprises a shelf region having a fourth depth and a fourth width and a step region having a fifth depth, wherein the shelf region is configured to allow the first liquid to expand in at least one dimension and has at least one inlet and at least one outlet, and wherein the shelf region is disposed between the second distal end and the step region.

7. The device of embodiment 1, wherein the barrier has a thickness of at least 1 μm.

8. The device of embodiment 7, wherein the barrier has a thickness of from about 1 μm to about 100 μm.

9. The device of embodiment 8, wherein the barrier has a thickness of from about 5 μm to about 50 μm.

10. The device of embodiment 1, wherein the droplet comprises a particle.

11. The device of embodiment 9, wherein the particle comprises a biological particle, a bead, or a combination thereof.

12. The device of embodiment 11, wherein the biological particle comprises a cell or one or more constituents of a cell.

13. The device of embodiment 11, wherein the biological particle comprises a matrix.

14. The device of embodiment 1, wherein the barrier is configured to operatively connect 2, 3, 4, 5, 6, 7, 8, 9, 10, or more electrodes with the first channel.

15. The device of embodiment 1, wherein the device comprises one or more cavities configured to receive the one or more electrodes and position the one or more electrodes adjacent to the barrier.

16. A system comprising the device of embodiment 1, the one or more electrodes, and a housing configured to position the one or more electrodes in operative connection with the first channel.

17. The system of embodiment 16, wherein at least one of the one or more electrodes is a dielectrophoretic electrode.

18. The system of embodiment 16, wherein at least one of the one or more electrodes comprises a fluid channel or a solid conductor.

19. The system of embodiment 16, further comprising a conductive fluid disposed between at least one of the one or more electrodes and the barrier.

20. The system of embodiment 19, wherein the conductive fluid is a gel.

21. A system comprising the device of embodiment 2, the one or more electrodes, and a housing configured to position the one or more electrodes in operative connection with the first channel.

22. A method of sorting droplets or particles comprising:
(a) providing the system of embodiment 21 comprising the continuous phase in the first channel and the barrier operatively connecting the one or more electrodes with the first channel;
(b) allowing the droplets or particles to enter the first channel; and
(c) sorting one or more of the droplets or particles into one or more of the two or more partitions in the collection region by applying an electromagnetic field to the droplets or particles via the one or more electrodes.

### Embodiment C

1. A device for detecting a droplet, wherein the device comprises:
(i) a droplet source configured to provide droplets within a continuous phase;
(ii) a deflecting surface in fluid communication with the droplet source, wherein the deflecting surface is configured to laterally deflect the droplet;
(iii) a detection region configured to allow detection of the droplet as it passes therethrough, and
(iv) a collection region configured for collection of the droplet after detection.

2. The device of embodiment 1, wherein the surface comprises one or more openings configured to shunt the continuous phase to a continuous phase reservoir.

3. The device of embodiment 1 or 2, wherein the surface is configured to deflect droplets having a density lower than the continuous phase.

4. The device of any one of embodiments 1-3, wherein the deflecting surface comprises a transparent material having a refractive index that about matches the refractive index of the continuous phase.

5. The device of any one of embodiments 1-4, wherein the deflecting surface is at an angle above a horizontal plane.

6. The device of embodiment 5, wherein the deflecting surface is at an angle between 0° and 80° above the horizontal plane.

7. The device of any one of embodiments 1-6, wherein the detection region comprises one or more reflectors.

8. The device of embodiment 7, wherein the deflecting surface comprises the reflector.

9. The device of embodiment 7 or 8, wherein the reflector is about horizontal.

10. The device of embodiment 9, further comprising a reservoir and an insert, wherein the insert comprises the deflecting surface and defines one or more boundaries of the collection region, and wherein the reflector is at or above the top of the reservoir.

11. The device of any one of embodiments 1-10, further comprising a reservoir and an insert, wherein the insert comprises the deflecting surface and defines a boundary of the collection region.

12. The device of embodiment 10 or 11, wherein the collection region is 10%-90% of the lateral area of the reservoir.

13. The device of any one of embodiments 10-12, wherein the reservoir is a cylindrical well.

14. The device of any one of embodiments 1-13, wherein the detection region is optically detectable.

15. A system comprising a plurality of the devices of any one of embodiments 1-14 and reagents.

16. The system of embodiment 15, wherein the plurality of devices are connected.

17. The system of embodiment 15 or 16, wherein the reagents comprise the continuous phase.

18. The system of embodiment 17, wherein the continuous phase is disposed in the detection region and all or a portion of the collection region, and wherein the continuous phase is in fluid communication with the droplet source and the deflecting surface.

19. A method of detecting a droplet comprising:
(i) providing the device of any one of embodiments 1-14 comprising a continuous phase in the detection region and all or a portion of the collection region, and wherein the continuous phase is in fluid communication with the droplet source and the deflecting surface;
(ii) allowing a droplet to enter the detection region; and
(iii) detecting the droplet in the detection region.

20. The method of embodiment 19, wherein the detection is optical.

21. The method of embodiment 20, wherein the optical detection is by imaging or video recording.

22. The method of any one of embodiments 19-21, wherein the droplet is aqueous or miscible with water, and wherein the droplet is immiscible with the continuous phase.

23. The method of embodiment 22, wherein the continuous phase is an oil.

### Embodiment D

1. A device for detecting a droplet or particle comprising:
(a) a droplet or particle source; and
(b) a detection region in fluid communication with the droplet or particle source;
wherein the detection region comprises a wave plate.

2. The device of embodiment 1, further comprising a polarizer configured to polarize light from a light source.

3. The device of embodiment 2, wherein the polarizer is a polarization beam splitter.

4. The device of embodiment 1, wherein the detection region comprises a reservoir.

5. The device of embodiment 1, wherein the wave plate is a quarter wave plate.

6. The device of embodiment 1, wherein the droplet or particle source comprises a droplet formation region configured to form droplets of a first liquid in a second liquid.

7. The device of embodiment 1, further comprising a reflector configured to reflect light passing through the wave plate.

8. A system for detecting a droplet or particle comprising the device of any one of embodiments 1-7, a light source, and a light detector.

9. The system of embodiment 8, further comprising a polarizer configured to polarize light from the light source in a first polarization and direct it to the detection region.

10. The system of embodiment 9, wherein the polarizer is a polarization beam splitter.

11. The system of embodiment 9, wherein the polarizer is configured to direct light polarized in a second polarization that is orthogonal to the first polarization to the detector.

12. A method of detecting a droplet or particle or a characteristic thereof comprising:
(a) providing the system of any one of embodiments 8-11;
(b) allowing the droplet or particle to enter the detection region;
(c) illuminating the droplet or particle in the detection region with light polarized in a first polarization, wherein the light passes through the wave plate; and
(d) measuring light that passes through the wave plate, thereby detecting the droplet or particle or the characteristic thereof.

13. The method of embodiment 12, wherein in step (c), the light passes through the wave plate and is reflected by a reflector back through the wave plate, thereby rotating the polarization to a second polarization orthogonal to the first polarization.

14. The method of embodiment 13, wherein light having the second polarization passes through the polarizer to the detector.

15. The method of embodiment 12, wherein the characteristic is the size of the droplet or particle, the velocity of the droplet or particle, the presence of one or more particles in the droplet, and/or the location of one or more particles in the droplet.

16. The method of embodiment 12, wherein the droplet comprises a particle.

17. The method of embodiment 16, wherein the characteristic of the droplet is a characteristic of the particle in the droplet.

18. The method of embodiment 16, wherein the particle comprises a biological particle, a bead, or a combination thereof.

19. The device of embodiment 18, wherein the biological particle comprises a cell or one or more constituents of a cell.

20. The method of embodiment 19, wherein the characteristic is a type of cell, a composition of the cell, and/or a size of the cell.

### Embodiment E

1. A device for detecting light from a droplet or particle, wherein the device comprises:
(a) a droplet or particle source;
(b) a detection channel in fluid communication with the droplet or particle source, the detection channel having a proximal end and a distal end;
(c) a light source or first light guide; and
(d) one or more detectors or second light guides,
   wherein the detection channel, the light source or first light guide, and the one or more detectors or second light guides are configured for the detection of light as a droplet or particle passes from the proximal end to the distal end of the detection channel.

2. The device of embodiment 1, further comprising (e) a collection region comprising two or more partitions configured for collection of the droplets or particles, wherein the collection region is in fluid communication with the first channel.

3. The device of embodiment 2, wherein the collection region comprises 3 or more partitions.

4. The device of embodiment 1, wherein the droplet or particle source comprises a droplet formation region.

5. The device of embodiment 1, wherein the one or more detectors or second light guides are placed on one side of the detection channel or multiple sides of the detection channel.

6. The device of embodiment 1, further comprising a controller operatively coupled to direct the transport of the droplet or particle through the detection channel.

7. A system for detecting light from a droplet or particle, the system comprising:
(a) a device for detecting light, comprising:
   (i) a light source or first light guide; or
   (ii) one or more detectors or second light guides; and
(b) a fluidic device, comprising:
   (i) a droplet or particle source;
   (ii) a detection channel in fluid communication with the droplet or particle source, the detection channel having a proximal end and a distal end,
wherein the system comprises the light source and the one or more detectors, and the fluidic device mates with the device for detecting light, so that the light source and the one or more detectors are configured to detect light as a droplet or particle passes from the proximal end to the distal end of the detection channel.

8. The system of embodiment 7, wherein the device for detecting light comprises the light source.

9. The system of embodiment 7, wherein the device for detecting light comprises the one or more detectors.

10. The system of embodiment 7, wherein the fluidic device comprises the first light guide and/or the one or more second light guides.

11. The system of embodiment 7, wherein the fluidic device does not comprise the light source, the first light guide, the one or more detectors, or the one or more second light guides.

12. A method of detecting a droplet or particle or a characteristic thereof, the method comprising:
(a) providing the device of embodiment 1 or system of embodiment 7;
(b) illuminating the droplet or particle as it passes along the detection channel from the proximal end to the distal end; and
(c) measuring light as the droplet or particle passes through the detection channel, thereby detecting the droplet or particle or the characteristic thereof.

13. The method of embodiment 12, wherein the characteristic is the size of the droplet or particle, the velocity of the droplet or particle, the presence of one or more particles in the droplet, and/or the location of one or more particles in the droplet.

14. The method of embodiment 12, wherein the droplet contains one or more particles.

15. The method of embodiment 14, wherein the one or more particles comprises a cell, a bead, or a combination thereof.

16. The method of embodiment 14, wherein the characteristic of the droplet is a characteristic of the particle in the droplet.

17. The method of embodiment 16, wherein the particle is a cell.

18. The method of embodiment 12, wherein the characteristic is:
(a) the type of cell;
(b) the composition of the cell; and/or
(c) the size of the cell.

### Embodiment F

1. A device comprising a droplet or particle source and a channel comprising a slanted portion, wherein the droplet or particle source provides droplets or particles that flow along the slanted portion, and wherein the slanted portion is aligned with a detector or lens configured for detection of the droplets or particles as the droplets or particles pass through a focal plane in the slanted portion.

2. The device of embodiment 1, wherein the slanted portion of the channel is at an angle from 1° to 80° to the focal plane.

3. The device of embodiment 2, wherein the slanted portion of the channel is at an angle from 20° to 40° to the focal plane.

4 The device of any one of embodiments 1-3, wherein at least a portion of the slanted portion has a constant depth.

5. The device of any one of embodiments 1-3, wherein at least a portion of the slanted portion has a variable depth.

6. The device of any one of embodiments 1-5, wherein the focal plane is horizontal.

7. The device of any one of embodiments 1-6, wherein the channel comprises a first portion running parallel to the focal plane, wherein the first portion of the channel is disposed between the droplet or particle source and the slanted portion of the channel.

8. The device of any one of embodiments 1-7, wherein the droplet or particle source comprises a droplet or particle formation region.

9. A system comprising the device of any one of embodiments 1-8 and a detector or lens, wherein the detector or lens is disposed for detection of the slanted portion of the channel.

10. A method of detecting a droplet, the method comprising:
(a) providing the device of any one of embodiments 1-8 and a detector, wherein the detector is disposed to detect the slanted portion of the channel, or the system of embodiment 9;
(b) providing a droplet or particle and passing the droplet or particle to the slanted portion of the channel; and
(c) detecting multiple cross sections of the droplet or particle as the droplet or particle flows along the slanted portion of the channel.

11. A device comprising a droplet or particle source, a channel, and a plurality of lenses or detectors having different focal planes, wherein the channel is aligned with the plurality of lenses or detectors to position the different focal planes for detection of droplets or particles as the droplets or particles flow along the channel.

12. The device of embodiment 11, wherein the plurality of lenses are micro-lenses.

13. The device of embodiment 11 or 12, wherein the plurality of lenses or detectors have different focal distances.

14. The device of any one of embodiments 11-13, wherein the droplet or particle source comprises a droplet or particle formation region.

15. A system comprising the device of any one of embodiments 11-14 and a plurality of lenses or detectors having different focal planes, wherein the plurality of lenses or detectors is disposed to detect droplets or particles at the different focal planes.

16. A method of detecting a droplet, the method comprising:
(a) providing the device of any one of embodiments 11-14 and one or more detectors or the system of embodiment 15;
(b) passing a droplet or particle from the droplet or particle source to the channel; and
(c) using the one or more detectors to detect the droplet or particle at the different focal planes as the droplet or particle flows along the channel.

17. The method of embodiment 16, wherein the droplet or particle source comprises a droplet or particle formation region.

18. A system comprising a device comprising a droplet or particle source and a channel, wherein the channel is aligned with a detector or lens configured for detection of droplets or particles formed in the droplet or particle source at a plurality of focal planes as the droplets or particles flow along the channel.

19. The system of embodiment 18, wherein the detector or lens is oscillated acoustically or piezoelectrically.

20. The system of embodiment 18 or 19, wherein the droplet or particle source comprises a droplet or particle formation region.

21. A method of detecting a droplet, the method comprising:
(a) providing the system of any one of embodiments 18-20;
(b) passing a droplet or particle from the droplet or particle source to the channel; and
(c) detecting the droplet or particle at the plurality of focal planes using the detector.

22. The method of embodiment 21, wherein the droplet or particle source comprises a droplet or particle formation region.

23. A method of detecting a droplet, the method comprising:
(a) providing a device comprising a droplet or particle source and a channel aligned with one or more detectors;
(b) passing a droplet or particle from the droplet or particle source to the channel; and
(c) detecting the droplet or particle with the one or more detectors at a plurality of focal planes, thereby detecting multiple planes of the droplet.

24. The method of embodiment 23, wherein the droplet or particle source comprises a droplet or particle formation region.

25. The method of embodiment 23 or 24, further comprising:
(d) determining a number of particles in the droplet.

26. The method of embodiment 25, wherein the droplet comprises one or more particles, one of which has a dimension that is less than 50% of the corresponding dimension of the droplet.

27. The method of embodiment 25 or 26, wherein the number of particles is a number of beads or biological particles.

28. The method of embodiment 27, wherein the droplet comprises a bead selected from the group consisting of a solid bead, a semi-solid bead, a gel bead, a polymeric bead, or a magnetic bead.

29. The method of embodiment 27 or 28, wherein the droplet comprises a biological particle selected from the group consisting of a cell, a constituent of a cell, or a virus.

30. The method of embodiment 29, wherein the biological particle is a cell, or an organelle.

31. The method of embodiment 30, wherein the cell is a prokaryotic cell or eukaryotic cell.

32. The method of embodiment 30, wherein the cell is a healthy tissue cell or a cancer cell.

33. A device comprising a droplet or particle source and a channel aligned with a focal plane of a detector or lens, wherein a portion of the channel is constricted to position droplets or particles in the focal plane of the detector or lens for detection of the droplets or particles as the droplets or particles flow along the channel.

34. The device of embodiment 33, wherein depth of the channel is reduced in the constricted portion of the channel.

35. The device of embodiment 33 or 34, wherein the droplet or particle source comprises a droplet or particle formation region.

36. A system comprising the device of any one of embodiments 33-35 and a detector or lens, wherein the device is configured to position droplets or particles in the focal plane of the detector or lens for detection.

37. A method of detecting a droplet, the method comprising:
(a) providing the device of any one of embodiments 33-35 and a detector or the system of embodiment 36;
(b) passing the droplet or particle to the constricted portion of the channel; and
(c) detecting the droplet or particle at the focal plane as the droplet or particle flows along the constricted portion of the channel.

38. The method of embodiment 37, wherein the droplet or particle source comprises a droplet or particle formation region.

39. The method of embodiment 37 or 38, wherein the droplet comprises one or more particles.

40. The method of embodiment 39, wherein the one or more particles are one or more beads and/or biological particles.

41. The method of embodiment 40, wherein the one or more beads are selected from the group consisting of solid beads, semi-solid beads, gel beads, polymeric beads, and magnetic beads.

42. The method of embodiment 40 or 41, wherein the one or more biological particles are selected from the group consisting of cells, constituents of a cell, and viruses.

43. The method of embodiment 39, wherein the droplet comprises a cell and a bead.

### Embodiment G

1. A system for detecting light simultaneously from a plurality of detection points in a microfluidic device, the system comprising:
(a) a plurality of light guides comprising a receiving end and a transmitting end, wherein the receiving end of each of the plurality of light guides is configured to receive light from one of the plurality of detection points; and
(b) a detector comprising an array sensor having a plurality of regions, wherein each region is configured to receive light transmitted from one of the transmitting ends of the plurality of light guides.

2. The system of embodiment 1, further comprising one or more light sources disposed to illuminate the plurality of detection points in the microfluidic device.

3. The system of embodiment 2, wherein the one or more light sources comprises a laser.

4. The system of any one of embodiments 1-3, wherein the plurality of light guides comprises wave guides.

5. The system of any one of embodiments 1-4, wherein the plurality of light guides comprises a plurality of lenses.

6. The system of embodiment 5, wherein each of the plurality of lenses is a micro-lens.

7. The system of any one of embodiments 1-6, wherein the plurality of light guides comprises multi-mode optical fibers.

8. The system of any one of embodiments 1-7, wherein the array sensor is a charge-coupled device (CCD) or a complementary metal oxide semiconductor (CMOS) sensor.

9. The system of any one of embodiments 1-8, wherein the system is programmed to produce a single digital image or a video from light transmitted by the plurality of light guides to the array sensor.

10. The system of any one of embodiments 1-9, further comprising an emission filter disposed between the array sensor and the plurality of detection points.

11. The system of any one of embodiments 1-10, wherein the detector records 2-2000 frames per second.

12. The system of any one of embodiments 1-11, further comprising a holder for the microfluidic device.

13. The system of any one of embodiments 1-11, further comprising the microfluidic device.

14. The system of embodiment 13, wherein the microfluidic device comprises a plurality of droplet or particle sources and the plurality of detection points are disposed to detect droplets or particles provided by the plurality of droplet or particle sources.

15. A method for detecting light simultaneously at a plurality of detection points in a microfluidic device, the method comprising:
(a) providing the system of any one of embodiments 1-14; and
(b) simultaneously detecting light from the plurality of detection points in the array sensor.

16. A method for simultaneously detecting a plurality of droplets or particles in a microfluidic device, the method comprising:
(a) providing the system of embodiment 14;
(b) passing a liquid comprising the plurality of droplets or particles to the plurality of detection points; and
(c) simultaneously detecting the light from the plurality of detection points using the array sensor, thereby simultaneously detecting the plurality of droplets or particles.

17. The method of embodiment 16, wherein step (c) comprises producing a digital image or a video of light detected from the plurality of detection points.

18. The method of embodiment 17, wherein the light detected is indicative of presence or absence of droplets or particles.

19. A system for detecting light simultaneously from a plurality of locations in a microfluidic device, the system comprising a linear detector aligned with the plurality of locations, wherein the linear detector is configured to detect light from the plurality of locations simultaneously and the locations are disposed to allow detection of non-overlapping signals.

20. The system of embodiment 19, wherein the linear detector comprises a CCD or a CMOS sensor.

21. The system of embodiment 19 or 20, wherein each of the plurality of locations is positioned at a distance of 50 μm to 2.0 cm from an adjacent location.

22. The system of any one of embodiments 19-21, wherein the plurality of locations is at least 2, 4, or 8 locations.

23. The system of any one of embodiments 19-22, wherein the linear detector comprises at least 12,000 pixels.

24. The system of embodiment 23, wherein the linear detector comprises at least 20,000 pixels.

25. The system of any one of embodiments 19-24, wherein the linear detector is a line scan camera.

26. The system of any one of embodiments 19-25, further comprising one or more light sources disposed to illuminate the plurality of locations in the microfluidic device.

27. The system of embodiment 26, wherein the one or more light sources comprises a laser.

28. The system of any one of embodiments 19-27, further comprising a holder for the microfluidic device.

29. The system of any one of embodiments 19-27, further comprising the microfluidic device.

30. The system of embodiment 29, wherein the microfluidic device comprises a plurality of droplet or particle sources and the plurality of locations are disposed to allow detection of droplets or particles produced by the plurality of droplet or particle sources.

31. A method for detecting light simultaneously from a plurality of locations in a microfluidic device, the method comprising:
(a) providing the system of any one of embodiments 19-30; and
(b) simultaneously detecting light from the plurality of locations using the linear detector.

32. A method for simultaneously detecting a plurality of droplets or particles in a microfluidic device, the method comprising:
(a) providing the system of embodiment 30;
(b) passing a liquid comprising the plurality of droplets or particles to the plurality of locations; and
(c) simultaneously detecting the light from the plurality of locations using the linear detector, thereby simultaneously detecting the plurality of droplets or particles.

33. The method of embodiment 32, wherein the light detected is indicative of presence or absence of droplets or particles.

34. The method of embodiment 18 or 33, wherein each of the droplets comprises one or more particles.

35. The method of embodiment 34, wherein each of the one or more particles comprises a biological particle and/or a bead.

36. The method of embodiment 35, wherein the biological particle is a cell.

37. The method of embodiment 36, wherein the cell comprises a fluorescent label.

38. The method of any one of embodiments 18, or 33-37, wherein the light detected is indicative of a number of particles in each of the droplets.

39. The method of any one of embodiments 16-18, or 32-38, further comprising sorting the plurality of droplets.

### Embodiment H

1. A system for detecting light in a microfluidic device, the system comprising:
(a) a stage comprising a location for placement of the microfluidic device;
(b) a light source disposed to transmit a beam of light to a detection point within the location and on a plane parallel to the stage, wherein the angle α of the beam to the plane is from 25° to 40° to the stage;
(c) an aspherical lens pair having an input focal point and an output focal point, wherein the aspherical lens pair is disposed to co-localize the input focal point with the detection point; and
(d) a detector configured to detect the light transmitted from the aspherical lens.

2. The system of embodiment 1, wherein the microfluidic device comprises a surface, wherein when the microfluidic device is present on the stage, the beam intersects the surface at angle α.

3. The system of embodiment 1 or 2, further comprising the microfluidic device.

4. A system for detecting light in a microfluidic device, the system comprising:
(a) a microfluidic device comprising a planar surface;
(b) a light source disposed to transmit a beam of light to a detection point within the microfluidic device, wherein the angle α of the beam to the planar surface is from 25° to 40°;
(c) an aspherical lens pair comprising an input focal point and an output focal point, wherein the aspherical lens pair is disposed to co-localize the input focal point with the detection point; and
(d) a detector configured to detect the light from the aspherical lens.

5. The system of any one of embodiments 1-4, further comprising an optical fiber comprising a receiving end and a transmitting end, wherein the receiving end is disposed to co-localize with the output focal point of the aspherical lens pair, and wherein the detector detects the light transmitted from the transmitting end of the optical fiber.

6. The system of embodiment 4 or 5, further comprising a stage comprising a location for placement of the microfluidic device.

7. The system of embodiment 6, wherein the stage is an X-Y-Z stage.

8. The system of any one of embodiments 1-7, wherein the light source is disposed above the detection point and angle α is above the stage.

9. The system of any one of embodiments 1-8, wherein angle α is from 30° to 35°.

10. The system of any one of embodiments 1-9, further comprising a dichroic mirror between the aspherical lens pair and the output focal point.

11. The system of embodiment 10, further comprising a reflected light detector configured to detect the reflected light from the dichroic mirror.

12. The system of any one of embodiments 5-11, wherein the optical fiber is a multi-mode optical fiber.

13. The system of any one of embodiments 1-12, wherein the light source is a laser.

14. The system of any one of embodiments 1-13, further comprising a polarizer.

15. The system of any one of embodiments 1-14, wherein the detector comprises an emission filter.

16. The system of any one of embodiments 1-15, wherein the optical detector is a camera.

17. The system of any one of embodiments 1-16, wherein the camera records 2-2000 frames per second.

18. A method of detecting light in a microfluidic device, the method comprising:
(a) providing the system of any one of embodiments 1-17;
(b) transmitting light from the light source to the detection point within the microfluidic device at angle α, wherein the light is redirected at the detection point, and wherein the redirected light is transmitted through the aspherical lens pair and the optical fiber; and
(c) detecting the light using the detector.

19. The method of embodiment 18, wherein the microfluidic device is a droplet-forming device.

20. The method of embodiment 19, wherein the light at the detection point is redirected by a droplet formed in the droplet forming device.

21. A method of detecting a droplet in a microfluidic device, the method comprising:
(a) providing the system of any one of embodiments 1-17;
(b) transmitting light from the light source to the detection point within the microfluidic device at angle α, wherein the light is redirected by a droplet at the detection point, and wherein the redirected light is transmitted through the aspherical lens pair and the optical fiber; and
(c) detecting the light using the detector, thereby detecting the droplet.

22. The method of embodiment 20 or 21, wherein the droplet comprises one or more particles.

23. The method of embodiment 22, wherein the one or more particles comprises a biological particle and/or a bead.

24. The method of embodiment 23, wherein the biological particle is a cell.

25. The method of embodiment 24, wherein the cell comprises a fluorescent label.

26. The method of any one of embodiments 22-25, further comprising, based on step (c), determining a number of particles in the droplet.

27. The method of any one of embodiments 21-26, wherein the droplet is aqueous or miscible with water, and wherein the droplet is immiscible with the continuous phase.

28. The method of any one of embodiments 21-27, further comprising sorting the droplet based on one or more characteristics of the droplet.

29. The method of any one of embodiments 18-28, wherein the detection is by image or video recording.

30. The system of any one of embodiments 1-17 or the method of any one of embodiments 18-29, wherein the microfluidic device is multiplexed.

### Embodiment I

1. A device for detecting a droplet or particle, wherein the device comprises:
(a) a droplet or particle source;
(b) a detection channel in fluid communication with the droplet or particle source, the detection channel having a proximal end and a distal end; and
(c) two or more electrodes,
wherein the detection channel and the two or more electrodes are configured to measure an electrical property of the droplet or particle as it passes from the proximal end to the distal end of the detection channel.

2. The device of embodiment 1, wherein the two or more electrodes are on the same side of the detection channel.

3. The device of embodiment 1, wherein two electrodes are on opposing sides of the detection channel.

4. The device of embodiment 3, wherein the two electrodes are directly opposite each other.

5. The device of embodiment 1, wherein the device comprises more than two electrodes.

6. The device of embodiment 1, wherein the detection channel has a width and a depth, and wherein the two or more electrodes have a width from 0.5% and 300% of the width or depth of the detection channel.

7. The device of embodiment 1, wherein the width of each of the two or more electrodes is from 0.5 μm to 300 μm.

8. The device of embodiment 1, wherein the lateral distance between the two or more electrodes is from 0.5 μm to 300 μm.

9. The device of embodiment 1, further comprising a sorting region in fluid communication with the detection channel, comprising a sorter configured to sort the droplet or particle based on the electrical property of the droplet.

10. The device of embodiment 1, wherein the droplet or particle source comprises a droplet formation region.

11. The device of embodiment 10, wherein the droplet formation region comprises a shelf region and a step region.

12. The device of embodiment 1, further comprising one or more additional detection channels, wherein the one or more additional detection channels each comprise two or more electrodes configured to measure an electrical property.

13. A method of detecting a characteristic of a droplet or particle, the method comprising:
(a) providing the device of embodiment 1;
(b) allowing a droplet or particle to pass through the detection channel from the proximal end to the distal end; and
(c) measuring an electrical property as the droplet or particle passes through the detection channel, thereby detecting the characteristic of the droplet or particle.

14. The method of embodiment 13, further comprising forming the droplet.

15. The method of embodiment 13, wherein the droplet comprises a particle.

16. The method of embodiment 15, wherein the particle is a bead or a biological particle.

17. The method of embodiment 13, wherein the droplet comprises a first particle and a second particle.

18. The method of embodiment 17, wherein the first particle is a bead and the second particle is a biological particle.

19. The method of embodiment 16 or 18, wherein the biological particle is a cell.

20. The method of embodiment 13, wherein the electrical property is impedance.

21. The method of embodiment 20, wherein the impedance is measured at one or more frequencies.

22. The method of embodiment 21, wherein the ratio of the difference in impedance of the droplet or particle at the one or more frequencies is indicative of the characteristic of the droplet or particle.

23. The method of embodiment 20, wherein the characteristic is the:
(i) size of the droplet or particle;
(ii) velocity of the droplet or particle;
(iii) presence or location of a particle in the droplet; or
(iv) composition of the droplet.

24. The method of embodiment 13, wherein the characteristic of the droplet is a characteristic of a particle in the droplet.

25. The method of embodiment 13, wherein the characteristic of the particle is:
(i) the type of cell;
(ii) the viability of the cell;
(iii) the size of the cell; and/or
(iv) a permeability of the cell.

26. The method of embodiment 13, further comprising sorting the droplet or particle after it passes through the detection channel based on the characteristic.

### Embodiment J

1. A device for sorting and/or detecting droplets or particles comprising:
(a) a droplet or particle source configured to provide droplets or particles within a first region with at least one cross-sectional dimension is greater than about 1 mm;
(b) a sorter configured to sort one or more of the droplets or particles and/or a detector configured to detect one or more of the droplets or particles; and
(c) a second region, in fluid communication with the first region, wherein each cross-sectional dimension of the collection region is less than about 1 mm.

2. The device of embodiment 1, wherein the second region comprises two or more partitions configured to receive the droplets or particles from the first region,

3. A method of sorting droplets or particles comprising:
(a) providing the device of embodiment 2;
(b) allowing the droplets or particles to enter the first region; and
(c) sorting one or more of the droplets or particles into one or more of the two or more partitions in the collection region.

4. The method of embodiment 3, further comprising detecting the droplets or particles prior to step (c).

5. The method of embodiment 4, wherein the detection provides feedback to the sorter.

6. The method of embodiment 5, wherein the feedback actuates the sorter to sort the one or more of the droplets or particles.

### OTHER EMBODIMENTS

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Other embodiments are in the claims.

What is claimed is:

1. A system for detecting light simultaneously from a plurality of detection points in a microfluidic device, the system comprising:
(a) a plurality of light guides comprising a receiving end and a transmitting end, wherein the receiving end of each of the plurality of light guides is configured to receive light from one of the plurality of detection points; and
(b) a detector comprising an array sensor having a plurality of regions, wherein each region is configured to receive light transmitted from one of the transmitting ends of the plurality of light guides.

2. The system of claim 1, further comprising one or more light sources disposed to illuminate the plurality of detection points in the microfluidic device.

3. The system of claim 2, wherein the one or more light sources comprises a laser.

4. The system of any one of claims 1-3, wherein the plurality of light guides comprises wave guides.

5. The system of any one of claims 1-4, wherein the plurality of light guides comprises a plurality of lenses.

6. The system of claim 5, wherein each of the plurality of lenses is a micro-lens.

7. The system of any one of claims 1-6, wherein the plurality of light guides comprises multi-mode optical fibers.

8. The system of any one of claims 1-7, wherein the array sensor is a charge-coupled device (CCD) or a complementary metal oxide semiconductor (CMOS) sensor.

9. The system of any one of claims 1-8, wherein the system is programmed to produce a single digital image or a video from light transmitted by the plurality of light guides to the array sensor.

10. The system of any one of claims 1-9, further comprising an emission filter disposed between the array sensor and the plurality of detection points.

11. The system of any one of claims 1-10, wherein the detector records 2-2000 frames per second.

12. The system of any one of claims 1-11, further comprising a holder for the microfluidic device.

13. The system of any one of claims 1-11, further comprising the microfluidic device.

14. The system of claim 13, wherein the microfluidic device comprises a plurality of droplet or particle sources and the plurality of detection points are disposed to detect droplets or particles provided by the plurality of droplet or particle sources.

15. A method for detecting light simultaneously at a plurality of detection points in a microfluidic device, the method comprising:
(a) providing the system of any one of claims 1-14; and
(b) simultaneously detecting light from the plurality of detection points in the array sensor.

16. A method for simultaneously detecting a plurality of droplets or particles in a microfluidic device, the method comprising:
(a) providing the system of claim 14;
(b) passing a liquid comprising the plurality of droplets or particles to the plurality of detection points; and
(c) simultaneously detecting the light from the plurality of detection points using the array sensor, thereby simultaneously detecting the plurality of droplets or particles.

17. The method of claim 16, wherein step (c) comprises producing a digital image or a video of light detected from the plurality of detection points.

18. The method of claim 17, wherein the light detected is indicative of presence or absence of droplets or particles.

19. A system for detecting light simultaneously from a plurality of locations in a microfluidic device, the system comprising a linear detector aligned with the plurality of locations, wherein the linear detector is configured to detect light from the plurality of locations simultaneously and the locations are disposed to allow detection of non-overlapping signals.

20. The system of claim 19, wherein the linear detector comprises a CCD or a CMOS sensor.

21. The system of claim 19 or 20, wherein each of the plurality of locations is positioned at a distance of 50 μm to 2.0 cm from an adjacent location.

22. The system of any one of claims 19-21, wherein the plurality of locations is at least 2, 4, or 8 locations.

23. The system of any one of claims 19-22, wherein the linear detector comprises at least 12,000 pixels.

24. The system of claim 23, wherein the linear detector comprises at least 20,000 pixels.

25. The system of any one of claims 19-24, wherein the linear detector is a line scan camera.

26. The system of any one of claims 19-25, further comprising one or more light sources disposed to illuminate the plurality of locations in the microfluidic device.

27. The system of claim 26, wherein the one or more light sources comprises a laser.

28. The system of any one of claims 19-27, further comprising a holder for the microfluidic device.

29. The system of any one of claims 19-27, further comprising the microfluidic device.

30. The system of claim 29, wherein the microfluidic device comprises a plurality of droplet or particle sources and the plurality of locations are disposed to allow detection of droplets or particles produced by the plurality of droplet or particle sources.

31. A method for detecting light simultaneously from a plurality of locations in a microfluidic device, the method comprising:
(a) providing the system of any one of claims 19-30; and
(b) simultaneously detecting light from the plurality of locations using the linear detector.

32. A method for simultaneously detecting a plurality of droplets or particles in a microfluidic device, the method comprising:
(a) providing the system of claim 30;
(b) passing a liquid comprising the plurality of droplets or particles to the plurality of locations; and
(c) simultaneously detecting the light from the plurality of locations using the linear detector, thereby simultaneously detecting the plurality of droplets or particles.

33. The method of claim 32, wherein the light detected is indicative of presence or absence of droplets or particles.

34. The method of claim 18 or 33, wherein each of the droplets comprises one or more particles.

35. The method of claim 34, wherein each of the one or more particles comprises a biological particle and/or a bead.

36. The method of claim 35, wherein the biological particle is a cell.

37. The method of claim 36, wherein the cell comprises a fluorescent label.

38. The method of any one of claim 18, or 33-37, wherein the light detected is indicative of a number of particles in each of the droplets.

39. The method of any one of claim 16-18, or 32-38, further comprising sorting the plurality of droplets.

40. A device for sorting droplets or particles comprising:
(a) a droplet or particle source configured to provide droplets or particles within a continuous phase;
(b) a sorting region, in fluid communication with the droplet or particle source, comprising a sorter configured to sort one or more of the droplets or particles; and
(c) a collection region, in fluid communication with the sorting region, comprising two or more partitions configured for collection of the droplets or particles after sorting.

41. The device of claim 40, wherein the droplet or particle source comprises a droplet or particle formation region configured to form droplets or particles.

42. The device of claim 40 or 41, wherein the sorter is configured to provide mechanical or electromagnetic force to move the one or more droplets or particles into the two or more partitions.

43. The device of any one of claims 40-42, wherein the sorter comprises a dielectrophoretic actuator, an acoustic actuator, a pneumatic actuator, a mechanical actuator, a bubble generator, an optical tweezer, a magnet, a thermal actuator, or an electrostatic charger.

44. The device of claim 43, wherein the dielectrophoretic actuator or the electrostatic charger comprises an electrode.

45. The device of claim 44, wherein the electrode comprises a fluid channel or a solid conductor.

46. The device of any one of claims 40-45, wherein the sorter comprises a divider.

47. The device of any one of claims 40-46, wherein the sorting region comprises a surface configured to laterally deflect one or more of the droplets or particles to one of the two or more partitions.

48. The device of claim 47, wherein the surface comprises one or more openings through which the one or more deflected droplets or particles pass.

49. The device of any one of claims 40-48, wherein the sorting region comprises a detection region to allow detection of the droplets or particles as they pass therethrough.

50. The device of claim 49, wherein the detection region is configured to provide feedback to the sorting region.

51. The device of claim 50, wherein the feedback is configured to actuate the sorter.

52. The device of any one of claims 40-51, wherein the collection region comprises 3, 4, 5, 6, 7, 8 9, 10, or more partitions.

53. The device of any one of claims 40-52, wherein the sorting region has a volume of at least 1 μL.

54. The device of any one of claims 40-53, wherein each cross-sectional dimension of the sorting region has a length of at least 1 mm.

55. The device of any one of claims 40-54, wherein the droplet comprises a particle.

56. The device of claim 55, wherein the particle comprises a biological particle, a bead, or a combination thereof.

57. The device of claim 56, wherein the biological particle comprises a cell or one or more constituents of a cell.

58. The device of claim 56 or 57, wherein the biological particle comprises a matrix.

59. A method of sorting droplets or particles comprising:
(a) providing the device of any one of claims 40-58 comprising the continuous phase in the sorting region;
(b) allowing the droplets or particles to enter the sorting region; and
(c) sorting one or more of the droplets or particles into one or more of the two or more partitions in the collection region.

60. The method of claim 59, wherein the sorter provides mechanical or electromagnetic force to sort the one or more droplets or particles into the two or more partitions.

61. The method of claim 59 or 60, wherein the sorter laterally deflects one or more of the droplets or particles to one of the two or more partitions.

62. The method of any one of claims 59-61, further comprising detecting the droplets or particles prior to step (c).

63. The method of claim 62, wherein the detection provides feedback to the sorter.

64. The method of claim 63, wherein the feedback actuates the sorter to sort the one or more of the droplets or particles.
